(19) United States
(12) Patent Application Publication
(10) Pub. No.: US 2025/0059179 A1
(43) Pub. Date: Feb. 20, 2025

(54) Heterocyclic Compounds and Methods of Use

(71) Applicant: AMGEN INC., Thousand Oaks, CA (US)

(72) Inventors: Brian Alan Lanman, Woodland Hills, CA (US); Ryan Paul Wurz, Newbury Park, CA (US); Wei Zhao, Westford, MA (US); Xiaofen Li, Thousand Oaks, CA (US); Michael M. Yamano, Thousand Oaks, CA (US); Yunxiao Li, Thousand Oaks, CA (US); Ning Chen, Thousand Oaks, CA (US); Sebastian Leth-Petersen, Copenhagen (DK); Kexue Li, Newbury Park, CA (US); Liping Pettus, Thousand Oaks, CA (US); Rene Rahimoff, Santa Monica, CA (US); Primali Vasundera Navaratne, Thousand Oaks, CA (US); Huan Rui, Los Angeles, CA (US)

(73) Assignee: AMGEN INC., Thousand Oaks, CA (US)

(21) Appl. No.: 18/696,948

(22) PCT Filed: Apr. 28, 2022
(86) PCT No.: PCT/US22/26624

§ 371 Date: Mar. 28, 2024

Related U.S. Application Data
(60) Provisional application No. 63/181,627, filed on Apr. 29, 2021.

(51) Int. Cl. C07D 417/14; A61K 31/517; A61K 31/5377; A61K 31/5383; A61K 31/5386; A61K 31/541; A61K 31/55; A61K 31/551; C07D 471/04; C07D 471/08; C07D 487/04; C07D 487/10; C07D 487/20; C07D 498/04; C07D 498/10; C07D 519/00

(52) U.S. Cl. CPC C07D 417/14; A61K 31/517; A61K 31/5377; A61K 31/5383; A61K 31/5386; A61K 31/541; A61K 31/55; A61K 31/551; C07D 471/04; C07D 471/08; C07D 487/04; C07D 487/10; C07D 487/20; C07D 498/04; C07D 498/10; C07D 519/00

(57) ABSTRACT

The present disclosure provides compounds useful for the inhibition of KRAS G12D. The compounds have a general Formula I: (I) wherein the variables of Formula I are defined herein. This disclosure also provides pharmaceutical compositions comprising the compounds, uses of the compounds, and compositions for treatment of, for example, cancer.

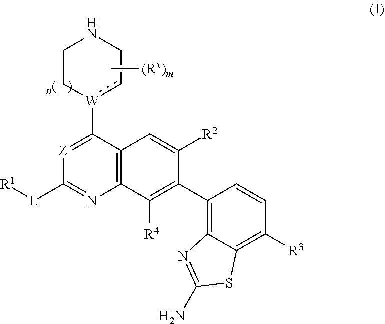

### FIELD

The present disclosure provides compounds having activity as inhibitors of G12D mutant KRAS protein. This disclosure also provides pharmaceutical compositions comprising the compounds, uses and methods of treating certain disorders, such as cancer, including but not limited to Non-Small Cell Lung Cancer (NSCLC), colorectal cancer and/or pancreatic cancer.

### BACKGROUND

From its identification as one of the first human oncogenes in 1982 (Der et al., 1982), KRAS (the Kirsten rat sarcoma viral oncogene homologue) has been the focus of extensive academic and industrial research, as a key node in the MAPK signal transduction pathway, as a transforming factor in a network of parallel effector pathways (e.g., PI3K/AKT) (Vojtek et al., 1998) and as a potential target for anti-cancer agents (Malumbres et al., 2003). Despite progress in the development of inhibitors of upstream and downstream nodes in the MAPK pathway (e.g., EGFR (Sridhar et al., 2003), BRAF (Holderfield et al., 2014) and MOK (Caunt et al., 2015), the KRAS protein has historically proven resistant to direct inhibition.

KRAS is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins have historically been thwarted by the absence of druggable pockets on the surface of the protein (Cox et al., 2014). In 2013, Shokat and colleagues identified covalent inhibitors of a common (O'Bryan, 2019) oncogenic mutant of KRAS, KRAS G12C, which bound to a previously unrecognized allosteric pocket on GDP-KRAS G12C and prevented its subsequent activation (Ostream et al., 2013). This discovery brought about significant new efforts in the KRAS inhibitor research, which have recently culminated in the entry of KRAS inhibitors in human clinical trials.

While some progress has been made on KRAS G12C inhibitors, there is a continued interest and effort to develop inhibitors of KRAS, particularly inhibitors of other KRAS such as KRAS G12D. Thus, there is a need to develop new inhibitors for KRAS G12D for the treatment of disorders, such as cancer.

### SUMMARY

In one aspect, the present application is directed to a compound of formula (I):

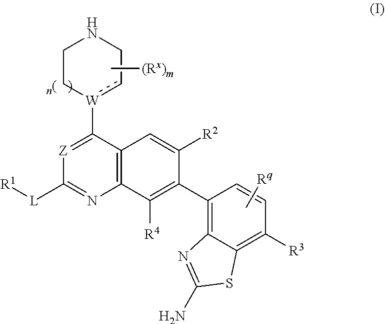

or tautomer thereof, or a pharmaceutically acceptable salt of said compound or said tautomer, wherein;
[symbol] is a single bond or a double bond;
W is C, CH or N, wherein when W is N, [symbol] is a single bond;
n is 0, 1, 2, or 3;
m is 0, 1, 2, 3 or 4;
each R^{x} is hydroxyl, oxo, cyano, C₁₋₄ alkyl, C₁₋₄ haloalkyl, C₁₋₄ alkoxy, —T—R^{y} or two R^{x} taken together with the carbon atoms to which they are attached can form a C₃₋₈ cycloalkyl or a bridged ring, wherein the bridge atoms are selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—;
Z is CH, CR′ or N;
R′ is halogen, cyano or C₁₋₄ alkyl;
L is a bond, —C₁₋₄ alkylene, —O—C₁₋₄ alkylene, —S—C₁₋₄ alkylene, —NR^{Z}—, —NR^{Z}—C₁₋₄ alkylene, —O— or —S—, wherein each —C₁₋₄ alkylene, —O—C₁₋₄ alkylene or —S—C₁₋₄ alkylene could be substituted by 0-2 occurrences of R^{b};
R¹ is —N(R^{a})₂, aryl, heteroaryl, C₃₋₈ cycloalkyl or heterocycloalkyl wherein each aryl, heteroaryl, cycloalkyl or heterocycloalkyl is further substituted with 0-3 occurrences of R⁵;
R² is hydrogen, halogen, C₁₋₄ alkyl, C₂₋₄ alkenyl or cyano;
R³ hydrogen, halogen, cyano, C₁₋₄ alkyl, C₁₋₄ haloalkyl or C₂₋₄ alkynyl;
R⁴ is hydrogen or halogen;
each R⁵ is halogen, C₁₋₄ alkyl, C₁₋₄ alkoxy, C₁₋₄ haloalkyl, —N(R^{w})₂, —(CH₂)ₚ, —OH, —C(O)—R^{z}, heteroaryl or heterocycloalkyl or two R⁵ taken together with the same carbon atom can form a spirocyclic heteroaryl or heterocycloalkyl wherein each heteroaryl or heterocycloalkyl is further substituted with 0-3 occurrences of R⁷;
p is 1, 2 or 3;
each R⁷ is hydroxyl, oxo, halogen, C₁₋₄ alkyl, C₁₋₄ alkoxy, —C(O)R^{z} or —C(O)OR^{z};
T is C₁₋₄ alkylene, —O— or —S—;
each R is hydrogen, C₁₋₄ alkyl or C₁₋₄ alkoxy;
each R^{b} is hydroxyl or C₁₋₄ alkyl;
each R^{v} is halogen or C₁₋₄ alkyl;
each R^{w} is hydrogen, C₁₋₄ alkyl, C₁₋₄ alkoxy or heterocycloalkyl;
R^{q} is hydrogen, halogen or C₁₋₄ alkyl;
R^{y} is halogen, hydroxyl, cyano or amino; and
R^{z} is hydrogen or C₁₋₄ alkyl.

In a second aspect, provided herein is a pharmaceutical composition comprising a compound of Formula I or a pharmaceutically acceptable salt of said compound and a pharmaceutically acceptable excipient.

In a third aspect, provided herein is a compound of Formula I, or a pharmaceutically acceptable salt of said compound, or the pharmaceutical composition as described herein for use in treating cancer.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein as embodiment 1 is a compound of formula (I):

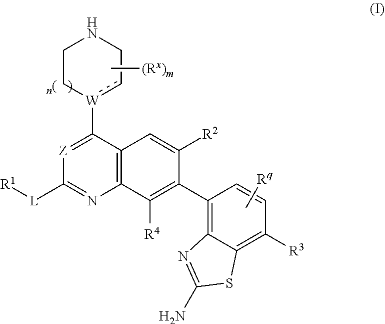

or tautomer thereof, or a pharmaceutically acceptable salt of said compound or said tautomer, wherein;
[symbol] is a single bond or a double bond;
W is C, CH or N, wherein when W is N, [symbol] is a single bond;
n is 0, 1, 2, or 3;
m is 0, 1, 2, 3 or 4;
each R^{x} is hydroxyl, oxo, cyano, C₁₋₄ alkyl, C₁₋₄ haloalkyl, C₁₋₄ alkoxy, —T—R^{y} or two R^{x} taken together with the carbon atoms to which they are attached can form a C₃₋₈ cycloalkyl or a bridged ring, wherein the bridge atoms are selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—;
Z is CH, CR′ or N;
R′ is halogen, cyano or C₁₋₄ alkyl;
L is a bond, —C₁₋₄ alkylene, —O—C₁₋₄ alkylene, —S—C₁₋₄ alkylene, —NR^{Z}—, —NR^{Z}—C₁₋₄ alkylene, —O— or —S—, wherein each —C₁₋₄ alkylene, —O—C₁₋₄ alkylene or —S—C₁₋₄ alkylene could be substituted by 0-2 occurrences of R^{b}; R¹ is —N(R)₂, aryl, heteroaryl, C₃₋₈ cycloalkyl or heterocycloalkyl wherein each aryl, heteroaryl, cycloalkyl or heterocycloalkyl is further substituted with 0-3 occurrences of R⁵;
R² is hydrogen, halogen, C₁₋₄ alkyl, C₂₋₄ alkenyl or cyano;
R³ hydrogen, halogen, cyano, C₁₋₄ alkyl, C₁₋₄ haloalkyl or C₂₋₄ alkynyl;
R⁴ is hydrogen or halogen;
each R⁵ is halogen, C₁₋₄ alkyl, C₁₋₄ alkoxy, C₁₋₄ haloalkyl, —N(R^{w})₂, —(CH₂)₂, —OH, —C(O)—R, heteroaryl or heterocycloalkyl or two R⁵ taken together with the same carbon atom can form a spirocyclic heteroaryl or heterocycloalkyl wherein each heteroaryl or heterocycloalkyl is further substituted with 0-3 occurrences of R⁷;
p is 1, 2 or 3;
each R⁷ is hydroxyl, oxo, halogen, C₁₋₄ alkyl, C₁₋₄ alkoxy, —C(O)R^{z} or —C(O)OR^{z};
T is C₁₋₄ alkylene, —O— or —S—;
each R^{a} is hydrogen, C₁₋₄ alkyl or C₁₋₄ alkoxy;
each R^{b} is hydroxyl or C₁₋₆ alkyl;
each R^{v} is halogen or C₁₋₄ alkyl;
each R^{w} is hydrogen, C₁₋₄ alkyl, C₁₋₄ alkoxy or heterocycloalkyl;
R^{q} is hydrogen, halogen or C₁₋₄ alkyl;
R^{y} is halogen, hydroxyl, cyano or amino; and
R^{z} is hydrogen or C₁₋₄ alkyl.

Provided herein as embodiment 2 is the compound according to embodiment 1, wherein Z is CH. Provided herein as embodiment 3 is the compound according to embodiment 1, wherein Z is N.

Provided herein as embodiment 4 is the compound according to any one of embodiments, 1-3, wherein W is C and [symbol] is a double bond. Provided herein as embodiment 5 is the compound according to any one of embodiments 1-3, wherein W is N.

Provided herein as embodiment 6 is the compound according to any one of embodiments 1-5, wherein n is 0. Provided herein as embodiment 7 is the compound according to embodiment 6, wherein m is 0. Provided herein as embodiment 8 is the compound according to any one of embodiments 1-5, wherein n is 0 and m is 0.

Provided herein as embodiment 9 is the compound according to embodiment 8, wherein

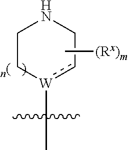

is

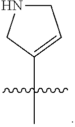 [SMILES: *C1=CCNC1]

Provided herein as embodiment 10 is the compound according to any one of embodiments 1-5, wherein n is 1. Provided herein as embodiment 11 is the compound according to embodiment 10, wherein m is 0. Provided herein as embodiment 12 is the compound according to embodiment 10, wherein m is 1. Provided herein as embodiment 13 is the compound according to any one of embodiments 1-5, wherein n is 1 and m is 0. Provided herein as embodiment 14 is the compound according to any one of embodiments 1-5, wherein n is 1 and m is 1.

Provided herein as embodiment 15 is the compound according to embodiment 14, wherein R^{x} is C₁₋₄ alkyl (e.g., methyl). Provided herein as embodiment 16 is the compound according to embodiment 14, wherein R^{x} is oxo. Provided herein as embodiment 17 is the compound according to embodiment 14, wherein R^{x} is C₁₋₄ alkyl (e.g., methyl). Provided herein as embodiment 18 is the compound according to embodiment 14, wherein R^{x} is C₁₋₄ haloalkyl (e.g., trifluoromethyl). Provided herein as embodiment 19 is the compound according to embodiment 14, wherein R^{x} is —T—R^{y}. Provided herein as embodiment 20 is the compound according to embodiment 19, wherein T is —C₁₋₄ alkylene (e.g., methylene or ethylene). Provided herein as embodiment 21 is the compound according to embodiment 20, wherein R^{y} is hydroxyl. Provided herein as embodiment 22 is the compound according to embodiment 20, wherein R^{y} is cyano. Provided herein as embodiment 23 is the compound according to embodiment 19, wherein —T—R^{y} is —CH₂OH. Provided herein as embodiment 24 is the compound according to embodiment 19, wherein —T—R^{y} is —CH₂CN. Provided herein as embodiment 25 is the compound according to embodiment 19, wherein —T—R^{y} is —CH₂CH₂OH.

Provided herein as embodiment 26 is the compound according to embodiment 14, wherein

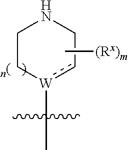

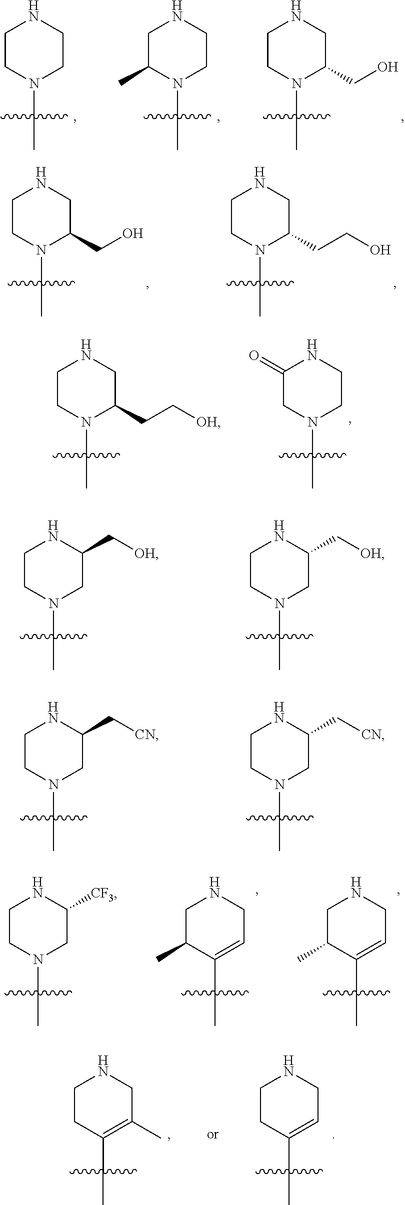

Provided herein as embodiment 27 is the compound according to any one of embodiments 1-5, wherein n is 1 and m is 2.

Provided herein as embodiment 28 is the compound according to embodiment 27, wherein two R^{x} taken together with the carbon atoms to which they are attached form a C₃₋₈ cycloalkyl ring (e.g., cyclopropyl or cyclopentyl). Provided herein as embodiment 29 is the compound according to embodiment 27, wherein two R^{x} taken together with the carbon atoms to which they are attached form a cyclopropyl ring. Provided herein as embodiment 30 is the compound according to embodiment 27, wherein two R^{x} taken together with the carbon atoms to which they are attached form a cyclopentyl ring.

Provided herein as embodiment 31 is the compound according to embodiment 27, wherein two R^{x} taken together can form a bridged ring, wherein the bridged atoms are selected from one of the following: —C₁₋₄ alkylene, —C₁₋₄ alkylene-O— or —C₁₋₄ alkylene-O—C₁₋₄ alkylene-. Provided herein as embodiment 32 is the compound according to embodiment 27, wherein two R^{x} taken together form a bridged ring, wherein the bridged atoms are —C₄ alkylene (e.g., methylene or ethylene). Provided herein as embodiment 33 is the compound according to embodiment 27, wherein two R^{x} taken together form a bridged ring, wherein the bridged atoms are —O—C₁₋₄ alkylene (e.g., —O-methylene- or -methylene-O—). Provided herein as embodiment 34 is the compound according to embodiment 27, wherein two R^{x} taken together form a bridged ring, wherein the bridged atoms are —C₁₋₄ alkylene-O—C₁₋₄ alkylene (e.g., -methylene-O-methylene).

Provided herein as embodiment 35 is the compound according to embodiment 27, wherein

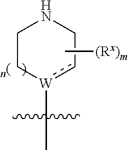

is

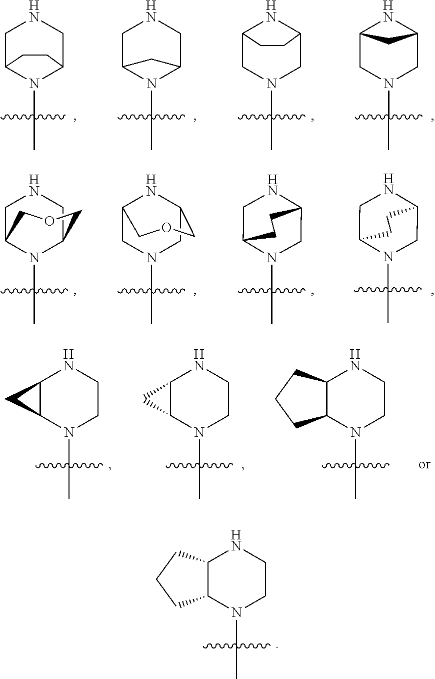

Provided herein as embodiment 36 is the compound according to any one of embodiments 1-5, wherein n is 1 and m is 3.

Provided herein as embodiment 37 is the compound according to embodiment 36, wherein one R^{x} is C₁₋₄ alkyl (e.g., methyl) and the other two R^{x} are taken together to form a bridged ring, wherein the bridged atoms are —C₁₋₄ alkylene (e.g., methylene or ethylene).

Provided herein as embodiment 38 is the compound according to embodiment 36, wherein one R^{x} is C₁₋₄ alkyl (e.g., methyl) and the other two R^{x} are taken together to form a bridged ring, wherein the bridged atoms are —C₁₋₄ alkylene (e.g., ethylene).

Provided herein as embodiment 39 is the compound according to embodiment 36, wherein one R^{x} is C₁₋₄ alkyl (e.g., methyl) and the other two R^{x} are taken together to form a bridged ring, wherein the bridged atoms are —C₁₋₄ alkylene (e.g., methylene).

Provided herein as embodiment 40 is the compound according to embodiment 36, wherein one R^{x} is C₁₋₄ alkyl (e.g., methyl) and the other two R^{x} are taken together to form a bridged ring, wherein the bridged atoms are —C₁₋₄ alkylene-O—C₁₋₄ alkylene- (e.g., methylene-O-methylene).

Provided herein as embodiment 41 is the compound according to embodiment 36, wherein one R^{x} is cyano and the other two R^{x} are taken together to form a bridged ring, wherein the bridged atoms are —C₁₋₄ alkylene (e.g., methylene or ethylene).

Provided herein as embodiment 42 is the compound according to embodiment 36, wherein one R^{x} is cyano and the other two R^{x} are taken together to form a bridged ring, wherein the bridged atoms are methylene.

Provided herein as embodiment 43 is the compound according to embodiment 36, wherein one R^{x} is cyano and the other two R^{x} are taken together to form a bridged ring, wherein the bridged atoms are ethylene.

Provided herein as embodiment 44 is the compound according to embodiment 36, wherein one R^{x} is oxo and the other two R^{x} are taken together to form a bridged ring, wherein the bridged atoms are —C₁₋₄ alkylene (e.g., methylene or ethylene).

Provided herein as embodiment 45 is the compound according to embodiment 36, wherein one R^{x} is oxo and the other two R^{x} are taken together to form a bridged ring, wherein the bridged atoms are ethylene.

Provided herein as embodiment 46 is the compound according to embodiment 36, wherein one R^{x} is cyano and the other two R^{x} are taken together to form a bridged ring, wherein the bridged atoms are —C₁₋₄ alkylene-O—C₁₋₄ alkylene- (e.g., methylene-O-methylene).

Provided herein as embodiment 47 is the compound according to embodiment 36, wherein one R^{x} is —T—R^{y} and the other two R^{x} are taken together to form a bridged ring, wherein the bridged atoms are —C₁₋₄ alkylene (e.g., methylene or ethylene). Provided herein as embodiment 48 is the compound according to embodiment 47, wherein one R^{x} is —T—R^{y} and the other two R^{x} are taken together to form a bridged ring, wherein the bridged atoms are methylene. Provided herein as embodiment 49 is the compound according to embodiment 47, wherein one R^{x} is —T—R^{y} and the other two R^{x} are taken together to form a bridged ring, wherein the bridged atoms are ethylene.

Provided herein as embodiment 50 is the compound according to any one of embodiments 47-49, wherein T is —C₁₋₄ alkylene (e.g., methylene). Provided herein as embodiment 51 is the compound according to embodiment 50, wherein R^{y} is hydroxyl. Provided herein as embodiment 52 is the compound according to embodiment 50, wherein R^{y} is cyano. Provided herein as embodiment 53 is the compound according to embodiment 50, wherein —T—R^{y} is —CH₂OH. Provided herein as embodiment 54 is the compound according to embodiment 50, wherein —T—R^{y} is —CH₂CN.

Provided herein as embodiment 55 is the compound according to embodiment 36, wherein —T—R^{y} is —CH₂OH and the other two R^{x} are taken together to form a bridged ring, wherein the bridge is —C₁₋₄ alkylene (e.g., methylene).

Provided herein as embodiment 56 is the compound according to embodiment 36, wherein —T—R^{y} is —CH₂CN and the other two R^{x} are taken together to form a bridged ring, wherein the bridge is —C₁₋₄ alkylene (e.g., methylene).

Provided herein as embodiment 57 is the compound according to embodiment 36, wherein —T—R^{y} is —CH₂OH and the other two R^{x} are taken together to form a bridged ring, wherein the bridged atoms are —C₁₋₄ alkylene (e.g., ethylene).

Provided herein as embodiment 58 is the compound according to embodiment 36, wherein —T—R^{y} is —CH₂CN and the other two R^{x} are taken together to form a bridged ring, wherein the bridged atoms are —C₁₋₄ alkylene (e.g., ethylene).

Provided herein as embodiment 59 is the compound according to embodiment 36, wherein one R is —T—R^{y} and the other two R^{x} are taken together to form a bridged ring, wherein the bridged atoms are —C₁₋₄ alkylene-O—C₁₋₄ alkylene (e.g., methylene-O-methylene). Provided herein as embodiment 60 is the compound according to embodiment 59, wherein one R^{x} is —T—R^{y} and the other two R^{x} are taken together to form a bridged ring, wherein the bridged atoms are -methylene-O-methylene-. Provided herein as embodiment 61 is the compound according to any one of embodiments 59-60, wherein T is —C₁₋₄ alkylene (e.g., methylene). Provided herein as embodiment 62 is the compound according to embodiment 61, wherein R^{y} is hydroxyl. Provided herein as embodiment 63 is the compound according to embodiment 61, wherein R^{y} is cyano. Provided herein as embodiment 64 is the compound according to embodiment 61, wherein —T—R^{y} is —CH₂OH. Provided herein as embodiment 65 is the compound according to embodiment 61, wherein —T—R^{y} is —CH₂CN.

Provided herein as embodiment 66 is the compound according to embodiment 36, wherein —T—R^{y} is —CH₂OH and the other two R^{x} are taken together to form a bridged ring, wherein the bridge is —C₁₋₄ alkylene-O—C₁₋₄ methylene (e.g., -methylene-O-methylene-).

Provided herein as embodiment 67 is the compound according to embodiment 36, wherein —T—R^{y} is —CH₂CN and the other two R^{x} are taken together to form a bridged ring, wherein the bridge is —C₁₋₄ alkylene-O—C₁₋₄ alkylene (e.g., -methylene-O-methylene-).

Provided herein as embodiment 68 is the compound according to embodiment 36, wherein

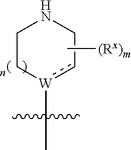

is

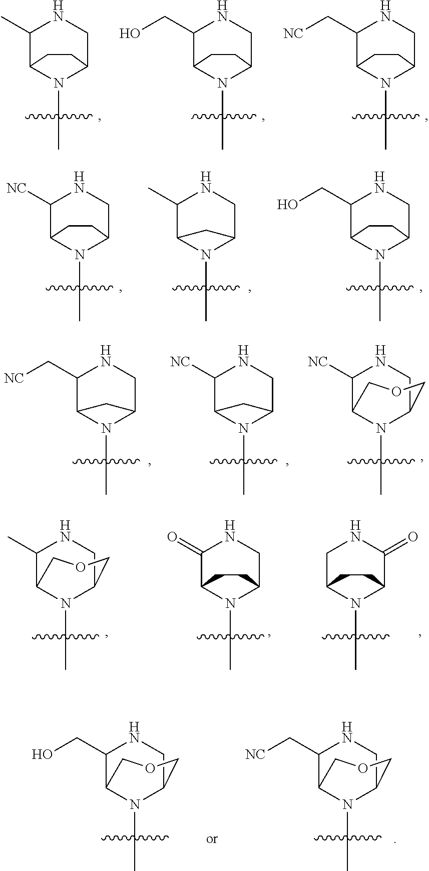

Provided herein as embodiment 69 is the compound according to any one of embodiments 1-5, wherein n is 1 and m is 4. Provided herein as embodiment 70 is the compound according to embodiment 69, wherein two R^{x} are each independently C₁₋₄ alkyl (e.g., methyl) and the other two R^{x} are taken together to form a bridged ring, wherein the bridged atoms are C₁₋₄ alkylene (e.g., methylene or ethylene). Provided herein as embodiment 71 is the compound according to embodiment 69, wherein two R^{x} are each independently C₁₋₄ alkyl (e.g., methyl) and the other two R^{x} are taken together to form a bridged ring, wherein the bridged atoms are methylene. Provided herein as embodiment 72 is the compound according to embodiment 69, wherein two R^{x} are each independently C₁₋₄ alkyl (e.g., methyl) and the other two R^{x} are taken together to form a bridged ring, wherein the bridged atoms are ethylene. Provided herein as embodiment 73 is the compound according to embodiment 69, wherein two R^{x} are each independently C₁₋₄ alkyl (e.g., methyl) and the other two R^{x} are taken together to form a bridged ring, wherein the bridged atoms are —C₁₋₄ alkylene-O—C₁₋₄ alkylene (e.g., -methylene-O-methylene).

Provided herein as embodiment 74 is the compound according to embodiment 69, wherein

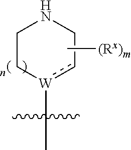

is

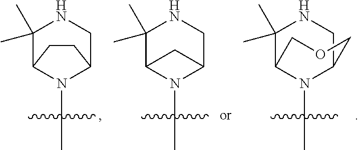

Provided herein as embodiment 75 is the compound according to any one of embodiments 1-5, wherein n is 2. Provided herein as embodiment 76 is the compound according to embodiment 75, wherein m is 0. Provided herein as embodiment 77 is the compound according to embodiment 75, wherein m is 1. Provided herein as embodiment 78 is the compound according to any one of embodiments 1-5, wherein n is 2 and m is 0. Provided herein as embodiment 79 is the compound according to any one of embodiments 1-5, wherein n is 2 and m is 1. Provided herein as embodiment 80 is the compound according to embodiment 79, wherein R^{x} is oxo.

Provided herein as embodiment 81 is the compound according to any one of embodiments 78-79, wherein

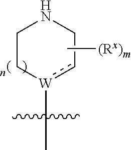

is

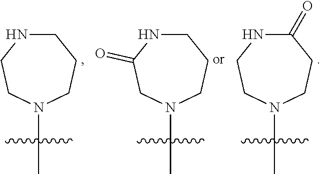

Provided herein as embodiment 82 is the compound according to any one of embodiments 1-5, where n is 2 and m is 2. Provided herein as embodiment 83 is the compound according to embodiment 82, wherein two R^{x} taken together can form a bridged ring, wherein the bridged atoms are selected from one of the following: —C₁₋₄ alkylene, —C₁₋₄ alkylene-O— or —C₁₋₄ alkylene-O—C₁₋₄ alkylene-. Provided herein as embodiment 84 is the compound according to embodiment 82, wherein two R^{x} taken together can form a bridged ring, wherein the bridged atoms are —C₁₋₄ alkylene (e.g., methylene or ethylene). Provided herein as embodiment 85 is the compound according to embodiment 82, wherein two R^{x} taken together can form a bridged ring, wherein the bridged atoms are methylene. Provided herein as embodiment 86 is the compound according to embodiment 82, wherein two R^{x} taken together can form a bridged ring, wherein the bridged atoms are ethylene.

Provided herein as embodiment 87 is the compound according to embodiment 82, wherein

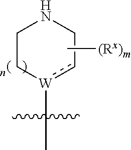

is

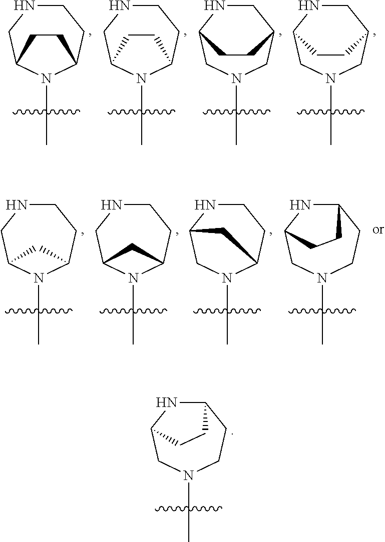

Provided herein as embodiment 88 is the compound according to any one of embodiments 1-5, wherein n is 2 and m is 3. Provided herein as embodiment 89 is the compound according to embodiment 88, wherein one R^{x} is halogen (e.g., fluorine) and other two R^{x} taken together can form a bridged ring, wherein the bridged atoms are selected from one of the following: —C₁₋₄ alkylene, —C₁₋₄ alkylene-O— or —C₁₋₄ alkylene-O—C₁₋₄ alkylene-. Provided herein as embodiment 90 is the compound according to embodiment 88, wherein one R^{x} is halogen (e.g., fluorine) and the other two R^{x} taken together can form a bridged ring, wherein the bridged atoms are —C₁₋₄ alkylene (e.g., methylene or ethylene). Provided herein as embodiment 91 is the compound according to embodiment 88, wherein one R^{x} is halogen (e.g., fluorine) and the other two R^{x} taken together can form a bridged ring, wherein the bridged atoms are ethylene.

Provided herein as embodiment 92 is the compound according to embodiment 88, wherein

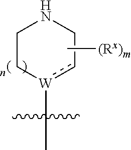

is

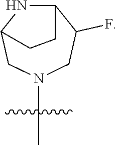 [SMILES: *N1CC2CCC(N2)C(F)C1]

Provided herein as embodiment 93 is the compound according to any one of embodiments 1-5, wherein n is 2 and m is 4. Provided herein as embodiment 94 is the compound according to embodiment 93, wherein two R are halogen (e.g., fluorine) and other two R^{x} taken together can form a bridged ring, wherein the bridged atoms are selected from one of the following: —C₁₋₄ alkylene, —C₁₋₄ alkylene-O— or —C₁₋₄ alkylene-O—C₁₋₄ alkylene-. Provided herein as embodiment 95 is the compound according to embodiment 93, wherein two R^{x} are halogen (e.g., fluorine) and the other two R^{x} taken together can form a bridged ring, wherein the bridged atoms are —C₁₋₄ alkylene (e.g., methylene or ethylene). Provided herein as embodiment 96 is the compound according to embodiment 93, wherein two R^{x} are halogen (e.g., fluorine) and the other two R^{x} taken together can form a bridged ring, wherein the bridged atoms are ethylene.

Provided herein as embodiment 97 is the compound according to embodiment 93, wherein

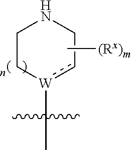

is

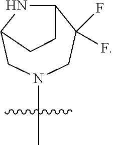 [SMILES: *N1CC2CCC(N2)C(F)(F)C1]

Provided herein as embodiment 98 is the compound according to any one of embodiments 1-97, wherein

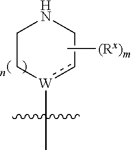

is

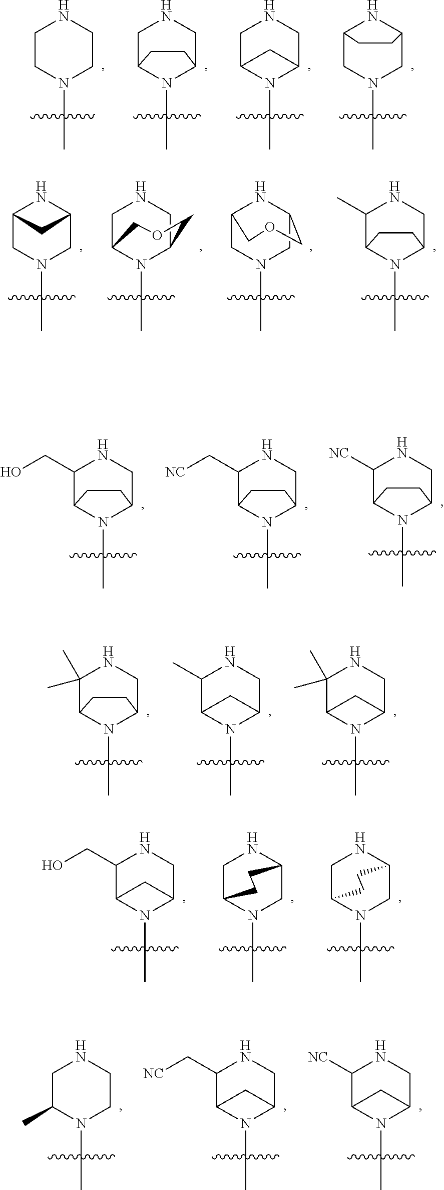

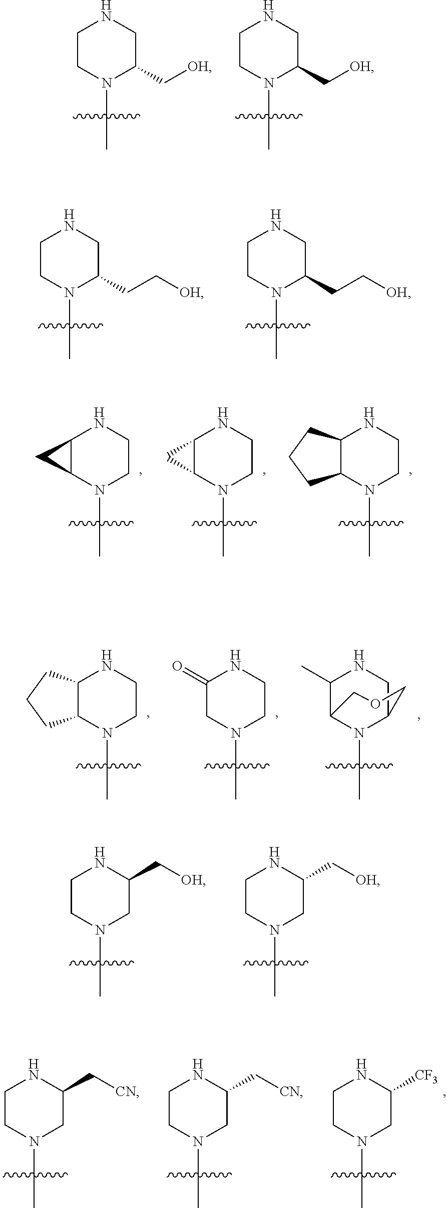

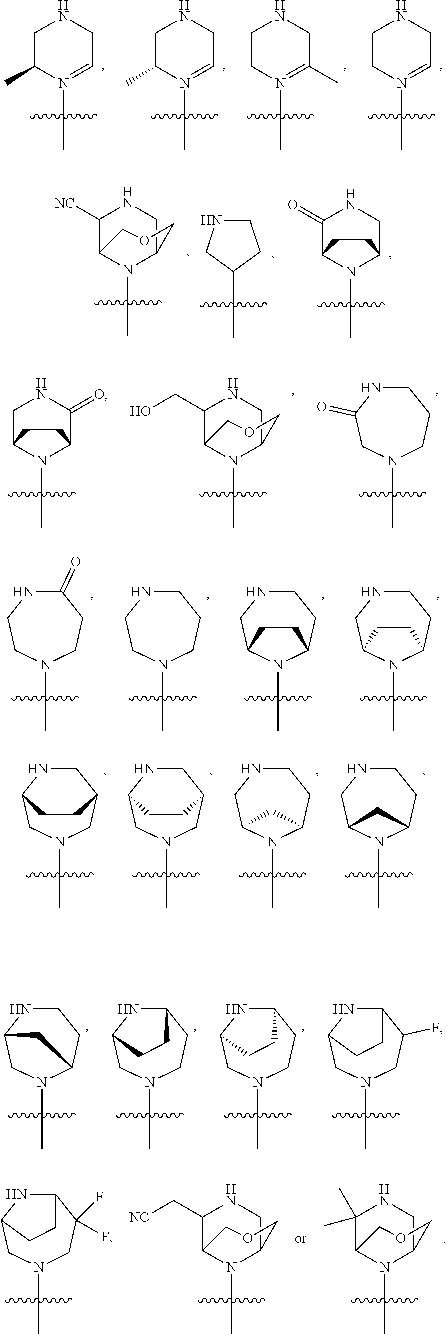

Provided herein as embodiment 99 is the compound according to embodiment 98,

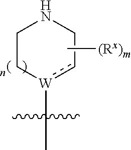

is

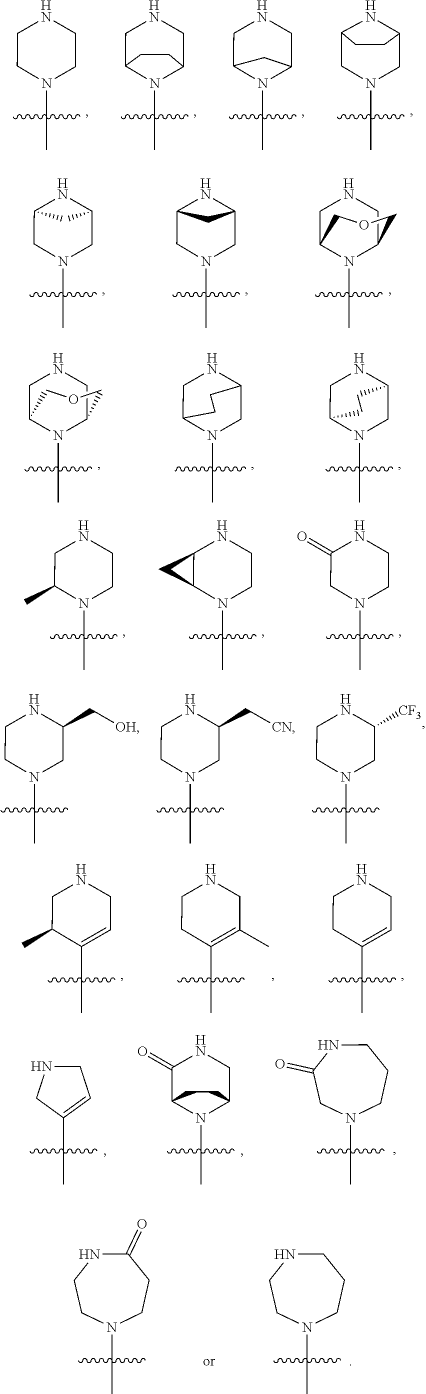

Provided herein as embodiment 100 is the compound according to embodiment 99 wherein

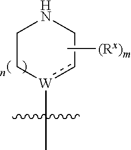

is

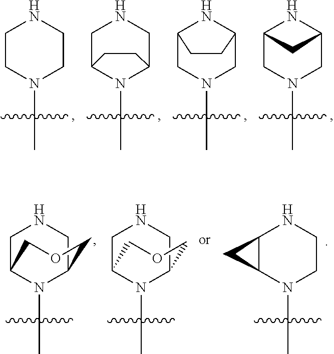

Provided herein as embodiment 101 is the compound according to any one of embodiments 1-100, wherein L is a bond, —C₁₋₄ alkylene, —NR^{z}—C₁₋₄ alkylene, —NR^{Z}—, —O— or —O—C₁₋₄ alkylene substituted with 0-2 occurrences of R^{b}.

Provided herein as embodiment 102 is the compound according to embodiment 101, wherein L is —C₁₋₄ alkylene (e.g., methylene or ethylene) substituted by 0-2 occurrences of R^{b}. Provided herein as embodiment 103 is the compound according to embodiment 101, wherein L is —C₁₋₄ alkylene (e.g., methylene or ethylene) substituted by 0 occurrences of R^{b}. Provided herein as embodiment 104 is the compound according to embodiment 103, wherein L is methylene substituted by 0 occurrences of R^{b}. Provided herein as embodiment 105 is the compound according to embodiment 103, wherein L is ethylene substituted by 0 occurrences of R^{b}.

Provided herein as embodiment 106 is the compound according to embodiment 101, wherein L is —O—C₁₋₄ alkylene (e.g., —O-methylene- or —O-ethylene-) substituted with 0-2 occurrences of R^{b}. Provided herein as embodiment 107 is the compound according to embodiment 106, wherein L is —O—C₁₋₄ alkylene (e.g., —O-methylene- or —O-ethylene-) substituted with 0 occurrences of R^{b}. Provided herein as embodiment 108 is the compound according to embodiment 107, wherein L is —O-methylene substituted with 0 occurrences of R^{b}. Provided herein as embodiment 109 is the compound according to embodiment 107, wherein L is —O-ethylene substituted with 0 occurrences of R^{b}. Provided herein as embodiment 110 is the compound according to embodiment 106, wherein L is —O—C₁₋₄ alkylene (e.g., —O-methylene- or —O-ethylene-) substituted with one occurrence of R^{b}. Provided herein as embodiment 111 is the compound according to embodiment 110, wherein L is —O-methylene substituted with one occurrence of R^{b}. Provided herein as embodiment 112 is the compound according to embodiment 110, wherein L is —O-ethylene substituted with one occurrence of R^{b}. Provided herein as embodiment 113 is the compound according to any one of embodiments 111 or 112, wherein R^{b} is C₁₋₄ alkyl (e.g., methyl). Provided herein as embodiment 114 is the compound according to embodiment 113, wherein L is

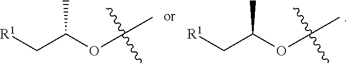

Provided herein as embodiment 115 is the compound according to embodiment 101, wherein L is —O—.

Provided herein as embodiment 116 is the compound according to embodiment 101, wherein L is —NR^{z}—C₁₋₄ alkylene-substituted with 0-2 occurrences of R^{b}. Provided herein as embodiment 117 is the compound according to embodiment 116, wherein L is —NR^{z}—C₁₋₄ alkylene-substituted with one occurrence of R^{b}. Provided herein as embodiment 118 is the compound according to embodiment 117, wherein R^{z} is hydrogen. Provided herein as embodiment 119 is the compound according to embodiment 117, wherein R^{b} is hydroxyl. Provided herein as embodiment 120 is the compound according to embodiment 117, wherein L is

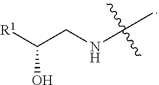

Provided herein as embodiment 121 is the compound according to embodiment 101, wherein L is a bond.

Provided herein as embodiment 122 is the compound according to embodiment 101, wherein L is —NR^{Z}—. Provided herein as embodiment 123 is the compound according to embodiment 122, wherein L is R^{z} is hydrogen. Provided herein as embodiment 124 is the compound according to embodiment 122, wherein L is —NH—.

Provided herein as embodiment 125 is the compound according to any one of embodiments 1-124, wherein R′ is heterocycloalkyl optionally substituted with 0-3 occurrences of R⁵.

Provided herein as embodiment 126 is the compound according to embodiment 125, wherein R¹ is 7-(hexahydro-1H-pyrrolizine) substituted with 0-3 occurrences of R⁵. Provided herein as embodiment 127 is the compound according to embodiment 126, wherein R¹ is 7-(hexahydro-1H-pyrrolizine) substituted with 0 occurrences of R⁵. Provided herein as embodiment 128 is the compound according to embodiment 127, wherein L—R¹ is

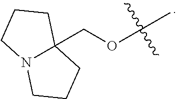 [SMILES: *OCC12CCCN1CCC2 ; "."]

Provided herein as embodiment 129 is the compound according to embodiment 126, wherein R¹ is 7-(hexahydro-1H-pyrrolizine) substituted with one occurrence of R⁵. Provided herein as embodiment 130 is the compound according to embodiment 129 wherein R⁵ is halogen (e.g., fluorine). Provided herein as embodiment 131 is the compound according to embodiment 129, wherein R⁵ is oxo. Provided herein as embodiment 132 is the compound according to embodiment 129, wherein R⁵ is —(CH₂)ₚ—OH. Provided herein as embodiment 133 is the compound according to embodiment 132, wherein p is 1. Provided herein as embodiment 134 is the compound according to embodiment 129, wherein R⁵ is —(CH₂)ₚ—OH and p is 1. Provided herein as embodiment 135 is the compound according to embodiment 129, wherein L—R¹ is

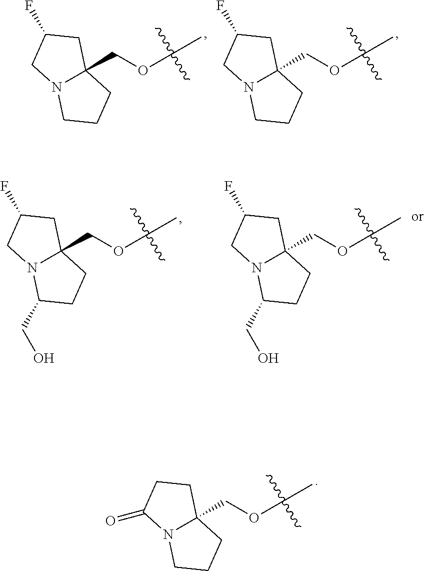

Provided herein as embodiment 136 is the compound according to embodiment 126, wherein R¹ is 7-(hexahydro-1H-pyrrolizine) substituted with 2 occurrences of R⁵. Provided herein as embodiment 137 is the compound according to embodiment 136, wherein both R⁵ are halogen (e.g., fluorine). Provided herein as embodiment 138 is the compound according to embodiment 136, wherein L—R¹ is

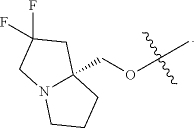 [SMILES: *OC[C@]12CCCN1CC(F)(F)C2 ; "."]

Provided herein as embodiment 139 is the compound according to embodiment 126, wherein R¹ is 8a-(octahydroindolizine) substituted with 0-3 occurrences of R⁵. Provided herein as embodiment 140 is the compound according to embodiment 139, wherein R¹ is 8a-(octahydroindolizine) substituted with 0 occurrences of R⁵. Provided herein as embodiment 141 is the compound according to embodiment 140, wherein L—R¹ is

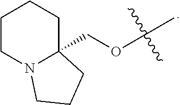 [SMILES: *OC[C@@]12CCCCN1CCC2 ; "."]

Provided herein as embodiment 142 is the compound according to embodiment 125, wherein R¹ is 2-pyrrolidine substituted with 0-3 occurrences of R⁵. Provided herein as embodiment 143 is the compound according to embodiment 142, wherein R¹ is 2-pyrrolidine substituted with 0 occurrences of R⁵.

Provided herein as embodiment 144 is the compound according to embodiment 142, wherein R¹ is 2-pyrrolidine substituted with one occurrence of R⁵. Provided herein as embodiment 145 is the compound according to embodiment 144, wherein R⁵ is halogen (e.g., fluorine). Provided herein as embodiment 146 is the compound according to embodiment 144, wherein R⁵ is C₁₋₄ alkyl (e.g., methyl). Provided herein as embodiment 147 is the compound according to embodiment 144, wherein R⁵ is oxo. Provided herein as embodiment 148 is the compound according to embodiment 144, wherein R⁵ is —C(O)R^{z}. Provided herein as embodiment 149 is the compound according to embodiment 148, wherein R^{z} is C₁₋₄ alkyl (e.g., methyl). Provided herein as embodiment 150 is the compound according to embodiment 148, wherein R⁵ is —C(O)-methyl. Provided herein as embodiment 151 is the compound according to embodiment 144, wherein R⁵ is C₁₋₄ haloalkyl (e.g., 2-fluoroethyl or 2,2-difluoroethyl). Provided herein as embodiment 152 is the compound according to embodiment 144, wherein L—R¹ is

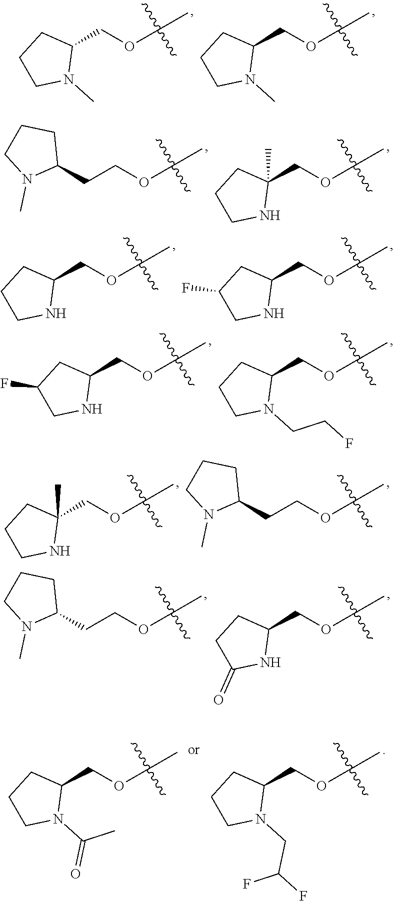

Provided herein as embodiment 153 is the compound according to embodiment 142, wherein R¹ is 2-pyrrolidine substituted with 2 occurrences of R⁵. Provided herein as embodiment 154 is the compound according to embodiment 153, wherein both R⁵ are halogen (e.g., fluorine). Provided herein as embodiment 155 is the compound according to embodiment 153, wherein one R⁵ is C₁₋₄ alkyl (e.g., methyl) and the other R⁵ is halogen (e.g., fluorine). Provided herein as embodiment 156 is the compound according to embodiment 153, wherein L—R¹ is

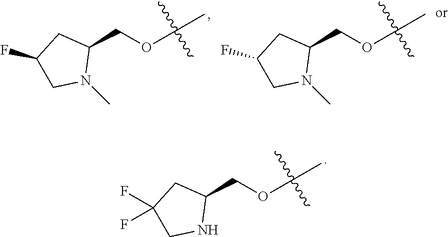

Provided herein as embodiment 157 is the compound according to embodiment 142, wherein R¹ is 2-pyrrolidine substituted with 3 occurrences of R⁵. Provided herein as embodiment 158 is the compound according to embodiment 157, wherein two R⁵ are halogen (e.g., fluorine) and the third R⁵ is C₁₋₄ alkyl (e.g., methyl). Provided herein as embodiment 159 is the compound according to embodiment 157, wherein L—R¹ is

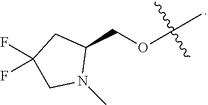 [SMILES: "." ; *OC[C@@H]1CC(F)(F)CN1C]

Provided herein as embodiment 160 is the compound according to embodiment 125, wherein R¹ is 3-pyrrolidine substituted with 0-3 occurrences of R⁵. Provided herein as embodiment 161 is the compound according to embodiment 160, wherein R¹ is 3-pyrrolidine substituted with one occurrence of R⁵. Provided herein as embodiment 162 is the compound according to embodiment 160, wherein R⁵ is C₁₋₄ alkyl (e.g., methyl). Provided herein as embodiment 163 is the compound according to embodiment 160, wherein L—R¹ is

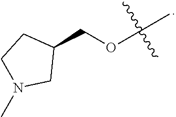 [SMILES: "." ; *OC[C@@H]1CCN(C)C1]

Provided herein as embodiment 164 is the compound according to embodiment 125, wherein R¹ is 2-azetidinyl substituted with 0-3 occurrences of R⁵. Provided herein as embodiment 165 is the compound according to embodiment 164, wherein R¹ is 2-azetidinyl substituted with one occurrence of R⁵. Provided herein as embodiment 166 is the compound according to embodiment 165, wherein R⁵ is C₁₋₄ alkyl (e.g., methyl). Provided herein as embodiment 167 is the compound according to embodiment 164, wherein L—R¹ is

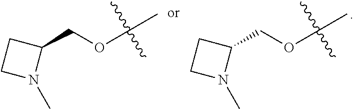

Provided herein as embodiment 168 is the compound according to embodiment 125, wherein R¹ is 2-piperidinyl substituted with 0-3 occurrences of R⁵. Provided herein as embodiment 169 is the compound according to embodiment 168, wherein R¹ is 2-piperidinyl substituted with one occurrence of R⁵. Provided herein as embodiment 170 is the compound according to embodiment 169, wherein R⁵ is C₁₋₄ alkyl (e.g., methyl). Provided herein as embodiment 171 is the compound according to embodiment 168, wherein L—R¹ is

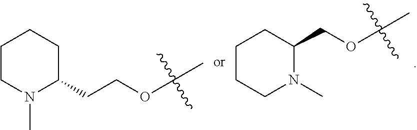

Provided herein as embodiment 172 is the compound according to embodiment 125, wherein R¹ is 4-piperidinyl substituted with 0-3 occurrences of R⁵. Provided herein as embodiment 173 is the compound according to embodiment 172, wherein R¹ is 4-piperidinyl substituted with one occurrence of R⁵. Provided herein as embodiment 174 is the compound according to embodiment 173, wherein R⁵ is C₁₋₄ alkyl (e.g., ethyl). Provided herein as embodiment 175 is the compound according to embodiment 172, wherein L—R¹ is

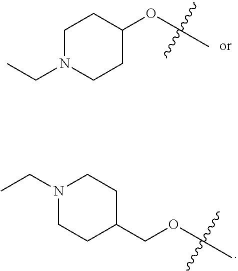

Provided herein as embodiment 176 is the compound according to embodiment 125, wherein R¹ is 1-(7-azabicyclo[2.2.1]heptanyl) substituted with 0-3 occurrences of R⁵. Provided herein as embodiment 177 is the compound according to embodiment 176, wherein R¹ is 1-(7-azabicyclo[2.2.1]heptanyl) substituted with 0 occurrences of R⁵. Provided herein as embodiment 178 is the compound according to embodiment 176, wherein L—R¹ is

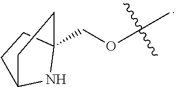

Provided herein as embodiment 179 is the compound according to embodiment 125, wherein R¹ is 6-(2,6-diazabicyclo[3.2.0]heptanyl) substituted with 0-3 occurrences of R⁵. Provided herein as embodiment 180 is the compound according to embodiment 179, wherein R¹ is 6-(2,6-diazabicyclo[3.2.0]heptanyl) substituted with 0 occurrences of R⁵. Provided herein as embodiment 181 is the compound according to embodiment 179, wherein R¹ is 6-((1S,5R)-2,6-diazabicyclo[3.2.0]heptanyl) substituted with 0-3 occurrences of R⁵. Provided herein as embodiment 182 is the compound according to embodiment 181, wherein R¹ is 6-((1S,5R)-2,6-diazabicyclo[3.2.0]heptanyl) substituted with 0 occurrences of R⁵. Provided herein as embodiment 183 is the compound according to embodiment 181, wherein L—R¹ is

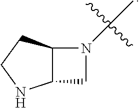 [SMILES: "." ; *N1C[C@@H]2NCC[C@H]21]

Provided herein as embodiment 184 is the compound according to embodiment 125, wherein R¹ is 3-(3,6-diazabicyclo[3.2.0]heptanyl) substituted with 0-3 occurrences of R⁵. Provided herein as embodiment 185 is the compound according to embodiment 184, wherein R¹ is 3-(3,6-diazabicyclo[3.2.0]heptanyl) substituted with 0 occurrences of R⁵. Provided herein as embodiment 186 is the compound according to embodiment 184, wherein L—R¹ is

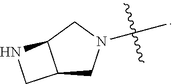 [SMILES: *N1C[C@H]2CN[C@H]2C1 ; "."]

Provided herein as embodiment 187 is the compound according to embodiment 125, wherein R¹ is 5-(octahydropyrrolo[3,4-b]pyrrolyl) substituted with 0-3 occurrences of R⁵. Provided herein as embodiment 188 is the compound according to embodiment 187, wherein R¹ is 5-(octahydropyrrolo[3,4-b]pyrrolyl) substituted with 0 occurrences of R⁵. Provided herein as embodiment 189 is the compound according to embodiment 187, wherein R¹ is 5-((3aS,6aS)-(octahydropyrrolo[3,4-b]pyrrolyl)) substituted with 0-3 occurrences of R⁵. Provided herein as embodiment 190 is the compound according to embodiment 189, wherein R¹ is 5-((3aS,6aS)-(octahydropyrrolo[3,4-b]pyrrolyl)) substituted with 0 occurrences of R⁵. Provided herein as embodiment 191 is the compound according to embodiment 189, wherein R¹ is 5-((3aS,6aS)-(octahydropyrrolo[3,4-b]pyrrolyl)) substituted with one occurrence of R⁵. Provided herein as embodiment 192 is the compound according to embodiment 191, wherein R⁵ is C₁₋₄ alkyl (e.g., methyl). Provided herein as embodiment 193 is the compound according to embodiment 187, wherein L—R¹ is

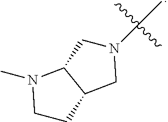 [SMILES: "." ; *N1C[C@@H]2CCN(C)[C@@H]2C1]

Provided herein as embodiment 194 is the compound according to embodiment 125, wherein R¹ is 4-(1,4-diazabicyclo[3.2.1]octanyl) substituted with 0-3 occurrences of R⁵. Provided herein as embodiment 195 is the compound according to embodiment 194, wherein R¹ is 4-(1,4-diazabicyclo[3.2.1]octanyl) substituted with 0 occurrences of R⁵. Provided herein as embodiment 196 is the compound according to embodiment 194, wherein R¹ is 4-((5S)-(1,4-diazabicyclo[3.2.1]octanyl)) substituted with 0-3 occurrences of R⁵. Provided herein as embodiment 197 is the compound according to embodiment 196, wherein R¹ is 4-((5S)-(1,4-diazabicyclo[3.2.1]octanyl)) substituted with 0 occurrences of R⁵. Provided herein as embodiment 198 is the compound according to embodiment 194, wherein L—R¹ is

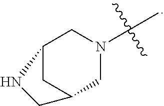 [SMILES: "." ; *N1C[C@@H]2CN[C@@H](C2)C1]

Provided herein as embodiment 199 is the compound according to embodiment 125, wherein R¹ is 3-(3,6-diazabicyclo[3.2.1]octanyl) substituted with 0-3 occurrences of R⁵. Provided herein as embodiment 200 is the compound according to embodiment 199, wherein R¹ is 3-(3,6-diazabicyclo[3.2.1]octanyl) substituted with 0 occurrences of R⁵. Provided herein as embodiment 201 is the compound according to embodiment 199, wherein R¹ is 3-((1S,5S)-(3,6-diazabicyclo[3.2.1]octanyl)) substituted with 0-3 occurrences of R⁵. Provided herein as embodiment 202 is the compound according to embodiment 201, wherein R¹ is 3-((1S,5S)-(3,6-diazabicyclo[3.2.1]octanyl)) substituted with 0 occurrences of R⁵. Provided herein as embodiment 203 is the compound according to embodiment 199, wherein L—R¹ is

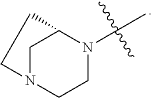 [SMILES: *N1CCN2CC[C@H]1C2 ; "."]

Provided herein as embodiment 204 is the compound according to embodiment 125, wherein R¹ is 1-(octahydro-1H-pyrrolo[3,2-b]piperidinyl) substituted with 0-3 occurrences of R⁵. Provided herein as embodiment 205 is the compound according to embodiment 204, wherein R¹ is 1-(octahydro-1H-pyrrolo[3,2-b]piperidinyl) substituted with one occurrence of R⁵. Provided herein as embodiment 206 is the compound according to embodiment 205, wherein R⁵ is C₁₋₄ alkyl (e.g., methyl). Provided herein as embodiment 207 is the compound according to embodiment 204, wherein R¹ is 1-((3aR,7aR)-(octahydro-1H-pyrrolo[3,2-b]piperidinyl)) substituted with 0-3 occurrences of R⁵. Provided herein as embodiment 208 is the compound according to embodiment 207, wherein R¹ is 1-((3aR,7aR)-(octahydro-1H-pyrrolo[3,2-b]piperidinyl)) substituted with one occurrence of R⁵. Provided herein as embodiment 209 is the compound according to embodiment 208, wherein R⁵ is C₁₋₄ alkyl (e.g., methyl). Provided herein as embodiment 210 is the compound according to embodiment 204, wherein L—R¹ is

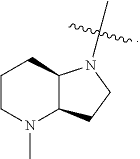 [SMILES: *N1CC[C@@H]2[C@H]1CCCN2C ; "."]

Provided herein as embodiment 211 is the compound according to embodiment 125, wherein R¹ is 6-(octahydropyrrolo[3,4-b][1,4]oxazinyl) substituted with 0-3 occurrences of R⁵. Provided herein as embodiment 212 is the compound according to embodiment 211, wherein R¹ is 6-(octahydropyrrolo[3,4-b][1,4]oxazinyl) substituted with one occurrence of R⁵. Provided herein as embodiment 213 is the compound according to embodiment 212, wherein R⁵ is C₁₋₄ alkyl (e.g., methyl).

Provided herein as embodiment 214 is the compound according to embodiment 211, wherein R¹ is 6-(4aS,7aS)-(octahydropyrrolo[3,4-b][1,4]oxazinyl) substituted with one occurrence of R⁵. Provided herein as embodiment 215 is the compound according to embodiment 214, wherein R⁵ is C₁₋₄ alkyl (e.g., methyl). Provided herein as embodiment 216 is the compound according to embodiment 211, wherein R¹ is 6-(4aS,7aR)-(octahydropyrrolo[3,4-b][1,4]oxazinyl) substituted with one occurrence of R⁵. Provided herein as embodiment 217 is the compound according to embodiment 216, wherein R⁵ is C₁₋₄ alkyl (e.g., methyl). Provided herein as embodiment 218 is the compound according to embodiment 211, wherein L—R¹ is

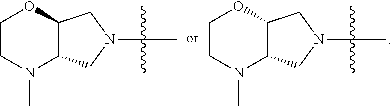 [SMILES: *N1C[C@@H]2OCCN(C)[C@H]2C1 ; "or" ; *N1C[C@H]2OCCN(C)[C@H]2C1 ; "."]

Provided herein as embodiment 219 is the compound according to embodiment 125, wherein R¹ is N-azetidinyl substituted with 0-3 occurrences of R⁵.

Provided herein as embodiment 220 is the compound according to embodiment 219, wherein R¹ is N-azetidinyl substituted with 0 occurrences of R⁵.

Provided herein as embodiment 221 is the compound according to embodiment 219, wherein R¹ is N-azetidinyl substituted with one occurrence of R⁵. Provided herein as embodiment 222 is the compound according to embodiment 221, wherein R⁵ is —N(R^{W})₂, heteroaryl or heterocycloalkyl, wherein each heteroaryl or heterocycloalkyl is substituted with 0-3 occurrences of R⁷. Provided herein as embodiment 223 is the compound according to embodiment 222, wherein R⁵ is heterocycloalkyl substituted with 0-3 occurrences of R⁷. Provided herein as embodiment 224 is the compound according to embodiment 223, wherein R⁵ is N-piperidinyl, N-pyrrolidinyl, N-piperazinyl, N-morpholinyl, N-azetidinyl, 7-(3-oxa-7,9-diazabicyclo[3.3.1]nonanyl), N-thiomorpholinyl or N-(thiomorpholinyl-1,1-dioxide), each of which is substituted with 0-3 occurrences of R⁷. Provided herein as embodiment 225 is the compound according to embodiment 223, wherein R⁵ is heterocycloalkyl substituted with 0 occurrences of R⁷. Provided herein as embodiment 226 is the compound according to embodiment 225, wherein R⁵ is N-piperidinyl, N-pyrrolidinyl, N-piperazinyl, N-morpholinyl, N-azetidinyl, 7-(3-oxa-7,9-diazabicyclo[3.3.1]nonanyl), N-thiomorpholinyl or N-(thiomorpholinyl-1,1-dioxide), each of which is substituted with 0 occurrences of R⁷. Provided herein as embodiment 227 is the compound according to embodiment 226, wherein R⁵ is N-piperidinyl, N-morpholinyl, 7-(3-oxa-7,9-diazabicyclo[3.3.1]nonanyl) or N-(thiomorpholinyl-1,1-dioxide), each of which is substituted with 0 occurrences of R⁷. Provided herein as embodiment 228 is the compound according to embodiment 227, wherein R⁵ is N-piperidinyl substituted with 0 occurrences of R⁷. Provided herein as embodiment 229 is the compound according to embodiment 227, wherein R⁵ is N-morpholinyl substituted with 0 occurrences of R⁷. Provided herein as embodiment 230 is the compound according to embodiment 227, wherein R⁵ is 7-(3-oxa-7,9-diazabicyclo[3.3.1]nonanyl) substituted with 0 occurrences of R⁵. Provided herein as embodiment 231 is the compound according to embodiment 227, wherein R⁵ is N-(thiomorpholinyl-1,1-dioxide) substituted with 0 occurrences of R⁷. Provided herein as embodiment 232 is the compound according to embodiment 221, wherein L—R¹ is

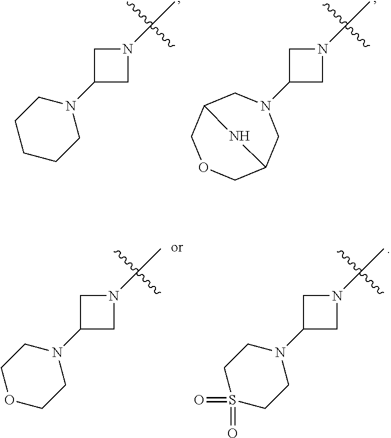

Provided herein as embodiment 233 is the compound according to embodiment 223, wherein R⁵ is heterocycloalkyl substituted with one occurrence of R⁷. Provided herein as embodiment 234 is the compound according to embodiment 233, wherein R⁵ is N-piperidinyl, N-pyrrolidinyl, N-piperazinyl, N-morpholinyl, N-azetidinyl, 7-(3-oxa-7,9-diazabicyclo[3.3.1]nonanyl), N-thiomorpholinyl or N-(thiomorpholinyl-1,1-dioxide), each of which is substituted with one occurrence of R⁷. Provided herein as embodiment 235 is the compound according to embodiment 234, wherein R⁵ is N-azetidinyl, N-piperidinyl or N-piperazinyl substituted with one occurrence of R⁷. Provided herein as embodiment 236 is the compound according to embodiment 234, wherein R⁵ is N-azetidinyl substituted with one occurrence of R⁷. Provided herein as embodiment 237 is the compound according to embodiment 234, wherein R⁵ is N-piperidinyl substituted with one occurrence of R⁷. Provided herein as embodiment 238 is the compound according to embodiment 234, wherein R⁵ is N-piperazinyl substituted with one occurrence of R⁷. Provided herein as embodiment 239 is the compound according to embodiment 234, wherein R⁷ is hydroxyl, halogen, C₁₋₄ alkoxy (e.g., methoxy) or C₁₋₄ alkyl (e.g., methyl or ethyl). Provided herein as embodiment 240 is the compound according to embodiment 239, wherein R⁷ is hydroxyl. Provided herein as embodiment 241 is the compound according to embodiment 239, wherein R⁷ is halogen (e.g., fluorine). Provided herein as embodiment 242 is the compound according to embodiment 239, wherein R⁷ is C₁₋₄ alkoxy (e.g., methoxy). Provided herein as embodiment 243 is the compound according to embodiment 239, wherein R⁷ is C₁₋₄ alkyl (e.g., ethyl). Provided herein as embodiment 244 is the compound according to embodiment 233, wherein L—R¹ is

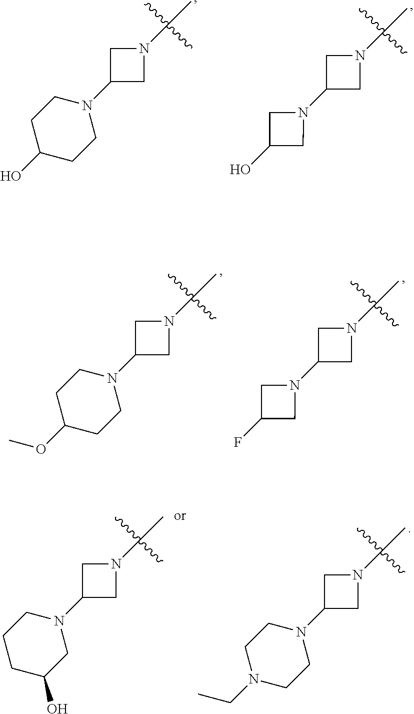

Provided herein as embodiment 245 is the compound according to embodiment 223, wherein R⁵ is heterocycloalkyl substituted with two occurrences of R⁷. Provided herein as embodiment 246 is the compound according to embodiment 245, wherein R⁵ is N-piperidinyl, N-pyrrolidinyl, N-piperazinyl, N-morpholinyl, N-azetidinyl, 7-(3-oxa-7,9-diazabicyclo[3.3.1]nonanyl), N-thiomorpholinyl or N-(thiomorpholinyl-1,1-dioxide), each of which is substituted with two occurrences of R⁷. Provided herein as embodiment 247 is the compound according to embodiment 246, wherein R⁵ is N-azetidinyl or N-morpholinyl substituted with two occurrences of R⁷. Provided herein as embodiment 248 is the compound according to embodiment 247, wherein R⁵ is N-morpholinyl substituted with two occurrences of R⁷. Provided herein as embodiment 249 is the compound according to embodiment 247, wherein R⁵ is N-azetidinyl substituted with two occurrences of R⁷. Provided herein as embodiment 250 is the compound according to embodiment 247, wherein each R⁷ is independently C₁₋₄ alkyl (e.g., methyl). Provided herein as embodiment 251 is the compound according to embodiment 247, wherein one R⁷ is hydroxyl and the other R⁷ is C₁₋₄ alkyl (e.g., methyl). Provided herein as embodiment 252 is the compound according to embodiment 245, wherein L—R¹ is

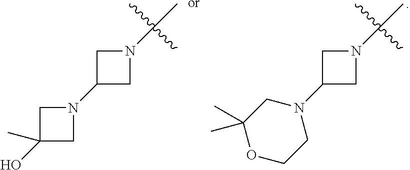 [SMILES: "or" ; "." ; *N1CC(N2CC(C)(O)C2)C1 ; *N1CC(N2CCOC(C)(C)C2)C1]

Provided herein as embodiment 253 is the compound according to embodiment 222, wherein R⁵ is —N(R^{w})₂. Provided herein as embodiment 254 is the compound according to embodiment 253, wherein both R^{w} is hydrogen. Provided herein as embodiment 255 is the compound according to embodiment 253, wherein one R^{w} is hydrogen and the other R^{w} is C₁₋₄ alkyl (e.g., methyl). Provided herein as embodiment 256 is the compound according to embodiment 253, wherein both R^{w} is C₁₋₄ alkyl (e.g., methyl). Provided herein as embodiment 257 is the compound according to embodiment 253, wherein one R^{w} is C₁₋₄ alkyl (e.g., methyl) and the other R^{w} is C₁₋₄ alkoxy (e.g., methoxy). Provided herein as embodiment 258 is the compound according to embodiment 253, wherein one R^{w} is C₁₋₄ alkyl (e.g., methyl) and the other R^{w} is heterocycloalkyl (e.g., 3-tetrahydrofuranyl or 2-oxetanyl). Provided herein as embodiment 259 is the compound according to embodiment 253, wherein one R^{w} is C₁₋₄ alkyl (e.g., methyl) and the other R^{w} is 3-tetrahydrofuranyl. Provided herein as embodiment 260 is the compound according to embodiment 253, wherein one R^{w} is C₁₋₄ alkyl (e.g., methyl) and the other R^{w} is 2-oxetanyl. Provided herein as embodiment 261 is the compound according to embodiment 253, wherein L—R¹ is

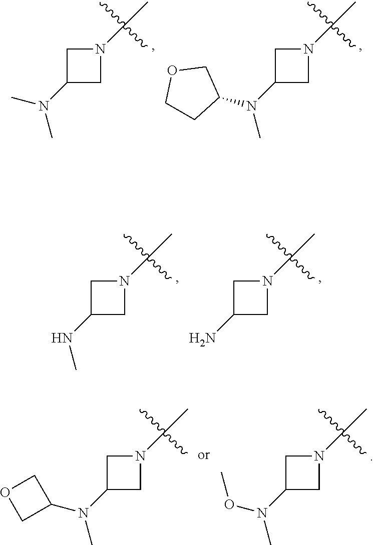

Provided herein as embodiment 262 is the compound according to embodiment 222, wherein R⁵ is heteroaryl substituted with 0-3 occurrences of R⁷. Provided herein as embodiment 263 is the compound according to embodiment 262, wherein R⁵ is 1-imidazolyl or 1-pyrazolyl substituted with 0-3 occurrences of R⁷. Provided herein as embodiment 264 is the compound according to embodiment 263, wherein R⁵ is 1-imidazolyl substituted with 0 occurrences of R⁵.

Provided herein as embodiment 265 is the compound according to embodiment 263, wherein R⁵ is 1-pyrazolyl substituted with one occurrence of R⁷. Provided herein as embodiment 266 is the compound according to embodiment 265, wherein R⁷ is —C(O)OR^{z}, wherein R^{z} is C₁₋₄ alkyl (e.g., ethyl). Provided herein as embodiment 267 is the compound according to embodiment 265, wherein R⁷ is —C(O)OEt. Provided herein as embodiment 268 is the compound according to embodiment 262, wherein L—R¹ is

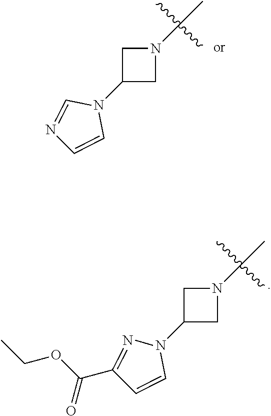 [SMILES: *N1CC(n2ccnc2)C1 ; "or" ; *N1CC(n2ccc(C(=O)OCC)n2)C1 ; "."]

Provided herein as embodiment 269 is the compound according to embodiment 219, wherein R¹ is N-azetidinyl substituted with two occurrences of R⁵. Provided herein as embodiment 270 is the compound according to embodiment 269, wherein one R⁵ is —N(R^{w})₂ and the other R⁵ is C₁₋₄ alkyl (e.g., methyl or ethyl). Provided herein as embodiment 271 is the compound according to embodiment 270, wherein both R^{w} are hydrogen. Provided herein as embodiment 272 is the compound according to embodiment 270, wherein both R^{w} are C₁₋₄ alkyl (e.g., methyl). Provided herein as embodiment 273 is the compound according to embodiment 270, wherein one R^{w} is hydrogen and the other R^{w} is C₁₋₄ alkyl (e.g., methyl). Provided herein as embodiment 274 is the compound according to embodiment 269, wherein one R⁵ is —NH₂ and the other R⁵ is methyl. Provided herein as embodiment 275 is the compound according to embodiment 269, wherein one R⁵ is —NH₂ and the other R⁵ is ethyl. Provided herein as embodiment 276 is the compound according to embodiment 269, wherein one R⁵ is —N(Me)₂ and the other R⁵ is methyl. Provided herein as embodiment 277 is the compound according to embodiment 269, wherein one R⁵ is —NH(Me) and the other R⁵ is methyl. Provided herein as embodiment 278 is the compound according to embodiment 269, wherein L—R¹ is

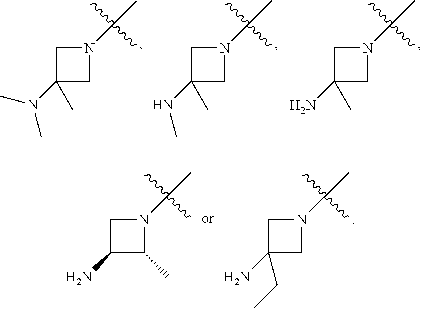

Provided herein as embodiment 279 is the compound according to embodiment 269, wherein two R⁵ taken together with the same carbon atom form a spirocyclic heteroaryl or heterocycloalkyl substituted with 0-3 occurrences of R⁷.

Provided herein as embodiment 280 is the compound according to embodiment 279, wherein two R⁵ taken together with the same carbon atom form a spirocyclic heterocycloalkyl (e.g., 2-azetidinyl, 2-pyrrolidinyl, 3-pyrrolidinyl or 5-(1,4-oxazepanyl)) substituted with 0-3 occurrences of R⁷.

Provided herein as embodiment 281 is the compound according to embodiment 280, wherein two R⁵ taken together with the same carbon atom form a spirocyclic heterocycloalkyl (e.g., 2-azetidinyl, 2-pyrrolidinyl or 3-pyrrolidinyl) substituted with 0 occurrences of R⁷.

Provided herein as embodiment 282 is the compound according to embodiment 281, wherein two R⁵ taken together with the same carbon atom form a spirocyclic 2-azetidinyl substituted with 0 occurrences of R⁷. Provided herein as embodiment 283 is the compound according to embodiment 281, wherein two R⁵ taken together with the same carbon atom form a spirocyclic 2-pyrrolidinyl substituted with 0 occurrences of R⁷. Provided herein as embodiment 284 is the compound according to embodiment 281, wherein two R⁵ taken together with the same carbon atom form a spirocyclic 3-pyrrolidinyl substituted with 0 occurrences of R⁷.

Provided herein as embodiment 285 is the compound according to embodiment 280, wherein two R⁵ taken together with the same carbon atom form a spirocyclic heterocycloalkyl (e.g., 5-(1,4-oxazepanyl)) substituted with one occurrence of R⁷. Provided herein as embodiment 286 is the compound according to embodiment 285, wherein R⁷ is oxo.

Provided herein as embodiment 287 is the compound according to embodiment 286, wherein two R⁵ taken together with the same carbon atom form a spirocyclic 5-(1,4-oxazepan-3-onyl). Provided herein as embodiment 288 is the compound according to embodiment 280, wherein two R⁵ taken together with the same carbon atom form a spirocyclic heterocycloalkyl (e.g., 2-azetidinyl) substituted with two occurrences of R⁷. Provided herein as embodiment 289 is the compound according to embodiment 288, wherein both R⁷ are halogen (e.g., fluorine).

Provided herein as embodiment 290 is the compound according to embodiment 279, wherein two R⁵ taken together with the same carbon atom form a spirocyclic heteroaryl (e.g., 5-(6,7-dihydro-5H-pyrrolo[1,2-a]imidazolyl) or 5-(5,6,7,8-tetrahydroimidazo[1,2-a]pyrimidinyl)) substituted with 0-3 occurrences of R⁷. Provided herein as embodiment 291 is the compound according to embodiment 290, wherein two R⁵ taken together with the same carbon atom form a spirocyclic heteroaryl (e.g., 5-(6,7-dihydro-5H-pyrrolo[1,2-a]imidazolyl) or 5-(5,6,7,8-tetrahydroimidazo[1,2-a]pyrimidinyl)) substituted with 0 occurrences of R⁷. Provided herein as embodiment 292 is the compound according to embodiment 290, wherein two R⁵ taken together with the same carbon atom form a spirocyclic heteroaryl (e.g., 5-(6,7-dihydro-5H-pyrrolo[1,2-a]imidazolyl) or 5-(5,6,7,8-tetrahydroimidazo[1,2-a]pyrimidinyl)) substituted with one occurrence of R⁷. Provided herein as embodiment 293 is the compound according to embodiment 292, wherein two R⁵ taken together with the same carbon atom form a spirocyclic 5-(6,7-dihydro-5H-pyrrolo[1,2-a]imidazolyl) substituted with one occurrence of R⁷. Provided herein as embodiment 294 is the compound according to embodiment 293, wherein R⁷ is hydroxyl. Provided herein as embodiment 295 is the compound according to embodiment 293, wherein two R⁵ taken together with the same carbon atom form a spirocyclic 5-(7-hydroxy-6,7-dihydro-5H-pyrrolo[1,2-a]imidazolyl).

Provided herein as embodiment 296 is the compound according to embodiment 290, wherein two R⁵ taken together with the same carbon atom form a spirocyclic heteroaryl (e.g., 5-(6,7-dihydro-5H-pyrrolo[1,2-a]imidazolyl) or 5-(5,6,7,8-tetrahydroimidazo[1,2-a]pyrimidinyl)) substituted with two occurrences of R⁷. Provided herein as embodiment 297 is the compound according to embodiment 296, wherein two R⁵ taken together with the same carbon atom form a spirocyclic 5-(5,6,7,8-tetrahydroimidazo[1,2-a]pyrimidinyl) substituted with two occurrences of R⁷. Provided herein as embodiment 298 is the compound according to embodiment 297, wherein one R⁷ is oxo and the other R⁷ is C₁₋₄ alkyl (e.g., methyl). Provided herein as embodiment 299 is the compound according to embodiment 297, wherein two R⁵ taken together with the same carbon atom form a spirocyclic 5-(8-methyl-5,6-dihydroimidazo[1,2-a]pyrimidin-7(8H)-onyl).

Provided herein as embodiment 300 is the compound according to embodiment 279, wherein L—R¹ is

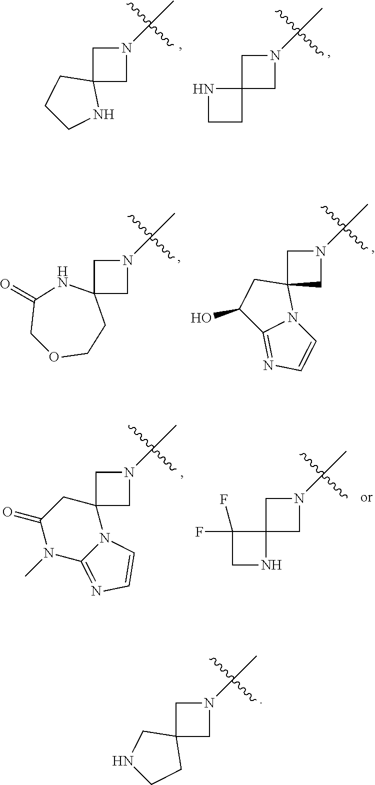

Provided herein as embodiment 301 is the compound according to embodiment 125, wherein R¹ is N-pyrrolidinyl substituted with 0-3 occurrences of R⁵. Provided herein as embodiment 302 is the compound according to embodiment 301, wherein R¹ is N-pyrrolidinyl substituted with 0 occurrences of R⁵. Provided herein as embodiment 303 is the compound according to embodiment 301, wherein R¹ is N-pyrrolidinyl substituted with one occurrence of R⁵. Provided herein as embodiment 304 is the compound according to embodiment 303, wherein R⁵ is —N(R^{w})₂. Provided herein as embodiment 305 is the compound according to embodiment 304, wherein both R^{w} are hydrogen. Provided herein as embodiment 306 is the compound according to embodiment 304, wherein one R^{w} is hydrogen and the other R^{w} is C₁₋₄ alkyl (e.g., methyl). Provided herein as embodiment 307 is the compound according to embodiment 304, wherein both R^{w} are C₁₋₄ alkyl (e.g., methyl).

Provided herein as embodiment 308 is the compound according to embodiment 301, wherein R¹ is N-pyrrolidinyl substituted with two occurrences of R⁵. Provided herein as embodiment 309 is the compound according to embodiment 308, wherein one R⁵ is —N(R^{w})₂ and the other R⁵ is C₁₋₄ alkyl (e.g., methyl). Provided herein as embodiment 310 is the compound according to embodiment 309, wherein both R^{w} are hydrogen. Provided herein as embodiment 311 is the compound according to embodiment 309, wherein one R⁵ is —NH₂ and the other R⁵ is methyl. Provided herein as embodiment 312 is the compound according to embodiment 308, wherein two R⁵ taken together with the same carbon atom form a spirocyclic heteroaryl or heterocycloalkyl substituted with 0-3 occurrences of R⁷. Provided herein as embodiment 313 is the compound according to embodiment 312, wherein two R⁵ taken together with the same carbon atom form a spirocyclic heterocycloalkyl (e.g., 2-azetidinyl) substituted with 0-3 occurrences of R⁷. Provided herein as embodiment 314 is the compound according to embodiment 313, wherein two R⁵ taken together with the same carbon atom form a spirocyclic heterocycloalkyl (e.g., 2-azetidinyl) substituted with 0 occurrences of R⁷. Provided herein as embodiment 315 is the compound according to embodiment 313, wherein two R⁵ taken together with the same carbon atom form a spirocyclic 2-azetidinyl substituted with 0 occurrences of R⁷. Provided herein as embodiment 316 is the compound according to embodiment 301, wherein L—R¹ is

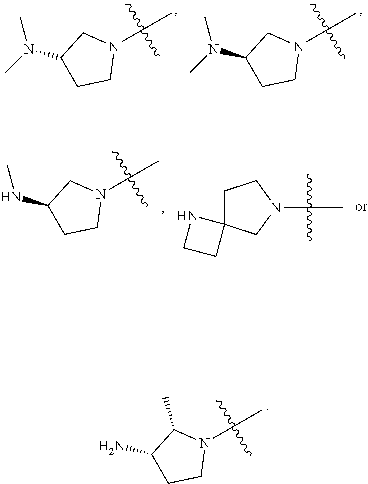

Provided herein as embodiment 317 is the compound according to embodiment 125 wherein R¹ is N-piperidinyl substituted with 0-3 occurrences of R⁵. Provided herein as embodiment 318 is the compound according to embodiment 317, wherein R¹ is N-piperidinyl substituted with one occurrence of R⁵. Provided herein as embodiment 319 is the compound according to embodiment 318, wherein R⁵ is heteroaryl substituted with 0-3 occurrences of R⁷. Provided herein as embodiment 320 is the compound according to embodiment 319, wherein R⁵ is 2-thiazolyl substituted with 0-3 occurrences of R⁷. Provided herein as embodiment 321 is the compound according to embodiment 320, wherein R⁵ is 2-thiazolyl substituted with 0 occurrences of R⁷. Provided herein as embodiment 322 is the compound according to embodiment 317, wherein L—R¹ is

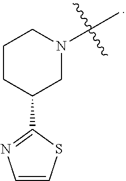 [SMILES: "." ; *N1CCC[C@@H](c2nccs2)C1]

Provided herein as embodiment 323 is the compound according to any one of embodiments 1-124, wherein R¹ is —N(R^{a})₂. Provided herein as embodiment 324 is the compound according to embodiment 323, wherein each R^{a} is C₁₋₄ alkyl (e.g., methyl). Provided herein as embodiment 325 is the compound according to embodiment 323, wherein R¹ is —N(R)₂ and each R^{a} is methyl. Provided herein as embodiment 326 is the compound according to embodiment 323, wherein L—R¹ is

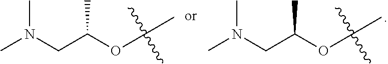

Provided herein as embodiment 327 is the compound according to any one of embodiments 1-124, wherein R¹ is heteroaryl (e.g., 5-thiazolyl) substituted with 0-3 occurrences of R⁵. Provided herein as embodiment 328 is the compound according to embodiment 327, wherein R¹ is heteroaryl (e.g., 5-thiazolyl) substituted with 0 occurrences of R⁵. Provided herein as embodiment 329 is the compound according to embodiment 327, wherein R¹ is 5-thiazolyl substituted with 0 occurrences of R⁵. Provided herein as embodiment 330 is the compound according to embodiment 327, wherein R¹ is 6-(4,5,6,7-tetrahydrobenzo[d]thiazolyl) substituted with 0-3 occurrences of R⁵. Provided herein as embodiment 331 is the compound according to embodiment 330, wherein R¹ is 6-(4,5,6,7-tetrahydrobenzo[d]thiazolyl) substituted with 0 occurrences of R⁵. Provided herein as embodiment 332 is the compound according to embodiment 327, wherein L—R¹ is

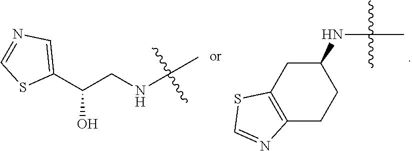 [SMILES: *NC[C@H](O)c1cncs1 ; "or" ; *N[C@H]1CCc2ncsc2C1 ; "."]

Provided herein as embodiment 333 is the compound according to any one of embodiments 1-124, wherein —L—R¹ is

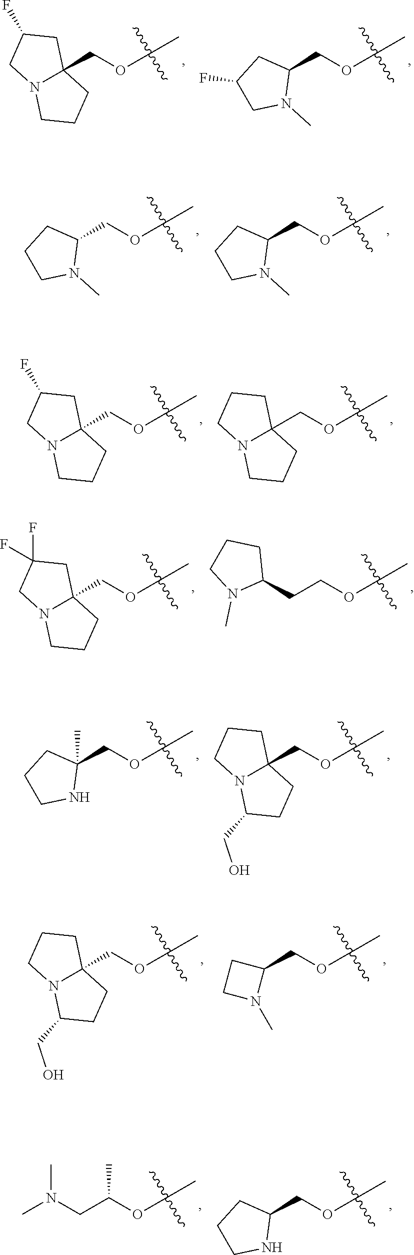

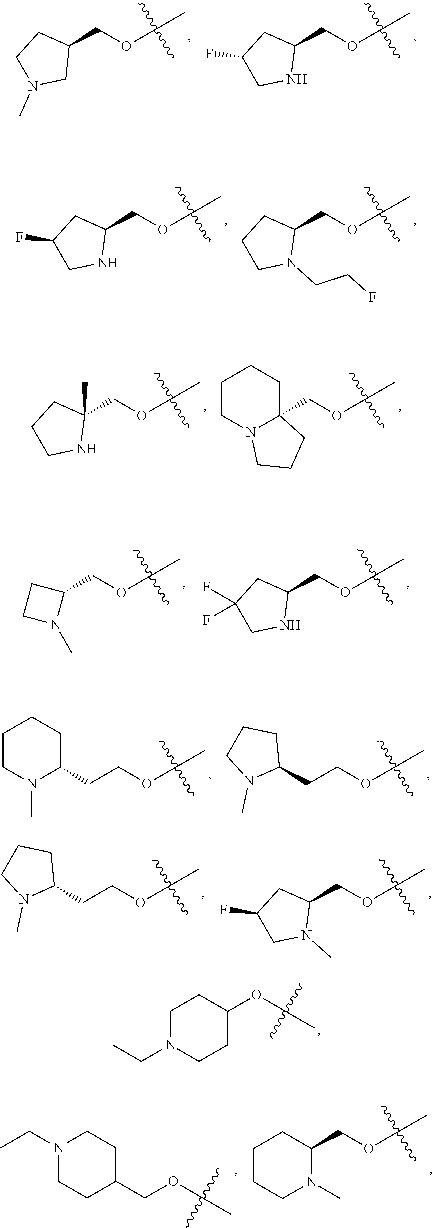

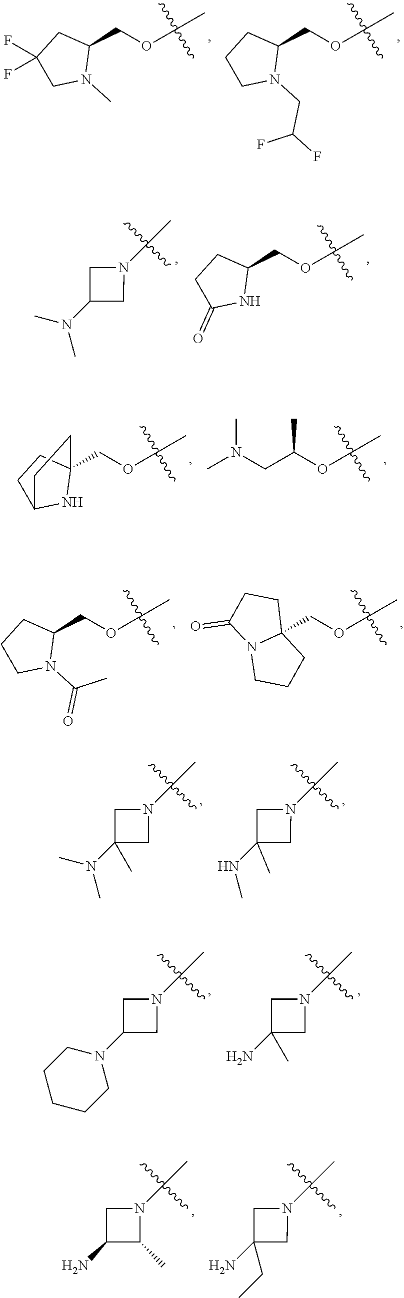

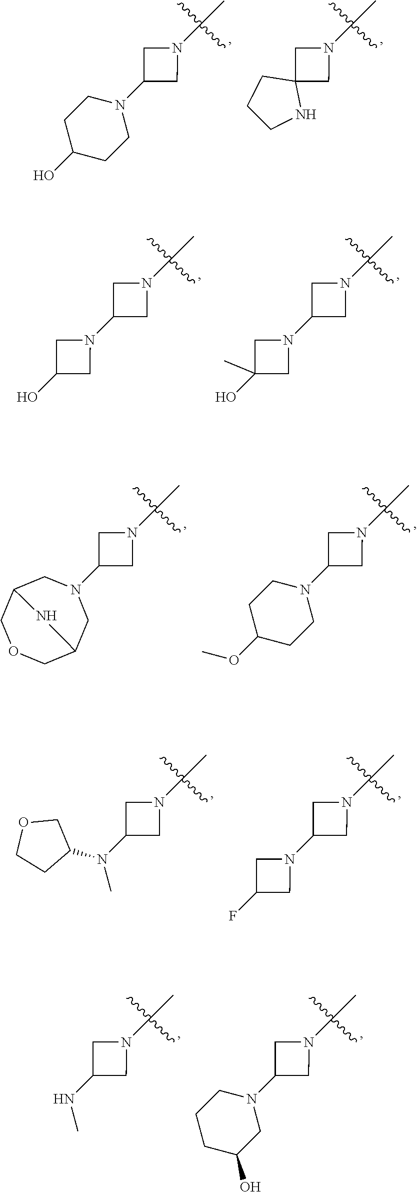

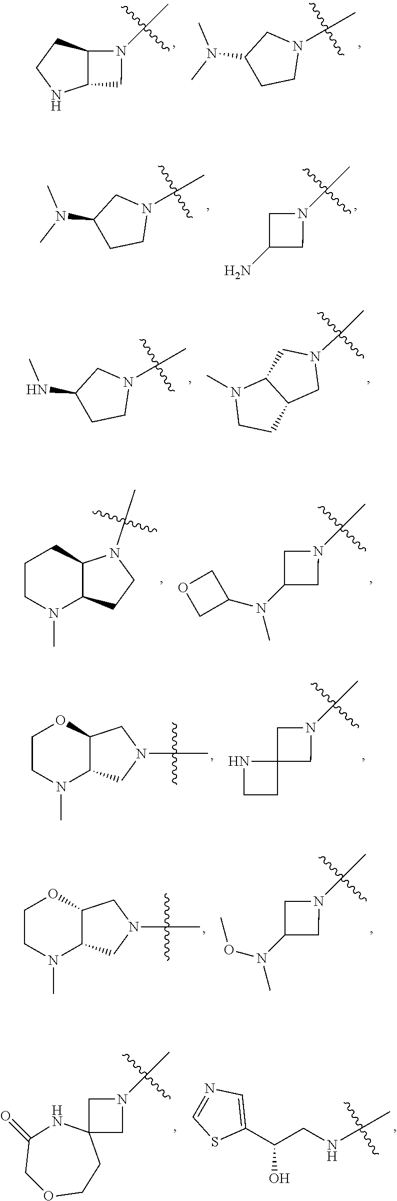

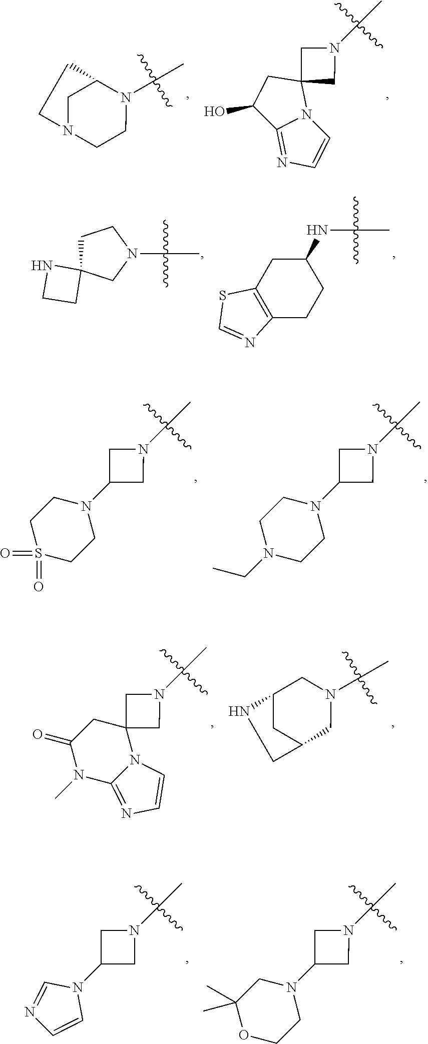

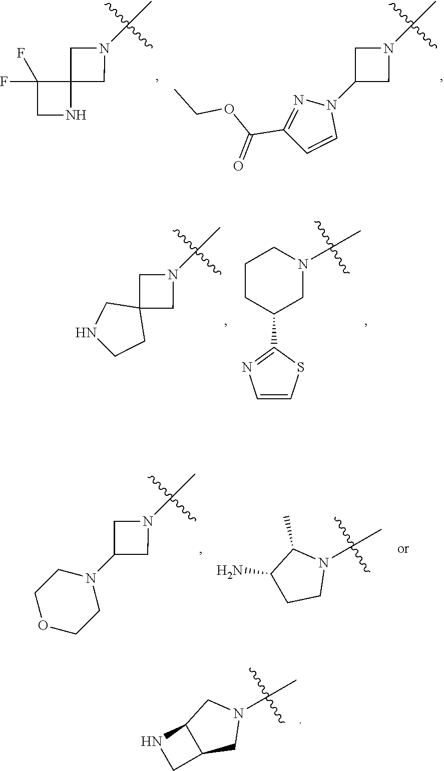

Provided herein as embodiment 334 is the compound according to embodiment 333, wherein —L—R¹ is

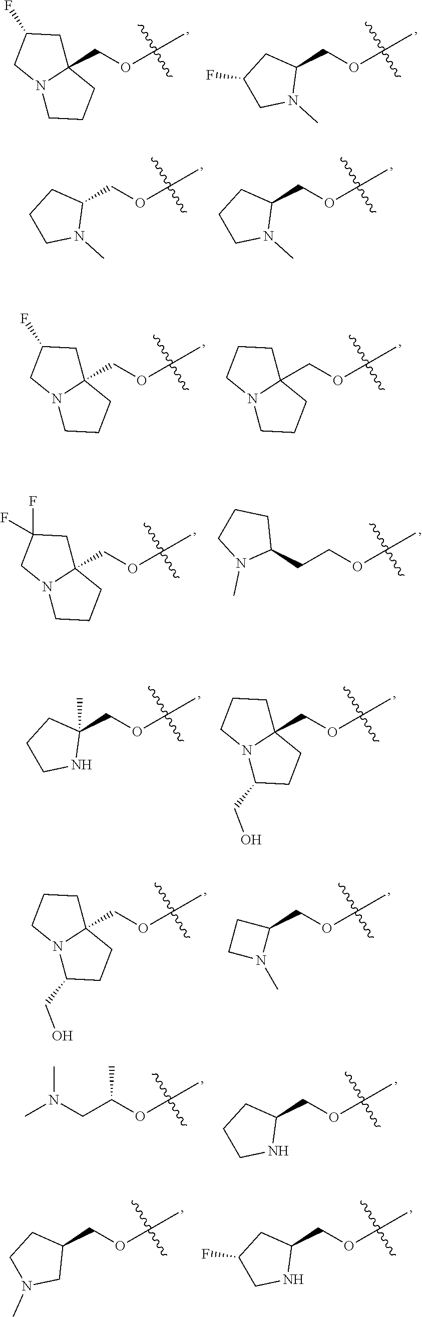

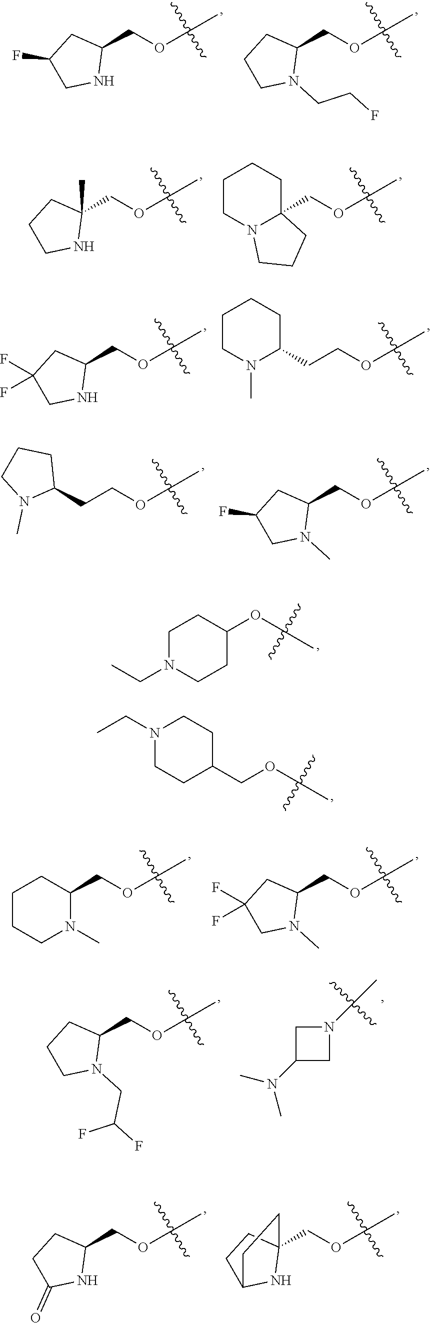

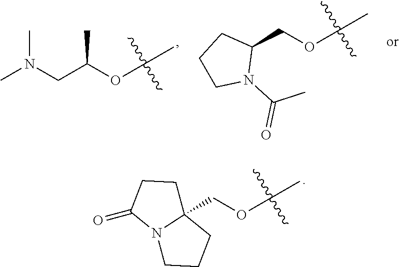

Provided herein as embodiment 335 is the compound according to embodiment 334, wherein —L—R¹ is

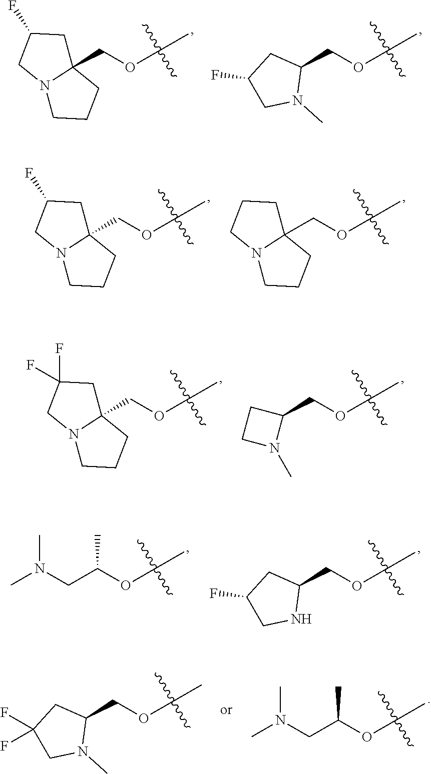

Provided herein as embodiment 336 is the compound according to any one of embodiments 1-335, wherein R² is halogen, C₁₋₄ alkyl, C₂₋₄ alkenyl or cyano. Provided herein as embodiment 337 is the compound according to embodiment 336, wherein R² is chlorine, methyl, ethyl, vinyl or cyano. Provided herein as embodiment 338 is the compound according to embodiment 336, wherein R² is chlorine. Provided herein as embodiment 339 is the compound according to embodiment 336, wherein R² is methyl or ethyl. Provided herein as embodiment 340 is the compound according to embodiment 339, wherein R² is methyl. Provided herein as embodiment 341 is the compound according to embodiment 339, wherein R² is ethyl. Provided herein as embodiment 342 is the compound according to embodiment 336, wherein R² is vinyl (i.e., 2-ethenyl). Provided herein as embodiment 343 is the compound according to embodiment 336, wherein R² is cyano.

Provided herein as embodiment 344 is the compound according to any one of embodiments 1-343, wherein R⁴ is halogen (e.g., fluorine).

Provided herein as embodiment 345 is the compound according to any one of embodiments 1-343, wherein R⁴ is fluorine.

Provided herein as embodiment 346 is the compound according to any one of embodiments 1-345, wherein R³ is hydrogen or halogen (e.g., fluorine). Provided herein as embodiment 347 is the compound according to any one of embodiments 1-345, wherein R³ is hydrogen. Provided herein as embodiment 348 is the compound according to any one of embodiments 1-345, wherein R³ is fluorine.

Provided herein as embodiment 349 is the compound according to any one of embodiments 1-348, wherein R^{q} is attached as illustrated in formula (IIa):

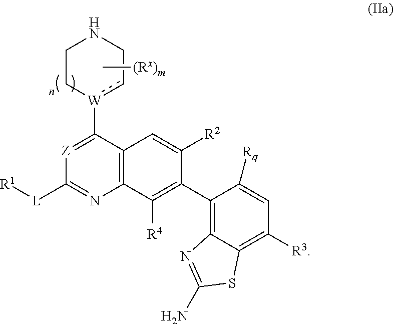

Provided herein as embodiment 350 is the compound according to any one of embodiments 1-348, wherein R^{q} is attached as illustrated in Formula (IIb):

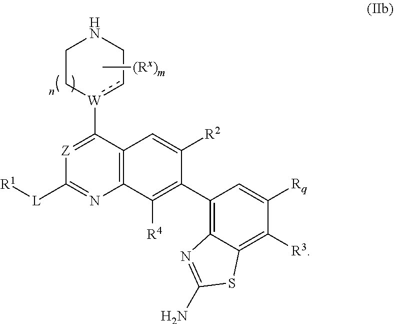

Provided herein as embodiment 351 is the compound according to any one of 1-350, wherein R^{q} is hydrogen. Provided herein as embodiment 352 is the compound according to any one of embodiments 1-350, wherein R^{q} is halogen (e.g., chlorine or fluorine). Provided herein as embodiment 353 is the compound according to any one of embodiments 1-350, wherein R^{q} is C₁₋₄ alkyl (e.g., methyl).

Provided herein as embodiment 354 is the compound according to embodiment 1, wherein the compound is selected from one of the following compounds:
4-(6-Chloro-8-fluoro-2-(((S)-1-methylpyrrolidin-2-yl)methoxy)-4-(3-(trifluoromethyl)piperazin-1-yl)quinazolin-7-yl)benzo[d]thiazol-2-amine;
4-(4-((1R,5S)-3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(7-(2-amino-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)-1,4-diazepan-2-one;
4-(4-(3,6-diazabicyclo[3.1.1]heptan-3-yl)-6-chloro-8-fluoro-2-(((S)-1-methylpyrrolidin-2-yl)methoxy)quinazolin-7-yl)benzo[d]thiazol-2-amine; 4-(4-((1R,5S)-3,6-diazabicyclo[3.1.1]heptan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,6-diazabicyclo[3.1.1]heptan-3-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(6-chloro-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-chloro-2-((2,2-difluorotetrahydro-1H-pyrrolizin-7a(5H)-yl)methoxy)-8-fluoroquinazolin-7-yl)-7-fluorobenzo[d]thiazol-2-amine;
4-(4-(3-oxa-7,9-diazabicyclo[3.3.1]nonan-9-yl)-6-chloro-8-fluoro-2-(((S)-1-methylpyrrolidin-2-yl)methoxy)quinazolin-7-yl)benzo[d]thiazol-2-amine;
4-(4-((1R,5S)-3,6-diazabicyclo[3.1.1]heptan-3-yl)-6-chloro-8-fluoro-2-(2-((S)-1-methylpyrrolidin-2-yl)ethoxy)quinazolin-7-yl)-7-fluorobenzo[d]thiazol-2-amine;
4-(7-(2-amino-7-fluorobenzo[d]thiazol-4-yl)-6-chloro-8-fluoro-2-((tetrahydro-1H-pyrrolizin-7a(5H)-yl)methoxy)quinazolin-4-yl)-1,4-diazepan-2-one;
1-(7-(2-amino-7-fluorobenzo[d]thiazol-4-yl)-6-chloro-8-fluoro-2-((tetrahydro-1H-pyrrolizin-7a(5H)-yl)methoxy)quinazolin-4-yl)-1,4-diazepan-5-one;
4-(4-((1R,5S)-3,6-diazabicyclo[3.1.1]heptan-3-yl)-6-chloro-2-((2,2-difluorotetrahydro-1H-pyrrolizin-7a(5H)-yl)methoxy)-8-fluoroquinazolin-7-yl)-7-fluorobenzo[d]thiazol-2-amine;
4-(4-((1S,4S)-2,5-diazabicyclo[2.2.2]octan-2-yl)-6-chloro-8-fluoro-2-(((S)-1-methylpyrrolidin-2-yl)methoxy)quinazolin-7-yl)benzo[d]thiazol-2-amine;
4-(6-chloro-8-fluoro-4-((S)-2-methylpiperazin-1-yl)-2-(((S)-1-methylpyrrolidin-2-yl)methoxy)quinazolin-7-yl)benzo[d]thiazol-2-amine;
4-(6-chloro-4-(1,4-diazepan-1-yl)-8-fluoro-2-(((S)-1-methylpyrrolidin-2-yl)methoxy)quinazolin-7-yl)benzo[d]thiazol-2-amine;
4-(4-(3,6-diazabicyclo[3.1.1]heptan-6-yl)-6-chloro-8-fluoro-2-(((S)-1-methylpyrrolidin-2-yl)methoxy)quinazolin-7-yl)benzo[d]thiazol-2-amine;
4-(7-(2-amino-7-fluorobenzo[d]thiazol-4-yl)-6-chloro-8-fluoro-2-(((2S,4R)-4-fluoro-1-methylpyrrolidin-2-yl)methoxy)quinazolin-4-yl)-1,4-diazepan-2-one;
4-(4-(2,5-diazabicyclo[4.1.0]heptan-2-yl)-6-chloro-8-fluoro-2-(((S)-1-methylpyrrolidin-2-yl)methoxy)quinazolin-7-yl)benzo[d]thiazol-2-amine;
1-(7-(2-amino-7-fluorobenzo[d]thiazol-4-yl)-6-chloro-8-fluoro-2-(((2S,4R)-4-fluoro-1-methylpyrrolidin-2-yl)methoxy)quinazolin-4-yl)-1,4-diazepan-5-one;
4-(7-(2-aminobenzo[d]thiazol-4-yl)-6-chloro-8-fluoro-2-(((S)-1-methylpyrrolidin-2-yl)methoxy)quinazolin-4-yl)-1,4-diazepan-2-one;
4-(7-(2-aminobenzo[d]thiazol-4-yl)-6-chloro-8-fluoro-2-(((S)-1-methylpyrrolidin-2-yl)methoxy)quinazolin-4-yl)piperazin-2-one;
1-(7-(2-aminobenzo[d]thiazol-4-yl)-6-chloro-8-fluoro-2-(((S)-1-methylpyrrolidin-2-yl)methoxy)quinazolin-4-yl)-1,4-diazepan-5-one; ((2R)-4-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)methanol;
2-((2S)-4-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)acetonitrile;
4-(4-((1R,4R)-2,5-diazabicyclo[2.2.2]octan-2-yl)-6-chloro-8-fluoro-2-(((S)-1-methylpyrrolidin-2-yl)methoxy)quinazolin-7-yl)benzo[d]thiazol-2-amine;
4-(6-chloro-4-(1,4-diazepan-1-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,6-diazabicyclo[3.2.1]octan-6-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,6-diazabicyclo[3.2.1]octan-6-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,6-diazabicyclo[3.2.2]nonan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,9-diazabicyclo[4.2.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,9-diazabicyclo[4.2.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(2,6-diazabicyclo[3.2.1]octan-6-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
2-((2S)-1-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)ethan-1-ol; ((2R)-1-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)methanol;
4-(4-((1S,4S)-2,5-diazabicyclo[2.2.2]octan-2-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(2,5-diazabicyclo[4.1.0]heptan-2-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(6-chloro-8-fluoro-2-(((2R,7aS)-2-fluorotetrahydro-1H-pyrrolizin-7a(5H)-yl)methoxy)-4-(octahydro-1H-cyclopenta[b]pyrazin-1-yl)quinazolin-7-yl)-7-fluorobenzo[d]thiazol-2-amine;
4-(4-((1R,5S)-3-oxa-7,9-diazabicyclo[3.3.1]nonan-7-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(4-((1R,5S)-3-oxa-7,9-diazabicyclo[3.3.1]nonan-7-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
7a-(((7-(2-aminobenzo[d]thiazol-4-yl)-6-chloro-8-fluoro-4-(piperazin-1-yl)quinazolin-2-yl)oxy)methyl)hexahydro-3H-pyrrolizin-3-one;
4-(6-chloro-8-fluoro-4-(piperazin-1-yl)-2-((tetrahydro-1H-pyrrolizin-7a(5H)-yl)methoxy)quinazolin-7-yl)benzo[d]thiazol-2-amine;
4-(6-chloro-8-fluoro-2-(((2R,7aS)-2-fluorotetrahydro-1H-pyrrolizin-7a(5H)-yl)methoxy)-4-(piperazin-1-yl)quinazolin-7-yl)benzo[d]thiazol-2-amine;
4-(6-chloro-8-fluoro-2-(((2R,7aR)-2-fluorotetrahydro-1H-pyrrolizin-7a(5H)-yl)methoxy)-4-(piperazin-1-yl)quinazolin-7-yl)benzo[d]thiazol-2-amine;
4-(6-chloro-8-fluoro-2-(((2S,4R)-4-fluoropyrrolidin-2-yl)methoxy)-4-(piperazin-1-yl)quinazolin-7-yl)benzo[d]thiazol-2-amine;
4-(6-chloro-2-(((S)-1-(dimethylamino)propan-2-yl)oxy)-8-fluoro-4-(piperazin-1-yl)quinazolin-7-yl)benzo[d]thiazol-2-amine;
4-(6-chloro-8-fluoro-2-(((2S,4R)-4-fluoro-1-methylpyrrolidin-2-yl)methoxy)-4-(piperazin-1-yl)quinazolin-7-yl)benzo[d]thiazol-2-amine;
((3R,7aR)-7a-(((7-(2-aminobenzo[d]thiazol-4-yl)-6-chloro-8-fluoro-4-(piperazin-1-yl)quinazolin-2-yl)oxy)methyl)hexahydro-1H-pyrrolizin-3-yl)methanol;
((3R,7aS)-7a-(((7-(2-aminobenzo[d]thiazol-4-yl)-6-chloro-8-fluoro-4-(piperazin-1-yl)quinazolin-2-yl)oxy)methyl)hexahydro-1H-pyrrolizin-3-yl)methanol;
4-(6-chloro-8-fluoro-2-(((S)-1-methyl)azetidin-2-yl)methoxy)-4-(piperazin-1-yl)quinazolin-7-yl)benzo[d]thiazol-2-amine;
4-(6-chloro-8-fluoro-2-(((R)-1-methylpyrrolidin-3-yl)methoxy)-4-(piperazin-1-yl)quinazolin-7-yl)benzo[d]thiazol-2-amine;
4-(6-chloro-2-((2,2-difluorotetrahydro-1H-pyrrolizin-7a(5H)-yl)methoxy)-8-fluoro-4-(piperazin-1-yl)quinazolin-7-yl)benzo[d]thiazol-2-amine;
4-(6-chloro-8-fluoro-2-(((2S,4S)-4-fluoropyrrolidin-2-yl)methoxy)-4-(piperazin-1-yl)quinazolin-7-yl)benzo[d]thiazol-2-amine;
4-(6-chloro-8-fluoro-2-(((S)-1-(2-fluoroethyl)pyrrolidin-2-yl)methoxy)-4-(piperazin-1-yl)quinazolin-7-yl)benzo[d]thiazol-2-amine;
4-(6-chloro-8-fluoro-2-((hexahydroindolizin-8a(1H)-yl)methoxy)-4-(piperazin-1-yl)quinazolin-7-yl)benzo[d]thiazol-2-amine;
4-(6-chloro-8-fluoro-2-(((R)-1-methyl)azetidin-2-yl)methoxy)-4-(piperazin-1-yl)quinazolin-7-yl)benzo[d]thiazol-2-amine;
4-(6-chloro-2-(((S)-4,4-difluoropyrrolidin-2-yl)methoxy)-8-fluoro-4-(piperazin-1-yl)quinazolin-7-yl)benzo[d]thiazol-2-amine;
4-(6-chloro-8-fluoro-2-(2-(1-methylpiperidin-2-yl)ethoxy)-4-(piperazin-1-yl)quinazolin-7-yl)benzo[d]thiazol-2-amine;
4-(6-chloro-8-fluoro-2-(2-((S)-1-methylpyrrolidin-2-yl)ethoxy)-4-(piperazin-1-yl)quinazolin-7-yl)benzo[d]thiazol-2-amine;
4-(6-chloro-8-fluoro-2-(((2S,4S)-4-fluoro-1-methylpyrrolidin-2-yl)methoxy)-4-(piperazin-1-yl)quinazolin-7-yl)benzo[d]thiazol-2-amine;
4-(6-chloro-2-((1-ethylpiperidin-4-yl)methoxy)-8-fluoro-4-(piperazin-1-yl)quinazolin-7-yl)benzo[d]thiazol-2-amine;
4-(6-chloro-8-fluoro-2-(((S)-1-methylpiperidin-2-yl)methoxy)-4-(piperazin-1-yl)quinazolin-7-yl)benzo[d]thiazol-2-amine;
4-(6-chloro-2-(((S)-4,4-difluoro-1-methylpyrrolidin-2-yl)methoxy)-8-fluoro-4-(piperazin-1-yl)quinazolin-7-yl)benzo[d]thiazol-2-amine;
4-(6-chloro-2-(((S)-1-(2,2-difluoroethyl)pyrrolidin-2-yl)methoxy)-8-fluoro-4-(piperazin-1-yl)quinazolin-7-yl)benzo[d]thiazol-2-amine;
4-(6-chloro-2-(3-(dimethylamino)azetidin-1-yl)-8-fluoro-4-(piperazin-1-yl)quinazolin-7-yl)benzo[d]thiazol-2-amine;
(5S)-5-(((7-(2-aminobenzo[d]thiazol-4-yl)-6-chloro-8-fluoro-4-(piperazin-1-yl)quinazolin-2-yl)oxy)methyl)pyrrolidin-2-one;
4-(6-chloro-2-(((R)-1-(dimethylamino)propan-2-yl)oxy)-8-fluoro-4-(piperazin-1-yl)quinazolin-7-yl)benzo[d]thiazol-2-amine;
4-(6-chloro-8-fluoro-2-((((S)-1-methylpyrrolidin-2-yl)methyl)amino)-4-(piperazin-1-yl)quinazolin-7-yl)benzo[d]thiazol-2-amine; 1-((2S)-2-(((7-(2-aminobenzo[d]thiazol-4-yl)-6-chloro-8-fluoro-4-(piperazin-1-yl)quinazolin-2-yl)oxy)methyl)pyrrolidin-1-yl)ethan-1-one;
4-(6-chloro-8-fluoro-4-(piperazin-1-yl)-2-(((S)-pyrrolidin-2-yl)methoxy)quinazolin-7-yl)benzo[d]thiazol-2-amine; 4-(6-chloro-8-fluoro-2-(((S)-2-methylpyrrolidin-2-yl)methoxy)-4-(piperazin-1-yl)quinazolin-7-yl)benzo[d]thiazol-2-amine;
4-(6-chloro-8-fluoro-2-(((R)-2-methylpyrrolidin-2-yl)methoxy)-4-(piperazin-1-yl)quinazolin-7-yl)benzo[d]thiazol-2-amine;
4-(2-(((1s,4s)-7-azabicyclo[2.2.1]heptan-1-yl)methoxy)-6-chloro-8-fluoro-4-(piperazin-1-yl)quinazolin-7-yl)benzo[d]thiazol-2-amine;
4-(6-Chloro-2-(3-(dimethylamino)-3-methyl)azetidin-1-yl)-8-fluoro-4-(piperazin-1-yl)quinazolin-7-yl)benzo[d]thiazol-2-amine;
4-[6-chloro-8-fluoro-2-[3-methyl-3-(methylamino)azetidin-1-yl]-4-piperazin-1-yl-quinazolin-7-yl]-1,3-benzothiazol-2-amine;
4-[6-chloro-8-fluoro-4-piperazin-1-yl-2-[3-(1-piperidyl)azetidin-1-yl]quinazolin-7-yl]-1,3-benzothiazol-2-amine;
4-[2-(3-amino-3-methyl-azetidin-1-yl)-6-chloro-8-fluoro-4-piperazin-1-yl-quinazolin-7-yl]-1,3-benzothiazol-2-amine;
4-[2-[(2R,3S)-3-amino-2-methyl-azetidin-1-yl]-6-chloro-8-fluoro-4-piperazin-1-yl-quinazolin-7-yl]-1,3-benzothiazol-2-amine;
4-[2-(3-amino-3-ethyl-azetidin-1-yl)-6-chloro-8-fluoro-4-piperazin-1-yl-quinazolin-7-yl]-1,3-benzothiazol-2-amine;
8-(7-(2-aminobenzo[d]thiazol-4-yl)-6-chloro-8-fluoro-2-(((S)-1-methylpyrrolidin-2-yl)methoxy)quinazolin-4-yl)-3,8-diazabicyclo[3.2.1]octan-2-one;
1-[1-[7-(2-amino-1,3-benzothiazol-4-yl)-6-chloro-8-fluoro-4-piperazin-1-yl-quinazolin-2-yl]azetidin-3-yl]piperidin-4-ol;
4-[6-chloro-2-(2,5-diazaspiro[3.4]octan-2-yl)-8-fluoro-4-piperazin-1-yl-quinazolin-7-yl]-1,3-benzothiazol-2-amine;
1-[1-[7-(2-amino-1,3-benzothiazol-4-yl)-6-chloro-8-fluoro-4-piperazin-1-yl-quinazolin-2-yl]azetidin-3-yl]azetidin-3-ol;
1-[1-[7-(2-amino-1,3-benzothiazol-4-yl)-6-chloro-8-fluoro-4-piperazin-1-yl-quinazolin-2-yl]azetidin-3-yl]-3-methyl-azetidin-3-ol;
4-(2-(3-((1R,5S)-3-oxa-7,9-diazabicyclo[3.3.1]nonan-7-yl)azetidin-1-yl)-6-chloro-8-fluoro-4-(piperazin-1-yl)quinazolin-7-yl)benzo[d]thiazol-2-amine;
4-[6-chloro-8-fluoro-2-[3-(4-methoxy-1-piperidyl)azetidin-1-yl]-4-piperazin-1-yl-quinazolin-7-yl]-1,3-benzothiazol-2-amine;
4-[6-chloro-8-fluoro-2-[3-[methyl-[(3R)-tetrahydrofuran-3-yl]amino]azetidin-1-yl]-4-piperazin-1-yl-quinazolin-7-yl]-1,3-benzothiazol-2-amine;
4-[6-chloro-8-fluoro-2-[3-(3-fluoroazetidin-1-yl)azetidin-1-yl]-4-piperazin-1-yl-quinazolin-7-yl]-1,3-benzothiazol-2-amine;
4-[6-chloro-8-fluoro-2-[3-(methylamino)azetidin-1-yl]-4-piperazin-1-yl-quinazolin-7-yl]-1,3-benzothiazol-2-amine;
1-[1-[7-(2-amino-1,3-benzothiazol-4-yl)-6-chloro-8-fluoro-4-piperazin-1-yl-quinazolin-2-yl]azetidin-3-yl]piperidin-3-ol;
4-[6-chloro-2-[(1S,5R)-2,6-diazabicyclo[3.2.0]heptan-6-yl]-8-fluoro-4-piperazin-1-yl-quinazolin-7-yl]-1,3-benzothiazol-2-amine;
4-[6-chloro-8-fluoro-4-piperazin-1-yl-2-[3-(dimethylamino)pyrrolidin-1-yl]quinazolin-7-yl]-1,3-benzothiazol-2-amine;
4-[6-chloro-2-[(3R)-3-(dimethylamino)pyrrolidin-1-yl]-8-fluoro-4-piperazin-1-yl-quinazolin-7-yl]-1,3-benzothiazol-2-amine;
4-[2-(3-aminoazetidin-1-yl)-6-chloro-8-fluoro-4-piperazin-1-yl-quinazolin-7-yl]-1,3-benzothiazol-2-amine;
4-[6-chloro-8-fluoro-4-piperazin-1-yl-2-[3-(methylamino)pyrrolidin-1-yl]quinazolin-7-yl]-1,3-benzothiazol-2-amine;
4-(6-chloro-8-fluoro-2-(1-methylhexahydropyrrolo[3,4-b]pyrrol-5(1H)-yl)-4-(piperazin-1-yl)quinazolin-7-yl)benzo[d]thiazol-2-amine;
4-(6-chloro-8-fluoro-2-(4-methyloctahydro-1H-pyrrolo[3,2-b]pyridin-1-yl)-4-(piperazin-1-yl)quinazolin-7-yl)benzo[d]thiazol-2-amine;
4-[6-chloro-8-fluoro-2-[3-[methyl(oxetan-3-yl)amino]azetidin-1-yl]-4-piperazin-1-yl-quinazolin-7-yl]-1,3-benzothiazol-2-amine;
4-(6-chloro-8-fluoro-2-(trans-4-methylhexahydropyrrolo[3,4-b][1,4]oxazin-6(2H)-yl)-4-(piperazin-1-yl)quinazolin-7-yl)benzo[d]thiazol-2-amine;
4-[6-chloro-2-(1,6-diazaspiro[3.3]heptan-6-yl)-8-fluoro-4-piperazin-1-yl-quinazolin-7-yl]-1,3-benzothiazol-2-amine;
4-(6-chloro-8-fluoro-2-(cis-4-methylhexahydropyrrolo[3,4-b][1,4]oxazin-6(2H)-yl)-4-(piperazin-1-yl)quinazolin-7-yl)benzo[d]thiazol-2-amine;
4-[6-chloro-8-fluoro-2-[3-[methoxy(methyl)amino]azetidin-1-yl]-4-piperazin-1-yl-quinazolin-7-yl]-1,3-benzothiazol-2-amine;
2-[7-(2-amino-1,3-benzothiazol-4-yl)-6-chloro-8-fluoro-4-piperazin-1-yl-quinazolin-2-yl]-8-oxa-2,5-diazaspiro[3.6]decan-6-one;
2-[[7-(2-amino-1,3-benzothiazol-4-yl)-6-chloro-8-fluoro-4-piperazin-1-yl-quinazolin-2-yl]amino]-1-thiazol-5-yl-ethanol;
4-[6-chloro-8-fluoro-4-piperazin-1-yl-2-[1,4-diazabicyclo[3.2.1]octan-4-yl]quinazolin-7-yl]-1,3-benzothiazol-2-amine;
1′-[7-(2-amino-1,3-benzothiazol-4-yl)-6-chloro-8-fluoro-4-piperazin-1-yl-quinazolin-2-yl]spiro[6,7-dihydropyrrolo[1,2-a]imidazole-5,3′-azetidine]-7-ol;
4-[6-chloro-8-fluoro-4-piperazin-1-yl-2-[1,7-diazaspiro[3.4]octan-7-yl]quinazolin-7-yl]-1,3-benzothiazol-2-amine;
4-[6-chloro-8-fluoro-4-piperazin-1-yl-2-[[(6S)-4,5,6,7-tetrahydro-1,3-benzothiazol-6-yl]amino]quinazolin-7-yl]-1,3-benzothiazol-2-amine;
4-[6-chloro-2-[3-(1,1-dioxo-1,4-thiazinan-4-yl)azetidin-1-yl]-8-fluoro-4-piperazin-1-yl-quinazolin-7-yl]-1,3-benzothiazol-2-amine;
4-[6-chloro-2-[3-(4-ethylpiperazin-1-yl)azetidin-1-yl]-8-fluoro-4-piperazin-1-yl-quinazolin-7-yl]-1,3-benzothiazol-2-amine;
1′-[7-(2-amino-1,3-benzothiazol-4-yl)-6-chloro-8-fluoro-4-piperazin-1-yl-quinazolin-2-yl]-8-methyl-spiro[6H-imidazo[1,2-a]pyrimidine-5,3′-azetidine]-7-one;
4-[6-chloro-8-fluoro-4-piperazin-1-yl-2-[3,6-diazabicyclo[3.2.1]octan-3-yl]quinazolin-7-yl]-1,3-benzothiazol-2-amine;
4-[6-chloro-2-[(1S,5S)-2,6-diazabicyclo[3.2.0]heptan-6-yl]-8-fluoro-4-piperazin-1-yl-quinazolin-7-yl]-1,3-benzothiazol-2-amine;
4-[6-chloro-8-fluoro-2-(3-imidazol-1-yl)azetidin-1-yl)-4-piperazin-1-yl-quinazolin-7-yl]-1,3-benzothiazol-2-amine;
4-[6-chloro-2-[3-(2,2-dimethylmorpholin-4-yl)azetidin-1-yl]-8-fluoro-4-piperazin-1-yl-quinazolin-7-yl]-1,3-benzothiazol-2-amine;
4-[6-chloro-2-(3,3-difluoro-1,6-diazaspiro[3.3]heptan-6-yl)-8-fluoro-4-piperazin-1-yl-quinazolin-7-yl]-1,3-benzothiazol-2-amine; ethyl 1-[1-[7-(2-amino-1,3-benzothiazol-4-yl)-6-chloro-8-fluoro-4-piperazin-1-yl-quinazolin-2-yl]azetidin-3-yl]pyrazole-3-carboxylate;
4-[6-chloro-2-(2,7-diazaspiro[3.4]octan-2-yl)-8-fluoro-4-piperazin-1-yl-quinazolin-7-yl]-1,3-benzothiazol-2-amine;
4-[6-chloro-8-fluoro-4-piperazin-1-yl-2-[3-thiazol-2-yl-1-piperidyl]quinazolin-7-yl]-1,3-benzothiazol-2-amine;
4-(4-((1R,5S)-3-Oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-chloro-8-fluoro-2-(((2R,7aR)-2-fluorotetrahydro-1H-pyrrolizin-7a(5H)-yl)methoxy)quinazolin-7-yl)-7-fluorobenzo[d]thiazol-2-amine;
4-(4-((1R,5S)-3-oxa-7,9-diazabicyclo[3.3.1]nonan-9-yl)-6-chloro-8-fluoro-2-(((2R,7aR)-2-fluorotetrahydro-1H-pyrrolizin-7a(5H)-yl)methoxy)quinazolin-7-yl)-7-fluorobenzo[d]thiazol-2-amine;
4-(4-((1R,5S)-3-oxa-7,9-diazabicyclo[3.3.1]nonan-9-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(4-((1R,5S)-3-oxa-7,9-diazabicyclo[3.3.1]nonan-9-yl)-6-chloro-2-((2,2-difluorotetrahydro-1H-pyrrolizin-7a(5H)-yl)methoxy)-8-fluoroquinazolin-7-yl)-7-fluorobenzo[d]thiazol-2-amine;
4-(6-chloro-8-fluoro-2-(((S)-1-methylpyrrolidin-2-yl)methoxy)-4-(piperazin-1-yl)quinazolin-7-yl)benzo[d]thiazol-2-amine;
4-(6-chloro-2-((1-ethylpiperidin-4-yl)oxy)-8-fluoro-4-(piperazin-1-yl)quinazolin-7-yl)benzo[d]thiazol-2-amine;
4-(6-chloro-8-fluoro-2-(((S)-1-methylpyrrolidin-2-yl)methoxy)-4-(piperazin-1-yl)quinazolin-7-yl)-6-methylbenzo[d]thiazol-2-amine;
4-(6-chloro-8-fluoro-2-(((S)-1-methylpyrrolidin-2-yl)methoxy)-4-(piperazin-1-yl)quinazolin-7-yl)-7-fluorobenzo[d]thiazol-2-amine;
4-(6-chloro-8-fluoro-2-(((S)-1-methylpyrrolidin-2-yl)methoxy)-4-(piperazin-1-yl)quinazolin-7-yl)-7-(trifluoromethyl)benzo[d]thiazol-2-amine;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-7-fluorobenzo[d]thiazol-2-amine;
4-(6,8-difluoro-2-(((S)-1-methylpyrrolidin-2-yl)methoxy)-4-(piperazin-1-yl)quinazolin-7-yl)benzo[d]thiazol-2-amine;
4-(4-((1R,5S)-3,8-diazabicyclo[3.2.1]octan-8-yl)-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-6-chloro-8-fluoro-2-(((2R,7aR)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-methylquinazolin-7-yl)-7-fluorobenzo[d]thiazol-2-amine;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6,8-difluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(6-Chloro-8-fluoro-2-(((S)-1-methylpyrrolidin-2-yl)methoxy)-4-(1,2,3,6-tetrahydropyridin-4-yl)quinazolin-7-yl)benzo[d]thiazol-2-amine;
4-(6-chloro-8-fluoro-4-(3-methyl-1,2,3,6-tetrahydropyridin-4-yl)-2-(((S)-1-methylpyrrolidin-2-yl)methoxy)quinazolin-7-yl)benzo[d]thiazol-2-amine;
4-(6-chloro-8-fluoro-4-(3-methyl-1,2,3,6-tetrahydropyridin-4-yl)-2-(((S)-1-methylpyrrolidin-2-yl)methoxy)quinazolin-7-yl)benzo[d]thiazol-2-amine;
4-(6-chloro-8-fluoro-4-(5-methyl-1,2,3,6-tetrahydropyridin-4-yl)-2-(((S)-1-methylpyrrolidin-2-yl)methoxy)quinazolin-7-yl)benzo[d]thiazol-2-amine;
4-(6-chloro-4-(2,5-dihydro-1H-pyrrol-3-yl)-8-fluoro-2-(((S)-1-methylpyrrolidin-2-yl)methoxy)quinazolin-7-yl)benzo[d]thiazol-2-amine; 4-(8-Fluoro-2-(((S)-1-methylpyrrolidin-2-yl)methoxy)-4-(piperazin-1-yl)-6-vinylquinazolin-7-yl)benzo[d]thiazol-2-amine;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-vinylquinazolin-7-yl)-7-fluorobenzo[d]thiazol-2-amine;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)-6-vinylquinazolin-7-yl)-7-fluorobenzo[d]thiazol-2-amine;
4-(6-Ethyl-8-fluoro-2-(((S)-1-methylpyrrolidin-2-yl)methoxy)-4-(piperazin-1-yl)quinazolin-7-yl)benzo[d]thiazol-2-amine;
4-(4-((1R,5S)-3,8-diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-ethyl-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(8-Fluoro-6-methyl-2-(((S)-1-methylpyrrolidin-2-yl)methoxy)-4-(piperazin-1-yl)quinazolin-7-yl)benzo[d]thiazol-2-amine;
7-(2-Aminobenzo[d]thiazol-4-yl)-8-fluoro-2-(((S)-1-methylpyrrolidin-2-yl)methoxy)-4-(piperazin-1-yl)quinazoline-6-carbonitrile;
7-(2-Amino-7-fluorobenzo[d]thiazol-4-yl)-6-chloro-8-fluoro-2-(((S)-1-methylpyrrolidin-2-yl)methoxy)-4-(piperazin-1-yl)quinoline-3-carbonitrile;
7-(2-aminobenzo[d]thiazol-4-yl)-6-chloro-8-fluoro-2-(((S)-1-methylpyrrolidin-2-yl)methoxy)-4-(piperazin-1-yl)quinoline-3-carbonitrile;
7-(2-amino-7-fluorobenzo[d]thiazol-4-yl)-4-((1R,5S)-3,8-diazabicyclo[3.2.1]octan-8-yl)-6-chloro-8-fluoro-2-(((2R)-2-fluorotetrahydro-1H-pyrrolizin-7a(5H)-yl)methoxy)quinoline-3-carbonitrile;
7-(2-amino-7-fluorobenzo[d]thiazol-4-yl)-4-((1R,5S)-3,8-diazabicyclo[3.2.1]octan-8-yl)-6-chloro-8-fluoro-2-(((2S,4R)-4-fluoro-1-methylpyrrolidin-2-yl)methoxy)quinoline-3-carbonitrile; or
4-(4-((1R,5S)-3,8-Diazabicyclo[3.2.1]octan-3-yl)-6-chloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-amine.

Provided herein as embodiment 355 is the compound according to embodiment 1, or a pharmaceutically acceptable salt thereof, wherein the compound is:

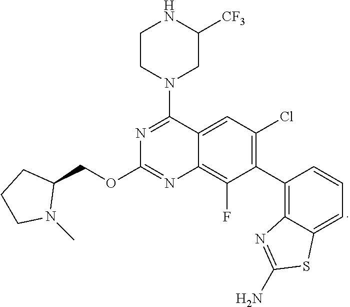 [SMILES: CN1CCC[C@H]1COc1nc(N2CCNC(C(F)(F)F)C2)c2cc(Cl)c(-c3cccc4sc(N)nc34)c(F)c2n1 ; "."]

Provided herein as embodiment 356 is the compound according to embodiment 1, or a pharmaceutically acceptable salt thereof, wherein the compound is:

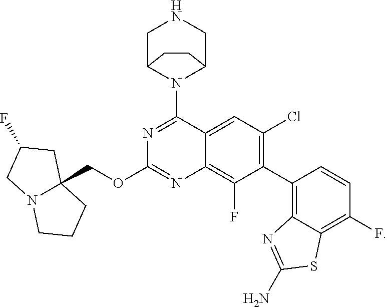 [SMILES: Nc1nc2c(-c3c(Cl)cc4c(N5C6CCC5CNC6)nc(OC[C@@]56CCCN5C[C@H](F)C6)nc4c3F)ccc(F)c2s1]

Provided herein as embodiment 357 is the compound according to embodiment 1, or a pharmaceutically acceptable salt thereof, wherein the compound is:

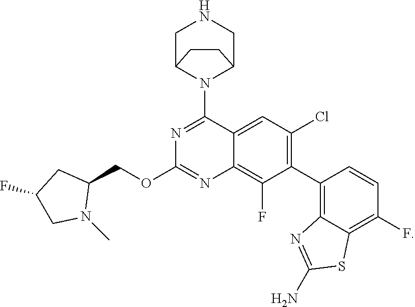 [SMILES: CN1C[C@H](F)C[C@H]1COc1nc(N2C3CCC2CNC3)c2cc(Cl)c(-c3ccc(F)c4sc(N)nc34)c(F)c2n1]

Provided herein as embodiment 358 is the compound according to embodiment 1, or a pharmaceutically acceptable salt thereof, wherein the compound is:

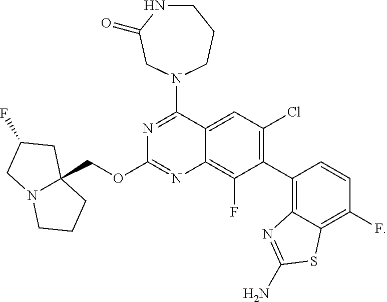 [SMILES: Nc1nc2c(-c3c(Cl)cc4c(N5CCCNC(=O)C5)nc(OC[C@@]56CCCN5C[C@H](F)C6)nc4c3F)ccc(F)c2s1]

Provided herein as embodiment 359 is the compound according to embodiment 1, or a pharmaceutically acceptable salt thereof, wherein the compound is:

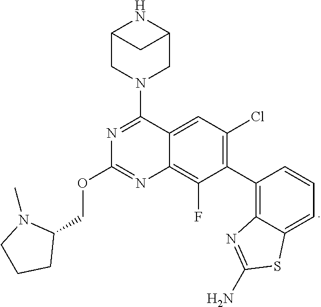 [SMILES: CN1CCC[C@H]1COc1nc(N2CC3CC(C2)N3)c2cc(Cl)c(-c3cccc4sc(N)nc34)c(F)c2n1 ; "."]

Provided herein as embodiment 360 is the compound according to embodiment 1, or a pharmaceutically acceptable salt thereof, wherein the compound is:

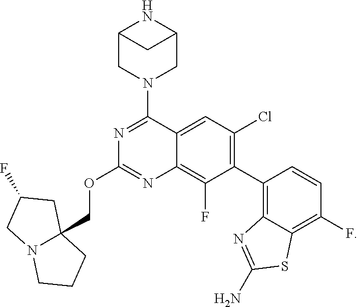 [SMILES: Nc1nc2c(-c3c(Cl)cc4c(N5CC6CC(C5)N6)nc(OC[C@@]56CCCN5C[C@H](F)C6)nc4c3F)ccc(F)c2s1]

Provided herein as embodiment 361 is the compound according to embodiment 1, or a pharmaceutically acceptable salt thereof, wherein the compound is:

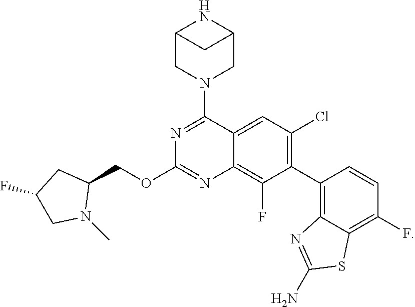 [SMILES: CN1C[C@H](F)C[C@H]1COc1nc(N2CC3CC(C2)N3)c2cc(Cl)c(-c3ccc(F)c4sc(N)nc34)c(F)c2n1]

Provided herein as embodiment 362 is the compound according to embodiment 1, or a pharmaceutically acceptable salt thereof, wherein the compound is:

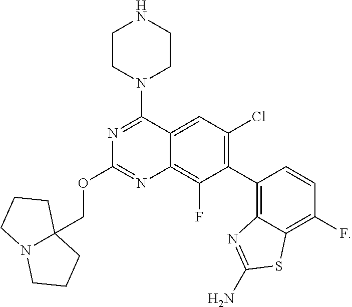 [SMILES: Nc1nc2c(-c3c(Cl)cc4c(N5CCNCC5)nc(OCC56CCCN5CCC6)nc4c3F)ccc(F)c2s1]

Provided herein as embodiment 363 is the compound according to embodiment 1, or a pharmaceutically acceptable salt thereof, wherein the compound is:

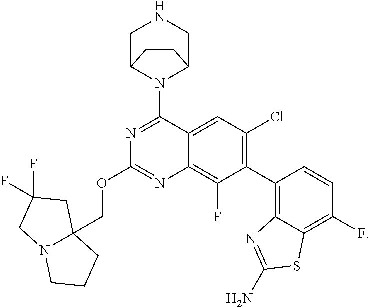 [SMILES: Nc1nc2c(-c3c(Cl)cc4c(N5C6CCC5CNC6)nc(OCC56CCCN5CC(F)(F)C6)nc4c3F)ccc(F)c2s1]

Provided herein as embodiment 364 is the compound according to embodiment 1, or a pharmaceutically acceptable salt thereof, wherein the compound is:

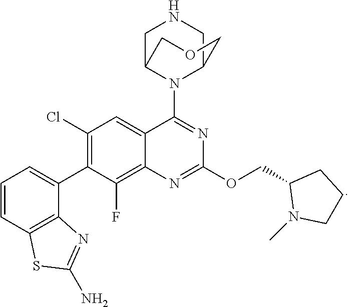 [SMILES: CN1CCC[C@H]1COc1nc(N2C3CNCC2COC3)c2cc(Cl)c(-c3cccc4sc(N)nc34)c(F)c2n1 ; "."]

Provided herein as embodiment 365 is the compound according to embodiment 1, or a pharmaceutically acceptable salt thereof, wherein the compound is:

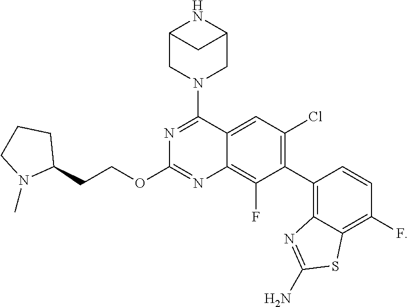 [SMILES: CN1CCC[C@H]1CCOc1nc(N2CC3CC(C2)N3)c2cc(Cl)c(-c3ccc(F)c4sc(N)nc34)c(F)c2n1]

Provided herein as embodiment 366 is the compound according to embodiment 1, or a pharmaceutically acceptable salt thereof, wherein the compound is:

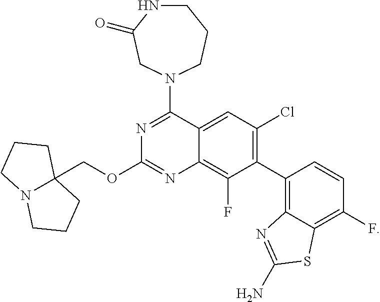 [SMILES: Nc1nc2c(-c3c(Cl)cc4c(N5CCCNC(=O)C5)nc(OCC56CCCN5CCC6)nc4c3F)ccc(F)c2s1]

Provided herein as embodiment 367 is the compound according to embodiment 1, or a pharmaceutically acceptable salt thereof, wherein the compound is:

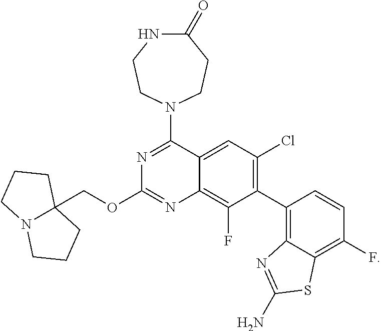 [SMILES: Nc1nc2c(-c3c(Cl)cc4c(N5CCNC(=O)CC5)nc(OCC56CCCN5CCC6)nc4c3F)ccc(F)c2s1]

Provided herein as embodiment 368 is the compound according to embodiment 1, or a pharmaceutically acceptable salt thereof, wherein the compound is:

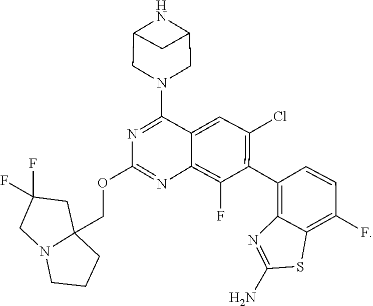 [SMILES: Nc1nc2c(-c3c(Cl)cc4c(N5CC6CC(C5)N6)nc(OCC56CCCN5CC(F)(F)C6)nc4c3F)ccc(F)c2s1]

Provided herein as embodiment 369 is the compound according to embodiment 1, or a pharmaceutically acceptable salt thereof, wherein the compound is:

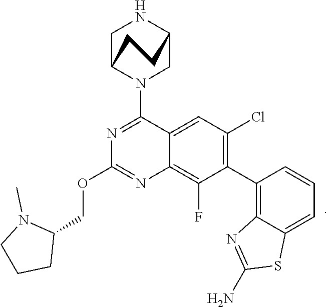

Provided herein as embodiment 370 is the compound according to embodiment 1, or a pharmaceutically acceptable salt thereof, wherein the compound is:

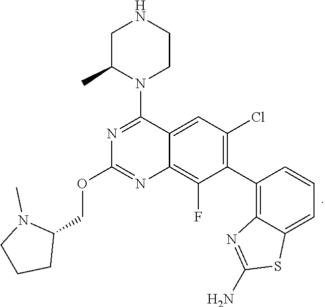 [SMILES: C[C@H]1CNCCN1c1nc(OC[C@@H]2CCCN2C)nc2c(F)c(-c3cccc4sc(N)nc34)c(Cl)cc12]

Provided herein as embodiment 371 is the compound according to embodiment 1, or a pharmaceutically acceptable salt thereof, wherein the compound is:

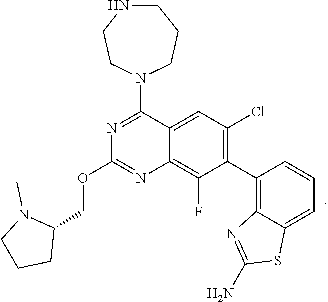 [SMILES: CN1CCC[C@H]1COc1nc(N2CCCNCC2)c2cc(Cl)c(-c3cccc4sc(N)nc34)c(F)c2n1]

Provided herein as embodiment 372 is the compound according to embodiment 1, or a pharmaceutically acceptable salt thereof, wherein the compound is:

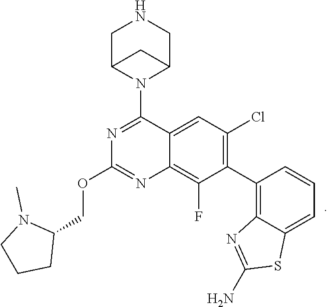

Provided herein as embodiment 373 is the compound according to embodiment 1, or a pharmaceutically acceptable salt thereof, wherein the compound is:

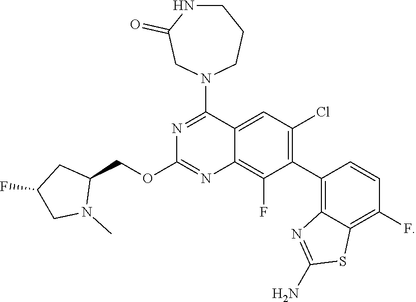 [SMILES: CN1C[C@H](F)C[C@H]1COc1nc(N2CCCNC(=O)C2)c2cc(Cl)c(-c3ccc(F)c4sc(N)nc34)c(F)c2n1]

Provided herein as embodiment 374 is the compound according to embodiment 1, or a pharmaceutically acceptable salt thereof, wherein the compound is:

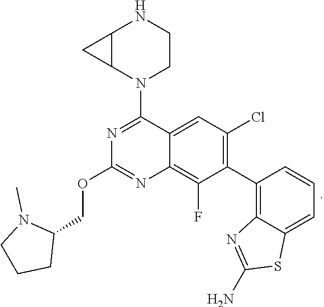 [SMILES: CN1CCC[C@H]1COc1nc(N2CCNC3CC32)c2cc(Cl)c(-c3cccc4sc(N)nc34)c(F)c2n1]

Provided herein as embodiment 375 is the compound according to embodiment 1, or a pharmaceutically acceptable salt thereof, wherein the compound is:

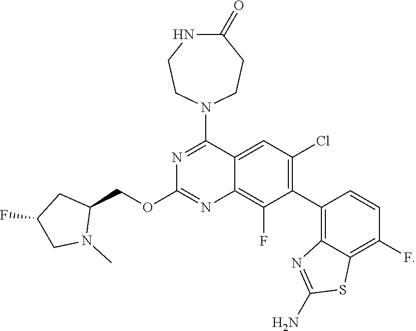 [SMILES: CN1C[C@H](F)C[C@H]1COc1nc(N2CCNC(=O)CC2)c2cc(Cl)c(-c3ccc(F)c4sc(N)nc34)c(F)c2n1]

Provided herein as embodiment 376 is the compound according to embodiment 1, or a pharmaceutically acceptable salt thereof, wherein the compound is:

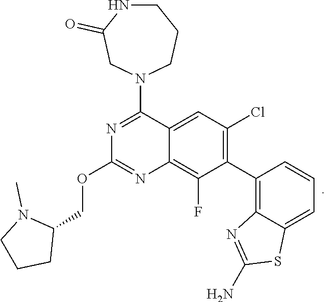 [SMILES: CN1CCC[C@H]1COc1nc(N2CCCNC(=O)C2)c2cc(Cl)c(-c3cccc4sc(N)nc34)c(F)c2n1]

Provided herein as embodiment 377 is the compound according to embodiment 1, or a pharmaceutically acceptable salt thereof, wherein the compound is:

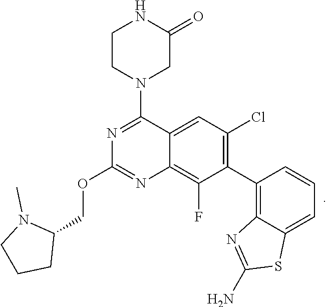 [SMILES: CN1CCC[C@H]1COc1nc(N2CCNC(=O)C2)c2cc(Cl)c(-c3cccc4sc(N)nc34)c(F)c2n1]

Provided herein as embodiment 378 is the compound according to embodiment 1, or a pharmaceutically acceptable salt thereof, wherein the compound is:

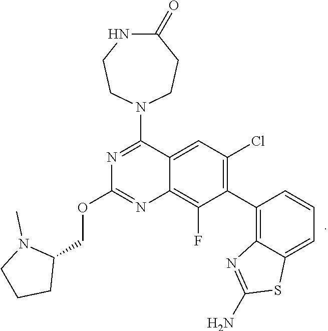 [SMILES: CN1CCC[C@H]1COc1nc(N2CCNC(=O)CC2)c2cc(Cl)c(-c3cccc4sc(N)nc34)c(F)c2n1]

Provided herein as embodiment 379 is the compound according to embodiment 1, or a pharmaceutically acceptable salt thereof, wherein the compound is:

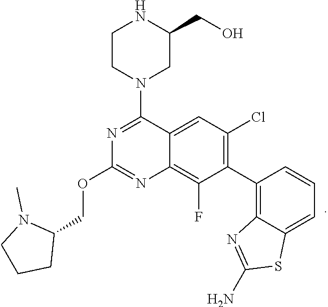 [SMILES: CN1CCC[C@H]1COc1nc(N2CCN[C@@H](CO)C2)c2cc(Cl)c(-c3cccc4sc(N)nc34)c(F)c2n1]

Provided herein as embodiment 380 is the compound according to embodiment 1, or a pharmaceutically acceptable salt thereof, wherein the compound is:

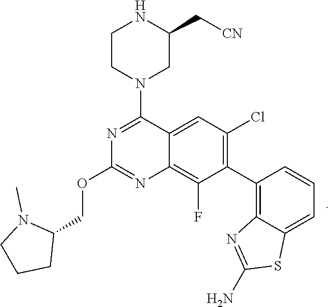 [SMILES: CN1CCC[C@H]1COc1nc(N2CCN[C@@H](CC#N)C2)c2cc(Cl)c(-c3cccc4sc(N)nc34)c(F)c2n1]

Provided herein as embodiment 381 is the compound according to embodiment 1, or a pharmaceutically acceptable salt thereof, wherein the compound is:

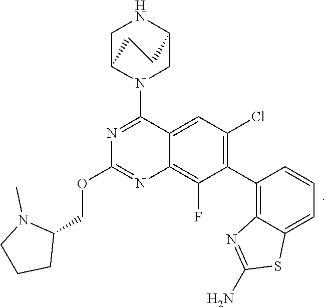

Provided herein as embodiment 382 is the compound according to embodiment 1, or a pharmaceutically acceptable salt thereof, wherein the compound is:

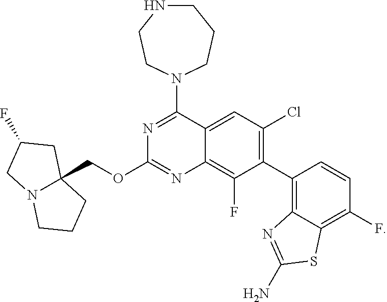 [SMILES: Nc1nc2c(-c3c(Cl)cc4c(N5CCCNCC5)nc(OC[C@@]56CCCN5C[C@H](F)C6)nc4c3F)ccc(F)c2s1]

Provided herein as embodiment 383 is the compound according to embodiment 1, or a pharmaceutically acceptable salt thereof, wherein the compound is:

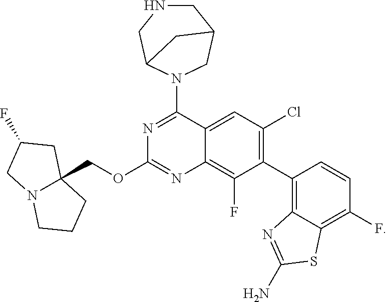 [SMILES: Nc1nc2c(-c3c(Cl)cc4c(N5CC6CNCC5C6)nc(OC[C@@]56CCCN5C[C@H](F)C6)nc4c3F)ccc(F)c2s1]

Provided herein as embodiment 384 is the compound according to embodiment 1, or a pharmaceutically acceptable salt thereof, wherein the compound is:

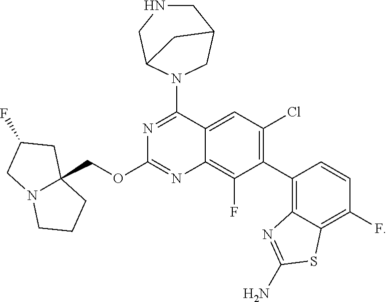 [SMILES: Nc1nc2c(-c3c(Cl)cc4c(N5CC6CNCC5C6)nc(OC[C@@]56CCCN5C[C@H](F)C6)nc4c3F)ccc(F)c2s1]

Provided herein as embodiment 385 is the compound according to embodiment 1, or a pharmaceutically acceptable salt thereof, wherein the compound is:

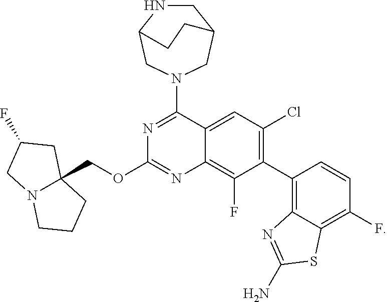 [SMILES: Nc1nc2c(-c3c(Cl)cc4c(N5CC6CCC(C5)NC6)nc(OC[C@@]56CCCN5C[C@H](F)C6)nc4c3F)ccc(F)c2s1]

Provided herein as embodiment 386 is the compound according to embodiment 1, or a pharmaceutically acceptable salt thereof, wherein the compound is:

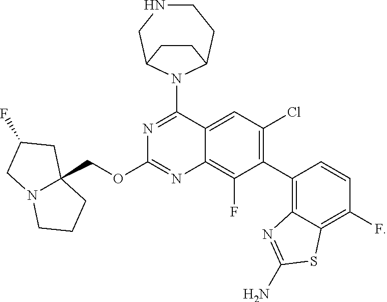 [SMILES: Nc1nc2c(-c3c(Cl)cc4c(N5C6CCNCC5CC6)nc(OC[C@@]56CCCN5C[C@H](F)C6)nc4c3F)ccc(F)c2s1]

Provided herein as embodiment 387 is the compound according to embodiment 1, or a pharmaceutically acceptable salt thereof, wherein the compound is:

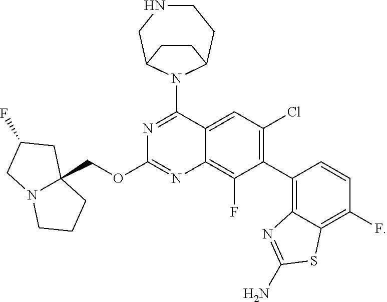 [SMILES: Nc1nc2c(-c3c(Cl)cc4c(N5C6CCNCC5CC6)nc(OC[C@@]56CCCN5C[C@H](F)C6)nc4c3F)ccc(F)c2s1]

Provided herein as embodiment 388 is the compound according to embodiment 1, or a pharmaceutically acceptable salt thereof, wherein the compound is:

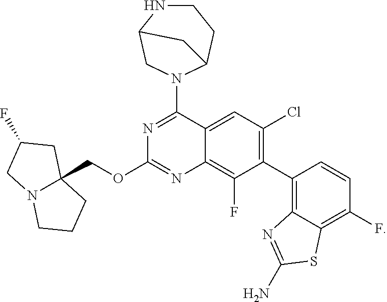 [SMILES: Nc1nc2c(-c3c(Cl)cc4c(N5CC6CC5CCN6)nc(OC[C@@]56CCCN5C[C@H](F)C6)nc4c3F)ccc(F)c2s1]

Provided herein as embodiment 389 is the compound according to embodiment 1, or a pharmaceutically acceptable salt thereof, wherein the compound is:

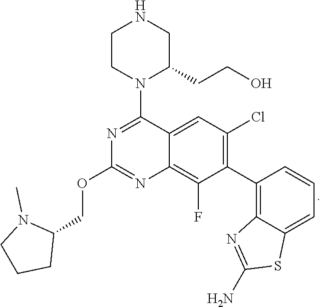 [SMILES: CN1CCC[C@H]1COc1nc(N2CCNC[C@@H]2CCO)c2cc(Cl)c(-c3cccc4sc(N)nc34)c(F)c2n1]

Provided herein as embodiment 390 is the compound according to embodiment 1, or a pharmaceutically acceptable salt thereof, wherein the compound is:

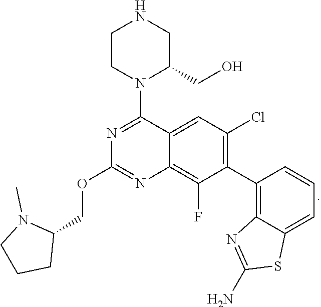 [SMILES: CN1CCC[C@H]1COc1nc(N2CCNC[C@@H]2CO)c2cc(Cl)c(-c3cccc4sc(N)nc34)c(F)c2n1]

Provided herein as embodiment 391 is the compound according to embodiment 1, or a pharmaceutically acceptable salt thereof, wherein the compound is:

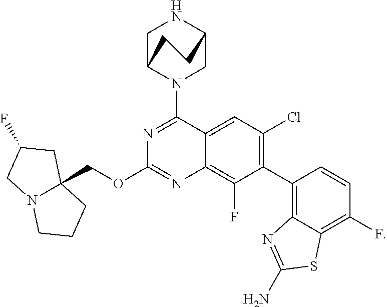 [SMILES: Nc1nc2c(-c3c(Cl)cc4c(N5C[C@@H]6CC[C@H]5CN6)nc(OC[C@@]56CCCN5C[C@H](F)C6)nc4c3F)ccc(F)c2s1]

Provided herein as embodiment 392 is the compound according to embodiment 1, or a pharmaceutically acceptable salt thereof, wherein the compound is:

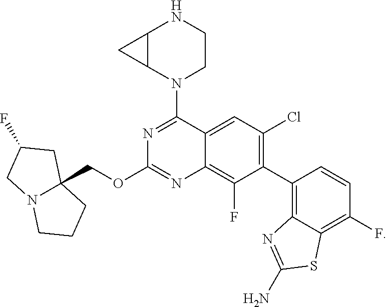 [SMILES: Nc1nc2c(-c3c(Cl)cc4c(N5CCNC6CC65)nc(OC[C@@]56CCCN5C[C@H](F)C6)nc4c3F)ccc(F)c2s1]

Provided herein as embodiment 393 is the compound according to embodiment 1, or a pharmaceutically acceptable salt thereof, wherein the compound is:

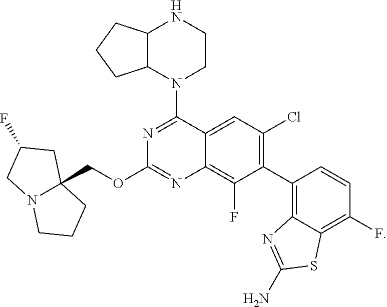 [SMILES: Nc1nc2c(-c3c(Cl)cc4c(N5CCNC6CCCC65)nc(OC[C@@]56CCCN5C[C@H](F)C6)nc4c3F)ccc(F)c2s1]

Provided herein as embodiment 394 is the compound according to embodiment 1, or a pharmaceutically acceptable salt thereof, wherein the compound is:

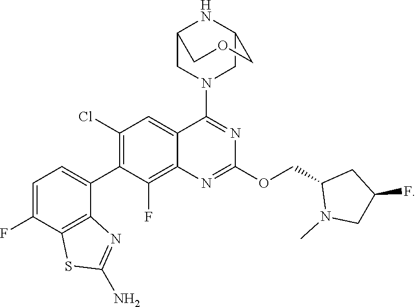 [SMILES: CN1C[C@H](F)C[C@H]1COc1nc(N2CC3COCC(C2)N3)c2cc(Cl)c(-c3ccc(F)c4sc(N)nc34)c(F)c2n1]

Provided herein as embodiment 395 is the compound according to embodiment 1, or a pharmaceutically acceptable salt thereof, wherein the compound is:

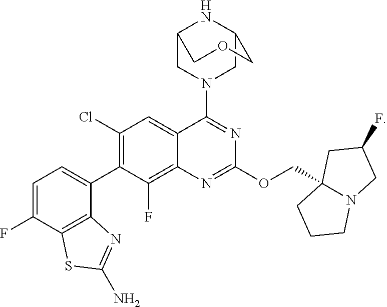 [SMILES: Nc1nc2c(-c3c(Cl)cc4c(N5CC6COCC(C5)N6)nc(OC[C@@]56CCCN5C[C@H](F)C6)nc4c3F)ccc(F)c2s1]

Provided herein as embodiment 396 is the compound according to embodiment 1, or a pharmaceutically acceptable salt thereof, wherein the compound is:

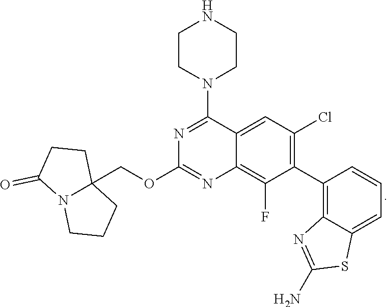 [SMILES: Nc1nc2c(-c3c(Cl)cc4c(N5CCNCC5)nc(OCC56CCCN5C(=O)CC6)nc4c3F)cccc2s1]

Provided herein as embodiment 397 is the compound according to embodiment 1, or a pharmaceutically acceptable salt thereof, wherein the compound is:

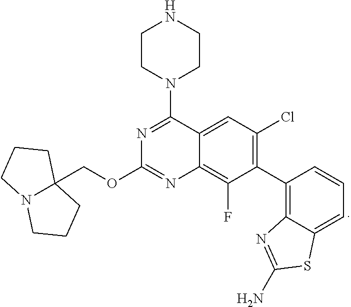 [SMILES: Nc1nc2c(-c3c(Cl)cc4c(N5CCNCC5)nc(OCC56CCCN5CCC6)nc4c3F)cccc2s1 ; "."]

Provided herein as embodiment 398 is the compound according to embodiment 1, or a pharmaceutically acceptable salt thereof, wherein the compound is:

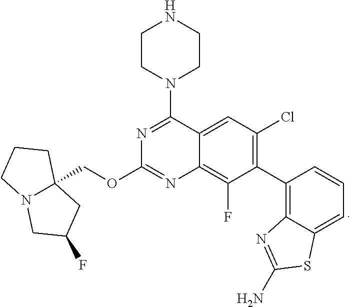 [SMILES: Nc1nc2c(-c3c(Cl)cc4c(N5CCNCC5)nc(OC[C@@]56CCCN5C[C@H](F)C6)nc4c3F)cccc2s1 ; "."]

Provided herein as embodiment 399 is the compound according to embodiment 1, or a pharmaceutically acceptable salt thereof, wherein the compound is:

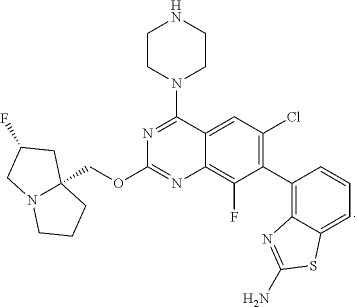 [SMILES: Nc1nc2c(-c3c(Cl)cc4c(N5CCNCC5)nc(OC[C@]56CCCN5C[C@H](F)C6)nc4c3F)cccc2s1 ; "."]

Provided herein as embodiment 400 is the compound according to embodiment 1, or a pharmaceutically acceptable salt thereof, wherein the compound is:

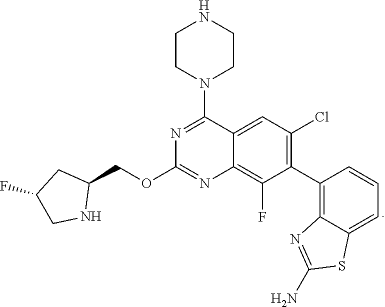 [SMILES: Nc1nc2c(-c3c(Cl)cc4c(N5CCNCC5)nc(OC[C@@H]5C[C@@H](F)CN5)nc4c3F)cccc2s1 ; "."]

Provided herein as embodiment 401 is the compound according to embodiment 1, or a pharmaceutically acceptable salt thereof, wherein the compound is:

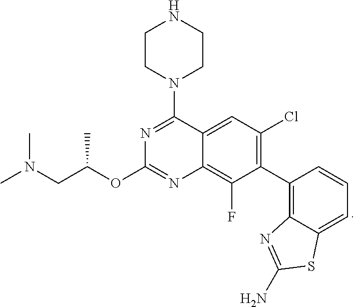 [SMILES: C[C@@H](CN(C)C)Oc1nc(N2CCNCC2)c2cc(Cl)c(-c3cccc4sc(N)nc34)c(F)c2n1 ; "."]

Provided herein as embodiment 402 is the compound according to embodiment 1, or a pharmaceutically acceptable salt thereof, wherein the compound is:

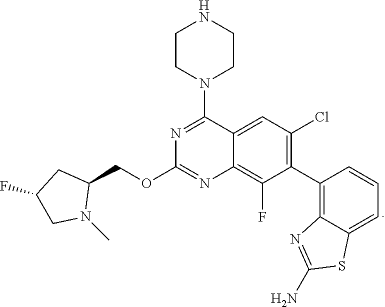 [SMILES: CN1C[C@H](F)C[C@H]1COc1nc(N2CCNCC2)c2cc(Cl)c(-c3cccc4sc(N)nc34)c(F)c2n1 ; "."]

Provided herein as embodiment 403 is the compound according to embodiment 1, or a pharmaceutically acceptable salt thereof, wherein the compound is:

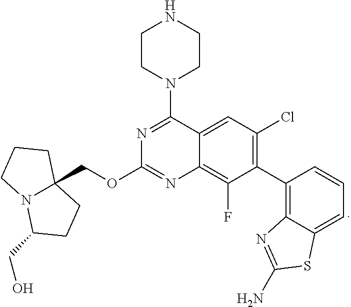 [SMILES: Nc1nc2c(-c3c(Cl)cc4c(N5CCNCC5)nc(OC[C@]56CCCN5[C@@H](CO)CC6)nc4c3F)cccc2s1 ; "."]

Provided herein as embodiment 404 is the compound according to embodiment 1, or a pharmaceutically acceptable salt thereof, wherein the compound is:

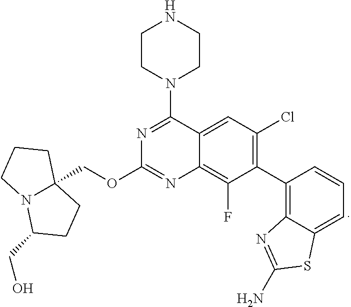 [SMILES: Nc1nc2c(-c3c(Cl)cc4c(N5CCNCC5)nc(OC[C@@]56CCCN5[C@@H](CO)CC6)nc4c3F)cccc2s1 ; "."]

Provided herein as embodiment 405 is the compound according to embodiment 1, or a pharmaceutically acceptable salt thereof, wherein the compound is:

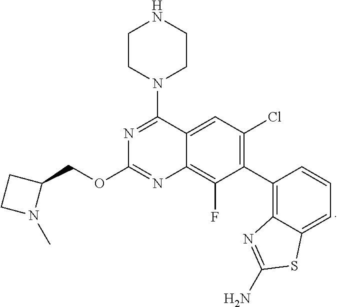 [SMILES: CN1CC[C@H]1COc1nc(N2CCNCC2)c2cc(Cl)c(-c3cccc4sc(N)nc34)c(F)c2n1 ; "."]

Provided herein as embodiment 406 is the compound according to embodiment 1, or a pharmaceutically acceptable salt thereof, wherein the compound is:

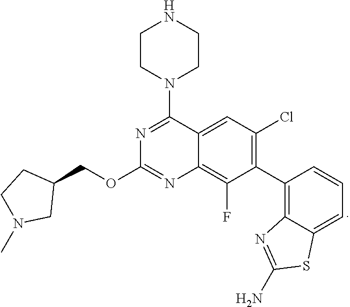 [SMILES: CN1CC[C@@H](COc2nc(N3CCNCC3)c3cc(Cl)c(-c4cccc5sc(N)nc45)c(F)c3n2)C1 ; "."]

Provided herein as embodiment 407 is the compound according to embodiment 1, or a pharmaceutically acceptable salt thereof, wherein the compound is:

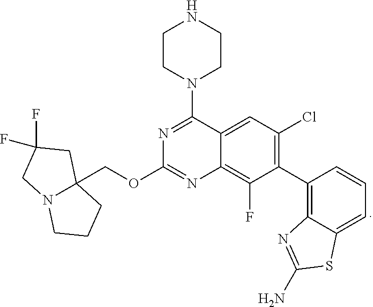 [SMILES: Nc1nc2c(-c3c(Cl)cc4c(N5CCNCC5)nc(OCC56CCCN5CC(F)(F)C6)nc4c3F)cccc2s1 ; "."]

Provided herein as embodiment 408 is the compound according to embodiment 1, or a pharmaceutically acceptable salt thereof, wherein the compound is:

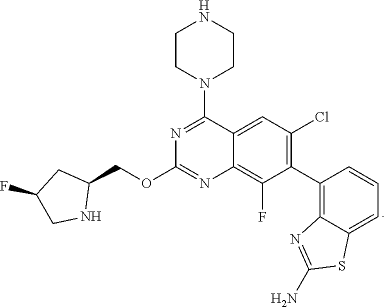 [SMILES: Nc1nc2c(-c3c(Cl)cc4c(N5CCNCC5)nc(OC[C@@H]5C[C@H](F)CN5)nc4c3F)cccc2s1 ; "."]

Provided herein as embodiment 409 is the compound according to embodiment 1, or a pharmaceutically acceptable salt thereof, wherein the compound is:

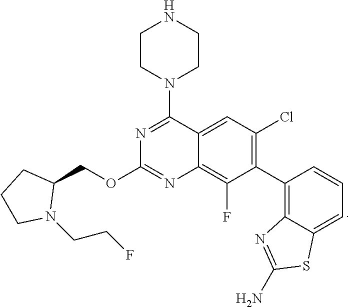 [SMILES: Nc1nc2c(-c3c(Cl)cc4c(N5CCNCC5)nc(OC[C@@H]5CCCN5CCF)nc4c3F)cccc2s1 ; "."]

Provided herein as embodiment 410 is the compound according to embodiment 1, or a pharmaceutically acceptable salt thereof, wherein the compound is:

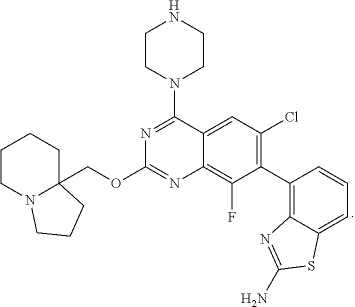 [SMILES: Nc1nc2c(-c3c(Cl)cc4c(N5CCNCC5)nc(OCC56CCCCN5CCC6)nc4c3F)cccc2s1 ; "."]

Provided herein as embodiment 411 is the compound according to embodiment 1, or a pharmaceutically acceptable salt thereof, wherein the compound is:

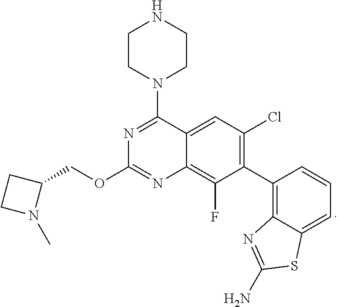 [SMILES: CN1CC[C@@H]1COc1nc(N2CCNCC2)c2cc(Cl)c(-c3cccc4sc(N)nc34)c(F)c2n1 ; "."]

Provided herein as embodiment 412 is the compound according to embodiment 1, or a pharmaceutically acceptable salt thereof, wherein the compound is:

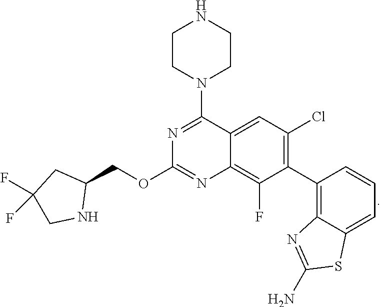 [SMILES: Nc1nc2c(-c3c(Cl)cc4c(N5CCNCC5)nc(OC[C@@H]5CC(F)(F)CN5)nc4c3F)cccc2s1]

Provided herein as embodiment 413 is the compound according to embodiment 1, or a pharmaceutically acceptable salt thereof, wherein the compound is:

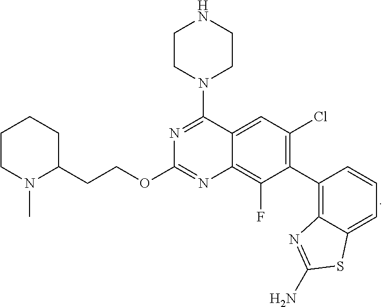 [SMILES: CN1CCCCC1CCOc1nc(N2CCNCC2)c2cc(Cl)c(-c3cccc4sc(N)nc34)c(F)c2n1]

Provided herein as embodiment 414 is the compound according to embodiment 1, or a pharmaceutically acceptable salt thereof, wherein the compound is:

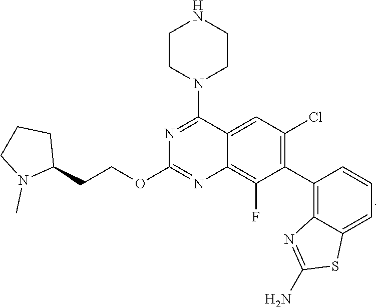 [SMILES: CN1CCC[C@H]1CCOc1nc(N2CCNCC2)c2cc(Cl)c(-c3cccc4sc(N)nc34)c(F)c2n1]

Provided herein as embodiment 415 is the compound according to embodiment 1, or a pharmaceutically acceptable salt thereof, wherein the compound is:

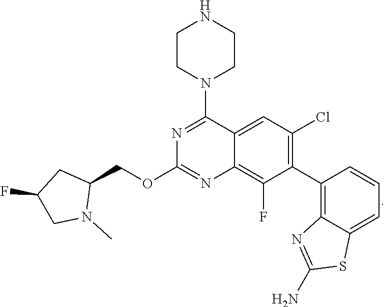 [SMILES: CN1C[C@@H](F)C[C@H]1COc1nc(N2CCNCC2)c2cc(Cl)c(-c3cccc4sc(N)nc34)c(F)c2n1]

Provided herein as embodiment 416 is the compound according to embodiment 1, or a pharmaceutically acceptable salt thereof, wherein the compound is:

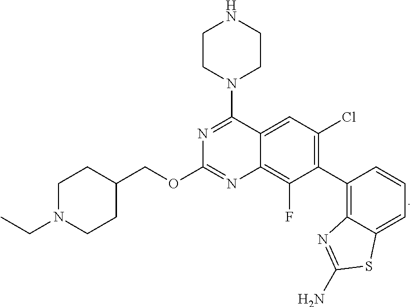 [SMILES: CCN1CCC(COc2nc(N3CCNCC3)c3cc(Cl)c(-c4cccc5sc(N)nc45)c(F)c3n2)CC1]

Provided herein as embodiment 417 is the compound according to embodiment 1, or a pharmaceutically acceptable salt thereof, wherein the compound is:

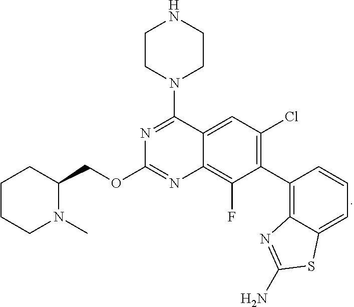 [SMILES: CN1CCCC[C@H]1COc1nc(N2CCNCC2)c2cc(Cl)c(-c3cccc4sc(N)nc34)c(F)c2n1]

Provided herein as embodiment 418 is the compound according to embodiment 1, or a pharmaceutically acceptable salt thereof, wherein the compound is:

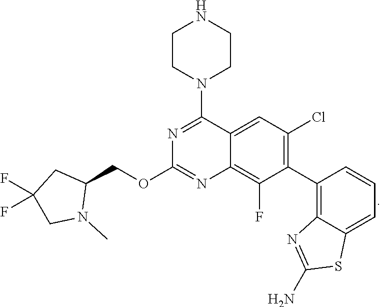 [SMILES: CN1CC(F)(F)C[C@H]1COc1nc(N2CCNCC2)c2cc(Cl)c(-c3cccc4sc(N)nc34)c(F)c2n1]

Provided herein as embodiment 419 is the compound according to embodiment 1, or a pharmaceutically acceptable salt thereof, wherein the compound is:

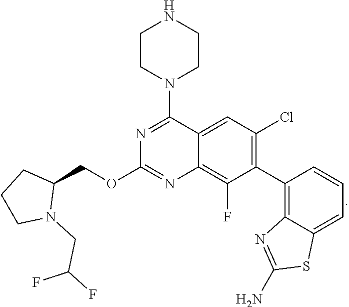 [SMILES: Nc1nc2c(-c3c(Cl)cc4c(N5CCNCC5)nc(OC[C@@H]5CCCN5CC(F)F)nc4c3F)cccc2s1]

Provided herein as embodiment 420 is the compound according to embodiment 1, or a pharmaceutically acceptable salt thereof, wherein the compound is:

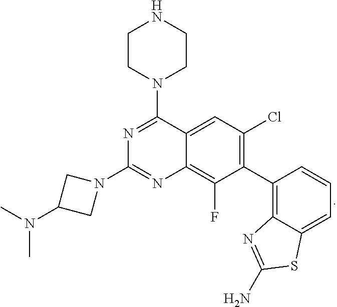 [SMILES: CN(C)C1CN(c2nc(N3CCNCC3)c3cc(Cl)c(-c4cccc5sc(N)nc45)c(F)c3n2)C1]

Provided herein as embodiment 421 is the compound according to embodiment 1, or a pharmaceutically acceptable salt thereof, wherein the compound is:

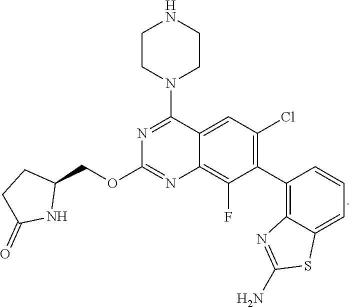 [SMILES: Nc1nc2c(-c3c(Cl)cc4c(N5CCNCC5)nc(OC[C@@H]5CCC(=O)N5)nc4c3F)cccc2s1]

Provided herein as embodiment 422 is the compound according to embodiment 1, or a pharmaceutically acceptable salt thereof, wherein the compound is:

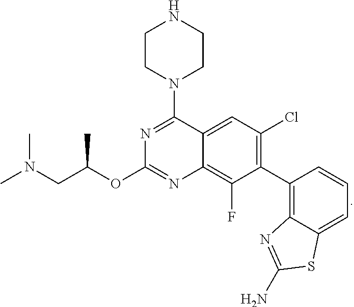 [SMILES: C[C@H](CN(C)C)Oc1nc(N2CCNCC2)c2cc(Cl)c(-c3cccc4sc(N)nc34)c(F)c2n1]

Provided herein as embodiment 423 is the compound according to embodiment 1, or a pharmaceutically acceptable salt thereof, wherein the compound is:

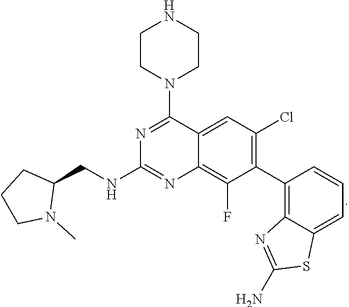 [SMILES: CN1CCC[C@H]1CNc1nc(N2CCNCC2)c2cc(Cl)c(-c3cccc4sc(N)nc34)c(F)c2n1]

Provided herein as embodiment 424 is the compound according to embodiment 1, or a pharmaceutically acceptable salt thereof, wherein the compound is:

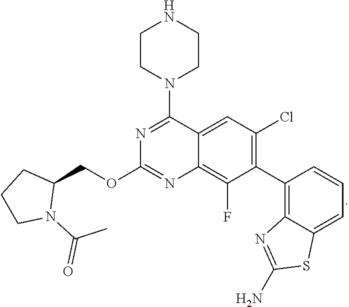 [SMILES: CC(=O)N1CCC[C@H]1COc1nc(N2CCNCC2)c2cc(Cl)c(-c3cccc4sc(N)nc34)c(F)c2n1]

Provided herein as embodiment 425 is the compound according to embodiment 1, or a pharmaceutically acceptable salt thereof, wherein the compound is:

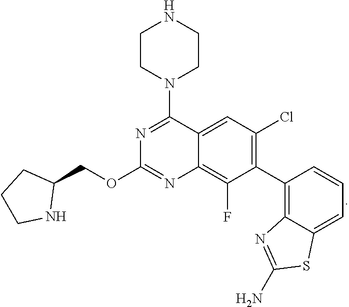 [SMILES: Nc1nc2c(-c3c(Cl)cc4c(N5CCNCC5)nc(OC[C@@H]5CCCN5)nc4c3F)cccc2s1]

Provided herein as embodiment 426 is the compound according to embodiment 1, or a pharmaceutically acceptable salt thereof, wherein the compound is:

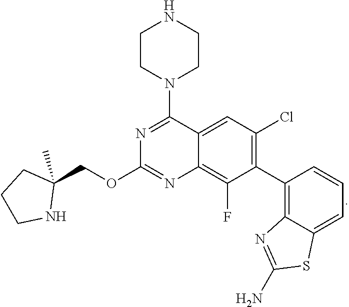 [SMILES: C[C@@]1(COc2nc(N3CCNCC3)c3cc(Cl)c(-c4cccc5sc(N)nc45)c(F)c3n2)CCCN1]

Provided herein as embodiment 427 is the compound according to embodiment 1, or a pharmaceutically acceptable salt thereof, wherein the compound is:

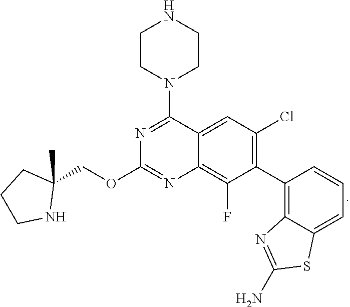 [SMILES: C[C@]1(COc2nc(N3CCNCC3)c3cc(Cl)c(-c4cccc5sc(N)nc45)c(F)c3n2)CCCN1]

Provided herein as embodiment 428 is the compound according to embodiment 1, or a pharmaceutically acceptable salt thereof, wherein the compound is:

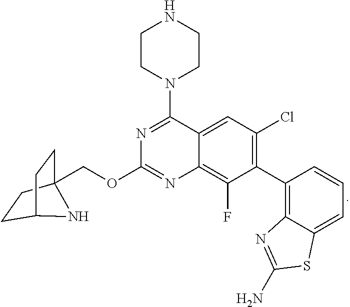 [SMILES: Nc1nc2c(-c3c(Cl)cc4c(N5CCNCC5)nc(OCC56CCC(CC5)N6)nc4c3F)cccc2s1]

Provided herein as embodiment 429 is the compound according to embodiment 1, or a pharmaceutically acceptable salt thereof, wherein the compound is:

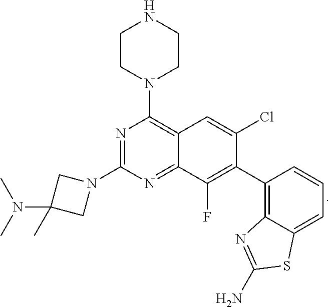 [SMILES: CN(C)C1(C)CN(c2nc(N3CCNCC3)c3cc(Cl)c(-c4cccc5sc(N)nc45)c(F)c3n2)C1]

Provided herein as embodiment 430 is the compound according to embodiment 1, or a pharmaceutically acceptable salt thereof, wherein the compound is:

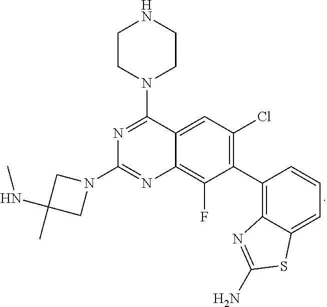 [SMILES: CNC1(C)CN(c2nc(N3CCNCC3)c3cc(Cl)c(-c4cccc5sc(N)nc45)c(F)c3n2)C1]

Provided herein as embodiment 431 is the compound according to embodiment 1, or a pharmaceutically acceptable salt thereof, wherein the compound is:

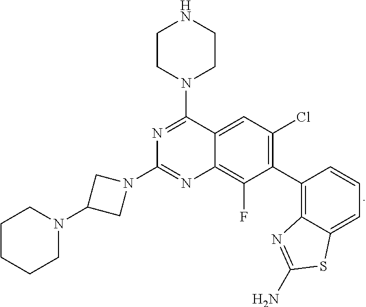 [SMILES: Nc1nc2c(-c3c(Cl)cc4c(N5CCNCC5)nc(N5CC(N6CCCCC6)C5)nc4c3F)cccc2s1]

Provided herein as embodiment 432 is the compound according to embodiment 1, or a pharmaceutically acceptable salt thereof, wherein the compound is:

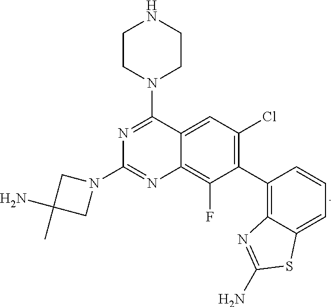 [SMILES: CC1(N)CN(c2nc(N3CCNCC3)c3cc(Cl)c(-c4cccc5sc(N)nc45)c(F)c3n2)C1]

Provided herein as embodiment 433 is the compound according to embodiment 1, or a pharmaceutically acceptable salt thereof, wherein the compound is:

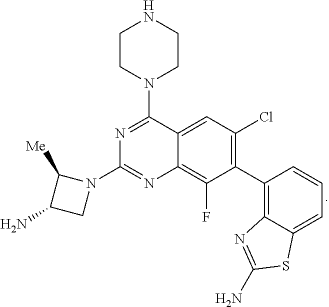 [SMILES: C[C@@H]1[C@@H](N)CN1c1nc(N2CCNCC2)c2cc(Cl)c(-c3cccc4sc(N)nc34)c(F)c2n1]

Provided herein as embodiment 434 is the compound according to embodiment 1, or a pharmaceutically acceptable salt thereof, wherein the compound is:

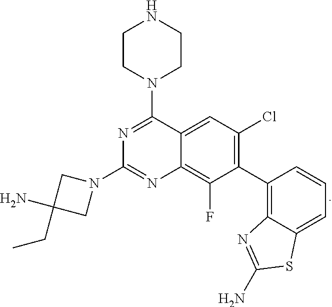 [SMILES: CCC1(N)CN(c2nc(N3CCNCC3)c3cc(Cl)c(-c4cccc5sc(N)nc45)c(F)c3n2)C1]

Provided herein as embodiment 435 is the compound according to embodiment 1, or a pharmaceutically acceptable salt thereof, wherein the compound is:

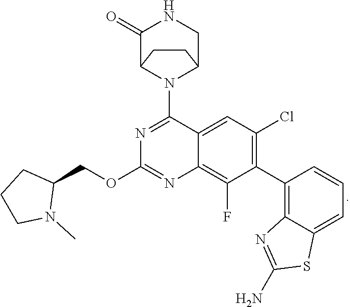 [SMILES: CN1CCC[C@H]1COc1nc(N2C3CCC2C(=O)NC3)c2cc(Cl)c(-c3cccc4sc(N)nc34)c(F)c2n1]

Provided herein as embodiment 436 is the compound according to embodiment 1, or a pharmaceutically acceptable salt thereof, wherein the compound is:

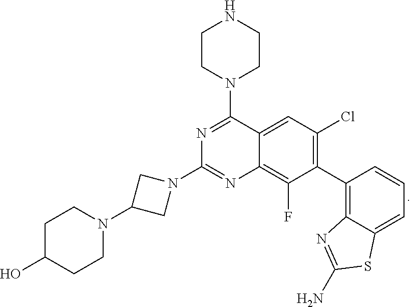 [SMILES: Nc1nc2c(-c3c(Cl)cc4c(N5CCNCC5)nc(N5CC(N6CCC(O)CC6)C5)nc4c3F)cccc2s1]

Provided herein as embodiment 437 is the compound according to embodiment 1, or a pharmaceutically acceptable salt thereof, wherein the compound is:

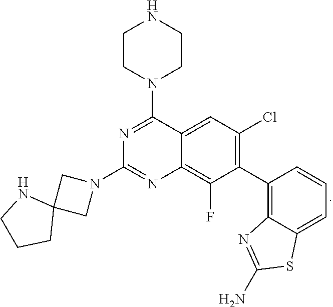 [SMILES: Nc1nc2c(-c3c(Cl)cc4c(N5CCNCC5)nc(N5CC6(CCCN6)C5)nc4c3F)cccc2s1]

Provided herein as embodiment 438 is the compound according to embodiment 1, or a pharmaceutically acceptable salt thereof, wherein the compound is:

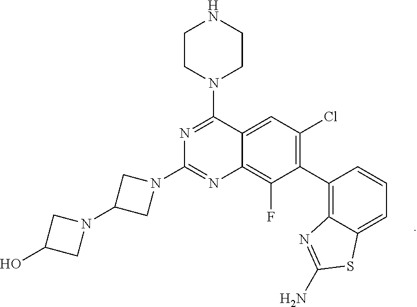 [SMILES: Nc1nc2c(-c3c(Cl)cc4c(N5CCNCC5)nc(N5CC(N6CC(O)C6)C5)nc4c3F)cccc2s1]

Provided herein as embodiment 439 is the compound according to embodiment 1, or a pharmaceutically acceptable salt thereof, wherein the compound is:

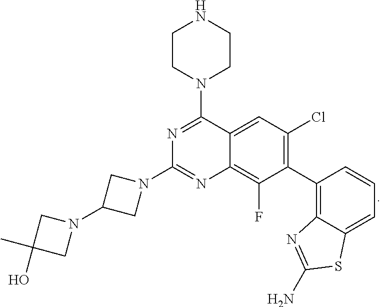 [SMILES: CC1(O)CN(C2CN(c3nc(N4CCNCC4)c4cc(Cl)c(-c5cccc6sc(N)nc56)c(F)c4n3)C2)C1]

Provided herein as embodiment 440 is the compound according to embodiment 1, or a pharmaceutically acceptable salt thereof, wherein the compound is:

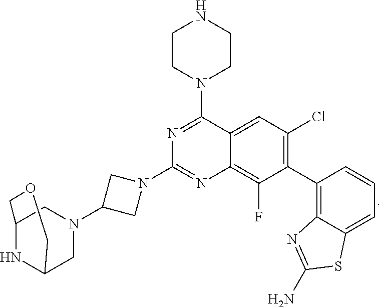 [SMILES: Nc1nc2c(-c3c(Cl)cc4c(N5CCNCC5)nc(N5CC(N6CC7COCC(C6)N7)C5)nc4c3F)cccc2s1]

Provided herein as embodiment 441 is the compound according to embodiment 1, or a pharmaceutically acceptable salt thereof, wherein the compound is:

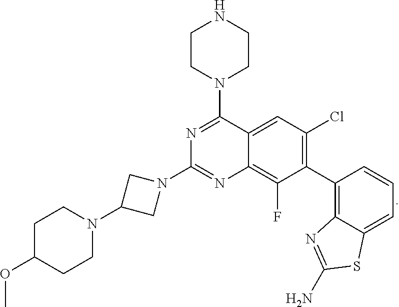 [SMILES: COC1CCN(C2CN(c3nc(N4CCNCC4)c4cc(Cl)c(-c5cccc6sc(N)nc56)c(F)c4n3)C2)CC1]

Provided herein as embodiment 442 is the compound according to embodiment 1, or a pharmaceutically acceptable salt thereof, wherein the compound is:

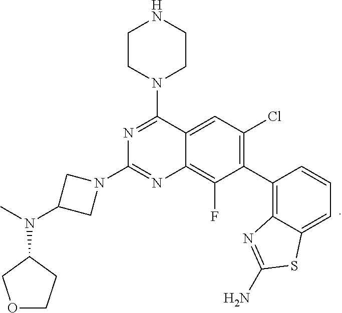

Provided herein as embodiment 443 is the compound according to embodiment 1, or a pharmaceutically acceptable salt thereof, wherein the compound is:

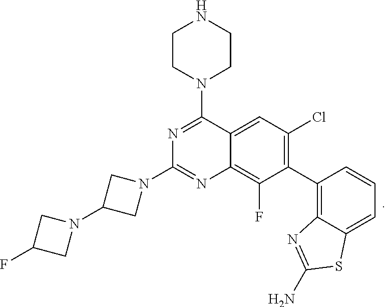

Provided herein as embodiment 444 is the compound according to embodiment 1, or a pharmaceutically acceptable salt thereof, wherein the compound is:

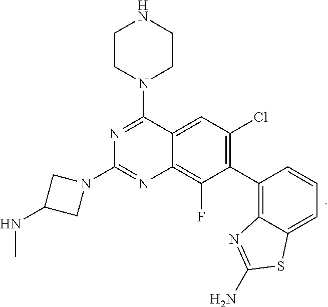

Provided herein as embodiment 445 is the compound according to embodiment 1, or a pharmaceutically acceptable salt thereof, wherein the compound is:

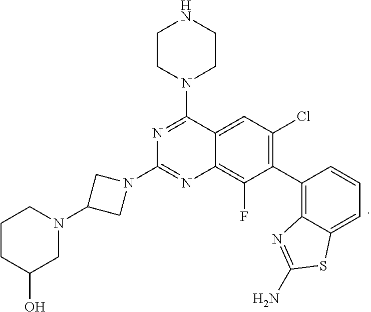

Provided herein as embodiment 446 is the compound according to embodiment 1, or a pharmaceutically acceptable salt thereof, wherein the compound is:

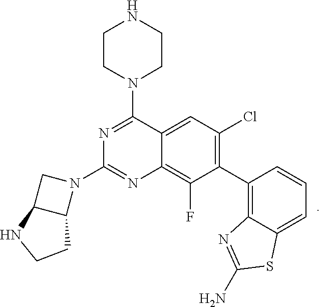

Provided herein as embodiment 447 is the compound according to embodiment 1, or a pharmaceutically acceptable salt thereof, wherein the compound is:

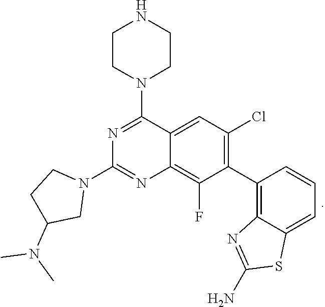

Provided herein as embodiment 448 is the compound according to embodiment 1, or a pharmaceutically acceptable salt thereof, wherein the compound is:

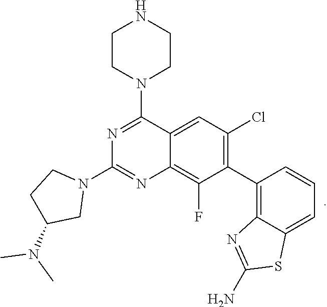

Provided herein as embodiment 449 is the compound according to embodiment 1, or a pharmaceutically acceptable salt thereof, wherein the compound is:

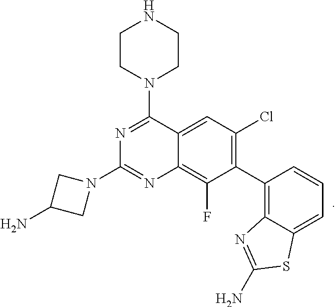

Provided herein as embodiment 450 is the compound according to embodiment 1, or a pharmaceutically acceptable salt thereof, wherein the compound is:

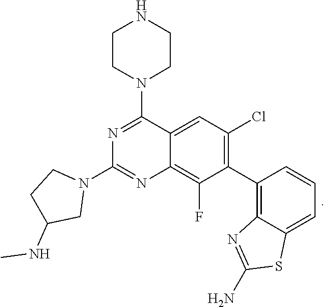

Provided herein as embodiment 451 is the compound according to embodiment 1, or a pharmaceutically acceptable salt thereof, wherein the compound is:

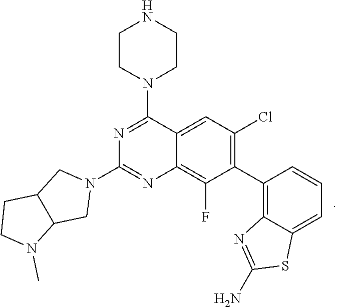

Provided herein as embodiment 452 is the compound according to embodiment 1, or a pharmaceutically acceptable salt thereof, wherein the compound is:

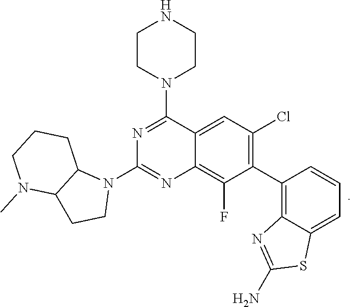

Provided herein as embodiment 453 is the compound according to embodiment 1, or a pharmaceutically acceptable salt thereof, wherein the compound is:

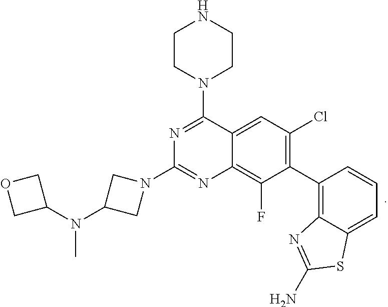

Provided herein as embodiment 454 is the compound according to embodiment 1, or a pharmaceutically acceptable salt thereof, wherein the compound is:

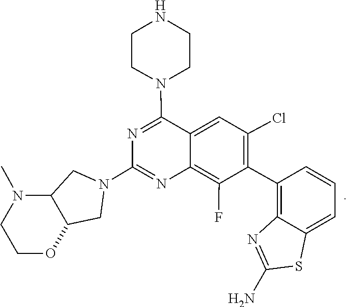

Provided herein as embodiment 455 is the compound according to embodiment 1, or a pharmaceutically acceptable salt thereof, wherein the compound is:

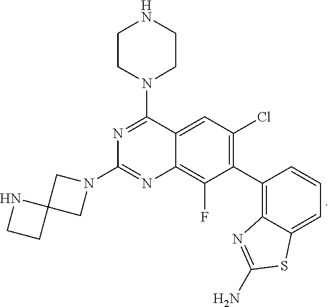

Provided herein as embodiment 456 is the compound according to embodiment 1, or a pharmaceutically acceptable salt thereof, wherein the compound is:

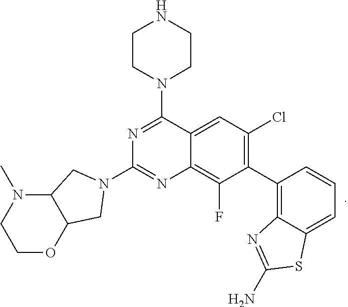

Provided herein as embodiment 457 is the compound according to embodiment 1, or a pharmaceutically acceptable salt thereof, wherein the compound is:

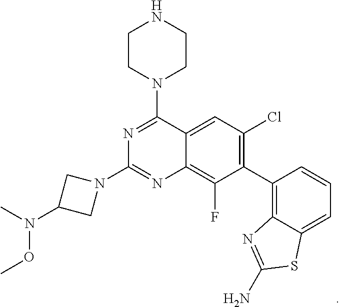 [SMILES: CON(C)C1CN(c2nc(N3CCNCC3)c3cc(Cl)c(-c4cccc5sc(N)nc45)c(F)c3n2)C1]

Provided herein as embodiment 458 is the compound according to embodiment 1, or a pharmaceutically acceptable salt thereof, wherein the compound is:

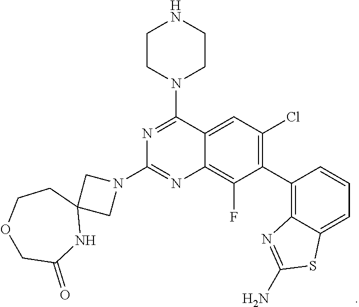 [SMILES: Nc1nc2c(-c3c(Cl)cc4c(N5CCNCC5)nc(N5CC6(CCOCC(=O)N6)C5)nc4c3F)cccc2s1]

Provided herein as embodiment 459 is the compound according to embodiment 1, or a pharmaceutically acceptable salt thereof, wherein the compound is:

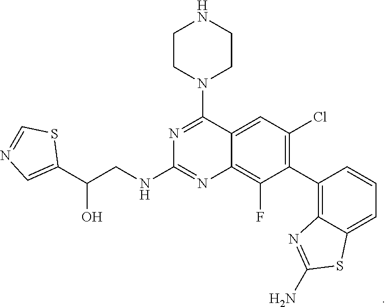 [SMILES: Nc1nc2c(-c3c(Cl)cc4c(N5CCNCC5)nc(NCC(O)c5cncs5)nc4c3F)cccc2s1]

Provided herein as embodiment 460 is the compound according to embodiment 1, or a pharmaceutically acceptable salt thereof, wherein the compound is:

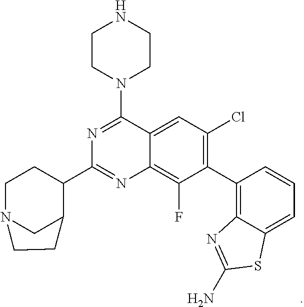 [SMILES: Nc1nc2c(-c3c(Cl)cc4c(N5CCNCC5)nc(C5CCN6CCC5C6)nc4c3F)cccc2s1]

Provided herein as embodiment 461 is the compound according to embodiment 1, or a pharmaceutically acceptable salt thereof, wherein the compound is:

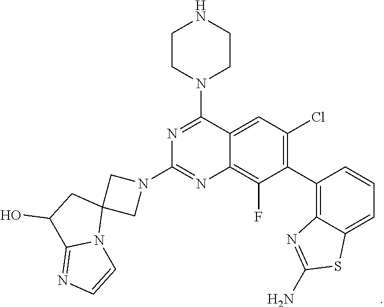 [SMILES: Nc1nc2c(-c3c(Cl)cc4c(N5CCNCC5)nc(N5CC6(CC(O)c7nccn76)C5)nc4c3F)cccc2s1]

Provided herein as embodiment 462 is the compound according to embodiment 1, or a pharmaceutically acceptable salt thereof, wherein the compound is:

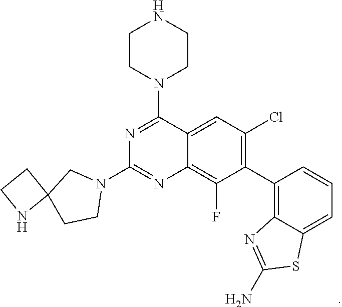 [SMILES: Nc1nc2c(-c3c(Cl)cc4c(N5CCNCC5)nc(N5CCC6(CCN6)C5)nc4c3F)cccc2s1]

Provided herein as embodiment 463 is the compound according to embodiment 1, or a pharmaceutically acceptable salt thereof, wherein the compound is:

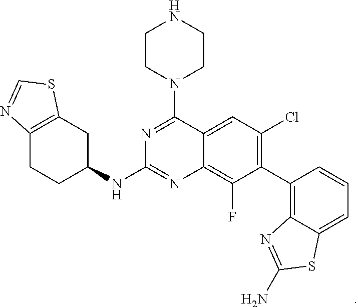 [SMILES: Nc1nc2c(-c3c(Cl)cc4c(N5CCNCC5)nc(N[C@H]5CCc6ncsc6C5)nc4c3F)cccc2s1]

Provided herein as embodiment 464 is the compound according to embodiment 1, or a pharmaceutically acceptable salt thereof, wherein the compound is:

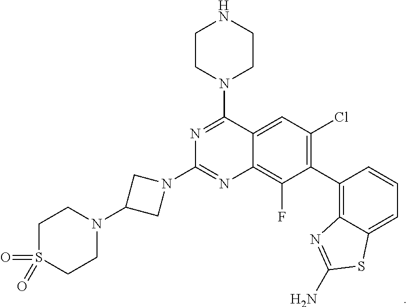 [SMILES: Nc1nc2c(-c3c(Cl)cc4c(N5CCNCC5)nc(N5CC(N6CCS(=O)(=O)CC6)C5)nc4c3F)cccc2s1]

Provided herein as embodiment 465 is the compound according to embodiment 1, or a pharmaceutically acceptable salt thereof, wherein the compound is:

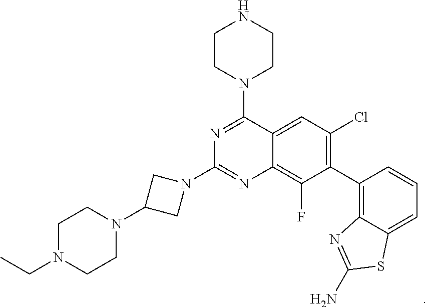 [SMILES: CCN1CCN(C2CN(c3nc(N4CCNCC4)c4cc(Cl)c(-c5cccc6sc(N)nc56)c(F)c4n3)C2)CC1]

Provided herein as embodiment 466 is the compound according to embodiment 1, or a pharmaceutically acceptable salt thereof, wherein the compound is:

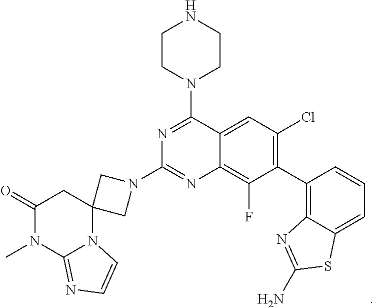 [SMILES: CN1C(=O)CC2(CN(c3nc(N4CCNCC4)c4cc(Cl)c(-c5cccc6sc(N)nc56)c(F)c4n3)C2)n2ccnc21]

Provided herein as embodiment 467 is the compound according to embodiment 1, or a pharmaceutically acceptable salt thereof, wherein the compound is:

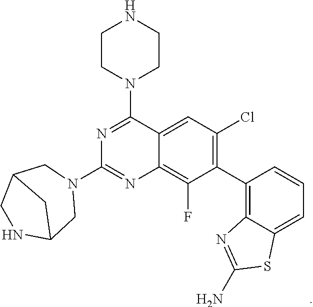 [SMILES: Nc1nc2c(-c3c(Cl)cc4c(N5CCNCC5)nc(N5CC6CNC(C6)C5)nc4c3F)cccc2s1]

Provided herein as embodiment 468 is the compound according to embodiment 1, or a pharmaceutically acceptable salt thereof, wherein the compound is:

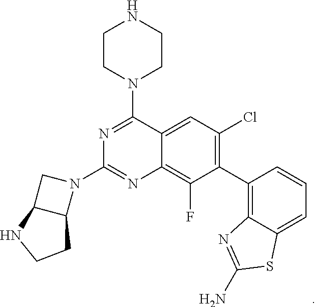 [SMILES: Nc1nc2c(-c3c(Cl)cc4c(N5CCNCC5)nc(N5C[C@@H]6NCC[C@@H]65)nc4c3F)cccc2s1]

Provided herein as embodiment 469 is the compound according to embodiment 1, or a pharmaceutically acceptable salt thereof, wherein the compound is:

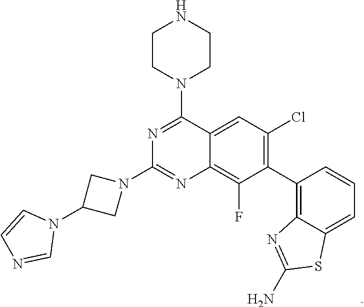 [SMILES: Nc1nc2c(-c3c(Cl)cc4c(N5CCNCC5)nc(N5CC(n6ccnc6)C5)nc4c3F)cccc2s1]

Provided herein as embodiment 470 is the compound according to embodiment 1, or a pharmaceutically acceptable salt thereof, wherein the compound is:

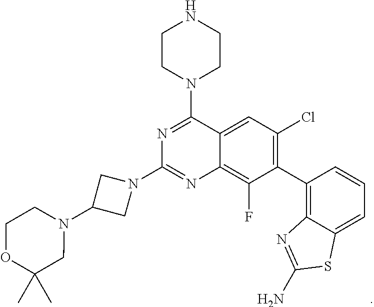 [SMILES: CC1(C)CN(C2CN(c3nc(N4CCNCC4)c4cc(Cl)c(-c5cccc6sc(N)nc56)c(F)c4n3)C2)CCO1]

Provided herein as embodiment 471 is the compound according to embodiment 1, or a pharmaceutically acceptable salt thereof, wherein the compound is:

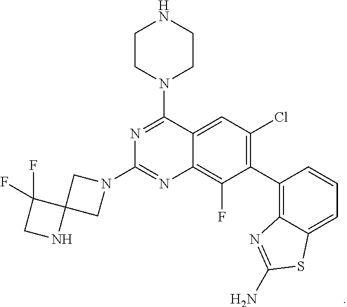 [SMILES: Nc1nc2c(-c3c(Cl)cc4c(N5CCNCC5)nc(N5CC6(C5)NCC6(F)F)nc4c3F)cccc2s1]

Provided herein as embodiment 472 is the compound according to embodiment 1, or a pharmaceutically acceptable salt thereof, wherein the compound is:

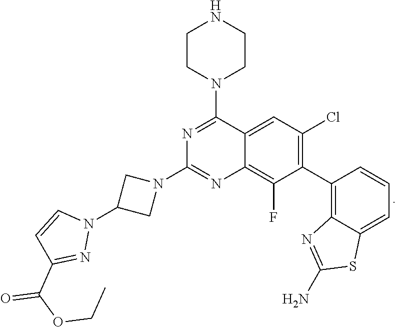 [SMILES: CCOC(=O)c1ccn(C2CN(c3nc(N4CCNCC4)c4cc(Cl)c(-c5cccc6sc(N)nc56)c(F)c4n3)C2)n1]

Provided herein as embodiment 473 is the compound according to embodiment 1, or a pharmaceutically acceptable salt thereof, wherein the compound is:

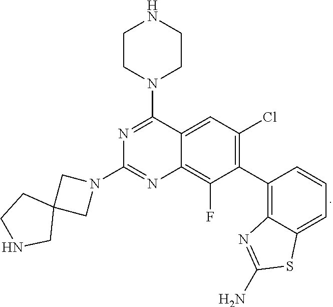 [SMILES: Nc1nc2c(-c3c(Cl)cc4c(N5CCNCC5)nc(N5CC6(CCNC6)C5)nc4c3F)cccc2s1]

Provided herein as embodiment 474 is the compound according to embodiment 1, or a pharmaceutically acceptable salt thereof, wherein the compound is:

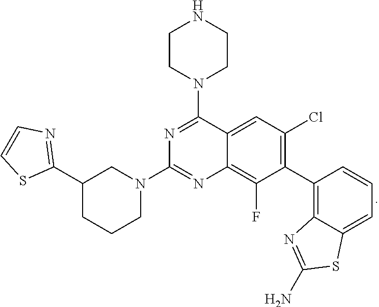 [SMILES: Nc1nc2c(-c3c(Cl)cc4c(N5CCNCC5)nc(N5CCCC(c6nccs6)C5)nc4c3F)cccc2s1]

Provided herein as embodiment 475 is the compound according to embodiment 1, or a pharmaceutically acceptable salt thereof, wherein the compound is:

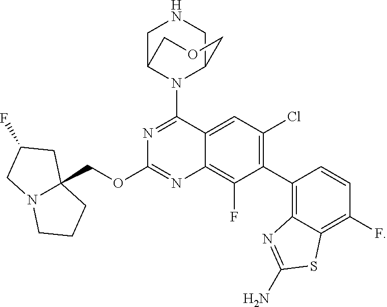 [SMILES: Nc1nc2c(-c3c(Cl)cc4c(N5C6CNCC5COC6)nc(OC[C@@]56CCCN5C[C@H](F)C6)nc4c3F)ccc(F)c2s1]

Provided herein as embodiment 476 is the compound according to embodiment 1, or a pharmaceutically acceptable salt thereof, wherein the compound is:

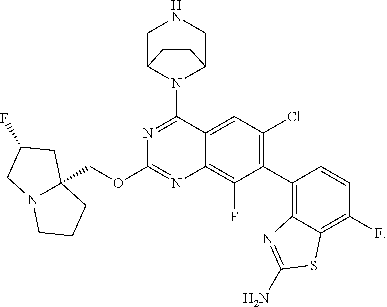 [SMILES: Nc1nc2c(-c3c(Cl)cc4c(N5C6CCC5CNC6)nc(OC[C@]56CCCN5C[C@H](F)C6)nc4c3F)ccc(F)c2s1]

Provided herein as embodiment 477 is the compound according to embodiment 1, or a pharmaceutically acceptable salt thereof, wherein the compound is:

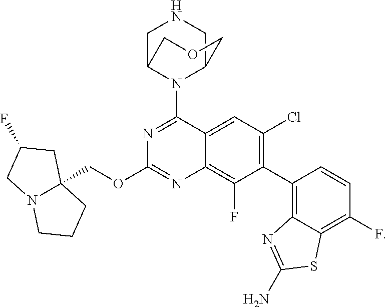 [SMILES: Nc1nc2c(-c3c(Cl)cc4c(N5C6CNCC5COC6)nc(OC[C@]56CCCN5C[C@H](F)C6)nc4c3F)ccc(F)c2s1]

Provided herein as embodiment 478 is the compound according to embodiment 1, or a pharmaceutically acceptable salt thereof, wherein the compound is:

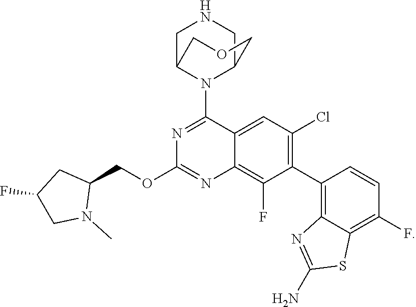 [SMILES: CN1C[C@H](F)C[C@H]1COc1nc(N2C3CNCC2COC3)c2cc(Cl)c(-c3ccc(F)c4sc(N)nc34)c(F)c2n1]

Provided herein as embodiment 479 is the compound according to embodiment 1, or a pharmaceutically acceptable salt thereof, wherein the compound is:

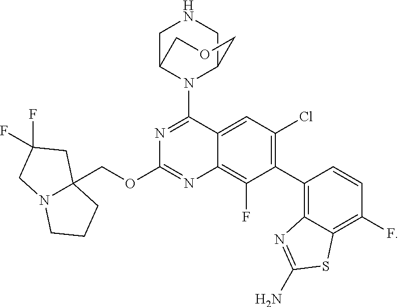 [SMILES: Nc1nc2c(-c3c(Cl)cc4c(N5C6CNCC5COC6)nc(OCC56CCCN5CC(F)(F)C6)nc4c3F)ccc(F)c2s1]

Provided herein as embodiment 480 is the compound according to embodiment 1, or a pharmaceutically acceptable salt thereof, wherein the compound is:

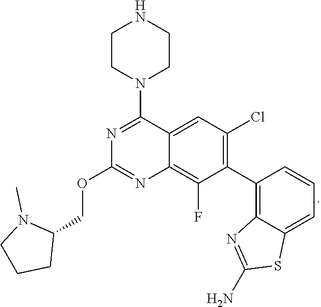 [SMILES: CN1CCC[C@H]1COc1nc(N2CCNCC2)c2cc(Cl)c(-c3cccc4sc(N)nc34)c(F)c2n1]

Provided herein as embodiment 481 is the compound according to embodiment 1, or a pharmaceutically acceptable salt thereof, wherein the compound is:

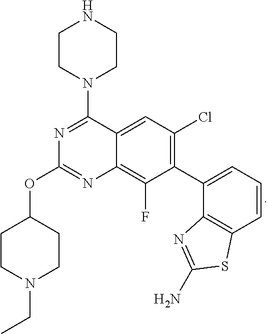 [SMILES: CCN1CCC(Oc2nc(N3CCNCC3)c3cc(Cl)c(-c4cccc5sc(N)nc45)c(F)c3n2)CC1]

Provided herein as embodiment 482 is the compound according to embodiment 1, or a pharmaceutically acceptable salt thereof, wherein the compound is:

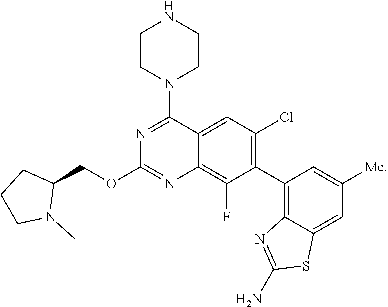 [SMILES: Cc1cc(-c2c(Cl)cc3c(N4CCNCC4)nc(OC[C@@H]4CCCN4C)nc3c2F)c2nc(N)sc2c1]

Provided herein as embodiment 483 is the compound according to embodiment 1, or a pharmaceutically acceptable salt thereof, wherein the compound is:

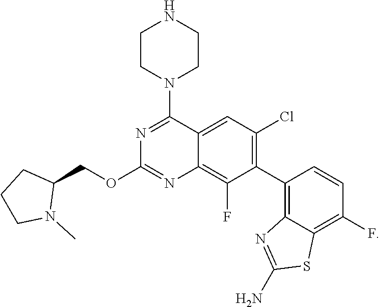 [SMILES: CN1CCC[C@H]1COc1nc(N2CCNCC2)c2cc(Cl)c(-c3ccc(F)c4sc(N)nc34)c(F)c2n1]

Provided herein as embodiment 484 is the compound according to embodiment 1, or a pharmaceutically acceptable salt thereof, wherein the compound is:

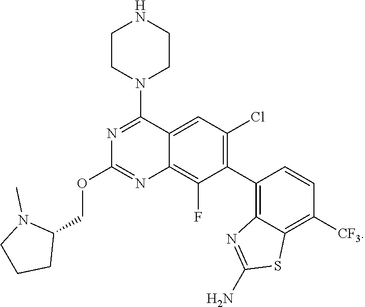 [SMILES: CN1CCC[C@H]1COc1nc(N2CCNCC2)c2cc(Cl)c(-c3ccc(C(F)(F)F)c4sc(N)nc34)c(F)c2n1]

Provided herein as embodiment 485 is the compound according to embodiment 1, or a pharmaceutically acceptable salt thereof, wherein the compound is:

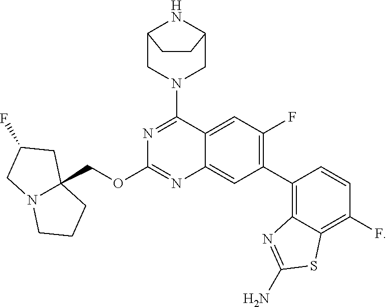 [SMILES: Nc1nc2c(-c3cc4nc(OC[C@@]56CCCN5C[C@H](F)C6)nc(N5CC6CCC(C5)N6)c4cc3F)ccc(F)c2s1]

Provided herein as embodiment 486 is the compound according to embodiment 1, or a pharmaceutically acceptable salt thereof, wherein the compound is:

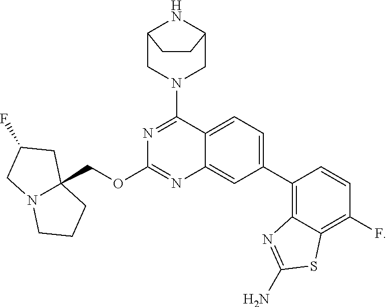 [SMILES: Nc1nc2c(-c3ccc4c(N5CC6CCC(C5)N6)nc(OC[C@@]56CCCN5C[C@H](F)C6)nc4c3)ccc(F)c2s1]

Provided herein as embodiment 487 is the compound according to embodiment 1, or a pharmaceutically acceptable salt thereof, wherein the compound is:

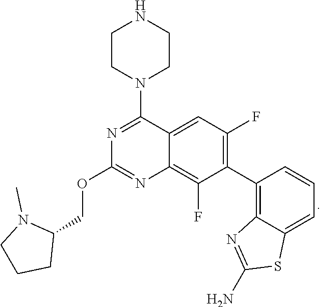 [SMILES: CN1CCC[C@H]1COc1nc(N2CCNCC2)c2cc(F)c(-c3cccc4sc(N)nc34)c(F)c2n1]

Provided herein as embodiment 488 is the compound according to embodiment 1, or a pharmaceutically acceptable salt thereof, wherein the compound is:

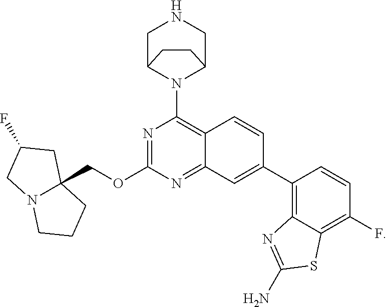 [SMILES: Nc1nc2c(-c3ccc4c(N5C6CCC5CNC6)nc(OC[C@@]56CCCN5C[C@H](F)C6)nc4c3)ccc(F)c2s1]

Provided herein as embodiment 489 is the compound according to embodiment 1, or a pharmaceutically acceptable salt thereof, wherein the compound is:

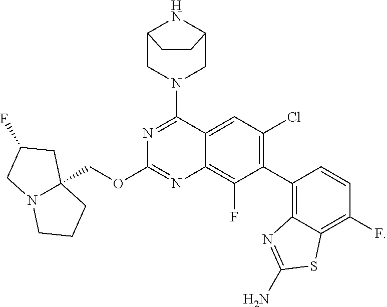 [SMILES: Nc1nc2c(-c3c(Cl)cc4c(N5CC6CCC(C5)N6)nc(OC[C@]56CCCN5C[C@H](F)C6)nc4c3F)ccc(F)c2s1]

Provided herein as embodiment 490 is the compound according to embodiment 1, or a pharmaceutically acceptable salt thereof, wherein the compound is:

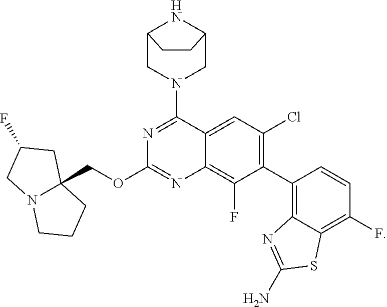 [SMILES: Nc1nc2c(-c3c(Cl)cc4c(N5CC6CCC(C5)N6)nc(OC[C@@]56CCCN5C[C@H](F)C6)nc4c3F)ccc(F)c2s1]

Provided herein as embodiment 491 is the compound according to embodiment 1, or a pharmaceutically acceptable salt thereof, wherein the compound is:

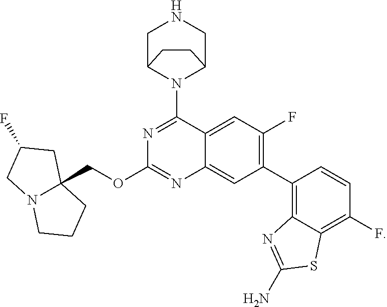 [SMILES: Nc1nc2c(-c3cc4nc(OC[C@@]56CCCN5C[C@H](F)C6)nc(N5C6CCC5CNC6)c4cc3F)ccc(F)c2s1]

Provided herein as embodiment 492 is the compound according to embodiment 1, or a pharmaceutically acceptable salt thereof, wherein the compound is:

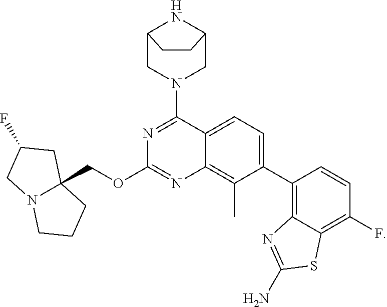 [SMILES: Cc1c(-c2ccc(F)c3sc(N)nc23)ccc2c(N3CC4CCC(C3)N4)nc(OC[C@@]34CCCN3C[C@H](F)C4)nc12]

Provided herein as embodiment 493 is the compound according to embodiment 1, or a pharmaceutically acceptable salt thereof, wherein the compound is:

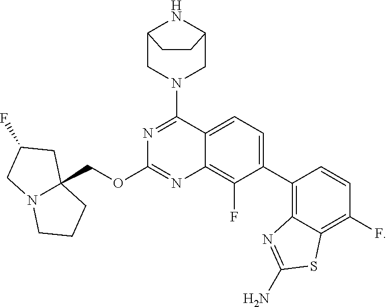 [SMILES: Nc1nc2c(-c3ccc4c(N5CC6CCC(C5)N6)nc(OC[C@@]56CCCN5C[C@H](F)C6)nc4c3F)ccc(F)c2s1]

Provided herein as embodiment 494 is the compound according to embodiment 1, or a pharmaceutically acceptable salt thereof, wherein the compound is:

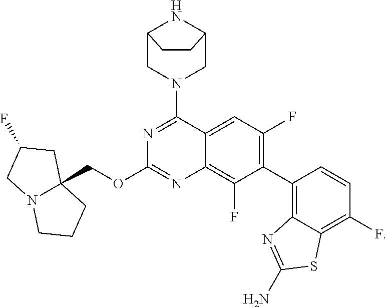 [SMILES: Nc1nc2c(-c3c(F)cc4c(N5CC6CCC(C5)N6)nc(OC[C@@]56CCCN5C[C@H](F)C6)nc4c3F)ccc(F)c2s1]

Provided herein as embodiment 495 is the compound according to embodiment 1, or a pharmaceutically acceptable salt thereof, wherein the compound is:

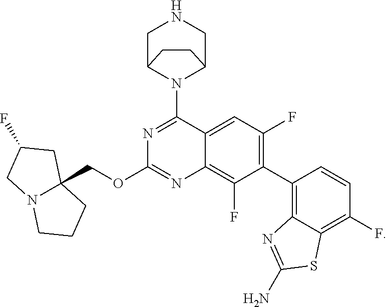 [SMILES: Nc1nc2c(-c3c(F)cc4c(N5C6CCC5CNC6)nc(OC[C@@]56CCCN5C[C@H](F)C6)nc4c3F)ccc(F)c2s1]

Provided herein as embodiment 496 is the compound according to embodiment 1, or a pharmaceutically acceptable salt thereof, wherein the compound is:

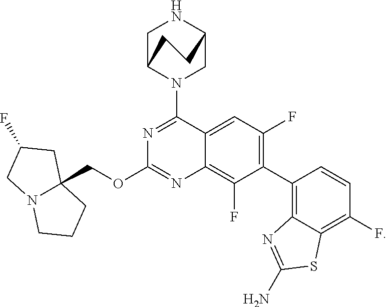 [SMILES: Nc1nc2c(-c3c(F)cc4c(N5C[C@@H]6CC[C@H]5CN6)nc(OC[C@@]56CCCN5C[C@H](F)C6)nc4c3F)ccc(F)c2s1]

Provided herein as embodiment 498 is the compound according to embodiment 1, or a pharmaceutically acceptable salt thereof, wherein the compound is:

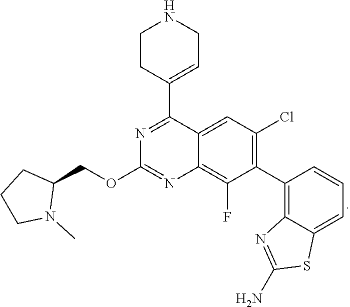 [SMILES: CN1CCC[C@H]1COc1nc(C2=CCNCC2)c2cc(Cl)c(-c3cccc4sc(N)nc34)c(F)c2n1]

Provided herein as embodiment 498 is the compound according to embodiment 1, or a pharmaceutically acceptable salt thereof, wherein the compound is:

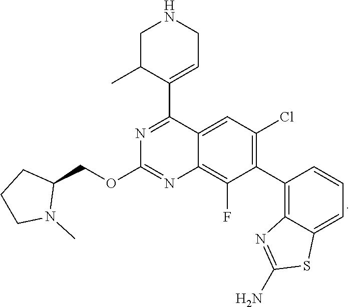 [SMILES: CC1CNCC=C1c1nc(OC[C@@H]2CCCN2C)nc2c(F)c(-c3cccc4sc(N)nc34)c(Cl)cc12]

Provided herein as embodiment 499 is the compound according to embodiment 1, or a pharmaceutically acceptable salt thereof, wherein the compound is:

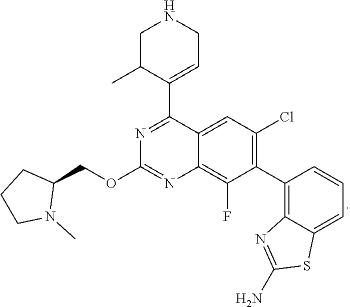 [SMILES: CC1CNCC=C1c1nc(OC[C@@H]2CCCN2C)nc2c(F)c(-c3cccc4sc(N)nc34)c(Cl)cc12]

Provided herein as embodiment 500 is the compound according to embodiment 1, or a pharmaceutically acceptable salt thereof, wherein the compound is:

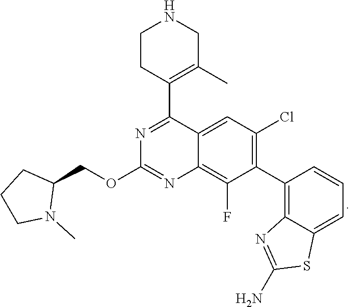 [SMILES: CC1=C(c2nc(OC[C@@H]3CCCN3C)nc3c(F)c(-c4cccc5sc(N)nc45)c(Cl)cc23)CCNC1]

Provided herein as embodiment 501 is the compound according to embodiment 1, or a pharmaceutically acceptable salt thereof, wherein the compound is:

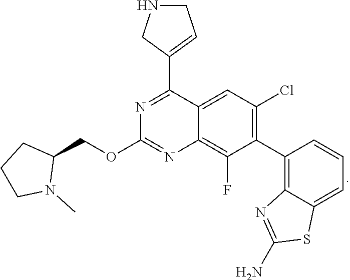 [SMILES: CN1CCC[C@H]1COc1nc(C2=CCNC2)c2cc(Cl)c(-c3cccc4sc(N)nc34)c(F)c2n1]

Provided herein as embodiment 502 is the compound according to embodiment 1, or a pharmaceutically acceptable salt thereof, wherein the compound is:

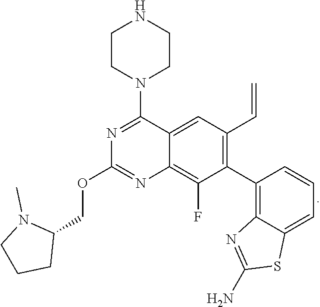 [SMILES: C=Cc1cc2c(N3CCNCC3)nc(OC[C@@H]3CCCN3C)nc2c(F)c1-c1cccc2sc(N)nc12]

Provided herein as embodiment 503 is the compound according to embodiment 1, or a pharmaceutically acceptable salt thereof, wherein the compound is:

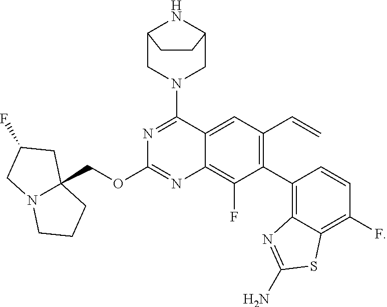 [SMILES: C=Cc1cc2c(N3CC4CCC(C3)N4)nc(OC[C@@]34CCCN3C[C@H](F)C4)nc2c(F)c1-c1ccc(F)c2sc(N)nc12]

Provided herein as embodiment 504 is the compound according to embodiment 1, or a pharmaceutically acceptable salt thereof, wherein the compound is:

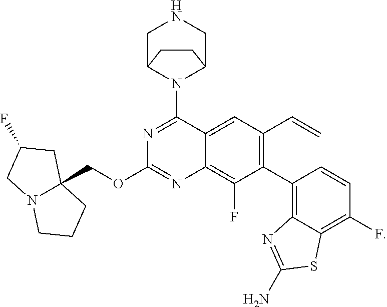 [SMILES: C=Cc1cc2c(N3C4CCC3CNC4)nc(OC[C@@]34CCCN3C[C@H](F)C4)nc2c(F)c1-c1ccc(F)c2sc(N)nc12]

Provided herein as embodiment 505 is the compound according to embodiment 1, or a pharmaceutically acceptable salt thereof, wherein the compound is:

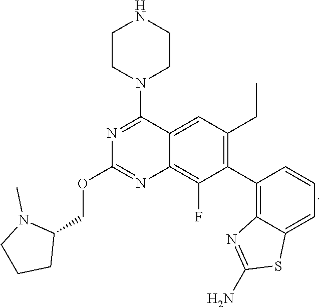 [SMILES: CCc1cc2c(N3CCNCC3)nc(OC[C@@H]3CCCN3C)nc2c(F)c1-c1cccc2sc(N)nc12]

Provided herein as embodiment 506 is the compound according to embodiment 1, or a pharmaceutically acceptable salt thereof, wherein the compound is:

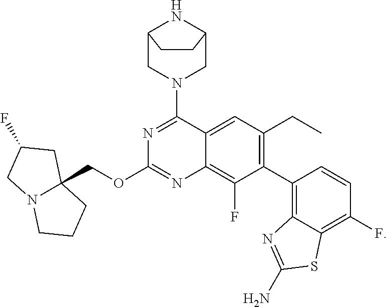 [SMILES: CCc1cc2c(N3CC4CCC(C3)N4)nc(OC[C@@]34CCCN3C[C@H](F)C4)nc2c(F)c1-c1ccc(F)c2sc(N)nc12]

Provided herein as embodiment 507 is the compound according to embodiment 1, or a pharmaceutically acceptable salt thereof, wherein the compound is:

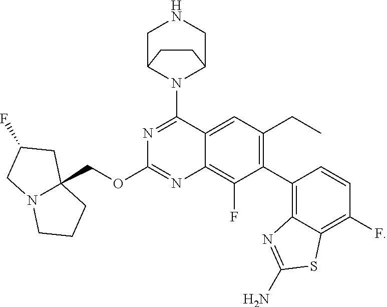 [SMILES: CCc1cc2c(N3C4CCC3CNC4)nc(OC[C@@]34CCCN3C[C@H](F)C4)nc2c(F)c1-c1ccc(F)c2sc(N)nc12]

Provided herein as embodiment 508 is the compound according to embodiment 1, or a pharmaceutically acceptable salt thereof, wherein the compound is:

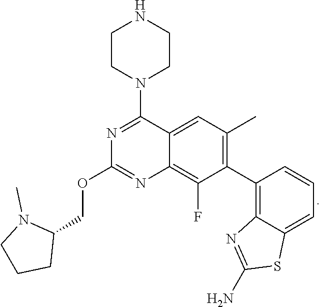 [SMILES: Cc1cc2c(N3CCNCC3)nc(OC[C@@H]3CCCN3C)nc2c(F)c1-c1cccc2sc(N)nc12]

Provided herein as embodiment 509 is the compound according to embodiment 1, or a pharmaceutically acceptable salt thereof, wherein the compound is:

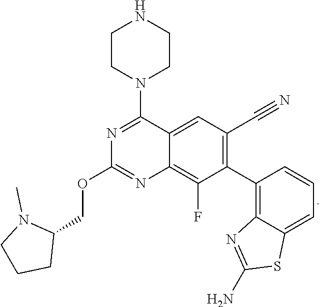 [SMILES: CN1CCC[C@H]1COc1nc(N2CCNCC2)c2cc(C#N)c(-c3cccc4sc(N)nc34)c(F)c2n1]

Provided herein as embodiment 510 is the compound according to embodiment 1, or a pharmaceutically acceptable salt thereof, wherein the compound is:

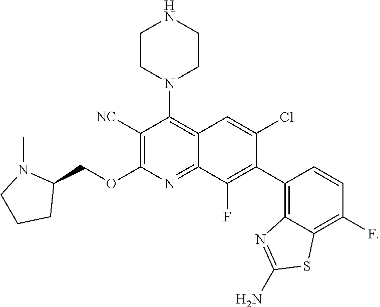 [SMILES: CN1CCC[C@@H]1COc1nc2c(F)c(-c3ccc(F)c4sc(N)nc34)c(Cl)cc2c(N2CCNCC2)c1C#N]

Provided herein as embodiment 511 is the compound according to embodiment 1, or a pharmaceutically acceptable salt thereof, wherein the compound is:

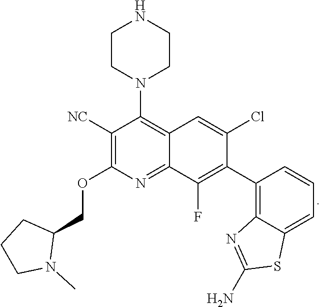 [SMILES: CN1CCC[C@H]1COc1nc2c(F)c(-c3cccc4sc(N)nc34)c(Cl)cc2c(N2CCNCC2)c1C#N]

Provided herein as embodiment 512 is the compound according to embodiment 1, or a pharmaceutically acceptable salt thereof, wherein the compound is:

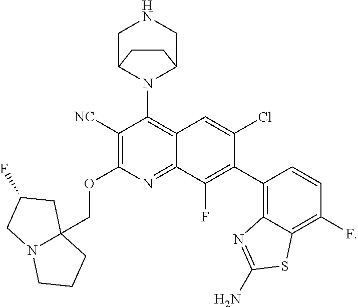 [SMILES: N#Cc1c(OCC23CCCN2C[C@H](F)C3)nc2c(F)c(-c3ccc(F)c4sc(N)nc34)c(Cl)cc2c1N1C2CCC1CNC2]

Provided herein as embodiment 513 is the compound according to embodiment 1, or a pharmaceutically acceptable salt thereof, wherein the compound is:

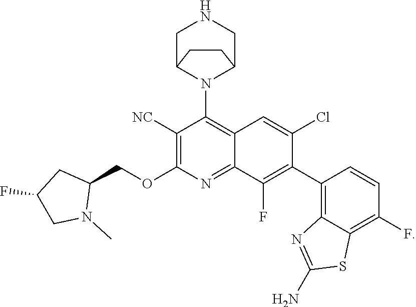 [SMILES: CN1C[C@H](F)C[C@H]1COc1nc2c(F)c(-c3ccc(F)c4sc(N)nc34)c(Cl)cc2c(N2C3CCC2CNC3)c1C#N]

Provided herein as embodiment 514 is the compound according to embodiment 1, or a pharmaceutically acceptable salt thereof, wherein the compound is:

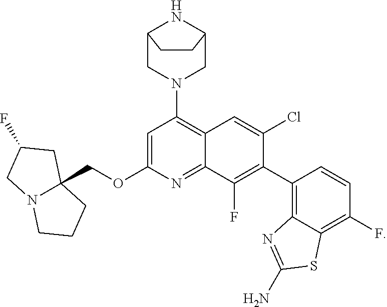 [SMILES: Nc1nc2c(-c3c(Cl)cc4c(N5CC6CCC(C5)N6)cc(OC[C@@]56CCCN5C[C@H](F)C6)nc4c3F)ccc(F)c2s1]

The foregoing merely summarizes certain aspects of this disclosure and is not intended, nor should it be construed, as limiting the disclosure in any way.

### Formulation, and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by G D Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as embodiment 515 is a pharmaceutical composition comprising the compound according to any one of embodiments 1-514, or a tautomer thereof, or a pharmaceutically acceptable salt of said compound or said tautomer, and a pharmaceutically acceptable excipient.

Provided herein as embodiment 516 is a compound according to any one of embodiments 1-514, or a tautomer thereof, or a pharmaceutically acceptable salt of said compound or said tautomer, or the pharmaceutical composition according to embodiment 515 for use as a medicament.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In one embodiment, the disclosure provides methods of using the compounds or pharmaceutical compositions of the present disclosure to treat disease conditions, including but not limited to conditions implicated by KRAS G12D mutation (e.g., cancer). The cancer types are non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

KRAS G12D mutations occur with the alteration frequencies shown in the table below (TCGA data sets;¹⁻³ For example, the table shows that 32.4% of subjects with pancreatic cancer have a cancer wherein one or more cells express KRAS G12D mutant protein. Accordingly, the compounds provided herein, which bind to KRAS^{G12D} (see Section entitled “Biological Evaluation” below) are useful for treatment of subjects having a cancer, including, but not limited to the cancers listed in the table below.

| | Alteration |
|---|---|
| Cancer Type | Frequency |
| Pancreatic Adenocarcinoma (PAAD) | 32.4 |
| Colon Adenocarcinoma (COAD) | 12.25 |
| Rectal adenocarcinoma (READ) | 8.03 |
| Uterine corpus endometrial carcinoma | 6.04 |
| (UCEC) | |
| Lung Adenocarcinoma (LUAD) | 3.53 |
| Plasma Cell Tumors | 2.92 |
| Stomach Adenocarcinoma (STAD) | 2.27 |
| Bladder urothelial carcinoma (BLCA) | 1.46 |
| Cervical Squamous carcinoma (CESC) | 1.38 |
| Kidney Adenocarcinoma | 1.07 |
| Thymic Cancer | 0.81 |
| Myeloid Leukemia (LAML) | 0.69 |
| Liver Hepatocellular Carcinoma (LIHC) | 0.55 |
| Glioblastoma multiforme (GBM) | 0.51 |
| Skin Cutaneous Melanoma (SKCM) | 0.43 |
| Bladder Cancer | 0.4 |
| Prostate Adenocarcinoma (PRAD) | 0.2 |
| Breast Invasive Carcinoma (BRCA) | 0.1 |

Provided herein as embodiment 517 is a compound according to any one of embodiments 1-514 or a pharmaceutically acceptable salt thereof, or the pharmaceutical composition according to embodiment 515 for use in treating cancer.

Provided herein as Embodiment 518 is a compound according to any one of Embodiments 1-514 or a pharmaceutically acceptable salt thereof, or the pharmaceutical composition according to Embodiment 515 for use in treating cancer, wherein one or more cells express KRAS G12D mutant protein.

Provided herein as Embodiment 519 is the compound or pharmaceutical composition for use of Embodiment 517 or 518, wherein the cancer is pancreatic cancer, colorectal cancer, non-small cell lung cancer, small bowel cancer, appendiceal cancer, cancer of unknown primary, endometrial cancer, mixed cancer types, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 520 is a use of the compound according to any one of Embodiments 1-514 or a pharmaceutically acceptable salt thereof, or the pharmaceutical composition according to Embodiment 515 in the preparation of a medicament for treating cancer.

Provided herein as Embodiment 521 is a use of the compound according to any one of Embodiments 1-514 or a pharmaceutically acceptable salt thereof, or the pharmaceutical composition according to Embodiment 515 in the preparation of a medicament for treating cancer, wherein one or more cells express KRAS G12D mutant protein.

Provided herein as Embodiment 522 is the use according to Embodiment 520 or 521, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 523 is a method of treating cancer in a subject in need thereof, the method comprising administering to the subject a therapeutically effective amount of the compound according to any one of to any one of Embodiments 1-514 or a pharmaceutically acceptable salt thereof.

Provided herein as Embodiment 524 is a method of treating cancer in a subject in need thereof, the method comprising administering to the subject a therapeutically effective amount of the compound according to any one of to any one of Embodiments 1-514 or a pharmaceutically acceptable salt thereof, wherein one or more cells express KRAS G12D mutant protein.

Provided herein as Embodiment 525 is the method according to Embodiment 523 or 524, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 526 is the method according to Embodiment 523 or 524, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

Provided herein as Embodiment 527 is the method according to Embodiment 526, wherein the cancer is non-small cell lung cancer.

Provided herein as Embodiment 528 is the method according to Embodiment 526, wherein the cancer is colorectal cancer.

Provided herein as Embodiment 529 is the method according to Embodiment 526, wherein the cancer is pancreatic cancer.

Provided herein as Embodiment 530 is the method according to anyone of Embodiments 523-529, wherein the subject has a cancer that was determined to have one or more cells expressing the KRAS G12D mutant protein prior to administration of the compound or a pharmaceutically acceptable salt thereof.

### Combination Therapy

The present disclosure also provides methods for combination therapies in which an agent known to modulate other pathways, or other components of the same pathway, or even overlapping sets of target enzymes are used in combination with a compound of the present disclosure or a pharmaceutically acceptable salt thereof. In one aspect, such therapy includes but is not limited to the combination of one or more compounds of the disclosure with chemotherapeutic agents, therapeutic antibodies, and radiation treatment, to provide a synergistic or additive therapeutic effect. See. e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

Provided herein as Embodiment 531 is the method according to anyone of Embodiments 523-530, which further comprises simultaneous, separate, or sequential administration of an effective amount of a second compound, wherein the second compound is an Aurora kinase A inhibitor, AKT inhibitor, arginase inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MCL-1 inhibitor, MEK inhibitor, mTOR inhibitor, PD-1 inhibitor, PD-L1 inhibitor, PI3K inhibitor, Raf kinase inhibitor, SHP2 inhibitor, SOS1 inhibitor, Src kinase inhibitor, or one or more chemotherapeutic agent.

In one embodiment, the second compound is administered as a pharmaceutically acceptable salt. In another embodiment the second compound is administered as a pharmaceutical composition comprising the second compound or a pharmaceutically acceptable salt thereof and a pharmaceutically acceptable excipient.

### Aurora Kinase A Inhibitors

Provided herein is the method according to anyone of Embodiments 523-530, which further comprises simultaneous, separate, or sequential administration of an effective amount of a second compound, wherein the second compound is an Aurora kinase A inhibitor.

Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03,7]tetradeca-1(14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino)pyrimidin-4-yl]-1,3-thiazol-2-amine), TAS-119, BI 811283, and TP607.

### AKT Inhibitors

Provided herein is the method according to anyone of Embodiments 523-530, which further comprises simultaneous, separate, or sequential administration of an effective amount of a second compound, wherein the second compound is an AKT inhibitor.

Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f][1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1(9),5-dien-8-one), ARQ 751 (N-(3-aminopropyl)-N-[(1R)-1-(3-anilino-7-chloro-4-oxoquinazolin-2-yl)but-3-ynyl]-3-chloro-2-fluorobenzamide), RX-0201, and LY2780301.

### Arginase Inhibitors

Provided herein is the method according to anyone of Embodiments 523-530, which further comprises simultaneous, separate, or sequential administration of an effective amount of a second compound, wherein the second compound is an arginase inhibitor.

Exemplary arginase inhibitors for use in the methods provided herein include, but are not limited to, numidargistat and CB 280.

### CDK4/6 Inhibitors

Provided herein is the method according to anyone of Embodiments 523-530, which further comprises simultaneous, separate, or sequential administration of an effective amount of a second compound, wherein the second compound is a CDK4/6 inhibitor.

The term “CDK 4/6” as used herein refers to cyclin dependent kinases (“CDK”) 4 and 6, which are members of the mammalian serine/threonine protein kinases.

The term “CDK 4/6 inhibitor” as used herein refers to a compound that is capable of negatively modulating or inhibiting all or a portion of the enzymatic activity of CDK 4 and/or 6.

Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In one embodiment, the CDK4/6 inhibitor is palbociclib.

### ErbB Family Inhibitors

Provided herein is the method according to anyone of Embodiments 523-530, which further comprises simultaneous, separate, or sequential administration of an effective amount of a second compound, wherein the second compound is an ErbB family inhibitor.

The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4).

The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members.

In one embodiment, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In one embodiment, the anti-EGFR antibody is cetuximab. In one embodiment, the anti-EGFR antibody is panitumumab.

In another embodiment the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine.

In yet another embodiment the ErbB family inhibitor is a HER3 inhibitor, e.g., an anti-HER3 antibody, such as HMBD-001 (Hummingbird Bioscience).

In one embodiment, the ErbB family inhibitor is a combination of an anti-EGFR antibody and anti-HER2 antibody.

In one embodiment, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino)but-2-enamide).

In one embodiment, the irreversible ErbB family inhibitor is afatinib. In one embodiment, the irreversible ErbB family inhibitor is dacomitinib.

In one embodiment, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide), AEE788 ((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine).

In one embodiment, the reversible ErbB family inhibitor is sapitinib. In one embodiment, the reversible ErbB family inhibitor is tarloxotinib.

### ERK Inhibitors

Provided herein is the method according to anyone of Embodiments 523-530, which further comprises simultaneous, separate, or sequential administration of an effective amount of a second compound, wherein the second compound is an ERK inhibitor.

Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl)thieno[2,3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187.

### FAK Inhibitors

Provided herein is the method according to anyone of Embodiments 523-530, which further comprises simultaneous, separate, or sequential administration of an effective amount of a second compound, wherein the second compound is a FAK inhibitor.

Exemplary FAK inhibitors for use in the methods provided herein include, but are not limited to, GSK2256098 (2-[[5-chloro-2-[(5-methyl-2-propan-2-ylpyrazol-3-yl)amino]pyridin-4-yl]amino]-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[[2-(2-methoxy-4-morpholin-4-ylanilino)-5-(trifluoromethyl)pyridin-4-yl]amino]-N-methylbenzamide), and APG-2449.

### FGFR Inhibitors

Provided herein is the method according to anyone of Embodiments 523-530, which further comprises simultaneous, separate, or sequential administration of an effective amount of a second compound, wherein the second compound is an FGFR inhibitor.

Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), debio 1347 ([5-amino-1-(2-methyl-3H-benzimidazol-5-yl)pyrazol-4-yl]-(1H-indol-2-yl)methanone), INCB062079, H₃B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

### Glutaminase Inhibitors

Provided herein is the method according to anyone of Embodiments 523-530, which further comprises simultaneous, separate, or sequential administration of an effective amount of a second compound, wherein the second compound is a glutaminase inhibitor.

Exemplary glutaminase inhibitors for use in the methods provided herein include, but are not limited to, telaglenastat, IPN60090, and OP 330.

### IGF-1R Inhibitors

Provided herein is the method according to anyone of Embodiments 523-530, which further comprises simultaneous, separate, or sequential administration of an effective amount of a second compound, wherein the second compound is an IGF-1R inhibitor.

Exemplary IGF-1R inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl)piperazin-1-yl]methyl]-2-[(E)-2-(1H-indazol-3-yl)ethenyl]phenyl]-3-methylthiophene-2-carboxamide), PL225B, AVE1642, and BIIB022.

### KIF18A Inhibitors

Provided herein is the method according to anyone of Embodiments 523-530, which further comprises simultaneous, separate, or sequential administration of an effective amount of a second compound, wherein the second compound is a KIF18A inhibitor.

Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety.

### MCL-1 Inhibitors

Provided herein is the method according to anyone of Embodiments 523-530, which further comprises simultaneous, separate, or sequential administration of an effective amount of a second compound, wherein the second compound is an MCL-1 inhibitor.

Exemplary MEK inhibitors for use in the methods provided herein include, but are not limited to, murizatoclax, tapotoclax, AZD 5991 ((3aR)-5-chloro-2,11,12,24,27,29-hexahydro-2,3,24,33-tetramethyl-22H-9,4,8-(metheniminomethyno)-14,20:26,23-dimetheno-10H,20H-pyrazolo[4,3-1][2,15,22,18,19]benzoxadithiadiazacyclohexacosine-32-carboxylic acid), MIK 665 ((αR)-α-[[(5S)-5-[3-Chloro-2-methyl-4-[2-(4-methyl-1-piperazinyl)ethoxy]phenyl]-6-(4-fluorophenyl)thieno[2,3-d]pyrimidin-4-yl]oxy]-2-[[2-(2-methoxyphenyl)-4-pyrimidinyl]methoxy]benzenepropanoic acid), and ABBV-467.

In one embodiment, the MCL-1 inhibitor is murizatoclax. In another embodiment, the MCL-1 inhibitor is tapotoclax.

### MEK Inhibitors

Provided herein is the method according to anyone of Embodiments 523-530, which further comprises simultaneous, separate, or sequential administration of an effective amount of a second compound, wherein the second compound is MEK inhibitor.

Exemplary MEK inhibitors for use in the methods provided herein include, but are not limited to, trametinib, cobimetinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy)imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554.

In one embodiment, the MEK inhibitor is trametinib.

mTOR Inhibitors

Provided herein is the method according to anyone of Embodiments 523-530, which further comprises simultaneous, separate, or sequential administration of an effective amount of a second compound, wherein the second compound is an mTOR inhibitor.

Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib, Torin-1 (1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine).

In one embodiment, the mTOR inhibitor is everolimus.

### PD-1 Inhibitors

Provided herein is the method according to anyone of Embodiments 523-530, which further comprises simultaneous, separate, or sequential administration of an effective amount of a second compound, wherein the second compound is a PD-1 inhibitor.

Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference.

In one embodiment, the PD-1 inhibitor is pembrolizumab. In another embodiment the PD-1 inhibitor is the Anti-PD-1 Antibody A.

### PD-L1 Inhibitors

Provided herein is the method according to anyone of Embodiments 523-530, which further comprises simultaneous, separate, or sequential administration of an effective amount of a second compound, wherein the second compound is a PD-L1 inhibitor.

Exemplary PD-L1 inhibitors for use in the methods provided herein include, but are not limited to, atezolizumab, avelumab, durvalumab, ZKAB001, TG-1501, SHR-1316, MSB2311, MDX-1105, KN035, IMC-001, HLX20, FAZ053, CS1001, CK-301, CBT-502, BGB-A333, BCD-135, and A167.

In one embodiment, the PD-L1 inhibitor is atezolizumab.

### PI3K Inhibitors

Provided herein is the method according to anyone of Embodiments 523-530, which further comprises simultaneous, separate, or sequential administration of an effective amount of a second compound, wherein the second compound is a PI3K inhibitor.

Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), ME-401 (N-[2-methyl-1-[2-(1-methylpiperidin-4-yl)phenyl]propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), SF1126 ((2S)-2-[[(2S)-3-carboxy-2-[[2-[[(2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate), XL147 (N-[3-(2,1,3-benzothiadiazol-5-ylamino)quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

### Raf Kinase Inhibitors

Provided herein is the method according to anyone of Embodiments 523-530, which further comprises simultaneous, separate, or sequential administration of an effective amount of a second compound, wherein the second compound is a Raf kinase inhibitor.

The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers.

The term “Raf kinase inhibitor” as used herein refers to a compound that is capable of negatively modulating or inhibiting all or a portion of the enzymatic activity of one or more member of the Raf family kinases, or is capable of disrupting Raf homodimer or heterodimer formation to inhibit activity.

In one embodiment, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5, -morpholino-6′-((tetrahydro-2H-pyran-4-yl)oxy)-[3,3′-bipyridin]-5-yl)-3-(trifluoromethyl)benzamide), LXH₂₅₄ (N-(3-(2-(2-hydroxyethoxy)-6-morpholinopyridin-4-yl)-4-methylphenyl)-2-(trifluoromethyl)isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), CCT196969 (1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide).

In one embodiment, the Raf kinase inhibitor is encorafenib. In one embodiment, the Raf kinase inhibitor is sorafenib. In one embodiment, the Raf kinase inhibitor is lifirafenib.

### SHP2 Inhibitors

Provided herein is the method according to anyone of Embodiments 523-530, which further comprises simultaneous, separate, or sequential administration of an effective amount of a second compound, wherein the second compound is a SHP2 inhibitor.

Exemplary SHP2 inhibitors for use in the methods provided herein include, but are not limited to, SHP-099 (6-(4-amino-4-methylpiperidin-1-yl)-3-(2,3-dichlorophenyl)pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine). In one embodiment, the SHP inhibitor for use in the methods provided herein is RMC-4630 (Revolution Medicine).

In another embodiment, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 2172652-48-9).

In another embodiment, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio]pyrazolo[1,5-a]pyrazin-4-yl]-3-methyl-2-oxa-8-azaspiro[4.5]decan-4-amine (CAS 2240982-57-2), 4-[(3S,4S)-4-amino-3-methyl-2-oxa-8-azaspiro[4.5]dec-8-yl]-7-(2,3-dichlorophenyl)-6-methyl-pyrazolo[l1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (3S,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6).

In one embodiment, the SHP inhibitor for use in the methods provided herein is (1R)-8-[5-(2,3-dichlorophenyl)-6-methylimidazo[1,5-a]pyrazin-8-yl]-8-azaspiro[4.5]decan-1-amine (CAS 2240981-78-4).

In another embodiment, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(1R)-1-amino-8-azaspiro[4.5]dec-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thio]-3-[(3S,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol.

In one embodiment, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5).

In one embodiment, the SHP2 inhibitor for use in the methods provided herein is an inhibitor disclosed in U.S. Pat. No. 10,590,090 B2, US 2020/017517 A1, US 2020/017511 A1, or WO 2019/075265 A1, each of which is herewith incorporated by reference in its entirety.

### SOS1 Inhibitors

Provided herein is the method according to anyone of Embodiments 523-530, which further comprises simultaneous, separate, or sequential administration of an effective amount of a second compound, wherein the second compound is an SOS1 inhibitor.

Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine), and BI 1701963.

### Src Kinase Inhibitors

Provided herein is the method according to anyone of Embodiments 523-530, which further comprises simultaneous, separate, or sequential administration of an effective amount of a second compound, wherein the second compound is a Src kinase inhibitor.

The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily.

The term “Src kinase inhibitor” as used herein refers to a compound that is capable of negatively modulating or inhibiting all or a portion of the enzymatic activity of one or more member of the Src kinases.

Exemplary Src kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl)acetamide), SU6656 ((Z)-N,N-dimethyl-2-oxo-3-((4,5,6,7-tetrahydro-1H-indol-2-yl)methylene)indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl)(2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl)acetamide).

In one embodiment, the Src kinase inhibitor is dasatinib. In one embodiment, the Src kinase inhibitor is saracatinib. In one embodiment, the Src kinase inhibitor is ponatinib. In one embodiment, the Src kinase inhibitor is vandetanib. In one embodiment, the Src kinase inhibitor is KX-01.

### Chemotherapeutic Agents

Provided herein is the method according to anyone of Embodiments 523-530, which further comprises simultaneous, separate, or sequential administration of an effective amount of a second compound, wherein the second compound is one or more chemotherapeutic agent.

Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions

The following definitions are provided to assist in understanding the scope of this disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. Further, this disclosure encompasses pharmaceutical compositions comprising mixtures of any of the compounds disclosed herein and one or more other active agents disclosed herein. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶CI, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S-nitro, cyano, carboxy, alkyl-O—C(O)—, carbamoyl, alkyl-S(O)—, sulfonyl, sulfonamido, phenyl, and heterocyclyl.

The terms “C₁₋₄ alkyl,” and “C₁₋₆ alkyl” as used herein refer to a straight or branched chain hydrocarbon containing from 1 to 4, and 1 to 6 carbon atoms, respectively. Representative examples of C₁₋₄ alkyl or C₁₋₆ alkyl include, but are not limited to, methyl, ethyl, n-propyl, iso-propyl, n-butyl, sec-butyl, iso-butyl, tert-butyl, pentyl and hexyl.

The terms “C₁₋₄ alkylene” and “C₁₋₆ alkylene” refer to a straight or branched divalent alkyl group as defined herein containing 1 to 4, and 1 to 6 carbon atoms, respectively. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene and the like.

The term “C₂₋₄ alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₂₋₄ alkynyl” as used herein refers to a saturated hydrocarbon containing 2 to 4 carbon atoms having at least one carbon-carbon triple bond. The term includes both straight and branched moieties Representative examples of C₃₋₆ alkynyl include, but are not limited to, ethynyl, 1-propynyl, 2-propynyl, 2-butynyl and 3-butynyl.

The term “C₁₋₄ alkoxy” or “C₁₋₆-alkoxy” as used herein refers to —OR^{#}, wherein R^{#}represents a C₁₋₄ alkyl group or C₁₋₆ alkyl group, respectively, as defined herein. Representative examples of C₁₋₄ alkoxy include, but are not limited to, methoxy, ethoxy, propoxy, iso-propoxy, and butoxy. Representative examples of C₁₋₆ alkoxy include, but are not limited to, ethoxy, propoxy, iso-propoxy, and butoxy.

The term “C₃₋₈ cycloalkyl” as used herein refers to a saturated carbocyclic molecule wherein the cyclic framework has 3 to 8 carbons. Representative examples of C₃₋₈ cycloalkyl include, but are not limited to, cyclopropyl and cyclobutyl.

The term “deutero” as used herein as a prefix to another term for a chemical group refers to a modification of the chemical group, wherein one or more hydrogen atoms are substituted with deuterium (“D” or “²H”). For example, the term “C₁₋₄ deuteroalkyl” refers to a C₁₋₄ alkyl as defined herein, wherein one or more hydrogen atoms are substituted with D. Representative examples of C₁₋₄ deuteroalkyl include, but are not limited to, —CH₂D, —CHD₂, —CD₃, —CH₂CD₃, —CDHCD₃, —CD₂CD₃, —CH(CD₃)₂, —CD(CHD₂)₂, and —CH(CH₂D)(CD₃).

The term “halogen” as used herein refers to —F, —CI, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₄ haloalkyl” refers to a C₁₋₄ alkyl as defined herein, wherein one or more hydrogen atoms are substituted with a halogen. Representative examples of C₁₋₄ haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃).

As used herein, the term “heteroaryl” refers to a 5-20 membered monocyclic- or bicyclic- or tricyclic-aromatic ring system, having 1 to 8 heteroatoms selected from N, O and S. In certain preferred aspects, the heteroaryl is a 5-10 membered ring system (e.g., 5-7 membered monocycle, an 8-10 membered bicycle or a 11-14 membered tricycle) or a 5-7 membered ring system. Exemplary monocyclic heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, and 5-pyrimidinyl. Exemplary bicyclic heteroaryl groups include 1-, 3-, 4-, 5-, 6-, 7-, or 8-isoquinolinyl, 2-, 3-, 4-, 5-, 6-, 7-, or 8-quinolinyl, 1-, 3-, 4-, 5-, 6-, 7-, or 8-isoquinolinyl, 1-, 2-, 4-, 5-, 6-, 7-, or 8-benzimidazolyl and 1-, 2-, 3-, 4-, 5-, 6-, 7-, or 8-indolyl.

The term “heteroaryl” also refers to a group in which a heteroaromatic ring is fused to one or more aryl, cycloaliphatic, or heterocyclyl rings.

As used herein, the term “heterocycle,” “heterocycloalkyl” or “heterocyclo” refers to a saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocyclyl can include fused or bridged rings as well as spirocyclic rings. Examples of heterocycles include tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, azetidine, thiazolidine, morpholine, and the like.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following schemes are the variables as defined for Formula I, unless otherwise noted. All starting materials are either commercially available, for example, from Merck Sigma-Aldrich Inc., Fluorochem Ltd, and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein.

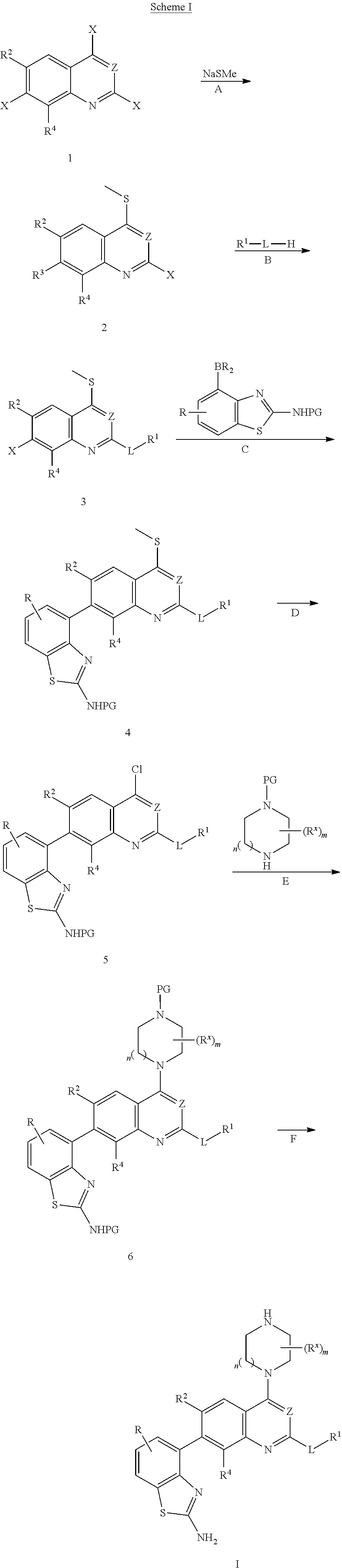

Compounds of Formula (I) can be prepared according to Scheme I. In step A, compound (1) is treated with sodium thiomethoxide in a solvent such as tetrahydrofuran to give compound (2). In step B, compound (2) is either pre-treated with a fluoride source such as potassium fluoride, or directly undergoes S_{N}Ar reaction with a nucleophile having the formula R¹—L—H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (3). In step C, compound (3) is coupled with an organometallic reagent derived from 2-aminobenzothiazole such as a boronic acid (ester) to provide compound (4). This coupling reaction proceeds in a solvent such as 1,4-dioxane and a catalyst such as Pd(dppf)Cl₂, with or without a base such as potassium phosphate. In step D, compound (4) is treated with sulfuryl chloride in a solvent such as dichloromethane to give compound (5). In step E, compound (5) undergoes S_{N}Ar reaction with optionally substituted mono-Boc protected amine in a solvent such as acetonitrile and in the presence of a base such as N,N-diisopropylethylamine to give compound (6). In step F, protecting groups are removed using conditions known in the art. For example, Boc can be removed with TFA or HCl.

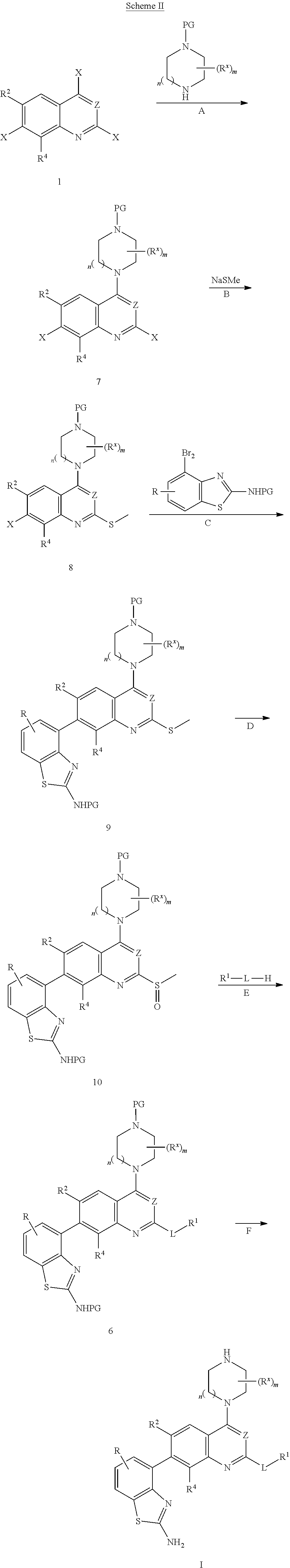

Compounds of Formula (I) can be prepared according to Scheme II. In step A, compound (1) undergoes S_{N}Ar reaction with optionally substituted mono-Boc protected amine in a solvent such as acetonitrile and in the presence of a base such as N,N-diisopropylethylamine to give compound (7). In step B, compound (7) is treated with sodium thiomethoxide in a solvent such as tetrahydrofuran to give compound (8). In step C, compound (8) is coupled with an organometallic reagent derived from 2-aminobenzothiazole such as a boronic acid (ester) to provide compound (9). This coupling reaction proceeds in a solvent such as 1,4-dioxane and a catalyst such as Pd(dppf)Cl₂, with or without a base such as potassium phosphate. In step D, compound (9) is treated with an oxidizing agent such as 3-chloroperoxybenzoic acid to give compound (10). In step E, compound (10) undergoes S_{N}Ar reaction with a nucleophile having the formula R¹—L—H in a solvent such as tetrahydrofuran in the presence of a base such as potassium tert-butoxide to give compound (6). In step F, protecting groups are removed using conditions known in the art. For example, Boc can be removed with TFA or HCl.

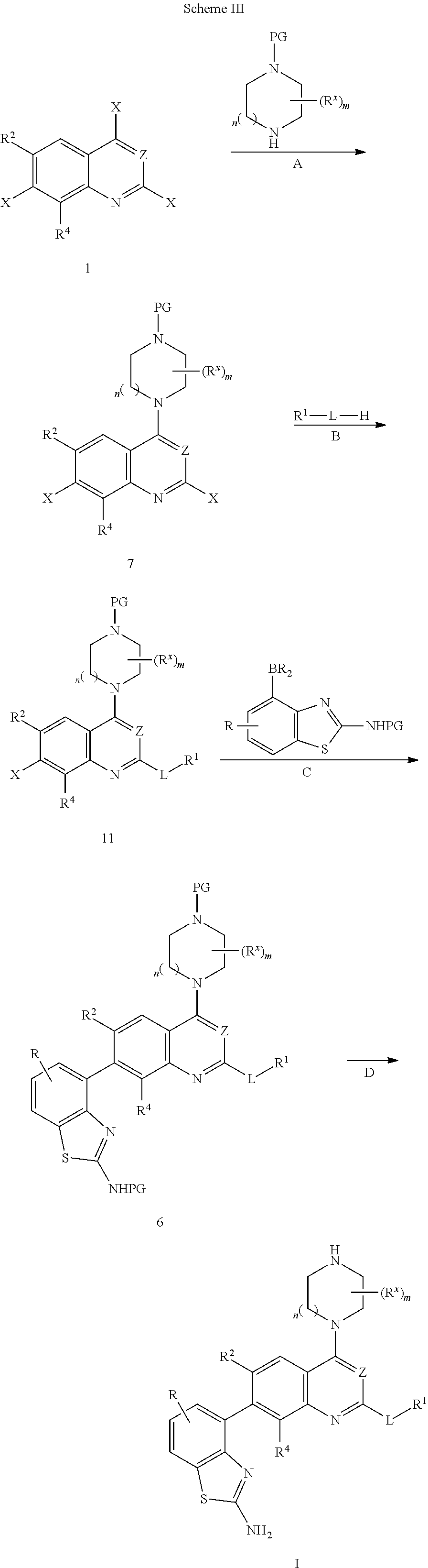

Compounds of Formula (I) can be prepared according to Scheme Ill. In step A, compound (1) undergoes S_{N}Ar reaction with optionally substituted mono-Boc protected amine in a solvent such as acetonitrile and in the presence of a base such as N,N-diisopropylethylamine to give compound (7). In step B, compound (7) is either pre-treated with a fluoride source such as potassium fluoride, or directly undergoes S_{N}Ar reaction with a nucleophile having the formula R¹—L—H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (11). In step C, compound (11) is coupled with an organometallic reagent derived from 2-aminobenzothiazole such as a boronic acid (ester) to provide compound (6). This coupling reaction proceeds in a solvent such as 1,4-dioxane and a catalyst such as Pd(dppf)Cl₂, with or without a base such as potassium phosphate. In step D, protecting groups are removed using conditions known in the art. For example, Boc can be removed with TFA or HCl.

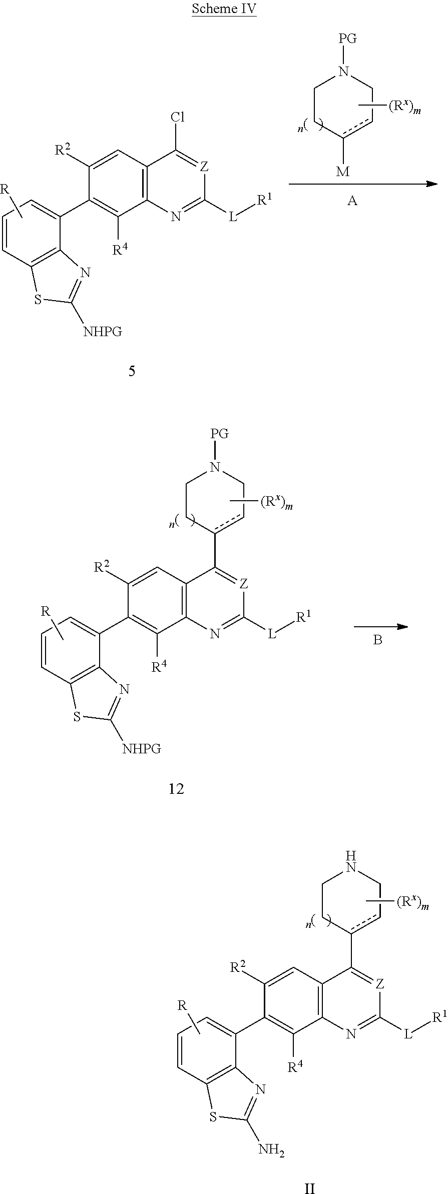

Compounds of Formula (H) can be prepared according to Scheme IV. In step A, compound (5) is coupled with an organometallic reagent, such as a boronic acid (ester) to give compound (12). This coupling reaction proceeds in a solvent such as 1,4-dioxane and a catalyst such as Pd(dppf)Cl₂, with or without a base such as potassium phosphate. In step B, protecting groups are removed using conditions known in the art. For example, Boc can be removed with TFA or HCl.

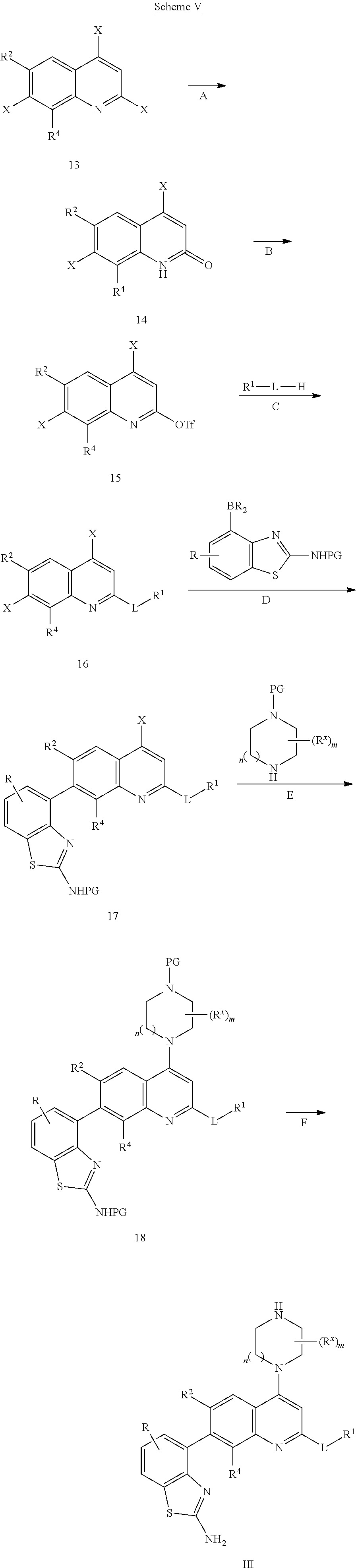

Compounds of Formula (III) can be prepared according to Scheme V. In step A, compound (13) is hydrolyzed in the presence of an acid such as sulfuric acid in a solvent such as 1,4-dioxane. In step B, compound (14) in converted to compound (15) by treatment with trifluoromethanesulfonic anhydride in the presence of abase such as N,N-diisopropylethylamine in a solvent such as dichloromethane. In step C, compound (15) undergoes S_{N}Ar reaction with a nucleophile having the formula R¹—L—H in a solvent such as tetrahydrofuran in the presence of a base such as sodium hydride to give compound (16). In step D, compound (16) is coupled with an organometallic reagent derived from 2-aminobenzothiazole such as a boronic acid (ester) to provide compound (17). This coupling reaction proceeds in a solvent such as 1,4-dioxane and a catalyst such as Pd(dppf)Cl₂, with or without a base such as potassium phosphate. In step E, compound (17) is coupled with an optionally substituted mono-Boc protected amine to give compound (18). The coupling reaction proceeds in a solvent such as 1,4-dioxane, with a catalyst such as Ruphos Pd G4, in the presence of a base such as cesium carbonate. In step F, protecting groups are removed using conditions known in the art. For example, Boc can be removed with

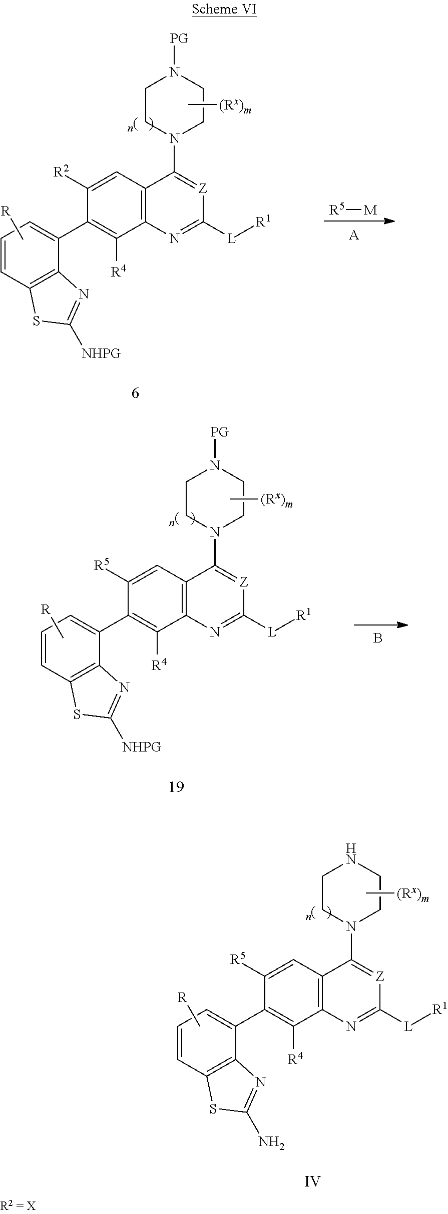

Compounds of Formula (IV) can be prepared according to Scheme VI. In step A, compound (6) is coupled with a nucleophile or an organometallic reagent such as a boronic acid (ester) to provide compound (19). This coupling reaction proceeds in a solvent such as 1,4-dioxane and a catalyst such as SPhos Pd G3, with or without a base such as potassium phosphate. In step B, protecting groups are removed using conditions known in the art. For example, Boc can be removed with TFA or HCl.

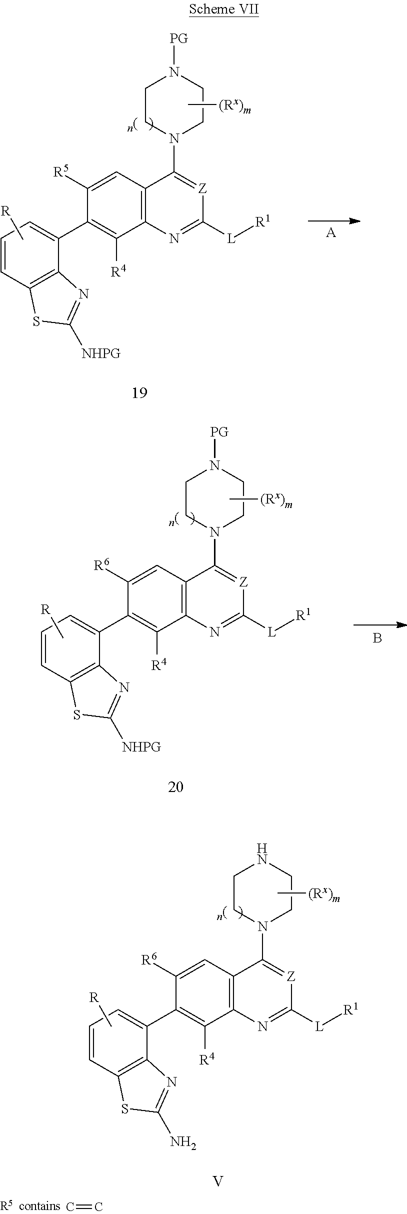

Compounds of Formula (V) can be prepared according to Scheme VII. In step A, compound (19) is reduced using a reductant such as hydrogen gas, with a catalyst such as Pd/C, in a solvent such as ethanol, to give compound (20). In step B, protecting groups are removed using conditions known in the art. For example, Boc can be removed with TFA or HCl.

### EXAMPLES

This section provides specific examples of compounds of Formula I and methods of making the same.

### LIST OF ABBREVIATIONS

TABLE 1

| | |
|---|---|
| AcOH | acetic acid |
| aq or aq. | aqueous |
| BOC or Boc | tert-butyloxycarbonyl |
| DABCO | 1,4-diazabicyclo[2.2.2]octane |
| DCM | dichloromethane |
| DMF | N,N-dimethylformamide |
| DMSO | dimethyl sulfoxide |
| Dppf, DPPF or | 1,1′-bis(diphenylphosphino)ferrocene |
| dppf | |
| eq or eq. or | equivalent |
| equiv. | |
| ESI or ES | electrospray ionization |
| Et | ethyl |
| EtOAc | ethyl acetate |
| g | gram(s) |
| h | hour(s) |
| HPLC | high pressure liquid chromatography |
| iPr | iso-propyl |
| iPr₂NEt or DIPEA | N-ethyl diisopropylamine (Hünig's base) |
| KOAc | potassium acetate |
| LC MS, LCMS, | liquid chromatography mass spectroscopy |
| LC-MS or LC/MS | |
| m/z | mass divided by charge |
| Me | methyl |
| MeCN | acetonitrile |
| MeOH | methanol |
| mg | milligrams |
| min | minutes |
| mL | milliliters |
| MS | mass spectra |
| NMR | nuclear magnetic resonance |
| Pd(dppf)Cl₂•DCM, | [1,1′- |
| Pd(dppf)Cl₂ | bis(diphenylphosphino)ferrocene]dichloropalladium |
| | (II), complex with dichloromethane |
| Pd(dtbpf)Cl₂ | [1,1′-Bis(di-tert- |
| | butylphosphino)ferrocene]dichloropalladium(II) |
| Pd(PPh₃)₄ | tetrakis(triphenylphosphine)palladium(0) |
| Ph | Phenyl |
| rbf | round-bottom flask |
| RP-HPLC | reverse phase high pressure liquid chromatography |
| RT or rt or r.t. | room temperature |
| sat. or satd. | saturated |
| SFC | supercritical fluid chromatography |
| TBAF | tetra-n-butylammonium fluoride |
| TEA or Et₃N | triethylamine |
| TFA | trifluoroacetic acid |
| THF | tetrahydrofuran |
| UV | Ultraviolet |

### General Analytical and Purification Methods

Provided in this section are descriptions of the general analytical and purification methods used to prepare the specific examples provided herein.

Chromatography: Unless otherwise indicated, crude product-containing residues were purified by passing the crude material or concentrate through either a Biotage or ISCO brand silica gel column pre-packed with flash silica (SiO2) and eluting the product off the column with a solvent gradient as indicated.

Preparative HPLC Method: Where so indicated, the compounds described herein were purified via reverse phase HPLC using Waters FractionLynx semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

Proton NMR Spectra: Unless otherwise indicated, all 1H NMR spectra were collected on a Bruker NMR Instrument at 300, 400 or 500 MHz. Where so characterized, all observed protons are reported as parts-per-million (ppm) downfield from tetramethylsilane (TMS) using the internal solvent peak as reference.

Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Preparation of Intermediates

### ((2R,7aR)-2-Fluorotetrahydro-1H-pyrrolizin-7a(5H)-yl)methanol (Intermediate A

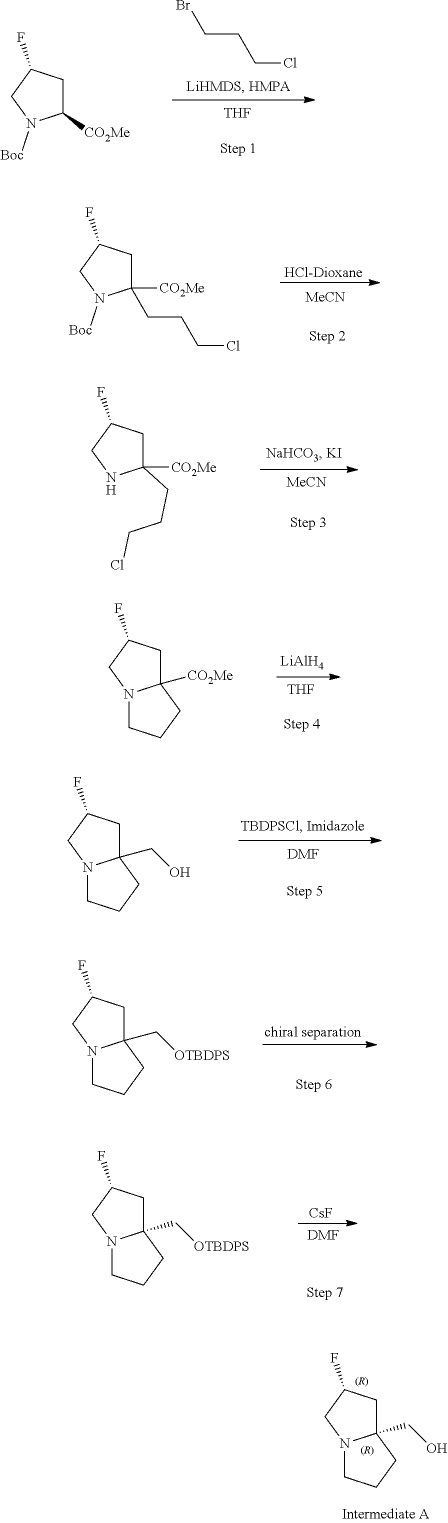

Step 1: 1-(tert-Butyl) 2-methyl (4R)-2-(3-chloropropyl)-4-fluoropyrrolidine-1,2-dicarboxylate. To a mixture of 1-tert-butyl 2-methyl (2S,4R)-4-fluoropyrrolidine-1,2-dicarboxylate (45.0 g, 182 mmol) and HMPA (42.4 g, 237 mmol, 41.6 mL) in THF (250 mL) was added LiHMDS (1.0 M, 237 mL) in portions at −70° C. under N₂. The mixture was stirred at −70° C. for 1 h. Then to the mixture was added 1-bromo-3-chloro-propane (143.3 g, 910 mmol, 90 mL) in portions at −70° C. under N₂. The resulting mixture was warmed to 15° C. and stirred for 5 h. TLC indicated the starting material was consumed completely. The mixture was poured into aq. NH₄Cl (1-L) and stirred for 20 min. The aqueous phase was extracted with EtOAc (500 mL×3). The combined organic phase was dried over anhydrous Na₂SO₄, filtered and concentrated under vacuum. The residue was purified by column chromatography on silica gel, eluting with petroleum ether/EtOAc (10/1 to 1/1) to give 1-(tert-butyl) 2-methyl (4R)-2-(3-chloropropyl)-4-fluoropyrrolidine-1,2-dicarboxylate (68.0 g, 210 mmol, 57% yield) as yellow oil.

Step 2: Methyl (4R)-2-(3-chloropropyl)-4-fluoropyrrolidine-2-carboxylate. To a mixture of 1-(tert-butyl) 2-methyl (4R)-2-(3-chloropropyl)-4-fluoropyrrolidine-1,2-dicarboxylate (34.0 g, 105 mmol) in CH₃CN (200 mL) was added HCl/dioxane (4 M, 150 mL) in one portion at 15° C. under N₂. The mixture was stirred at 15° C. for 2 h. TLC indicated the material was consumed completely. The mixture was concentrated under reduced pressure at 45° C. Crude methyl (4R)-2-(3-chloropropyl)-4-fluoro-pyrrolidine-2-carboxylate (55.0 g, crude) was obtained and used directly in the next step.

Step 3: Methyl (2R)-2-fluorotetrahydro-1H-pyrrolizine-7a(5H)-carboxylate. To a mixture of methyl (4R)-2-(3-chloropropyl)-4-fluoro-pyrrolidine-2-carboxylate (55.0 g, 211 mmol) in CH₃CN (550 mL) was added NaHCO₃ (88.8 g, 1.06 mol, 41 mL) and KI (3.51 g, 21.1 mmol) in one portion at 15° C. under N₂. The mixture was stirred at 50° C. for 12 h. TLC indicated the material was consumed completely and one new spot formed. The mixture was filtered and the filter cake was washed with EtOAc (100 mL×3). The filtrate was concentrated in vacuum. The residue was purified by column chromatography on silica gel, eluting with petroleum ether/EtOAc (3/1 to 0/1) to give methyl (2R)-2-fluorotetrahydro-1H-pyrrolizine-7a(5H)-carboxylate (27.0 g, 144 mmol, 49% yield) as yellow oil.

Step 4: ((2R)-2-Fluorotetrahydro-1H-pyrrolizin-7a(5H)-yl)methanol. To a mixture of methyl (2R)-2-fluorotetrahydro-1H-pyrrolizine-7a(5H)-carboxylate (10.0 g, 53.4 mmol) in THF (100 mL) was added LiAlH₄ (4.05 g, 107 mmol) in portions at −40° C. Then the mixture was stirred at −40° C. for 1 h. TLC showed the reaction was completed. To the reaction mixture was added Na₂SO₄·H₂O (20 g) slowly in portions at 0° C. The mixture was diluted with THF (50 mL) and filtered. The filter cake was washed with THF (300 mL). The organic layer was concentrated under vacuum. Crude ((2R)-2-fluorotetrahydro-1H-pyrrolizin-7a(5H)-yl)methanol (10.0 g, crude) as colorless oil was obtained and used directly in the next step.

Step 5: (2R)-7a-(((tert-Butyldiphenylsilyl)oxy)methyl)-2-fluorohexahydro-1H-pyrrolizine. To a mixture of ((2R)-2-fluorotetrahydro-1H-pyrrolizin-7a(5H)-yl)methanol (20.0 g, 126 mmol) and imidazole (34.2 g, 503 mmol) in DMF (25 mL) was added TBDPSC1 (69.1 g, 251 mmol, 64.54 mL). Then the mixture was stirred at 20° C. for 3 h. TLC (EtOAc:MeOH=6:1) showed the reaction was complete. The mixture was diluted with H₂O (100 mL) and extracted with EtOAc (400 mL×2). The combined organic layers were washed with brine (150 mL), dried over Na₂SO₄, filtered and concentrated under reduced pressure. The resulting residue was purified by column chromatography on silica gel, eluting with petroleum ether/EtOAc (20/1 to 10/1) to give (2R)-7a-(((tert-butyldiphenylsilyl)oxy)methyl)-2-fluorohexahydro-1H-pyrrolizine (11.0 g, 17.7 mmol, 26% yield over two steps) as colorless oil. m/z (ESI, +ve ion): 398.3 (M+H)⁺.

Step 6: (2R,7aR)-7a-(((tert-Butyldiphenylsilyl)oxy)methyl)-2-fluorohexahydro-1H-pyrrolizine. (2R)-7a-(((tert-butyldiphenylsilyl)oxy)methyl)-2-fluorohexahydro-1H-pyrrolizine (6.50 g, 16.4 mmol) was separated by column chromatography on silica gel, eluting with petroleum ether/EtOAc (7/1 to 0/1) to give (2R,7aR)-7a-(((tert-butyldiphenylsilyl)oxy)methyl)-2-fluorohexahydro-1H-pyrrolizine (2.35 g, 5.66 mmol, 36% yield, 96% purity) as yellow oil.

Step 7: ((2R,7aR)-2-Fluorotetrahydro-1H-pyrrolizin-7a(5H)-yl)methanol. To a mixture of (2R,7aR)-7a-(((tert-butyldiphenylsilyl)oxy)methyl)-2-fluorohexahydro-1H-pyrrolizine (500 mg, 1.26 mmol) in DMF (5 mL) was added CsF (1.91 g, 12.6 mmol) in one portion at 15° C. under N₂. The mixture was stirred at 70° C. for 72 h. Another batch with 9 g of (2R,7aR)-7a-(((tert-butyldiphenylsilyl)oxy)methyl)-2-fluorohexahydro-1H-pyrrolizine was set up following the same procedure. The two batches were combined for the purification. The crude product was purified by column chromatography on silica gel, eluting with EtOAc/methanol (6/1 to 1/1, with NH₃H₂O additive) to give ((2R,7aR)-2-fluorotetrahydro-1H-pyrrolizin-7a(5H)-yl)methanol (2.60 g, 16.3 mmol, 68% yield) as yellow oil. ¹H NMR (400 MHz, CHLOROFORM-d) δ ppm 5.22 (dt, J=54 Hz, 4.6 Hz, 1H), 3.47-3.31 (m, 3H), 3.00-2.98 (m, 1H), 2.88-2.75 (m, 1H), 2.67-2.65 (m, 1H), 2.25-2.15 (m, 1H), 1.92-1.80 (m, 4H), 1.60-1.55 (m, 1H).

### (2-((tert-Butoxycarbonyl)amino)benzo[d]thiazol-4-yl)boronic acid (Intermediate B

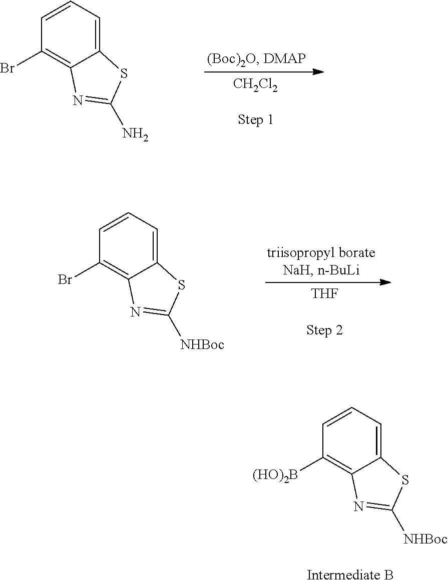 [SMILES: Nc1nc2c(Br)cccc2s1 ; "CH2Cl2" ; CC(C)(C)OC(=O)Nc1nc2c(Br)cccc2s1 ; CC(C)(C)OC(=O)Nc1nc2c(B(O)O)cccc2s1 ; "THF"]

Step 1: tert-Butyl (4-bromobenzo[d]thiazol-2-yl)carbamate. To a mixture of 4-bromo-1,3-benzothiazol-2-amine (50 g, 218 mmol) and Boc₂O (42.9 g, 196 mmol, 45.1 mL) in DCM (2-L) was added DMAP (1.87 g, 15.3 mmol) in one portion at 20° C. under N₂. The mixture was stirred at 20° C. for 12 h. The reaction mixture was diluted with water (1 L). The organic layer was separated, dried over anhydrous Na₂SO₄, filtered and concentrated under reduced pressure to give a residue. The residue was purified by column chromatography on silica gel, eluting with petroleum ether/EtOAc (1/0 to 30/1) to obtain tert-butyl (4-bromobenzo[d]thiazol-2-yl)carbamate (40 g, 122 mmol, 56% yield) as white solid.

Step 2: (2-((tert-Butoxycarbonyl)amino)benzo[d]thiazol-4-yl)boronic acid. tert-Butyl (4-bromobenzo[d]thiazol-2-yl)carbamate (50.0 g, 152 mmol) was dissolved in THF (675 mL). NaH (60/6, 9.11 g, 228 mmol) was added in portions at 20° C. under N₂. The mixture was stirred at 20° C. for 10 min, then cooled to −78° C. To the mixture was added dropwise n-butyllithium (2.5 M, 91.1 mL). The reaction mixture was stirred at −78° C. for 25 min. Triisopropyl borate (85.7 g, 456 mmol, 105 mL) was added dropwise and the reaction was stirred for 25 min. The reaction mixture was warm to 20° C. and stirred for 1 h. The reaction mixture was quenched by addition of H₂O 500 mL at 0° C., and extracted with EtOAc (800 mL×3). The combined organic layers were dried over anhydrous Na₂SO₄, filtered and concentrated under reduced pressure to give a residue. The residue was stirred in petroleum ether (500 mL) for 10 min. The mixture was filtered and the filter cake was dried under reduced pressure. The compound (2-((tert-butoxycarbonyl)amino)benzo[d]thiazol-4-yl)boronic acid (20.6 g, 70.0 mmol, 46% yield) was obtained as a yellow solid. m/z (ESI, +ve ion): 295.1 (M+H)⁺.

### (2-Amino-6-methylbenzo[d]thiazol-4-yl)boronic acid (Intermediate C

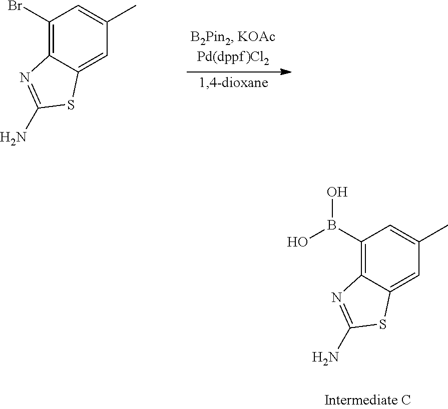

An 8-mL vial was charged with bis(pinacolato)diboron (247 mg, 0.971 mmol, Sigma-Aldrich Corporation), [1,1′-bis(diphenylphosphino)ferrocene]dichloropalladium(II) (118 mg, 0.162 mmol, Sigma-Aldrich Corporation), potassium acetate (119 mg, 1.214 mmol, Sigma-Aldrich Corporation), 4-bromo-6-methylbenzo[d]thiazol-2-amine, and 1,4-dioxane (4047 μL). The reaction was stirred at 80° C. overnight. The mixture was filtered through a plug of Celite, rinsed with EtOAc. The combined organic layer was dried in vacuo and the crude product was used in the next step without further purification. m/z (ESI, +ve ion): 209.0 (M+H)⁺.

### (2-amino-7-(trifluoromethyl)benzo[d]thiazol-4-yl)boronic acid (Intermediate D

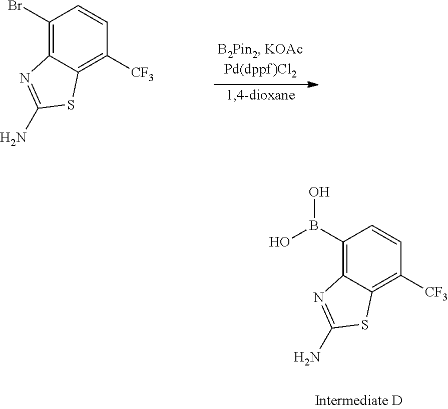 [SMILES: Nc1nc2c(Br)ccc(C(F)(F)F)c2s1 ; Nc1nc2c(B(O)O)ccc(C(F)(F)F)c2s1]

Intermediate D was synthesized in a manner similar to Intermediate C, using 4-bromo-7-(trifluoromethyl)benzo[d]thiazol-2-amine. m/z (ESI, +ve ion): 262.9 (M+H)⁺.

### tert-Butyl (4-(6-chloro-8-fluoro-2-(((S)-1-methylpyrrolidin-2-yl)methoxy)-4-(methylthio)quinazolin-7-yl)benzo[d]thiazol-2-yl)carbamate (Intermediate E

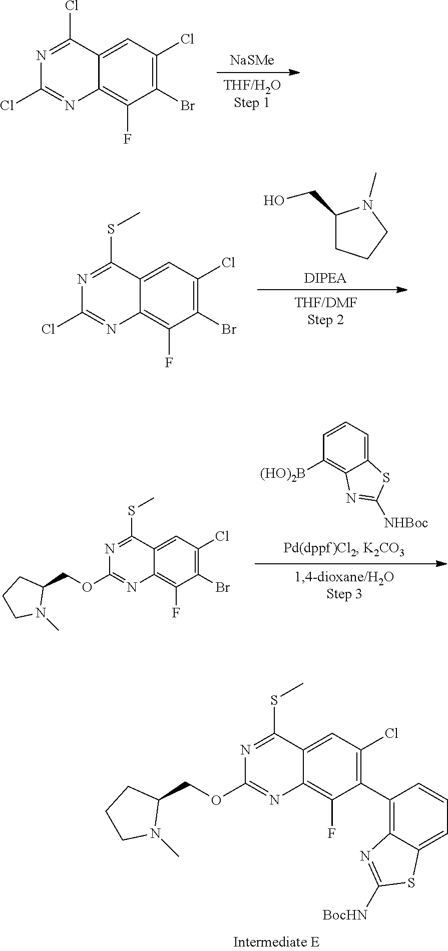

Step 1: 7-Bromo-2,6-dichloro-8-fluoro-4-(methylthio)quinazoline. NaSMe (6.40 g, 18.3 mmol, 5.82 mL, 20% purity) was added to the solution of 7-bromo-2,4,6-trichloro-8-fluoro-quinazoline (6.00 g, 18.2 mmol) in THF (120 mL) at 0° C. under N₂. The mixture was stirred at 0° C. for 1 h. The reaction mixture was diluted with water 150 mL, and extracted with EtOAc 300 mL. The organic layer was concentrated under reduced pressure to give a residue. The desired compound 7-bromo-2,6-dichloro-8-fluoro-4-(methylthio)quinazoline (4.35 g, 12.7 mmol, 70% yield) was obtained as a yellow solid after drying the residue in vacuo. m/z (ESI, +ve ion): 342.8 (M+H)⁺.

Step 2: (S)-7-Bromo-6-chloro-8-fluoro-2-((1-methylpyrrolidin-2-yl)methoxy)-4-(methylthio)quinazoline. To a mixture of 7-bromo-2,6-dichloro-8-fluoro-4-(methylthio)quinazoline (9.90 g, 29.0 mmol) and [(2S)-1-methylpyrrolidin-2-yl]methanol (5.00 g, 43.4 mmol, 5.16 mL) in THF (120 mL) and DMF (60.5 mL) was added DIPEA (7.48 g, 57.9 mmol, 10.1 mL) in one portion at 20° C. under N₂. The mixture was stirred at 80° C. for 10 h. The reaction mixture was diluted with water (200 mL) and extracted with EtOAc (200 mL×3). The combined organic layers were dried over anhydrous Na₂SO₄, filtered and concentrated under reduced pressure to give a residue. The residue was purified by column chromatography on silica gel, eluting with petroleum ether/EtOAc (1/0 to 0/1). The compound (S)-7-bromo-6-chloro-8-fluoro-2-((1-methylpyrrolidin-2-yl)methoxy)-4-(methylthio)quinazoline (5.48 g, 13.0 mmol, 45% yield) was obtained as a yellow solid. m/z (ESI, +ve ion): 422.0 (M+H)⁺.

Step 3: tert-Butyl (4-(6-chloro-8-fluoro-2-(((S)-1-methylpyrrolidin-2-yl)methoxy)-4-(methylthio)quinazolin-7-yl)benzo[d]thiazol-2-yl)carbamate. To a mixture of (S)-7-bromo-6-chloro-8-fluoro-2-((1-methylpyrrolidin-2-yl)methoxy)-4-(methylthio)quinazoline (4.50 g, 10.7 mmol) and [2-(tert-butoxycarbonylamino)-1,3-benzothiazol-4-yl]boronic acid (3.78 g, 12.8 mmol) in dioxane (90 mL) and H₂O (9 mL) was added K₂CO₃ (4.43 g, 32.1 mmol) in one portion at 20° C. under N₂. Then Pd(dppf)Cl₂ (0.78 g, 1.07 mmol) was added to the mixture. The reaction mixture was stirred at 100° C. for 10 h. The reaction mixture was diluted with water (50 mL) and extracted with EtOAc (60 mL×3). The combined organic layers were dried over anhydrous Na₂SO₄, filtered and concentrated under reduced pressure to give a residue. The residue was purified by column chromatography on silica gel, eluting with petroleum ether/EtOAc (1/0 to 0/1). tert-Butyl (4-(6-chloro-8-fluoro-2-(((S)-1-methylpyrrolidin-2-yl)methoxy)-4-(methylthio)quinazolin-7-yl)benzo[d]thiazol-2-yl)carbamate (4.60 g, 7.80 mmol, 73% yield) was obtained as a yellow solid. m/z (ESI, +ve ion): 590.2 (M+H)⁺.

TABLE 1
Additional Intermediates Prepared in an Analogous Manner to Intermediate E.

| | | | MS |
|---|---|---|---|
| | | | m/z (ESI, +ve |
| Int. # | Chemical Structure | Name | ion) |
| Intermediate F | 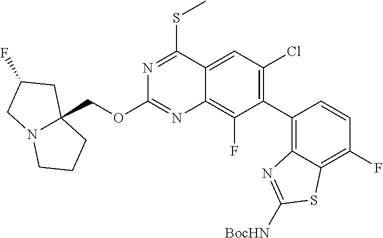 [SMILES: CSc1nc(OC[C@@]23CCCN2C[C@H](F)C3)nc2c(F)c(-c3ccc(F)c4sc(NC(=O)OC(C)(C)C)nc34)c(Cl)cc12] | tert-butyl (4-(6-chloro-8- fluoro-2-(((2R,7aS)-2- fluorotetrahydro-1H- pyrrolizin-7a(5H)- yl)methoxy)-4- (methylthio)quinazolin-7-yl)- 7-fluorobenzo[d]thiazol-2- yl)carbamate | 652.0 (M + H)⁺ |
| Intermediate G | 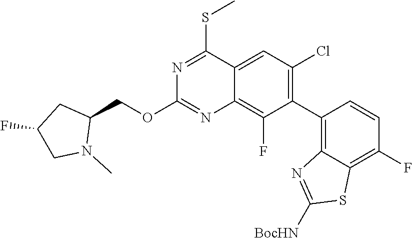 [SMILES: CSc1nc(OC[C@@H]2C[C@@H](F)CN2C)nc2c(F)c(-c3ccc(F)c4sc(NC(=O)OC(C)(C)C)nc34)c(Cl)cc12] | tert-butyl (4-(6-chloro-8- fluoro-2-(((2S,4R)-4-fluoro-1- methylpyrrolidin-2- yl)methoxy)-4- (methylthio)quinazolin-7-yl)- 7-fluorobenzo[d]thiazol-2- yl)carbamate | 626.0 (M + H)⁺ |
| Intermediate H | 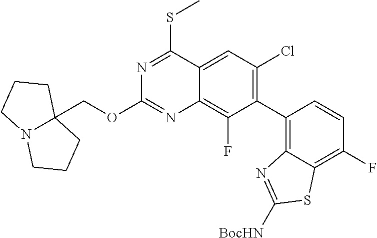 [SMILES: CSc1nc(OCC23CCCN2CCC3)nc2c(F)c(-c3ccc(F)c4sc(NC(=O)OC(C)(C)C)nc34)c(Cl)cc12] | tert-butyl (4-(6-chloro-8- fluoro-4-(methylthio)-2- ((tetrahydro-1H-pyrrolizin- 7a(5H)- yl)methoxy)quinazolin-7-yl)- 7-fluorobenzo[d]thiazol-2- yl)carbamate | 634.0 (M + H)⁺ |
| Intermediate I | 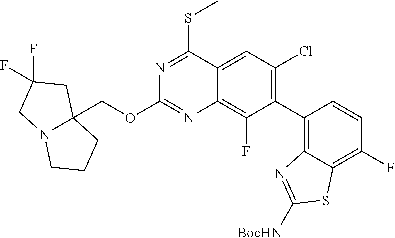 [SMILES: CSc1nc(OCC23CCCN2CC(F)(F)C3)nc2c(F)c(-c3ccc(F)c4sc(NC(=O)OC(C)(C)C)nc34)c(Cl)cc12] | tert-butyl (4-(6-chloro-2-((2,2- difluorotetrahydro-1H- pyrrolizin-7a(5H)- yl)methoxy)-8-fluoro-4- (methylthio)quinazolin-7-yl)- 7-fluorobenzo[d]thiazol-2- yl)carbamate | 670.0 (M + H)⁺ |
| Intermediate J | 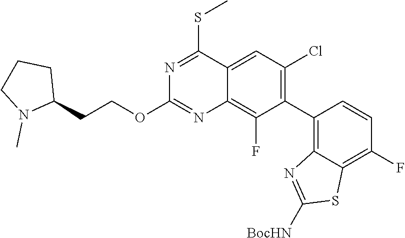 [SMILES: CSc1nc(OCC[C@@H]2CCCN2C)nc2c(F)c(-c3ccc(F)c4sc(NC(=O)OC(C)(C)C)nc34)c(Cl)cc12] | tert-butyl (4-(6-chloro-8- fluoro-2-(2-((S)-1- methylpyrrolidin-2-yl)ethoxy)- 4-(methylthio)quinazolin-7- yl)-7-fluorobenzo[d]thiazol-2- yl)carbamate | 622.0 (M + H)⁺ |

### tert-Butyl 4-(7-(2-((tert-butoxycarbonyl)amino)benzo[d]thiazol-4-yl)-6-chloro-8-fluoro-2-(methylsulfinyl)quinazolin-4-yl)piperazine-1-carboxylate (Intermediate K

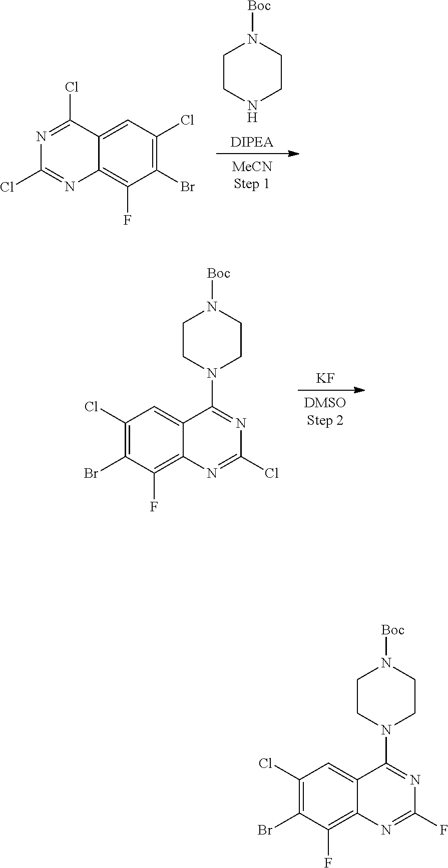

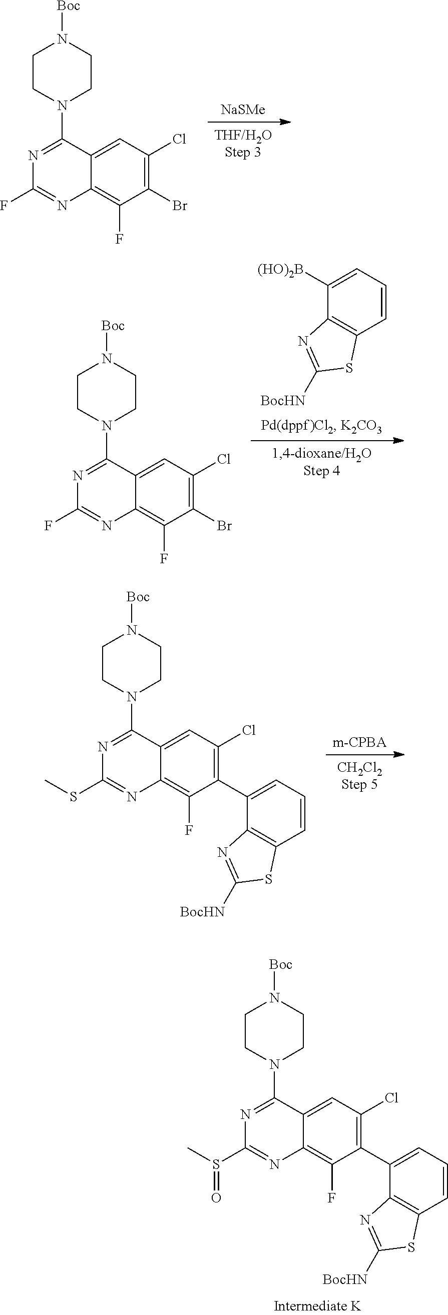

Step 1: tert-Butyl 4-(7-bromo-2,6-dichloro-8-fluoroquinazolin-4-yl)piperazine-1-carboxylate. To a mixture of 7-bromo-2,4,6-trichloro-8-fluoro-quinazoline (20.0 g, 60.5 mmol,) in MeCN (240 mL) was added DIPEA (23.5 g, 183 mmol, 31.6 mL) and tert-butyl piperazine-1-carboxylate (11.3 g, 60.5 mmol). Then the mixture was stirred at 25° C. for 1 h to give an orange suspension. The suspension was combined with another batch (5 g scale). The reaction mixture was filtered and the filter cake was washed with 15 ml, of MeCN, dried in vacuum to give tert-butyl 4-(7-bromo-2,6-dichloro-8-fluoro-quinazolin-4-yl)piperazine-1-carboxylate (32.0 g, crude) as yellow solid.

Step 2: tert-Butyl 4-(7-bromo-6-chloro-2,8-difluoroquinazolin-4-yl)piperazine-1-carboxylate. To a mixture of tert-butyl 4-(7-bromo-2,6-dichloro-8-fluoro-quinazolin-4-yl)piperazine-1-carboxylate (16.0 g, 33.3 mmol) in DMSO (150 mL) was added KF (19.4 g, 333 mmol, 7.81 mL). Then the mixture was heated at 120° C. for 12 h. LCMS showed the reaction was completed. Another batch with the same scale was combined for the purification. The reaction mixture was diluted with H₂O (500 mL) and EtOAc (800 mL), and extracted with EtOAc (500 mL×3). The combined organic layers were washed with brine (500 mL), dried over anhydrous Na₂SO₄, filtered and concentrated under reduced pressure to give a residue. The residue was purified by column chromatography on silica gel, eluting with petroleum ether/EtOAc (100/1 to 1/1) to give tert-butyl 4-(7-bromo-6-chloro-2,8-difluoro-quinazolin-4-yl)piperazine-1-carboxylate (19.0 g, 4.09 mmol, 54% yield over 2 steps) as yellow solid. m/z (ESI, +ve ion): 463.0 (M+H)⁺.

Step 3: tert-Butyl 4-(7-bromo-6-chloro-8-fluoro-2-(methylthio)quinazolin-4-yl)piperazine-1-carboxylate. To a solution of tert-butyl 4-(7-bromo-6-chloro-2,8-difluoro-quinazolin-4-yl)piperazine-1-carboxylate (7.0 g, 15.1 mmol) in THF (180 mL) was added NaSMe (5.29 g, 15.1 mmol, 4.81 mL, 20% purity) at 0° C. The mixture was stirred at 25° C. for 12 h. The reaction mixture was diluted with H₂O (500 mL) and extracted with EtOAc (200 mL×3). The combined organic layers were dried over anhydrous Na₂SO₄, filtered and concentrated under reduced pressure to give a residue. The residue was purified by column chromatography on silica gel, eluting with petroleum ether/EtOAc (1/0 to 5/1). tert-Butyl 4-(7-bromo-6-chloro-8-fluoro-2-methylsulfanyl-quinazolin-4-yl) piperazine-1-carboxylate (5.0 g, 10.2 mmol, 67% yield) was obtained as a yellow solid.

Step 4: tert-Butyl 4-(7-(2-((tert-butoxycarbonyl)amino)benzo[d]thiazol-4-yl)-6-chloro-8-fluoro-2-(methylthio)quinazolin-4-yl)piperazine-1-carboxylate. To a solution of tert-butyl 4-(7-bromo-6-chloro-8-fluoro-2-methylsulfanyl-quinazolin-4-yl)piperazine-1-carboxylate (5.00 g, 10.2 mmol), [2-(tert-butoxycarbonylamino)-1,3-benzothiazol-4-yl]boronic acid (3.29 g, 11.2 mmol) and K₃PO₄ (4.32 g, 20.3 mmol) in dioxane (116 mL) and H₂O (23 mL) was added Pd(dppf)Cl₂ (0.74 g, 1.02 mmol) under N₂. The mixture was stirred at 95° C. for 7 h under N₂. The reaction mixture was concentrated under reduced pressure to give a residue. The residue was purified by column chromatography on silica gel, eluting with petroleum ether/EtOAc (1/0 to 3/1). Compound tert-butyl 4-(7-(2-((tert-butoxycarbonyl)amino)benzo[d]thiazol-4-yl)-6-chloro-8-fluoro-2-(methylthio)quinazolin-4-yl)piperazine-1-carboxylate (3.35 g, 5.07 mmol, 50% yield) was obtained as a white solid. m/z (ESI, +ve ion): 661.1 (M+H)⁺.

Step 5: tert-Butyl 4-(7-(2-((tert-butoxycarbonyl)amino)benzo[d]thiazol-4-yl)-6-chloro-8-fluoro-2-(methylsulfinyl)quinazolin-4-yl)piperazine-1-carboxylate. To a solution of tert-butyl 4-(7-(2-((tert-butoxycarbonyl)amino)benzo[d]thiazol-4-yl)-6-chloro-8-fluoro-2-(methylthio)quinazolin-4-yl)piperazine-1-carboxylate (6.7 g, 10.1 mmol) in DCM (48 mL) was added m-CPBA (2.46 g, 12.2 mmol, 85% purity). The mixture was stirred at 0° C. for 1 h. The reaction mixture was quenched by addition saturated aq. Na₂SO₃ (500 mL) and extracted with DCM (100 mL×3). The combined organic layers were dried over anhydrous Na₂SO₄, filtered and concentrated under reduced pressure to give a residue. The residue was purified by column chromatography on silica gel, eluting with petroleum ether/EtOAc (1/1 to 1/0). tert-Butyl 4-(7-(2-(tert-butoxycarbonylamino)-1,3-benzothiazol-4-yl)-6-chloro-8-fluoro-2-methylsulfinyl-quinazolin-4-yl)piperazine-1-carboxylate (5.46 g, 7.82 mmol, 80% yield) was obtained as white solid. m/z (ESI, +ve ion): 677.1 (M+H)⁺.

### 7-Bromo-2,4,6-trichloro-8-fluoroquinoline (Intermediate L

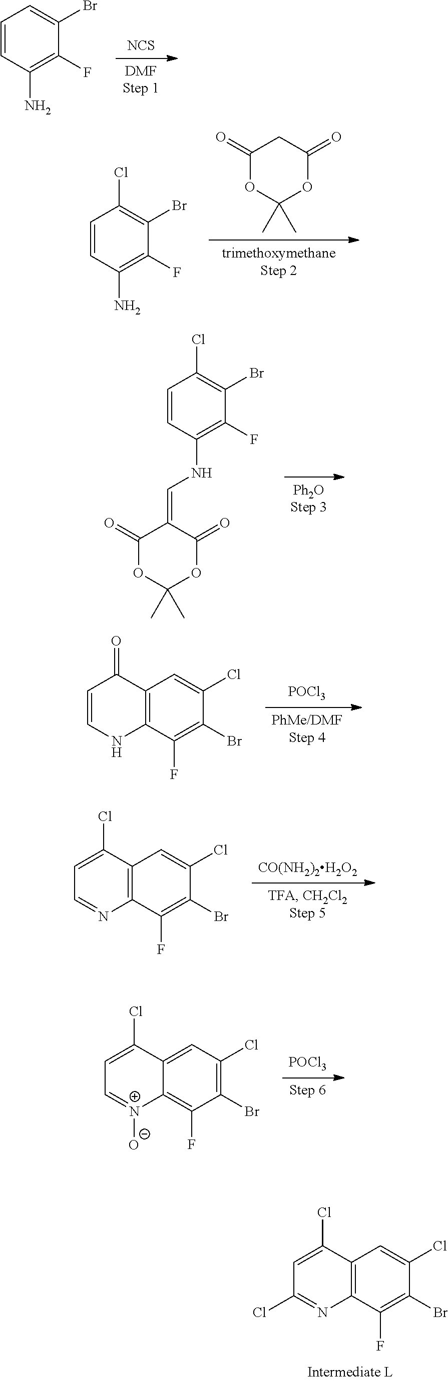

Step 1: 3-Bromo-4-chloro-2-fluoroaniline. To a mixture of 3-bromo-2-fluoroaniline (100 g, 526 mmol) in DMF (1000 mL) was added NCS (73.79 g, 553 mmol) in one portion at 25° C. under N₂. The mixture was stirred at 25° C. for 3 h. The reaction mixture was diluted with H₂O (3000 mL) and extracted with EtOAc (700 mL×3). The combined organic layers were washed with brine (500 mL×3), dried over Na₂SO₄, filtered and concentrated under reduced pressure to give a residue. The residue was purified by column chromatography on silica gel, eluting with petroleum ether/ethyl acetate=1/0 to 5/1. The desired product (60 g, 267 mmol, 51% yield) was obtained as a yellow solid.

Step 2: 5-(((3-Bromo-4-chloro-2-fluorophenyl)amino)methylene)-2,2-dimethyl-1,3-dioxane-4,6-dione. 2,2-Dimethyl-1,3-dioxane-4,6-dione (14.13 g, 98 mmol) was added to trimethoxymethane (47.28 g, 446 mmol, 48.84 mL) and the mixture was warmed at 110° C. over a period of 0.5 h under N₂. The resulting solution was stirred at 85° C. for 1.5 h. 3-Bromo-4-chloro-2-fluoroaniline (20 g, 89 mmol) was added. The solution was stirred at 85° C. for another 1 h. The reaction was cooled to 25° C., then filtered. The filter cake was dried. Crude 5-(((3-bromo-4-chloro-2-fluorophenyl)amino)methylene)-2,2-dimethyl-1,3-dioxane-4,6-dione (25 g, 66 mmol, 74% yield) was obtained as off-yellow solid, which was used in the next step without further manipulation.

Step 3: 7-Bromo-6-chloro-8-fluoroquinolin-4(1H)-one. 5-(((3-Bromo-4-chloro-2-fluorophenyl)amino)methylene)-2,2-dimethyl-1,3-dioxane-4,6-dione (10 g, 26 mmol) in diphenyl ether (44.96 g, 264 mmol, 42.02 mL) was stirred at 180° C. for 40 min. The reaction was cooled to 25° C. Petroleum ether (200 ml) was added and the reaction was stirred for 10 min. The suspension was filtered and the filter cake was dried. The residue was purified by column chromatography on silica gel, eluting with petroleum ether/ethyl acetate=30/1 to 1/1. The desired product (1.83 g, 6.63 mmol, 25% yield) was obtained as brown solid.

Step 4: To a mixture of 7-bromo-6-chloro-8-fluoroquinolin-4(1H)-one (2.5 g, 9.04 mmol) in toluene (5 mL) was added POCl₃ (5.55 g, 36.17 mmol, 3.36 mL), DMF (6.61 mg, 90.42 umol, 6.96 μL) at 25° C. under N₂. The mixture was stirred at 110° C. for 3 h. The reaction mixture was concentrated under reduced pressure to remove POCl₃ and toluene. The residue was diluted with aq. NaHCO₃ (20 mL), extracted with EtOAc (20 mL×3), filtered and concentrated under reduced pressure to give a residue. The residue was purified by column chromatography on silica gel, eluting with petroleum ether/ethyl acetate=100/1 to 200/1. The desired product (2.3 g, 7.64 mmol, 85% yield) was obtained as white solid. m/z (ESI): 295.9 (M+H)⁺.

Step 5: 7-Bromo-4,6-dichloro-8-fluoroquinoline 1-oxide. To a mixture of 7-bromo-4,6-dichloro-8-fluoroquinoline (12 g, 40.69 mmol) in DCM (240 mL) was added urea hydrogen peroxide (22.96 g, 244.12 mmol) and TFA (55.67 g, 488.24 mmol, 36 mL) in one portion at 0° C. under N₂. The mixture was stirred at 0° C. for 30 min and then at 40° C. for 23.5 h. The reaction mixture was poured into H₂O (250 mL) at 15° C., and then extracted with DCM (150 mL×3). The combined organic layers were washed with sat. Na₂SO₃ (200 mL×3), dried over Na₂SO₄, and filtered. The filtrate was concentrated under reduced pressure to give a residue. The residue was purified by column chromatography on silica gel, eluting with petroleum ether/ethyl acetate=100/1 to 0/1 to give the product (6 g, 19.30 mmol, 47% yield) as white solid. m/z (ESI): 309.9 (M+H)⁺.

Step 6: 7-Bromo-2,4,6-trichloro-8-fluoroquinoline. The mixture of 7-Bromo-4,6-dichloro-8-fluoroquinoline 1-oxide (6.3 g, 20 mmol) in POCl₃ (46.60 g, 304 mmol, 28 mL) was stirred at 110° C. for 1 h. The reaction mixture was concentrated under reduced pressure. The residue was diluted with EtOAc (50 mL) and poured into H₂O (100 mL) at 15° C. and then extracted with EtOAc (70 mL×3). The combined organic layers were dried over Na₂SO₄ and filtered. The filtrate was concentrated under reduced pressure to give a residue. The residue was purified by column chromatography on silica gel, eluting with petroleum ether/ethyl acetate=1/0 to 0/1 to give 7-bromo-2,4,6-trichloro-8-fluoroquinoline (5 g, 15.12 mmol, 73% yield) as white solid. m/z (ESI): 329.7 (M+H)⁺.

### Experimental Procedures

### 4-(6-Chloro-8-fluoro-2-(((S)-1-methylpyrrolidin-2-yl)methoxy)-4-(3-(trifluoromethyl)piperazin-1-yl)quinazolin-7-yl)benzo[d]thiazol-2-amine (Example 1

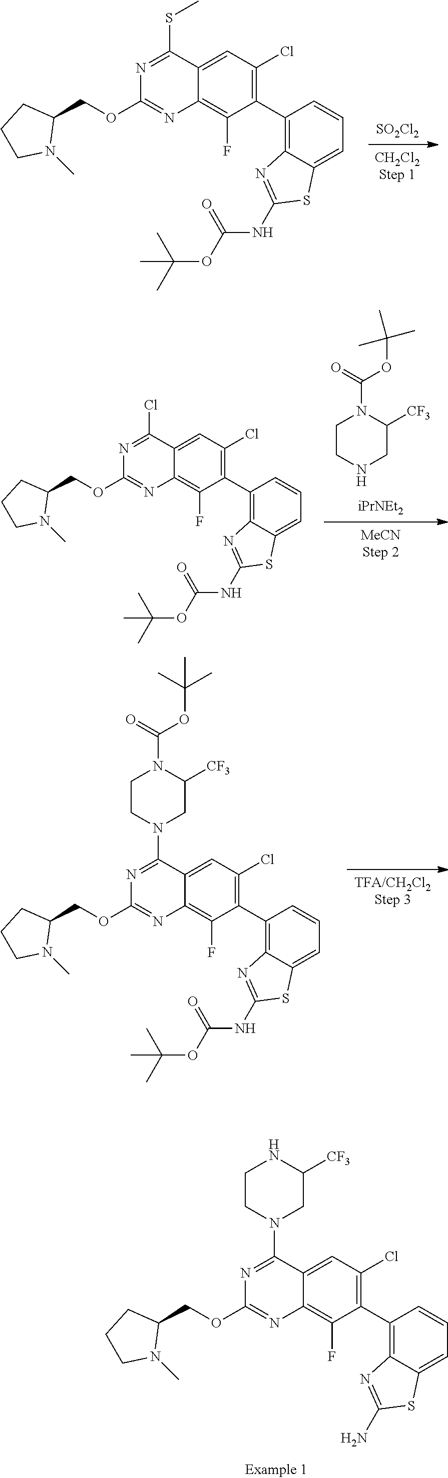

Step 1: tert-Butyl (4-(4,6-dichloro-8-fluoro-2-(((S)-1-methylpyrrolidin-2-yl)methoxy)quinazolin-7-yl)benzo[d]thiazol-2-yl)carbamate. To a solution of tert-butyl (4-(6-chloro-8-fluoro-2-(((S)-1-methylpyrrolidin-2-yl)methoxy)-4-(methylthio)quinazolin-7-yl)benzo[d]thiazol-2-yl)carbamate (60 mg, 0.10 mmol) in DCM (2 mL) at 0° C. was added sulfuryl chloride (1.0 M solution in DCM, 0.31 mL, 0.31 mmol, Sigma-Aldrich Corporation) slowly. The reaction mixture was stirred at 0° C. for 1 h. The reaction mixture was concentrated without heating and used directly in the next step. m/z (ESI, +ve ion): 578.0 (M+H⁺).

Step 2: tert-Butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)-2-(trifluoromethyl)piperazine-1-carboxylate. To a solution of tert-butyl (4-(4,6-dichloro-8-fluoro-2-(((S)-1-methylpyrrolidin-2-yl)methoxy)quinazolin-7-yl)benzo[d]thiazol-2-yl)carbamate (20 mg, 0.034 mmol) in acetonitrile (1 mL) was added Hunig's base (13 mg, 0.018 mL, 0.10 mmol, Sigma-Aldrich Corporation) and 2-trifluoromethyl-piperazine-1-carboxylic acid tert-butyl ester HCl (17 mg, 0.068 mmol, Anichem Inc.). The resulting mixture was stirred at room temperature for 2 h. The reaction mixture was purified by reverse phase HPLC to afford tert-butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)-2-(trifluoromethyl)piperazine-1-carboxylate as white solid. m/z (ESI, +ve ion): 796.2 (M+H)⁺.

Step 3: 4-(6-Chloro-8-fluoro-2-(((S)-1-methylpyrrolidin-2-yl)methoxy)-4-(3-(trifluoromethyl)piperazin-1-yl)quinazolin-7-yl)benzo[d]thiazol-2-amine. tert-Butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)-2-(trifluoromethyl)piperazine-1-carboxylate was dissolved in 0.5 mL DCM. Trifluoroacetic acid (0.77 g, 0.5 mL, 6.71 mmol, Sigma-Aldrich Corporation) was added and the reaction mixture was stirred at room temperature for 1 h. The reaction mixture was concentrated under reduced pressure and purified by reverse phase HPLC to afford 4-(6-chloro-8-fluoro-2-(((S)-1-methylpyrrolidin-2-yl)methoxy)-4-(3-(trifluoromethyl)piperazin-1-yl)quinazolin-7-yl)benzo[d]thiazol-2-amine (8.4 mg, 0.014 mmol, 41% yield) as white solid. m/z (ESI, +ve ion): 596.2 (M+H)⁺. ¹H NMR (400 MHz, METHANOL-d4) δ ppm 8.04 (d, J=1.5 Hz, 1H), 7.87 (dt, J=7.5, 1.5 Hz, 1H), 7.35-7.42 (m, 2H), 4.89-4.97 (m, 1H), 4.58-4.82 (m, 2H), 4.38-4.45 (m, 1H), 4.18 (td, J=6.8, 3.6 Hz, 1H), 3.87-3.98 (m, 1H), 3.56-3.81 (m, 3H), 3.35-3.50 (m, 1H), 3.20-3.31 (m, 2H), 3.11 (s, 3H), 2.43 (br d, J=7.3 Hz, 1H), 2.23 (br s, 1H), 2.05-2.19 (m, 2H).

TABLE 2
Additional Examples. Prepared in an Analogous Manner to Example 1.

| | | | MS | |
|---|---|---|---|---|
| | | | m/z | |
| | | | (ESI, | |
| Ex. | | | +ve | |
| # | Structure | Name | ion) | ¹H NMR |
| 2 | 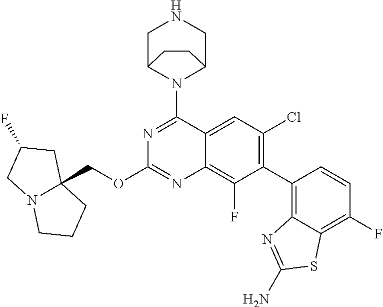 [SMILES: Nc1nc2c(-c3c(Cl)cc4c(N5C6CCC5CNC6)nc(OC[C@@]56CCCN5C[C@H](F)C6)nc4c3F)ccc(F)c2s1] | 4-(4-((1R,5S)-3,8- diazabicyclo[3.2.1] octan-8-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 616.2 (M + H)⁺ | ¹H NMR (400 MHz, METHANOL-d4) δ ppm 8.03 (d, J = 1.7 Hz, 1 H), 7.26 (ddd, J = 8.5, 5.4, 1.6 Hz, 1 H), 7.05 (t, J = 8.9 Hz, 1 H), 5.44-5.74 (m, 1 H), 5.20 (br s, 2 H), 4.66-4.76 (m, 2 H), 3.85-4.07 (m, 3 H), 3.65- 3.76 (m, 2 H), 3.43-3.53 (m, 3 H), 2.72 (br t, J = 3.6 Hz, 1 H), 2.56-2.67 (m, 1 H), 2.29-2.49 (m, 5 H), 2.08-2.26 (m, 3 H). |
| 3 | 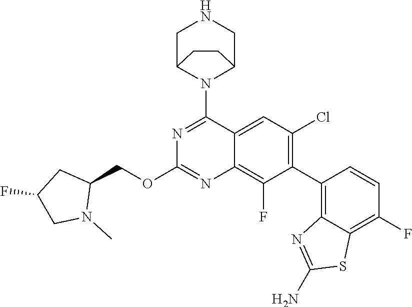 [SMILES: CN1C[C@H](F)C[C@H]1COc1nc(N2C3CCC2CNC3)c2cc(Cl)c(-c3ccc(F)c4sc(N)nc34)c(F)c2n1] | 4-(4-((1R,5S)-3,8- diazabicyclo[3.2.1] octan-8-yl)-6-chloro- 8-fluoro-2- (((2S,4R)-4-fluoro- 1-methylpyrrolidin- 2-yl)methoxy) quinazolin-7-yl)-7- fluorobenzo[d] thiazol-2-amine | 590.2 (M + H)⁺ | ¹H NMR (400 MHz, METHANOL-d4) δ ppm 8.04 (t, J = 1.5 Hz, 1 H), 7.26 (ddd, J = 8.6, 5.2, 3.6 Hz, 1 H), 7.05 (t, J = 8.9 Hz, 1 H), 5.38-5.58 (m, 1 H), 5.18 (br s, 2 H), 4.93-5.06 (m, 1 H), 4.64-4.78 (m, 1 H), 4.21- 4.33 (m, 1 H), 3.98-4.20 (m, 1 H), 3.61-3.77 (m, 3 H), 3.47 (br d, J = 11.3 Hz, 2 H), 3.20 (s, 3 H), 2.63-2.75 (m, 1 H), 2.27-2.42 (m, 3 H), 2.09-2.23 (m, 2 H). |
| 4 | 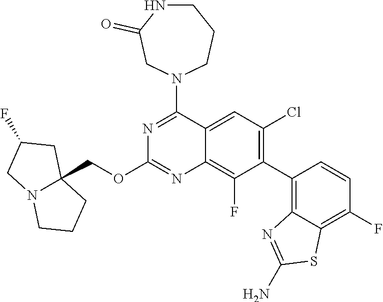 [SMILES: Nc1nc2c(-c3c(Cl)cc4c(N5CCCNC(=O)C5)nc(OC[C@@]56CCCN5C[C@H](F)C6)nc4c3F)ccc(F)c2s1] | 4-(7-(2-amino-7- fluorobenzo[d] thiazol-4-yl)-6- chloro-8- fluoro-2-(((2R,7aS)- 2-fluorotetrahydro- 1H-pyrrolizin- 7a(5H)- yl)methoxy) quinazolin-4- yl)-1,4- diazepan-2-one | 618.2 (M + H)⁺ | ¹H NMR (400 MHz, METHANOL-d4) δ ppm 8.19 (d, J = 1.7 Hz, 1 H), 7.27 (ddd, J = 8.4, 5.4, 1.0 Hz, 1 H), 7.06 (t, J = 8.6 Hz, 1 H), 5.48-5.68 (m, 1 H), 4.64- 4.84 (m, 2 H), 4.50 (s, 2 H), 4.29 (br t, J = 6.0 Hz, 2 H), 3.84-4.08 (m, 3 H), 3.42- 3.53 (m, 1 H), 3.15-3.31 (m, 2 H), 2.55-2.74 (m, 2 H), 2.17-2.46 (m, 6 H). |
| 5 | 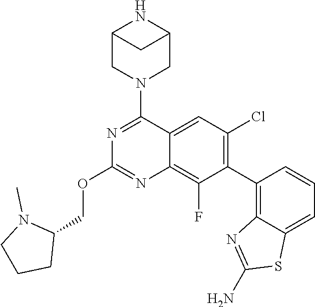 [SMILES: CN1CCC[C@H]1COc1nc(N2CC3CC(C2)N3)c2cc(Cl)c(-c3cccc4sc(N)nc34)c(F)c2n1] | 4-(4-(3,6- diazabicyclo[3.1.1] heptan-3-yl)-6- chloro-8-fluoro-2- (((S)-1- methylpyrrolidin-2- yl)methoxy) quinazolin-7- yl)benzo[d]thiazol- 2-amine | 540.2 (M + H)⁺ | ¹H NMR (400 MHz, METHANOL-d4) δ ppm 8.43 (d, J = 1.7 Hz, 1 H), 7.89 (dt, J = 7.4, 1.8 Hz, 1 H), 7.37-7.45 (m, 2 H), 4.88 - 4.98 (m, 1 H), 4.79-4.86 (m, 3 H), 4.57-4.74 (m, 5 H), 3.91 (br s, 1 H), 3.76 (br d, J = 6.9 Hz, 1 H), 3.21-3.31 (m, 1 H), 3.05-3.19 (m, 4 H), 2.42 (br d, J = 6.3 Hz, 1 H), 2.22 (br s, 1 H), 2.01- 2.18 (m, 3 H). |
| 6 | 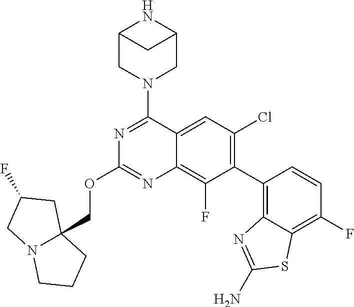 [SMILES: Nc1nc2c(-c3c(Cl)cc4c(N5CC6CC(C5)N6)nc(OC[C@@]56CCCN5C[C@H](F)C6)nc4c3F)ccc(F)c2s1] | 4-(4-((1R,5S)-3,6- diazabicyclo[3.1.1] heptan-3-yl)-6- chloro-8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 601.6 (M + H)⁺ | ¹H NMR (400 MHz, METHANOL-d4) δ ppm 8.41 (d, J = 1.7 Hz, 1 H), 7.29 (ddd, J = 8.4, 5.4, 1.9 Hz, 1 H), 7.07 (t, J = 8.8 Hz, 1 H), 5.43-5.74 (m, 1 H), 4.59- 4.86 (m, 8 H), 4.07 (dd, J = 14.1, 3.0 Hz, 1 H), 3.84- 4.00 (m, 2 H), 3.43-3.53 (m, 1 H), 3.10-3.19 (m, 1 H), 2.55-2.77 (m, 2 H), 2.30-2.49 (m, 3 H), 2.11- 2.23 (m, 1 H), 2.04 (d, J = 10.0 Hz, 1 H). |
| 7 | 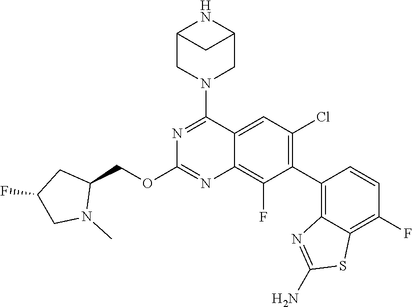 [SMILES: CN1C[C@H](F)C[C@H]1COc1nc(N2CC3CC(C2)N3)c2cc(Cl)c(-c3ccc(F)c4sc(N)nc34)c(F)c2n1] | 4-(4-((1R,5S)-3,6- diazabicyclo[3.1.1] heptan-3-yl)-6- chloro-8-fluoro-2- (((2S,4R)-4-fluoro- 1-methylpyrrolidin- 2-yl)methoxy) quinazolin-7-yl)-7- fluorobenzo[d] thiazol-2-amine | 576.2 (M + H)⁺ | ¹H NMR (400 MHz, METHANOL-d4) δ ppm 8.41 (s, 1 H), 7.28 (ddd, J = 8.5, 5.3, 4.4 Hz, 1 H), 7.06 (td, J = 8.8, 2.3 Hz, 1 H), 5.34-5.63 (m, 1 H), 5.01 (ddd, J = 13.3, 8.2, 2.2 Hz, 1 H), 4.73-4.85 (m, 3 H), 4.57-4.68 (m, 4 H), 4.22- 4.33 (m, 1 H), 3.96-4.19 (m, 1 H), 3.61-3.76 (m, 1 H), 3.11-3.22 (m, 4 H), 2.63-2.75 (m, 1 H), 2.30- 2.50 (m, 1 H), 2.02-2.07 (m, 1 H). |
| 8 | 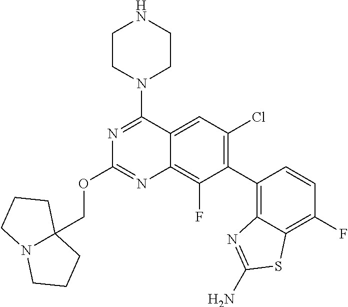 [SMILES: Nc1nc2c(-c3c(Cl)cc4c(N5CCNCC5)nc(OCC56CCCN5CCC6)nc4c3F)ccc(F)c2s1] | 4-(6-chloro-8- fluoro-4-(piperazin- 1-yl)-2-((tetrahydro- 1H-pyrrolizin- 7a(5H)- yl)methoxy) quinazolin-7-yl)-7- fluorobenzo[d] thiazol-2-amine | 572.2 (M + H)⁺ | ¹H NMR (400 MHz, METHANOL-d4) δ ppm 8.02 (s, 1 H), 7.25 (dd, J = 8.5, 5.4 Hz, 1 H), 7.05 (t, J = 8.8 Hz, 1 H), 4.69 (s, 2 H), 4.11-4.19 (m, 4 H), 3.66-3.77 (m, 2 H), 3.47- 3.57 (m, 4 H), 3.35-3.38 (m, 1 H), 3.24-3.31 (m, 1 H), 2.10-2.38 (m, 8 H). |
| 9 | 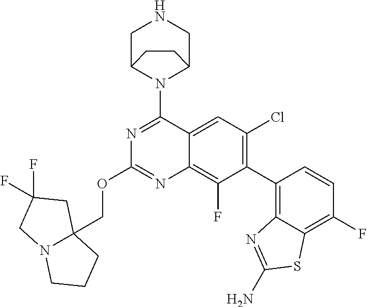 [SMILES: Nc1nc2c(-c3c(Cl)cc4c(N5C6CCC5CNC6)nc(OCC56CCCN5CC(F)(F)C6)nc4c3F)ccc(F)c2s1] | 4-(4-((1R,5S)-3,8- diazabicyclo[3.2.1] octan-8-yl)-6-chloro- 2-((2,2- difluorotetrahydro- 1H-pyrrolizin- 7a(5H)- yl)methoxy)-8- fluoroquinazolin- 7-yl)-7- fluorobenzo[d] thiazol-2-amine | 634.0 (M + H)⁺ | ¹H NMR (400 MHz, METHANOL-d4) δ ppm 8.04 (d, J = 1.3 Hz, 1 H), 7.27 (ddd, J = 8.4, 5.4, 1.3 Hz, 1 H), 7.06 (t, J = 8.8 Hz, 1 H), 5.21 (br d, J = 6.3 Hz, 2 H), 4.72-4.81 (m, 2 H), 4.21- 4.33 (m, 1 H), 3.84-4.01 (m, 2 H), 3.64-3.77 (m, 2 H), 3.42-3.54 (m, 3 H), 2.80-3.06 (m, 2 H), 2.48 (dt, J = 11.6, 5.9 Hz, 1 H), 2.15-2.42 (m, 7 H). |
| 10 | 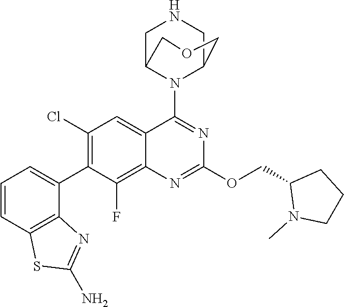 [SMILES: CN1CCC[C@H]1COc1nc(N2C3CNCC2COC3)c2cc(Cl)c(-c3cccc4sc(N)nc34)c(F)c2n1] | 4-(4-(3-oxa-7,9- diazabicyclo[3.3.1] nonan-9-yl)-6-chloro- 8-fluoro-2-(((S)-1- methylpyrrolidin-2- yl)methoxy) quinazolin-7- yl)benzo[d] thiazol-2-amine | 570.2 (M + H)⁺ | ¹H NMR (400 MHz, METHANOL-d4) δ ppm 7.87 (s, 1 H), 7.84 (d, J = 7.3 Hz, 1 H), 7.30-7.38 (m, 2 H), 4.88-4.97 (m, 1 H), 4.78 (br s, 2 H), 4.67-4.76 (m, 1 H), 4.24-4.33 (m, 4 H), 3.92 (br d, J = 5.4 Hz, 1 H), 3.72-3.85 (m, 5 H), 3.21-3.31 (m, 1 H), 3.12 (s, 3 H), 2.36-2.48 (m, 1 H), 2.05-2.27 (m, 3 H). |
| 11 | 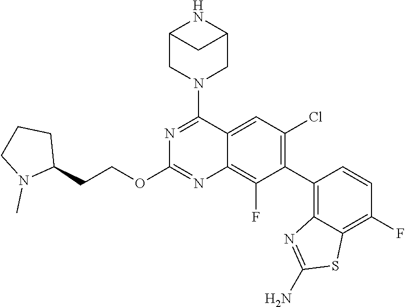 [SMILES: CN1CCC[C@H]1CCOc1nc(N2CC3CC(C2)N3)c2cc(Cl)c(-c3ccc(F)c4sc(N)nc34)c(F)c2n1] | 4-(4-((1R,5S)-3,6- diazabicyclo[3.1.1] heptan-3-yl)-6- chloro-8-fluoro-2- (2-((S)-1- methylpyrrolidin-2- yl)ethoxy) quinazolin-7-yl)-7- fluorobenzo[d] thiazol-2-amine | 572.0 (M + H)⁺ | ¹H NMR (400 MHz, METHANOL-d4) δ ppm 8.38 (s, 1 H), 7.26 (dd, J = 8.5, 5.4 Hz, 1 H), 7.05 (t, J = 8.8 Hz, 1 H), 4.79-4.84 (m, 2 H), 4.57-4.73 (m, 6 H), 3.67-3.78 (m, 1 H), 3.59 (br dd, J = 7.6, 4.7 Hz, 1 H), 3.10-3.29 (m, 2 H), 3.01 (s, 3 H), 2.34-2.56 (m, 2 H), 2.03-2.23 (m, 4 H), 1.89-2.01 (m, 1 H). |
| 12 | 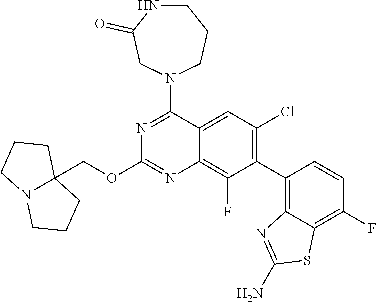 [SMILES: Nc1nc2c(-c3c(Cl)cc4c(N5CCCNC(=O)C5)nc(OCC56CCCN5CCC6)nc4c3F)ccc(F)c2s1] | 4-(7-(2-amino-7- fluorobenzo[d] thiazol-4-yl)-6- chloro-8- fluoro-2- ((tetrahydro-1H- pyrrolizin-7a(5H)- yl)methoxy) quinazolin-4-yl)- 1,4- diazepan-2-one | 600.2 (M + H)⁺ | ¹H NMR (400 MHz, METHANOL-d4) δ ppm 8.21 (s, 1 H), 7.30 (dd, J = 8.3, 5.4 Hz, 1 H), 7.08 (t, J = 8.8 Hz, 1 H), 4.68 (s, 2 H), 4.52 (s, 2 H), 4.30 (br t, J = 5.8 Hz, 2 H), 3.67-3.76 (m, 2 H), 3.16-3.32 (m, 4 H), 2.08-2.36 (m, 10 H). |
| 13 | 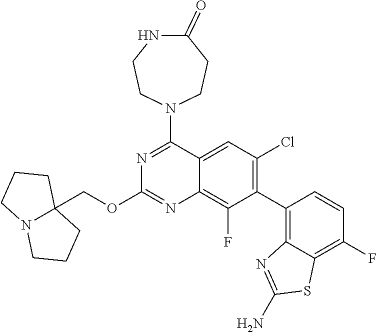 [SMILES: Nc1nc2c(-c3c(Cl)cc4c(N5CCNC(=O)CC5)nc(OCC56CCCN5CCC6)nc4c3F)ccc(F)c2s1] | 1-(7-(2-amino-7- fluorobenzo [d] thiazol-4-yl)-6- chloro-8- fluoro-2- ((tetrahydro-1H- pyrrolizin-7a(5H)- yl)methoxy) quinazolin-4-yl)- 1,4- diazepan-5-one | 600.2 (M + H)⁺ | ¹H NMR (400 MHz, METHANOL-d4) δ ppm 8.00-8.03 (m, 1 H), 7.29 (dd, J = 8.3, 5.4 Hz, 1 H), 7.08 (t, J = 8.8 Hz, 1 H), 4.71 (d, J = 2.1 Hz, 2 H), 4.11- 4.23 (m, 4 H), 3.61-3.77 (m, 4 H), 3.30-3.33 (m, 2 H), 3.08 (t, J = 6.3 Hz, 2 H), 2.10-2.39 (m, 8 H). |
| 14 | 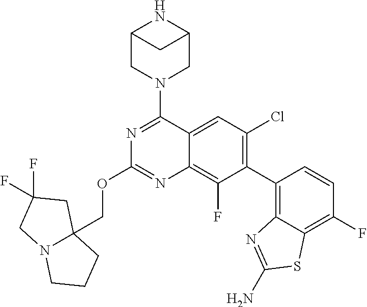 [SMILES: Nc1nc2c(-c3c(Cl)cc4c(N5CC6CC(C5)N6)nc(OCC56CCCN5CC(F)(F)C6)nc4c3F)ccc(F)c2s1] | 4-(4-((1R,5S)-3,6- diazabicyclo[3.1.1] heptan-3-yl)-6- chloro-2-((2,2- difluorotetrahydro- 1H-pyrrolizin- 7a(5H)- yl)methoxy)-8- fluoroquinazolin-7- yl)-7- fluorobenzo[d] thiazol-2-amine | 620.0 (M + H)⁺ | ¹H NMR (400 MHz, METHANOL-d4) δ ppm 8.42 (s, 1 H), 7.27 (ddd, J = 8.4, 5.4, 2.3 Hz, 1 H), 7.05 (t, J = 8.8 Hz, 1 H), 4.73-4.84 (m, 4 H), 4.57-4.68 (m, 4 H), 4.22-4.33 (m, 1 H), 3.84-4.00 (m, 2 H), 3.45-3.52 (m, 1 H), 3.10- 3.18 (m, 1 H), 2.80-3.04 (m, 2 H), 2.21-2.49 (m, 4 H), 2.02-2.08 (m, 1 H). |
| 15 | 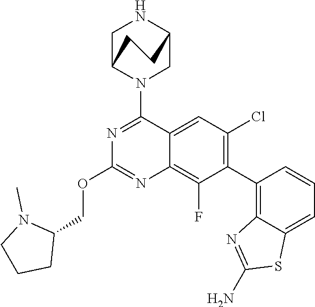 | 4-(4-((15,4S)-2,5- diazabicyclo[2.2.2] octan-2-yl)-6-chloro- 8-fluoro-2-(((S)-1- methylpyrrolidin-2- yl)methoxy) quinazolin-7- yl)benzo[d]thiazol- 2-amine | 544.0 (M + H)⁺ | ¹H NMR (400 MHz, METHANOL-d4) δ ppm 8.12 (s, 1 H), 7.85 (ddd, J = 7.6, 4.4, 1.6 Hz, 1 H), 7.30-7.39 (m, 2 H), 5.13 (br s, 1 H), 4.88-4.96 (m, 1 H), 4.66 (ddd, J = 13.1, 6.4, 5.0 Hz, 1 H), 4.52 (dt, J = 12.3, 2.4 Hz, 1 H), 4.38 (ddd, J = 12.4, 5.3, 2.2 Hz, 1 H), 3.97-4.01 (m, 1 H), 3.77- 3.93 (m, 2 H), 3.70-3.77 (m, 1 H), 3.61 (ddd, J = 12.7, 5.5, 2.1 Hz, 1 H), 3.19-3.31 (m, 1 H), 3.10 (s, 3 H), 2.35- 2.54 (m, 2 H), 2.04-2.30 (m, 6 H). |
| 16 | 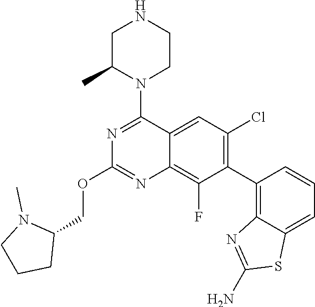 [SMILES: C[C@H]1CNCCN1c1nc(OC[C@@H]2CCCN2C)nc2c(F)c(-c3cccc4sc(N)nc34)c(Cl)cc12] | 4-(6-chloro-8- fluoro-4-((S)-2- methylpiperazin-1- yl)-2-(((S)-1- methylpyrrolidin-2- yl)methoxy) quinazolin-7- yl)benzo[d]thiazol- 2-amine | 542.0 (M + H)⁺ | ¹H NMR (400 MHz, METHANOL-d4) δ ppm 7.95-8.00 (m, 1 H), 7.89 (ddd, J = 7.5, 3.9, 1.6 Hz, 1 H), 7.37-7.46 (m, 2 H), 4.89-4.99 (m, 2 H), 4.67- 4.84 (m, 1 H), 4.37 (br dd, J = 14.1, 11.0 Hz, 1 H), 3.86- 3.98 (m, 2 H), 3.67-3.82 (m, 1 H), 3.35-3.59 (m, 4 H), 3.20-3.31 (m, 1 H), 3.07-3.16 (m, 3 H), 2.36- 2.49 (m, 1 H), 2.18-2.29 (m, 1 H), 2.05-2.18 (m, 2 H), 1.62 (dd, J = 7.0, 4.1 Hz, 3 H). |
| 17 | 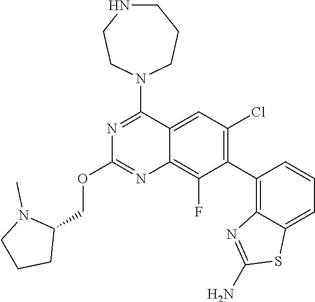 [SMILES: CN1CCC[C@H]1COc1nc(N2CCCNCC2)c2cc(Cl)c(-c3cccc4sc(N)nc34)c(F)c2n1] | 4-(6-chloro-4-(1,4- diazepan-1-yl)-8- fluoro-2-(((S)-1- methylpyrrolidin-2- yl)methoxy) quinazolin-7- yl)benzo[d] thiazol-2-amine | 542.2 (M + H)⁺ | ¹H NMR (400 MHz, METHANOL-d4) δ ppm 8.14 (s, 1 H), 7.91 (dt, J=7.8, 1.6 Hz, 1 H), 7.39-7.48 (m, 2 H), 4.90-4.98 (m, 1 H), 4.65-4.83 (m, 1 H), 4.24- 4.38 (m, 4 H), 3.90 (br s, 1 H), 3.60-3.82 (m, 3 H), 3.35-3.51 (m, 2 H), 3.19- 3.31 (m, 1 H), 3.11 (s, 3 H), 2.37-2.49 (m, 3 H), 2.22 (br s, 1 H), 2.05-2.18 (m, 2 H). |
| 18 | 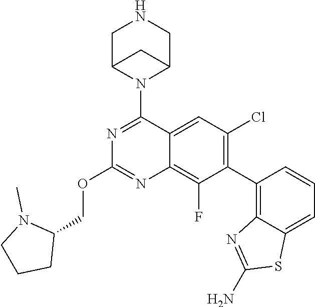 [SMILES: CN1CCC[C@H]1COc1nc(N2C3CNCC2C3)c2cc(Cl)c(-c3cccc4sc(N)nc34)c(F)c2n1] | 4-(4-(3,6- diazabicyclo[3.1.1] heptan-6-yl)-6- chloro-8-fluoro-2- (((S)-1- methylpyrrolidin-2- yl)methoxy) quinazolin-7- yl)benzo[d]thiazol- 2-amine | 540.2 (M + H)⁺ | ¹H NMR (400 MHz, METHANOL-d4) δ ppm 7.87-7.93 (m, 2 H), 7.40- 7.45 (m, 1 H), 7.38 (d, J = 5.6 Hz, 1 H), 5.24 (br d, J = 16.8 Hz, 2 H), 4.91-5.04 (m, 1 H), 4.68 (dd, J = 13.0, 6.3 Hz, 1 H), 3.94-4.13 (m, 2 H), 3.83-3.94 (m, 1 H), 3.68- 3.80 (m, 3 H), 3.20-3.31 (m, 2 H), 3.11 (s, 3 H), 2.36- 2.47 (m, 1 H), 2.17-2.27 (m, 1 H), 2.12 (br d, J = 10.2 Hz, 3 H). |
| 19 | 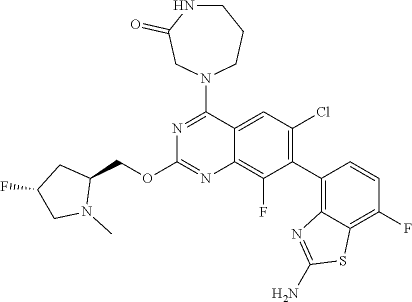 [SMILES: CN1C[C@H](F)C[C@H]1COc1nc(N2CCCNC(=O)C2)c2cc(Cl)c(-c3ccc(F)c4sc(N)nc34)c(F)c2n1] | 4-(7-(2-amino-7- fluorobenzo[d] thiazol-4-yl)- 6-chloro-8- fluoro-2-(((2S,4R)- 4-fluoro-1- methylpyrrolidin-2- yl)methoxy) quinazolin-4-yl)-1,4- diazepan-2-one | 592.0 (M + H)⁺ | ¹H NMR (400 MHz, METHANOL-d4) δ ppm 8.25 (s, 1 H), 7.33 (dd, J = 8.4, 5.3 Hz, 1 H), 7.11 (t, J = 8.9 Hz, 1 H), 5.36-5.65 (m, 1 H), 4.94-5.05 (m, 1 H), 4.68-4.84 (m, 1 H), 4.54 (s, 2 H), 4.22-4.37 (m, 3 H), 3.98-4.20 (m, 1 H), 3.57-3.80 (m, 1 H), 3.17- 3.30 (m, 5 H), 2.66-2.79 (m, 1 H), 2.33-2.50 (m, 1 H), 2.27 (br d, J = 5.4 Hz, 2 H). |
| 20 | 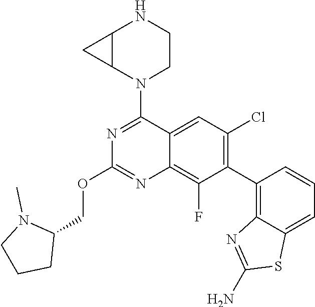 [SMILES: CN1CCC[C@H]1COc1nc(N2CCNC3CC32)c2cc(Cl)c(-c3cccc4sc(N)nc34)c(F)c2n1] | 4-(4-(2,5- diazabicyclo[4.1.0] heptan-2-yl)-6- chloro-8-fluoro-2- (((S)-1- methylpyrrolidin-2- yl)methoxy) quinazolin-7- yl)benzo[d]thiazol- 2-amine | 540.2 (M + H)⁺ | ¹H NMR (400 MHz, METHANOL-d4) δ ppm 8.67 (dd, J = 4.0, 1.0 Hz, 1 H), 7.87 (dq, J = 7.1, 2.0 Hz, 1 H), 7.34-7.43 (m, 2 H), 4.90-4.98 (m, 1 H), 4.77- 4.84 (m, 1 H), 4.64-4.75 (m, 1 H), 3.71-3.94 (m, 3 H), 3.56-3.71 (m, 1 H), 3.35-3.49 (m, 3 H), 3.19- 3.31 (m, 1 H), 3.11 (s, 3 H), 2.35-2.48 (m, 1 H), 2.22 (br s, 1 H), 2.04-2.17 (m, 2 H), 1.57-1.73 (m, 2 H). |
| 21 | 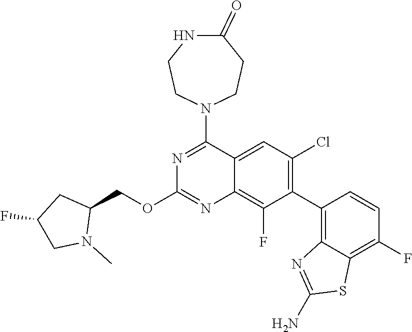 [SMILES: CN1C[C@H](F)C[C@H]1COc1nc(N2CCNC(=O)CC2)c2cc(Cl)c(-c3ccc(F)c4sc(N)nc34)c(F)c2n1] | 1-(7-(2-amino-7- fluorobenzo[d] thiazol-4-yl)- 6-chloro-8- fluoro-2-(((2S,4R)- 4-fluoro-1- methylpyrrolidin-2- yl)methoxy) quinazolin-4- yl)-1,4- diazepan-5-one | 592.0 (M + H)⁺ | ¹H NMR (400 MHz, METHANOL-d4) δ ppm 8.03 (s, 1 H), 7.32 (ddd, J = 8.3, 5.5, 2.4 Hz, 1 H), 7.11 (td, J = 8.8, 2.3 Hz, 1 H), 5.38-5.63 (m, 1 H), 4.97- 5.08 (m, 1 H), 4.73-4.85 (m, 1 H), 4.26-4.40 (m, 1 H), 3.98-4.22 (m, 5 H), 3.55-3.76 (m, 3 H), 3.21 (s, 3 H), 3.07 (t, J = 6.2 Hz, 2 H), 2.65-2.77 (m, 1 H), 2.34- 2.54 (m, 1 H). |
| 22 | 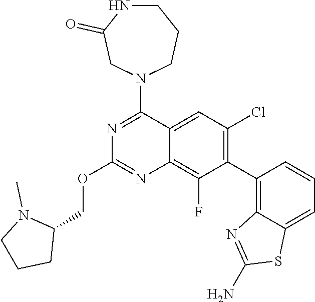 [SMILES: CN1CCC[C@H]1COc1nc(N2CCCNC(=O)C2)c2cc(Cl)c(-c3cccc4sc(N)nc34)c(F)c2n1] | 4-(7-(2- aminobenzo[d] thiazol-4-yl)- 6-chloro-8- fluoro-2-(((S)-1- methylpyrrolidin-2- yl)methoxy) quinazolin-4-yl)-1,4- diazepan-2-one | 556.2 (M + H)⁺ | ¹H NMR (400 MHz, METHANOL-d4) δ ppm 8.22 (s, 1 H), 7.89 (dt, J = 7.3, 0.8 Hz, 1 H), 7.37-7.45 (m, 2 H), 4.89-4.97 (m, 1 H), 4.60-4.77 (m, 1 H), 4.50 (s, 2 H), 4.29 (t, J = 6.0 Hz, 2 H), 3.89 (br s, 1 H), 3.62-3.83 (m, 1 H), 3.18-3.31 (m, 3 H), 3.09 (s, 3 H), 2.36-2.50 (m, 1 H), 2.17-2.32 (m, 3 H), 2.01-2.17 (m, 2 H). |
| 23 | 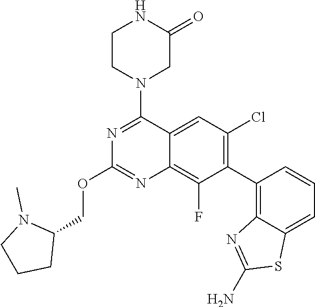 [SMILES: CN1CCC[C@H]1COc1nc(N2CCNC(=O)C2)c2cc(Cl)c(-c3cccc4sc(N)nc34)c(F)c2n1] | 4-(7-(2- aminobenzo[d] thiazol-4-yl)- 6-chloro-8- fluoro-2-(((S)-1- methylpyrrolidin-2- yl)methoxy) quinazolin-4- yl)piperazin-2- one | 542.2 (M + H)⁺ | ¹H NMR (400 MHz, METHANOL-d4) δ ppm 8.09 (d, J = 1.5 Hz, 1 H), 7.88 (d, J = 7.0 Hz, 1 H), 7.37- 7.44 (m, 2 H), 4.88-4.97 (m, 1 H), 4.64-4.83 (m, 1 H), 4.60 (s, 2 H), 4.18-4.31 (m, 2 H), 3.91 (br s, 1 H), 3.69-3.82 (m, 1 H), 3.61 (t, J = 5.1 Hz, 2 H), 3.20-3.31 (m, 1 H), 3.11 (s, 3 H), 2.36- 2.48 (m, 1 H), 2.23 (br s, 1 H), 2.05-2.19 (m, 2 H). |
| 24 | 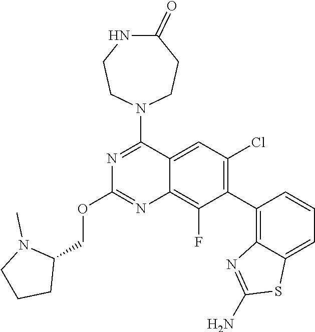 [SMILES: CN1CCC[C@H]1COc1nc(N2CCNC(=O)CC2)c2cc(Cl)c(-c3cccc4sc(N)nc34)c(F)c2n1] | 1-(7-(2- aminobenzo[d] thiazol-4-yl)- 6-chloro-8- fluoro-2-(((S)-1- methylpyrrolidin-2- yl)methoxy) quinazolin-4-yl)-1,4- diazepan-5-one | 556.0 (M + H)⁺ | ¹H NMR (400 MHz, METHANOL-d4) δ ppm 8.03 (d, J = 1.7 Hz, 1 H), 7.89 (d, J = 7.3 Hz, 1 H), 7.37- 7.45 (m, 2 H), 4.89-4.97 (m, 1 H), 4.64-4.74 (m, 1 H), 4.07-4.22 (m, 4 H), 3.86-4.02 (m, 1 H), 3.71- 3.83 (m, 1 H), 3.59-3.71 (m, 2 H), 3.20-3.31 (m, 1 H), 3.02-3.16 (m, 5 H), 2.37-2.49 (m, 1 H), 2.05- 2.28 (m, 3 H). |
| 25 | 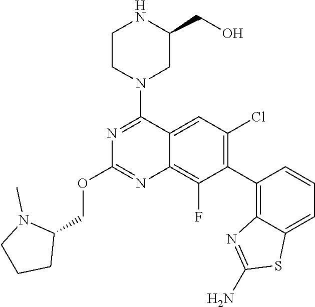 [SMILES: CN1CCC[C@H]1COc1nc(N2CCN[C@@H](CO)C2)c2cc(Cl)c(-c3cccc4sc(N)nc34)c(F)c2n1] | ((2R)-4-(7-(2- aminobenzo[d] thiazol-4-yl)- 6-chloro-8- fluoro-2-(((S)-1- methylpyrrolidin-2- yl)methoxy) quinazolin-4- yl)piperazin-2- yl)methanol | 558.2 (M + H)⁺ | ¹H NMR (400 MHz, METHANOL-d4) δ ppm 8.06 (s, 1 H), 7.90 (dt, J = 7.6, 1.9 Hz, 1 H), 7.38-7.47 (m, 2 H), 4.89-4.98 (m, 1 H), 4.80-4.85 (m, 1 H), 4.71 (dt, J = 12.8, 6.2 Hz, 1 H), 4.47-4.60 (m, 2 H), 3.87- 3.97 (m, 2 H), 3.63-3.84 (m, 5 H), 3.43-3.60 (m, 2 H), 3.21-3.31 (m, 1 H), 3.12 (d, J = 2.1 Hz, 3 H), 2.35-2.49 (m, 1 H), 2.05- 2.28 (m, 3 H). |
| 26 | 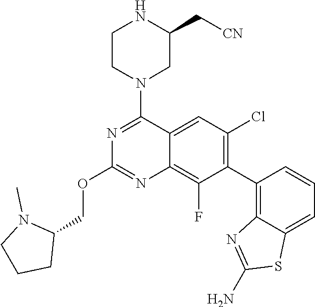 [SMILES: CN1CCC[C@H]1COc1nc(N2CCN[C@@H](CC#N)C2)c2cc(Cl)c(-c3cccc4sc(N)nc34)c(F)c2n1] | 2-((2S)-4-(7-(2- aminobenzo[d] thiazol-4-yl)- 6-chloro-8- fluoro-2-(((S)-1- methylpyrrolidin-2- yl)methoxy) quinazolin-4- yl)piperazin-2- yl)acetonitrile | 567.2 (M + H)⁺ | ¹H NMR (400 MHz, METHANOL-d4) δ ppm 8.07 (s, 1 H), 7.89 (dt, J = 7.7, 1.6 Hz, 1 H), 7.36-7.46 (m, 2 H), 4.90-4.99 (m, 1 H), 4.64-4.81 (m, 2 H), 4.54 (br d, J = 14.4 Hz, 1 H), 4.02- 4.13 (m, 1 H), 3.84-3.97 (m, 2 H), 3.58-3.83 (m, 3 H), 3.35-3.55 (m, 1 H), 3.22-3.31 (m, 1 H), 3.08- 3.18 (m, 5 H), 2.43 (br d, J = 6.7 Hz, 1 H), 2.05-2.28 (m, 3 H). |
| 27 | 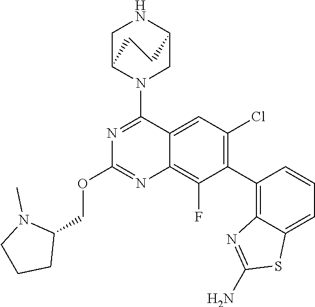 [SMILES: CN1CCC[C@H]1COc1nc(N2C[C@H]3CC[C@@H]2CN3)c2cc(Cl)c(-c3cccc4sc(N)nc34)c(F)c2n1] | 4-(4-((1R,4R)-2,5- diazabicyclo[2.2.2] octan-2-yl)-6-chloro- 8-fluoro-2-(((S)-1- methylpyrrolidin-2- yl)methoxy) quinazolin-7- yl)benzo[d]thiazol- 2-amine | 554.2 (M + H)⁺ | ¹H NMR (400 MHz, METHANOL-d4) δ ppm 8.10-8.14 (m, 1 H), 7.87 (dd, J = 7.5, 1.5 Hz, 1 H), 7.33-7.41 (m, 2 H), 5.13 (br s, 1 H), 4.88-4.97 (m, 1 H), 4.64-4.82 (m, 1 H), 4.53 (br d, J = 12.3 Hz, 1 H), 4.37 (dt, J = 12.3, 2.7 Hz, 1 H), 3.99 (br d, J = 2.5 Hz, 1 H), 3.71- 3.94 (m, 3 H), 3.49-3.69 (m, 1 H), 3.20-3.31 (m, 1 H), 3.10 (s, 3 H), 2.35-2.54 (m, 2 H), 2.04-2.30 (m, 6 H). |
| 28 | 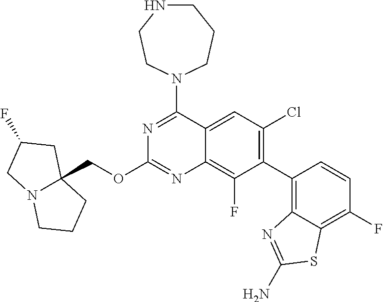 [SMILES: Nc1nc2c(-c3c(Cl)cc4c(N5CCCNCC5)nc(OC[C@@]56CCCN5C[C@H](F)C6)nc4c3F)ccc(F)c2s1] | 4-(6-chloro-4-(1,4- diazepan-1-yl)-8- fluoro-2-(((2R,7aS)- 2-fluorotetrahydro- 1H-pyrrolizin- 7a(5H)- yl)methoxy) quinazolin-7-yl)-7- fluorobenzo[d] thiazol-2-amine | 604.1 (M + H)⁺ | ¹H NMR (400 MHz, METHANOL-d4) δ ppm 8.07-8.18 (m, 1 H), 7.22- 7.30 (m, 1 H), 6.98-7.11 (m, 1 H), 5.46-5.68 (m, 1 H), 4.69 (s, 2 H), 4.22-4.41 (m, 4 H), 3.82-4.10 (m, 3 H), 3.61-3.75 (m, 2 H), 3.44-3.54 (m, 1 H), 3.37- 3.44 (m, 2 H), 2.55-2.76 (m, 2 H), 2.31-2.50 (m, 5 H), 2.09-2.24 (m, 1 H). |
| 29 | 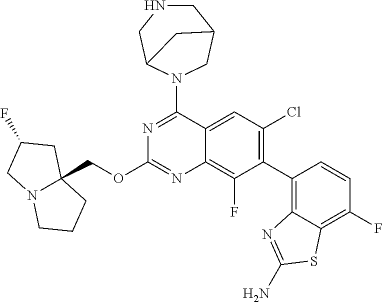 [SMILES: Nc1nc2c(-c3c(Cl)cc4c(N5CC6CNCC5C6)nc(OC[C@@]56CCCN5C[C@H](F)C6)nc4c3F)ccc(F)c2s1] | 4-(4-(3,6- diazabicyclo[3.2.1] octan-6-yl)- 6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine Diastereoisomer A | 616.1 (M + H)⁺ | ¹H NMR (400 MHz, METHANOL-d4) δ ppm 8.31 (d, J = 1.7 Hz, 1 H), 7.19-7.31 (m, 1 H), 7.06 (s, 1 H), 5.44-5.69 (m, 1 H), 5.15-5.26 (m, 1 H), 4.71 (s, 2 H), 4.50-4.60 (m, 1 H), 4.33-4.45 (m, 1 H), 3.79- 4.10 (m, 4 H), 3.40-3.56 (m, 3 H), 3.35-3.38 (m, 1 H), 2.97-3.11 (m, 1 H), 2.52-2.81 (m, 2 H), 2.26- 2.51 (m, 4 H), 2.07-2.24 (m, 2 H). |
| 30 | 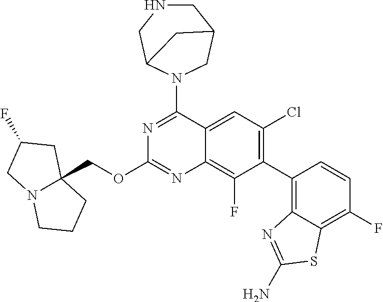 [SMILES: Nc1nc2c(-c3c(Cl)cc4c(N5CC6CNCC5C6)nc(OC[C@@]56CCCN5C[C@H](F)C6)nc4c3F)ccc(F)c2s1] | 4-(4-(3,6- diazabicyclo[3.2.1] octan-6-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine Diastereoisomer B | 616.1 (M + H)⁺ | ¹H NMR (400 MHz, METHANOL-d4) δ ppm 8.26-8.35 (m, 1 H), 7.19- 7.32 (m, 1 H), 6.98-7.11 (m, 1 H), 5.46-5.69 (m, 1 H), 5.14-5.27 (m, 1 H), 4.60-4.78 (m, 2 H), 4.47- 4.58 (m, 1 H), 4.23-4.39 (m, 1 H), 3.79-4.11 (m, 4 H), 3.36-3.54 (m, 4 H), 2.98-3.19 (m, 1 H), 2.54- 2.79 (m, 2 H), 2.23-2.50 (m, 4 H), 2.05-2.23 (m, 2 H). |
| 31 | 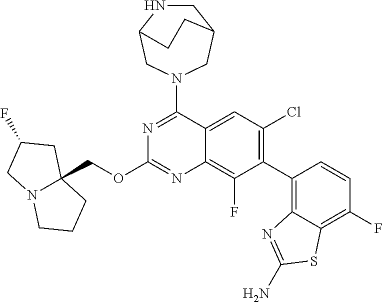 [SMILES: Nc1nc2c(-c3c(Cl)cc4c(N5CC6CCC(C5)NC6)nc(OC[C@@]56CCCN5C[C@H](F)C6)nc4c3F)ccc(F)c2s1] | 4-(4-(3,6- diazabicyclo[3.2.2] nonan-3-yl)- 6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 630.2 (M + H)⁺ | ¹H NMR (400 MHz, METHANOL-d4) δ ppm 8.01 (d, J = 1.5 Hz, 1 H), 7.21-7.32 (m, 1 H), 7.05 (s, 1 H), 5.45-5.68 (m, 1 H), 4.91-5.07 (m, 1 H), 4.66- 4.76 (m, 2 H), 4.52-4.64 (m, 1 H), 4.05-4.17 (m, 2 H), 3.81-4.04 (m, 4 H), 3.60-3.70 (m, 1 H), 3.44- 3.59 (m, 2 H), 2.51-2.81 (m, 3 H), 2.29-2.51 (m, 3 H), 2.06-2.25 (m, 3 H), 1.80-1.99 (m, 2 H). |
| 32 | 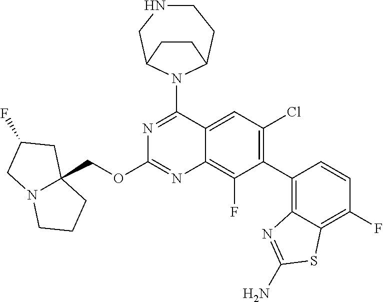 [SMILES: Nc1nc2c(-c3c(Cl)cc4c(N5C6CCNCC5CC6)nc(OC[C@@]56CCCN5C[C@H](F)C6)nc4c3F)ccc(F)c2s1] | 4-(4-(3,9- diazabicyclo[4.2.1] nonan-9-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine Diastereoisomer A | 630.2 (M + H)⁺ | ¹H NMR (400 MHz, METHANOL-d4) δ ppm 8.04-8.12 (m, 1 H), 7.21- 7.30 (m, 1 H), 6.99-7.11 (m, 1 H), 5.71-5.81 (m, 1 H), 5.46-5.66 (m, 1 H), 5.33-5.42 (m, 1 H), 4.63- 4.75 (m, 2 H), 3.76-4.09 (m, 4 H), 3.36-3.59 (m, 4 H), 2.50-2.79 (m, 4 H), 2.29-2.49 (m, 4 H), 2.05- 2.25 (m, 4 H). |
| 33 | 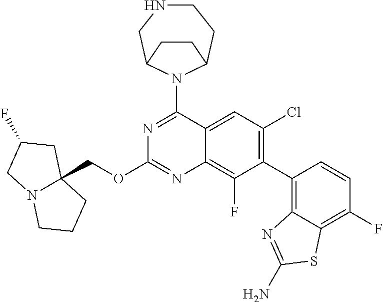 [SMILES: Nc1nc2c(-c3c(Cl)cc4c(N5C6CCNCC5CC6)nc(OC[C@@]56CCCN5C[C@H](F)C6)nc4c3F)ccc(F)c2s1] | 4-(4-(3,9- diazabicyclo[4.2.1] nonan-9-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo [d] thiazol-2-amine Diastereoisomer B | 630.2 (M + H)⁺ | ¹H NMR (400 MHz, METHANOL-d4) δ ppm 8.07 (d, J = 1.0 Hz, 1 H), 7.20-7.32 (m, 1 H), 7.05 (d, J = 9.0 Hz, 1 H), 5.69-5.81 (m, 1 H), 5.47-5.67 (m, 1 H), 5.35-5.44 (m, 1 H), 4.62-4.78 (m, 2 H), 3.82- 4.11 (m, 3 H), 3.72-3.81 (m, 1 H), 3.36-3.59 (m, 4 H), 2.30-2.76 (m, 8 H), 2.02-2.24 (m, 4 H). |
| 34 | 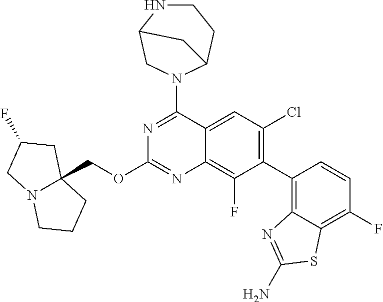 [SMILES: Nc1nc2c(-c3c(Cl)cc4c(N5CC6CC5CCN6)nc(OC[C@@]56CCCN5C[C@H](F)C6)nc4c3F)ccc(F)c2s1] | 4-(4-(2,6- diazabicyclo[3.2.1] octan-6-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 616.2 (M + H)⁺ | ¹H NMR (400 MHz, METHANOL-d4) δ ppm 8.24 (br d, J = 1.7 Hz, 1 H), 7.20-7.33 (m, 1 H), 7.06 (d, J = 0.6 Hz, 1 H), 5.45-5.67 (m, 1 H), 5.20-5.33 (m, 1 H), 4.58-4.78 (m, 3 H), 4.44-4.52 (m, 1 H), 4.38 (s, 1 H), 3.92 (br d, J = 11.3 Hz, 3 H), 3.40-3.58 (m, 2 H), 3.21-3.30 (m, 1 H), 2.60 (br d, J = 4.0 Hz, 3 H), 2.23- 2.51 (m, 5 H), 2.07-2.23 (m, 2 H). |
| 35 | 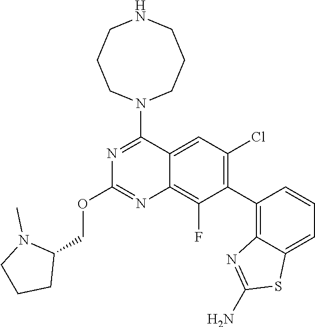 [SMILES: CN1CCC[C@H]1COc1nc(N2CCCNCCC2)c2cc(Cl)c(-c3cccc4sc(N)nc34)c(F)c2n1] | 4-(6-chloro-4-(1,5- diazocan-1-yl)-8- fluoro-2-(((S)-1- methylpyrrolidin-2- yl)methoxy) quinazolin-7- yl)benzo[d]thiazol- 2-amine | 556.2 (M + H)⁺ | ¹H NMR (400 MHz, METHANOL-d4) δ ppm 8.13 (d, J = 1.5 Hz, 1 H), 7.86 (dt, J = 7.6, 1.6 Hz, 1 H), 7.32-7.40 (m, 2 H), 4.88-4.96 (m, 1 H), 4.64-4.75 (m, 1 H), 4.20-4.31 (m, 4 H), 3.83-3.96 (m, 1 H), 3.68- 3.81 (m, 1 H), 3.42 (t, J = 5.5 Hz, 4 H), 3.21-3.31 (m, 1 H), 3.10 (s, 3 H), 2.36-2.51 (m, 5 H), 2.17-2.28 (m, 1 H), 2.05-2.17 (m, 2 H). |
| 36 | 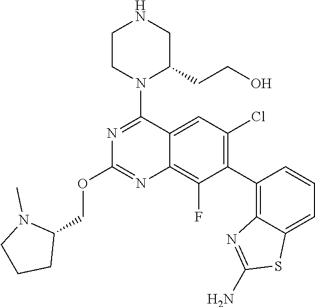 [SMILES: CN1CCC[C@H]1COc1nc(N2CCNC[C@@H]2CCO)c2cc(Cl)c(-c3cccc4sc(N)nc34)c(F)c2n1] | 2-((2S)-1-(7-(2- aminobenzo[d] thiazol-4-yl)- 6-chloro-8- fluoro-2-(((S)-1- methylpyrrolidin-2- yl)methoxy) quinazolin-4- yl)piperazin-2- yl)ethan-1-ol | 572.2 (M + H)⁺ | ¹H NMR (400 MHz, METHANOL-d4) δ ppm 8.17 (d, J = 1.3 Hz, 1 H), 7.83 (dt, J = 7.7, 1.3 Hz, 1 H), 7.26-7.35 (m, 2 H), 4.88-5.01 (m, 2 H), 4.70-4.83 (m, 2 H), 3.80-3.98 (m, 3 H), 3.58-3.80 (m, 3 H), 3.35- 3.50 (m, 3 H), 3.22-3.31 (m, 2 H), 3.07-3.18 (m, 3 H), 2.34-2.49 (m, 3 H), 2.20-2.29 (m, 1 H), 2.05- 2.19 (m, 2 H). |
| 37 | 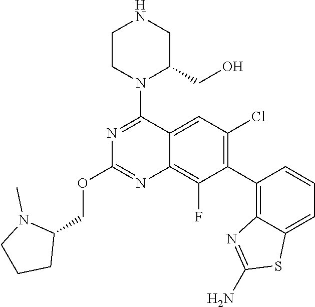 [SMILES: CN1CCC[C@H]1COc1nc(N2CCNC[C@@H]2CO)c2cc(Cl)c(-c3cccc4sc(N)nc34)c(F)c2n1] | ((2R)-1-(7-(2- aminobenzo[d] thiazol-4-yl)- 6-chloro-8- fluoro-2-(((S)-1- methylpyrrolidin-2- yl)methoxy) quinazolin-4- yl)piperazin-2- yl)methanol | 558.2 (M + H)⁺ | ¹H NMR (400 MHz, METHANOL-d4) δ ppm 8.33 (dd, J = 3.3, 1.7 Hz, 1 H), 7.82 (d, J = 7.2 Hz, 1 H), 7.26-7.34 (m, 2 H), 4.88- 5.05 (m, 2 H), 4.70-4.84 (m, 3 H), 3.86-3.98 (m, 2 H), 3.64-3.80 (m, 2 H), 3.49-3.60 (m, 2 H), 3.22- 3.31 (m, 3 H), 3.07-3.19 (m, 3 H), 2.39-2.49 (m, 1 H), 2.24 (br dd, J = 6.8, 5.3 Hz, 1 H), 2.05-2.19 (m, 2 H). |
| 38 | 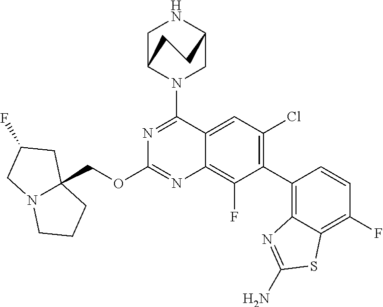 [SMILES: Nc1nc2c(-c3c(Cl)cc4c(N5C[C@@H]6CC[C@H]5CN6)nc(OC[C@@]56CCCN5C[C@H](F)C6)nc4c3F)ccc(F)c2s1] | 4-(4-((15,4S)-2,5- diazabicyclo[2.2.2] octan-2-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 616.2 (M + H)⁺ | ¹H NMR (400 MHz, METHANOL-d4) δ ppm 8.10-8.14 (m, 1 H), 7.25- 7.31 (m, 1 H), 7.04-7.11 (m, 1 H), 5.44-5.69 (m, 1 H), 5.17 (br s, 1 H), 4.66- 4.76 (m, 2 H), 4.49-4.60 (m, 1 H), 4.40 (dd, J = 12.4, 2.0 Hz, 1 H), 3.81-4.08 (m, 5 H), 3.56-3.66 (m, 1 H), 3.43-3.53 (m, 1 H), 2.55- 2.80 (m, 2 H), 2.31-2.48 (m, 4 H), 2.07-2.29 (m, 4 H). |
| 39 | 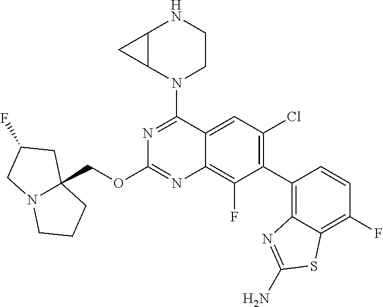 [SMILES: Nc1nc2c(-c3c(Cl)cc4c(N5CCNC6CC65)nc(OC[C@@]56CCCN5C[C@H](F)C6)nc4c3F)ccc(F)c2s1] | 4-(4-(2,5- diazabicyclo[4.1.0] heptan-2-yl)-6- chloro-8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 602.2 (M + H)⁺ | ¹H NMR (400 MHz, METHANOL-d4) δ ppm 8.66 (s, 1 H), 7.25 (dddd, J = 8.5, 7.0, 5.4, 1.9 Hz, 1 H), 7.04 (td, J = 8.8, 1.5 Hz, 1 H), 5.43-5.69 (m, 1 H), 4.83- 4.87 (m, 1 H), 4.64-4.80 (m, 3 H), 3.78-4.12 (m, 4 H), 3.58-3.64 (m, 1 H), 3.35-3.52 (m, 4 H), 2.57- 2.75 (m, 2 H), 2.32-2.48 (m, 3 H), 2.10-2.21 (m, 1 H), 1.70 (q, J = 7.9 Hz, 1 H), 1.58-1.64 (m, 1 H). |
| 40 | 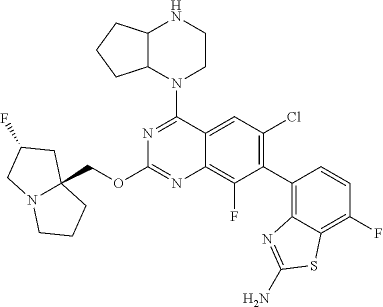 [SMILES: Nc1nc2c(-c3c(Cl)cc4c(N5CCNC6CCCC65)nc(OC[C@@]56CCCN5C[C@H](F)C6)nc4c3F)ccc(F)c2s1] | 4-(6-chloro-8- fluoro-2-(((2R,7aS)- 2-fluorotetrahydro- 1H-pyrrolizin- 7a(5H)- yl)methoxy)-4- (octahydro-1H- cyclopenta[b] pyrazin-1-yl) quinazolin-7- yl)-7- fluorobenzo[d] thiazol-2-amine | 630.2 (M + H)⁺ | ¹H NMR (400 MHz, METHANOL-d4) δ ppm 7.98 (d, J = 5.9 Hz, 1 H), 7.25 (dddd, J = 8.4, 5.4, 4.0, 1.3 Hz, 1 H), 7.04 (t, J = 8.8 Hz, 1 H), 5.47-5.69 (m, 1 H), 4.99-5.07 (m, 1 H), 4.67- 4.80 (m, 3 H), 4.50-4.59 (m, 1 H), 3.73-4.09 (m, 5 H), 3.35-3.56 (m, 3 H), 2.57-2.77 (m, 2 H), 2.33- 2.48 (m, 3 H), 2.08-2.31 (m, 5 H), 1.88-2.01 (m, 2 H). |
| 41 | 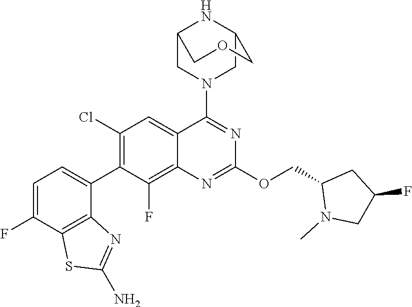 [SMILES: CN1C[C@H](F)C[C@H]1COc1nc(N2CC3COCC(C2)N3)c2cc(Cl)c(-c3ccc(F)c4sc(N)nc34)c(F)c2n1] | 4-(4-((1R,5S)-3-oxa- 7,9- diazabicyclo[3.3.1] nonan-7-yl)- 6-chloro- 8-fluoro-2- (((2S,4R)-4-fluoro- 1-methylpyrrolidin- 2-yl)methoxy) quinazolin-7-yl)-7- fluorobenzo[d] thiazol-2-amine | 605.8 (M + H)⁺ | ¹H NMR (400 MHz, METHANOL-d4) δ ppm 7.99-8.06 (m, 1 H), 7.21- 7.31 (m, 1 H), 6.99-7.09 (m, 1 H), 5.37-5.59 (m, 1 H), 5.12-5.25 (m, 2 H), 4.94-5.06 (m, 1 H), 4.68- 4.76 (m, 1 H), 3.96-4.37 (m, 8 H), 3.59-3.76 (m, 3 H), 3.15-3.24 (m, 3 H), 2.61-2.79 (m, 1 H), 2.24- 2.54 (m, 1 H). |
| 42 | 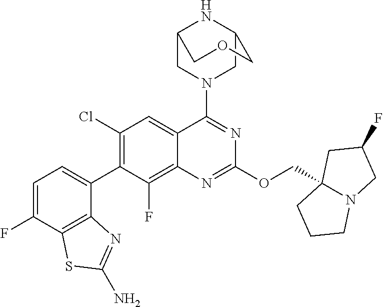 [SMILES: Nc1nc2c(-c3c(Cl)cc4c(N5CC6COCC(C5)N6)nc(OC[C@@]56CCCN5C[C@H](F)C6)nc4c3F)ccc(F)c2s1] | 4-(4-((1R,5S)-3-oxa- 7,9- diazabicyclo[3.3.1] nonan-7-yl)-6-chloro- 8-fluoro-2- (((2R,7aS)-2- fluorotetrahydro- 1H-pyrrolizin- 7a(5H)- yl)methoxy) quinazolin-7-yl)-7- fluorobenzo[d] thiazol-2-amine | 631.7 (M + H)⁺ | ¹H NMR (400 MHz, METHANOL-d4) δ ppm 8.03 (d, J = 1.5 Hz, 1 H), 7.25 (s, 1 H), 7.04 (t, J = 8.8 Hz, 1 H), 5.46-5.71 (m, 1 H), 5.11-5.28 (m, 2 H), 4.62- 4.76 (m, 2 H), 3.81-4.29 (m, 9 H), 3.66-3.79 (m, 2 H), 3.42-3.56 (m, 1 H), 2.53-2.81 (m, 2 H), 2.28- 2.51 (m, 3 H), 2.06-2.25 (m, 1 H). |

### 7a-(((7-(2-aminobenzo[d]thiazol-4-yl)-6-chloro-8-fluoro-4-(piperazin-1-yl)quinazolin-2-yl)oxy)methyl)hexahydro-3H-pyrrolizin-3-one (Example 43

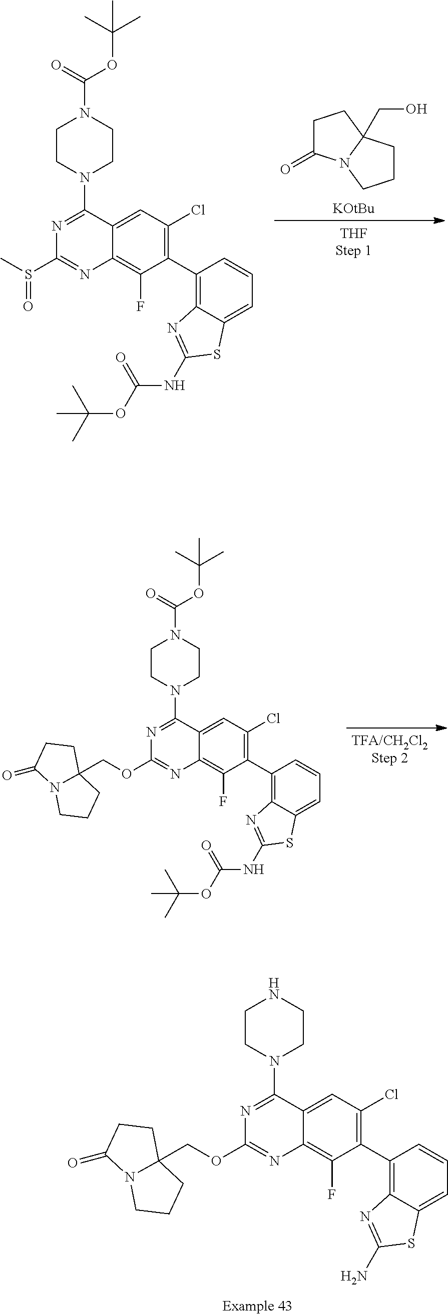

Step 1: tert-Butyl 4-(7-(2-((tert-butoxycarbonyl)amino)benzo[d]thiazol-4-yl)-6-chloro-8-fluoro-2-((3-oxotetrahydro-1H-pyrrolizin-7a(5H)-yl)methoxy)quinazolin-4-yl)piperazine-1-carboxylate. To a solution of 7a-(hydroxymethyl)hexahydro-3H-pyrrolizin-3-one (8.3 mg, 0.053 mmol, PharmaBlock) in tetrahydrofuran (0.5 mL) at 0° C. was added potassium tert-butoxide (1.0 M in THF, 0.071 mL, 0.071 mmol, Sigma-Aldrich Corporation). The mixture was then added to the solution of tert-butyl 4-(7-(2-((tert-butoxycarbonyl)amino)benzo[d]thiazol-4-yl)-6-chloro-8-fluoro-2-(methylsulfinyl)quinazolin-4-yl)piperazine-1-carboxylate (24 mg, 0.035 mmol) in tetrahydrofuran (0.5 mL) at 0° C. The resulting mixture was stirred at 0° C. for 10 min. The reaction mixture was purified by reverse phase HPLC to afford tert-butyl 4-(7-(2-((tert-butoxycarbonyl)amino)benzo[d]thiazol-4-yl)-6-chloro-8-fluoro-2-((3-oxotetrahydro-1H-pyrrolizin-7a(5H)-yl)methoxy)quinazolin-4-yl)piperazine-1-carboxylate as white solid. m/z (ESI, +ve ion): 768.2 (M+H)⁺.

Step 2: 7a-(((7-(2-Aminobenzo[d]thiazol-4-yl-6-chloro-8-fluoro-4-(piperazin-1-yl)quinazolin-2-yl)oxy)methyl)hexahydro-3H-pyrrolizin-3-one. tert-Butyl 4-(7-(2-((tert-butoxycarbonyl)amino)benzo[d]thiazol-4-yl)-6-chloro-8-fluoro-2-((3-oxotetrahydro-1H-pyrrolizin-7a(5H)-yl)methoxy)quinazolin-4-yl)piperazine-1-carboxylate was dissolved in 0.5 mL dichloromethane. Trifluoroacetic acid (0.77 g, 0.5 mL, 6.71 mmol, Sigma-Aldrich Corporation) was added and the reaction mixture was stirred at room temperature for 1 h. The reaction mixture was concentrated under reduced pressure and purified by reverse phase HPLC to afford 7a-(((7-(2-aminobenzo[d]thiazol-4-yl)-6-chloro-8-fluoro-4-(piperazin-1-yl)quinazolin-2-yl)oxy)methyl)hexahydro-3H-pyrrolizin-3-one (13 mg, 0.023 mmol, 65% yield) as white solid. m/z (ESI, +ve ion): 568.2 (M+Na)+. ¹HNMR(400 MHz, METHANOL-d4) δ ppm 8.02 (s, 1H), 7.90 (d, 8.57.5 Hz, 1H), 7.39-7.47 (m, 2H), 4.70 (dd, J=11.1, 8.6 Hz, 1H), 4.48 (d, 7511.4 Hz, 1H), 4.13 (br s, 4H), 3.57-3.70 (m, 1H), 3.46-3.56 (m, 4H), 2.99-3.16 (m, 2H), 2.40-2.50 (m, 2H), 2.05-32.32 (m, 4H), 1.74H-1.83 (m, 1H).

TABLE 3
Additional Examples. Prepared in an Analogous Manner to Example 43.

| | | | MS | |
|---|---|---|---|---|
| | | | m/z | |
| | | | (ESI, +ve | |
| Ex. # | Structure | Name | ion) | 1H NMR |
| 44 | 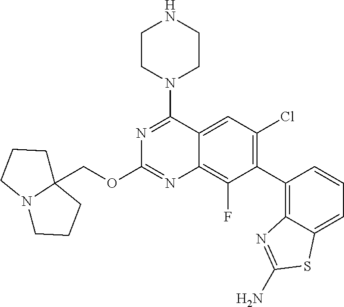 [SMILES: Nc1nc2c(-c3c(Cl)cc4c(N5CCNCC5)nc(OCC56CCCN5CCC6)nc4c3F)cccc2s1] | 4-(6-chloro-8- fluoro-4- (piperazin-1- yl)-2- ((tetrahydro- 1H-pyrrolizin- 7a(5H)- yl)methoxy) quinazolin-7- yl)benzo[d] thiazol-2-amine | 554.2 (M + H)⁺ | ¹H NMR (400 MHz, METHANOL-d4) δ ppm 8.05 (d, J = 1.5 Hz, 1 H), 7.89 (dd, J = 7.7, 1.5 Hz, 1 H), 7.37-7.44 (m, 2 H), 4.66-4.76 (m, 2 H), 4.10- 4.21 (m, 4 H), 3.67-3.76 (m, 2 H), 3.47-3.57 (m, 4 H), 3.35-3.43 (m, 1 H), 3.27-3.31 (m, 1 H), 2.10- 2.37 (m, 8 H). |
| 45 | 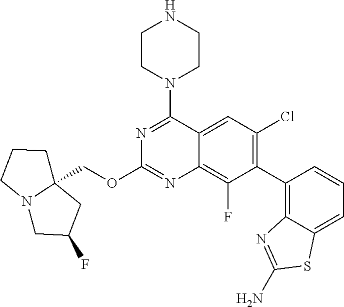 [SMILES: Nc1nc2c(-c3c(Cl)cc4c(N5CCNCC5)nc(OC[C@@]56CCCN5C[C@H](F)C6)nc4c3F)cccc2s1] | 4-(6-chloro-8- fluoro-2- (((2R,7aS)-2- fluorotetra- hydro-1H- pyrrolizin- 7a(5H)- yl)methoxy)-4- (piperazin-1- yl)quinazolin- 7- yl)benzo[d] thiazol-2-amine | 572.2 (M + H)⁺ | ¹H NMR (400 MHz, METHANOL-d4) δ ppm 8.04 (s, 1 H), 7.85 (d, J = 7.3 Hz, 1 H), 7.30-7.38 (m, 2 H), 5.42-5.70 (m, 1 H), 4.67-4.77 (m, 2 H), 4.12-4.20 (m, 4 H), 3.84- 4.08 (m, 3 H), 3.46-3.57 (m, 5 H), 2.57-2.77 (m, 2 H), 2.32-2.49 (m, 3 H), 2.19 (br dd, J = 8.9, 6.0 Hz, 1 H). |
| 46 | 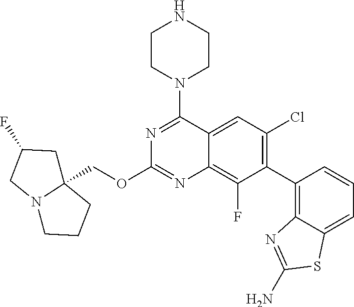 [SMILES: Nc1nc2c(-c3c(Cl)cc4c(N5CCNCC5)nc(OC[C@]56CCCN5C[C@H](F)C6)nc4c3F)cccc2s1] | 4-(6-chloro-8- fluoro-2- (((2R,7aR)-2- fluorotetra- hydro-1H- pyrrolizin- 7a(5H)- yl)methoxy)-4- (piperazin-1- yl)quinazolin- 7- yl)benzo[d] thiazol-2-amine | 572.0 (M + H)⁺ | ¹H NMR (400 MHz, METHANOL-d4) δ ppm 8.05 (s, 1 H), 7.90 (d, J = 7.1 Hz, 1 H), 7.38-7.46 (m, 2 H), 5.39-5.65 (m, 1 H), 4.73-4.85 (m, 2 H), 4.03-4.21 (m, 5 H), 3.46- 3.71 (m, 7 H), 2.67-2.78 (m, 1 H), 2.38-2.57 (m, 2 H), 2.25-2.35 (m, 2 H), 2.13-2.24 (m, 1 H). |
| 47 | 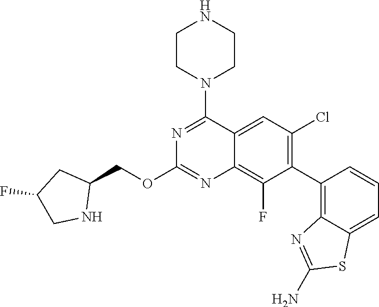 [SMILES: Nc1nc2c(-c3c(Cl)cc4c(N5CCNCC5)nc(OC[C@@H]5C[C@@H](F)CN5)nc4c3F)cccc2s1] | 4-(6-chloro-8- fluoro-2- (((2S,4R)-4- fluoro- pyrrolidin-2- yl)methoxy)-4- (piperazin-1- yl)quinazolin- 7- yl)benzo[d] thiazol-2-amine | 532.0 (M + H)⁺ | ¹H NMR (400 MHz, METHANOL-d4) δ ppm 8.03 (s, 1 H), 7.85 (d, J = 7.7 Hz, 1 H), 7.32-7.39 (m, 2 H), 5.38-5.68 (m, 1 H), 4.89-4.95 (m, 1 H), 4.69 (dd, J = 12.5, 7.2 Hz, 1 H), 4.35-4.43 (m, 1 H), 4.13 (br s, 4 H), 3.62- 3.78 (m, 2 H), 3.52 (br s, 4 H), 2.56-2.68 (m, 1 H), 2.18-2.36 (m, 1 H). |
| 48 | 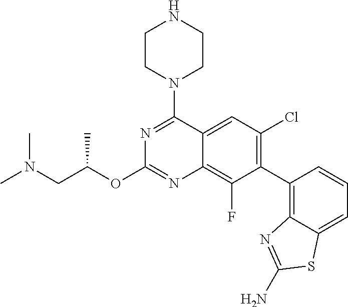 [SMILES: C[C@@H](CN(C)C)Oc1nc(N2CCNCC2)c2cc(Cl)c(-c3cccc4sc(N)nc34)c(F)c2n1] | 4-(6-chloro-2- (((S)-1- (dimethylamino) propan-2- yl)oxy)-8- fluoro-4- (piperazin-1- yl)quinazolin- 7- yl)benzo[d] thiazol-2-amine | 516.2 (M + H)⁺ | ¹H NMR (400 MHz, METHANOL-d4) δ ppm 8.03 (s, 1 H), 7.87 (dt, J = 6.9, 2.0 Hz, 1 H), 7.33- 7.43 (m, 2 H), 5.67-5.75 (m, 1 H), 4.13 (br d, J = 4.4 Hz, 4 H), 3.63 (dd, J = 13.8, 9.8 Hz, 1 H), 3.43-3.56 (m, 5 H), 3.01 (s, 6 H), 1.52 (dd, J = 6.2, 1.5 Hz, 3 H). |
| 49 | 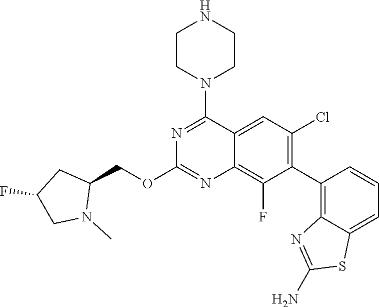 [SMILES: CN1C[C@H](F)C[C@H]1COc1nc(N2CCNCC2)c2cc(Cl)c(-c3cccc4sc(N)nc34)c(F)c2n1] | 4-(6-chloro-8- fluoro-2- (((2S,4R)-4- fluoro-1- methyl- pyrrolidin-2- yl)methoxy)-4- (piperazin-1- yl)quinazolin- 7- yl)benzo[d] thiazol-2-amine | 546.0 (M + H)⁺ | ¹H NMR (400 MHz, METHANOL-d4) δ ppm 8.04 (t, J = 1.7 Hz, 1 H), 7.87 (ddd, J = 7.4, 3.2, 1.7 Hz, 1 H), 7.33-7.42 (m, 2 H), 5.38-5.59 (m, 1 H), 4.95-5.07 (m, 1 H), 4.72- 4.82 (m, 1 H), 4.21-4.33 (m, 1 H), 4.05-4.21 (m, 4 H), 3.98-4.11 (m, 1 H), 3.59-3.76 (m, 1 H), 3.46- 3.57 (m, 4 H), 3.19-3.31 (m, 3 H), 2.64-2.75 (m, 1 H), 2.30-2.52 (m, 1 H). |
| 50 | 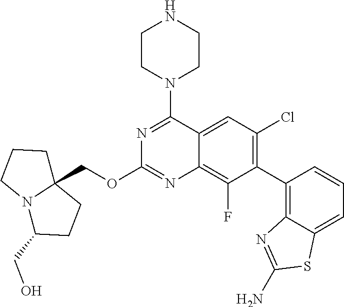 [SMILES: Nc1nc2c(-c3c(Cl)cc4c(N5CCNCC5)nc(OC[C@]56CCCN5[C@@H](CO)CC6)nc4c3F)cccc2s1] | ((3R,7aR)-7a- (((7-(2- aminobenzo[d] thiazol-4-yl)-6- chloro-8- fluoro-4- (piperazin-1- yl)quinazolin- 2- yl)oxy)methyl) hexahydro-1H- pyrrolizin-3- yl)methanol | 583.6 (M + H)⁺ | ¹H NMR (400 MHz, METHANOL-d4) δ ppm 8.05 (d, J = 1.5 Hz, 1 H), 7.90 (ddd, J = 7.5, 5.9, 1.7 Hz, 1 H), 7.38-7.48 (m, 2 H), 4.79 (dd, J = 12.5, 5.9 Hz, 1 H), 4.68 (dd, J = 12.6, 8.7 Hz, 1 H), 4.10-4.22 (m, 4 H), 3.73-3.95 (m, 2 H), 3.47-3.69 (m, 7 H), 2.46-2.56 (m, 1 H), 2.08- 2.33 (m, 7 H). |
| 51 | 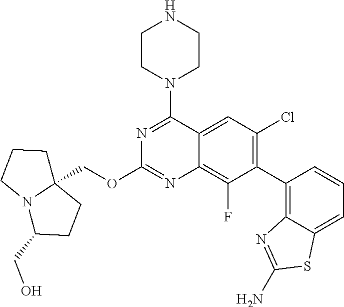 [SMILES: Nc1nc2c(-c3c(Cl)cc4c(N5CCNCC5)nc(OC[C@@]56CCCN5[C@@H](CO)CC6)nc4c3F)cccc2s1] | ((3R,7aS)-7a- (((7-(2- aminobenzo[d] thiazol-4-yl)-6- chloro-8- fluoro-4- (piperazin-1- yl)quinazolin- 2- yl)oxy)methyl) hexahydro-1H- pyrrolizin-3- yl)methanol | 583.6 (M + H)⁺ | ¹H NMR (400 MHz, METHANOL-d4) δ ppm 8.04 (d, J = 1.5 Hz, 1 H), 7.88 (ddd, J = 7.5, 6.1, 1.7 Hz, 1 H), 7.34-7.44 (m, 2 H), 4.75-4.84 (m, 1 H), 4.67 (t, J = 12.3 Hz, 1 H), 4.10-4.21 (m, 4 H), 3.73- 3.94 (m, 2 H), 3.47-3.69 (m, 7 H), 2.45-2.57 (m, 1 H), 2.05-2.32 (m, 7 H). |
| 52 | 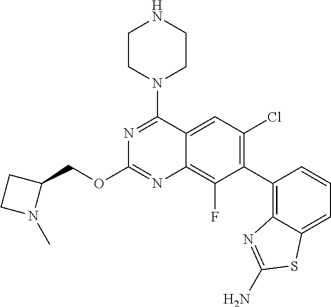 [SMILES: CN1CC[C@H]1COc1nc(N2CCNCC2)c2cc(Cl)c(-c3cccc4sc(N)nc34)c(F)c2n1] | 4-(6-chloro-8- fluoro-2-(((S)- 1- methylazetidin- 2-yl)methoxy)- 4-(piperazin-1- yl)quinazolin- 7- yl)benzo[d] thiazol-2-amine | 514.2 (M + H)⁺ | ¹H NMR (400 MHz, METHANOL-d4) δ ppm 8.04 (s, 1 H), 7.86 (dt, J = 7.5, 1.6 Hz, 1 H), 7.32- 7.40 (m, 2 H), 4.88-5.01 (m, 1 H), 4.74-4.84 (m, 2 H), 4.24 (dt, J = 9.6, 4.9 Hz, 1 H), 4.11-4.18 (m, 4 H), 4.01 (q, J = 9.6 Hz, 1 H), 3.47-3.57 (m, 4 H), 3.03 (s, 3 H), 2.56-2.71 (m, 2 H). |
| 53 | 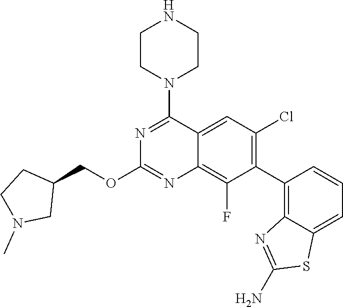 [SMILES: CN1CC[C@@H](COc2nc(N3CCNCC3)c3cc(Cl)c(-c4cccc5sc(N)nc45)c(F)c3n2)C1] | 4-(6-chloro-8- fluoro-2-(((R)- 1- methyl- pyrrolidin-3- yl)methoxy)-4- (piperazin-1- yl)quinazolin- 7- yl)benzo[d] thiazol-2-amine | 528.0 (M + H)⁺ | ¹H NMR (400 MHz, METHANOL-d4) δ ppm 8.02 (s, 1 H), 7.88 (dd, J = 7.6, 1.6 Hz, 1 H), 7.36- 7.44 (m, 2 H), 4.47-4.64 (m, 2 H), 4.14 (br s, 4 H), 3.69-3.95 (m, 2 H), 3.45- 3.59 (m, 4 H), 3.34-3.43 (m, 1 H), 3.05-3.31 (m, 2 H), 3.00 (br s, 3 H), 2.27- 2.56 (m, 1 H), 1.96-2.23 (m, 1 H). |
| 54 | 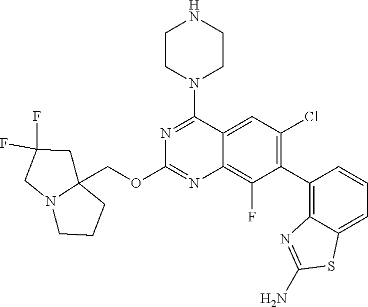 [SMILES: Nc1nc2c(-c3c(Cl)cc4c(N5CCNCC5)nc(OCC56CCCN5CC(F)(F)C6)nc4c3F)cccc2s1] | 4-(6-chloro-2- ((2,2- difluorotetra- hydro-1H- pyrrolizin- 7a(5H)- yl)methoxy)-8- fluoro-4- (piperazin-1- yl)quinazolin- 7- yl)benzo[d] thiazol-2-amine | 612.0 (M + Na)⁺ | ¹H NMR (400 MHz, METHANOL-d4) δ ppm 8.04 (d, J = 1.5 Hz, 1 H), 7.83 (dd, J = 7.3, 1.3 Hz, 1 H), 7.28-7.36 (m, 2 H), 4.73-4.83 (m, 2 H), 4.11- 4.31 (m, 5 H), 3.83-4.01 (m, 2 H), 3.45-3.58 (m, 5 H), 2.94-3.05 (m, 1 H), 2.80-2.94 (m, 1 H), 2.22- 2.51 (m, 4 H). |
| 55 | 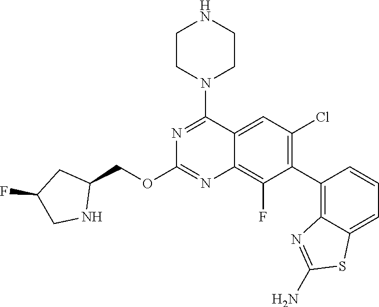 [SMILES: Nc1nc2c(-c3c(Cl)cc4c(N5CCNCC5)nc(OC[C@@H]5C[C@H](F)CN5)nc4c3F)cccc2s1] | 4-(6-chloro-8- fluoro-2- (((2S,4S)-4- fluoro- pyrrolidin-2- yl)methoxy)-4- (piperazin-1- yl)quinazolin- 7- yl)benzo[d] thiazol-2-amine | 532.2 (M + H)⁺ | ¹H NMR (400 MHz, METHANOL-d4) δ ppm 8.00-8.05 (m, 1 H), 7.86 (d, J = 8.2 Hz, 1 H), 7.32- 7.41 (m, 2 H), 5.39-5.61 (m, 1 H), 4.65 (ddd, J = 12.0, 9.7, 2.3 Hz, 1 H), 4.28-4.37 (m, 1 H), 4.06- 4.18 (m, 4 H), 3.69-3.83 (m, 1 H), 3.44-3.60 (m, 6 H), 2.62-2.79 (m, 1 H), 2.24-2.37 (m, 1 H). |
| 56 | 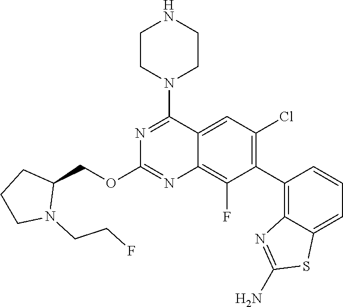 [SMILES: Nc1nc2c(-c3c(Cl)cc4c(N5CCNCC5)nc(OC[C@@H]5CCCN5CCF)nc4c3F)cccc2s1] | 4-(6-chloro-8- fluoro-2-(((S)- 1-(2- fluoroethyl) pyrrolidin-2- yl)methoxy)-4- (piperazin-1- yl)quinazolin- 7- yl)benzo[d] thiazol-2-amine | 560.2 (M + H)⁺ | ¹H NMR (400 MHz, METHANOL-d4) δ ppm 8.04 (t, J = 1.6 Hz, 1 H), 7.85 (d, J = 7.8 Hz, 1 H), 7.32-7.39 (m, 2 H), 4.88- 4.96 (m, 2 H), 4.71-4.84 (m, 2 H), 3.96-4.19 (m, 5 H), 3.85 (br s, 1 H), 3.47- 3.73 (m, 4 H), 3.33-3.46 (m, 3 H), 2.39-2.49 (m, 1 H), 2.05-2.28 (m, 3 H). |
| 57 | 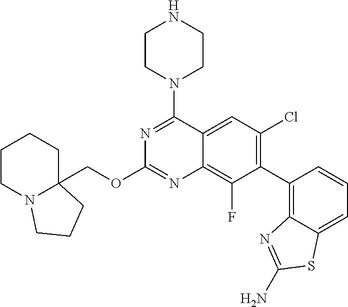 [SMILES: Nc1nc2c(-c3c(Cl)cc4c(N5CCNCC5)nc(OCC56CCCCN5CCC6)nc4c3F)cccc2s1] | 4-(6-chloro-8- fluoro-2- ((hexahydro- indolizin- 8a(1H)- yl)methoxy)-4- (piperazin-1- yl)quinazolin- 7- yl)benzo[d] thiazol-2-amine | 568.2 (M + H)⁺ | ¹H NMR (400 MHz, METHANOL-d4) δ ppm 8.02-8.06 (m, 1 H), 7.88 (d, J = 7.3 Hz, 1 H), 7.35- 7.43 (m, 2 H), 4.88-4.93 (m, 1 H), 4.72 (dd, J = 12.5, 7.5 Hz, 1 H), 4.08-4.20 (m, 4 H), 3.48-3.72 (m, 7 H), 3.24-3.32 (m, 1 H), 2.13-2.36 (m, 4 H), 1.89- 2.08 (m, 3 H), 1.76-1.88 (m, 3 H). |
| 58 | 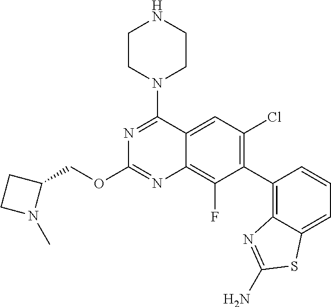 [SMILES: CN1CC[C@@H]1COc1nc(N2CCNCC2)c2cc(Cl)c(-c3cccc4sc(N)nc34)c(F)c2n1] | 4-(6-chloro-8- fluoro-2-(((R)- 1- methylazetidin- 2-yl)methoxy)- 4-(piperazin-1- yl)quinazolin- 7- yl)benzo[d] thiazol-2-amine | 514.2 (M + H)⁺ | ¹H NMR (400 MHz, METHANOL-d4) δ ppm 8.04 (s, 1 H), 7.85 (dt, J = 7.5, 1.6 Hz, 1 H), 7.32- 7.40 (m, 2 H), 4.88-4.98 (m, 1 H), 4.74-4.84 (m, 2 H), 4.25 (td, J = 9.7, 4.8 Hz, 1 H), 4.11-4.18 (m, 4 H), 4.01 (q, J = 9.5 Hz, 1 H), 3.47-3.58 (m, 4 H), 3.03 (s, 3 H), 2.56-2.71 (m, 2 H). |
| 59 | 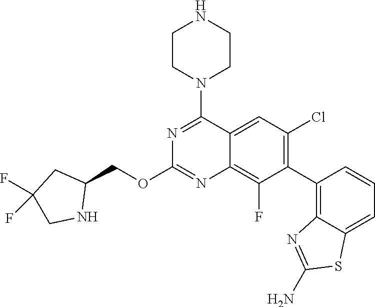 [SMILES: Nc1nc2c(-c3c(Cl)cc4c(N5CCNCC5)nc(OC[C@@H]5CC(F)(F)CN5)nc4c3F)cccc2s1] | 4-(6-chloro-2- (((S)-4,4- difluoro- pyrrolidin-2- yl)methoxy)-8- fluoro-4- (piperazin-1- yl)quinazolin- 7- yl)benzo[d] thiazol-2-amine | 550.0 (M + H)⁺ | ¹H NMR (400 MHz, METHANOL-d4) δ ppm 8.03 (d, J = 1.5 Hz, 1 H), 7.86 (dd, J = 7.5, 1.7 Hz, 1 H), 7.32-7.40 (m, 2 H), 4.89-4.98 (m, 1 H), 4.72- 4.83 (m, 1 H), 4.48 (dtd, J = 10.3, 7.5, 7.5, 3.1 Hz, 1 H), 4.13 (dd, J = 6.3, 4.0 Hz, 4 H), 3.73-3.99 (m, 2 H), 3.45-3.58 (m, 4 H), 2.87 (dq, J = 14.8, 7.3 Hz, 1 H), 2.56-2.71 (m, 1 H), 1.37-1.41 (m, 1 H). |
| 60 | 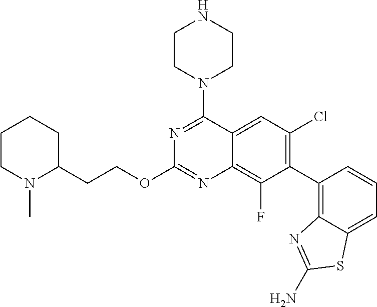 [SMILES: CN1CCCCC1CCOc1nc(N2CCNCC2)c2cc(Cl)c(-c3cccc4sc(N)nc34)c(F)c2n1] | 4-(6-chloro-8- fluoro-2-(2-(1- methylpiperidin- 2-yl)ethoxy)- 4-(piperazin-1- yl)quinazolin- 7- yl)benzo[d] thiazol-2-amine | 555.6 (M + H)⁺ | ¹H NMR (400 MHz, METHANOL-d4) δ ppm 8.03 (d, J = 1.5 Hz, 1 H), 7.90 (dd, J = 7.6, 1.4 Hz, 1 H), 7.38-7.46 (m, 2 H), 4.55-4.75 (m, 2 H), 4.13 (br dd, J = 5.7, 3.7 Hz, 4 H), 3.46-3.58 (m, 5 H), 3.10 (td, J = 12.6, 3.0 Hz, 1 H), 3.01 (s, 2 H), 2.93 (s, 1 H), 2.50 (ddd, J = 8.3, 6.4, 3.9 Hz, 1 H), 2.13-2.32 (m, 2 H), 1.75-1.99 (m, 4 H), 1.59-1.73 (m, 2 H). |
| 61 | 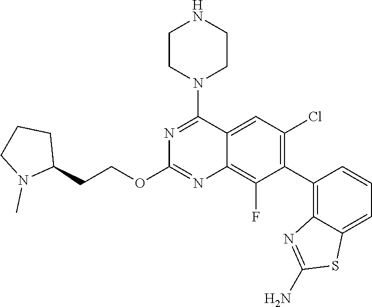 [SMILES: CN1CCC[C@H]1CCOc1nc(N2CCNCC2)c2cc(Cl)c(-c3cccc4sc(N)nc34)c(F)c2n1] | 4-(6-chloro-8- fluoro-2-(2- ((S)-1- methyl- pyrrolidin- 2-yl)ethoxy)- 4-(piperazin-1- yl)quinazolin- 7- yl)benzo[d] thiazol-2-amine | 542.2 (M + H)⁺ | ¹H NMR (400 MHz, METHANOL-d4) δ ppm 8.01 (d, J = 1.5 Hz, 1 H), 7.87 (dd, J = 7.5, 1.7 Hz, 1 H), 7.34-7.42 (m, 2 H), 4.58-4.72 (m, 2 H), 4.12 (br dd, J = 6.1, 3.8 Hz, 4 H), 3.73 (ddd, J = 11.9, 7.7, 5.3 Hz, 1 H), 3.46-3.62 (m, 5 H), 3.15-3.31 (m, 1 H), 3.00 (s, 3 H), 2.34- 2.58 (m, 2 H), 2.03-2.23 (m, 3 H), 1.89-2.01 (m, 1 H). |
| 62 | 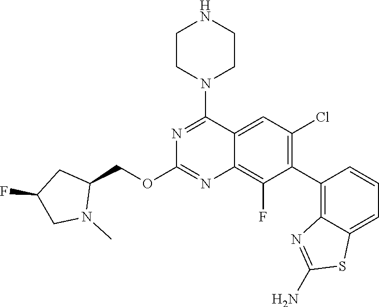 [SMILES: CN1C[C@@H](F)C[C@H]1COc1nc(N2CCNCC2)c2cc(Cl)c(-c3cccc4sc(N)nc34)c(F)c2n1] | 4-(6-chloro-8- fluoro-2- (((2S,4S)-4- fluoro-1- methyl- pyrrolidin-2- yl)methoxy)-4- (piperazin-1- yl)quinazolin- 7- yl)benzo[d] thiazol-2-amine | 546.2 (M + H)⁺ | ¹H NMR (400 MHz, METHANOL-d4) δ ppm 8.05 (s, 1 H), 7.88 (dt, J = 7.5, 1.4 Hz, 1 H), 7.36- 7.44 (m, 2 H), 5.38-5.57 (m, 1 H), 4.92-5.02 (m, 1 H), 4.67-4.81 (m, 1 H), 4.15 (br d, J = 3.3 Hz, 5 H), 3.95-4.07 (m, 1 H), 3.44- 3.60 (m, 5 H), 3.20 (s, 3 H), 2.79-2.97 (m, 1 H), 2.31-2.45 (m, 1 H). |
| 63 | 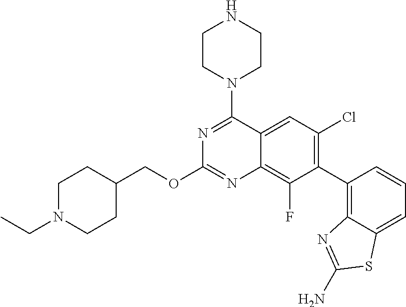 [SMILES: CCN1CCC(COc2nc(N3CCNCC3)c3cc(Cl)c(-c4cccc5sc(N)nc45)c(F)c3n2)CC1] | 4-(6-chloro-2- ((1- ethylpiperidin- 4-yl)methoxy)- 8-fluoro-4- (piperazin-1- yl)quinazolin- 7- yl)benzo[d] thiazol-2-amine | 556.0 (M + H)⁺ | ¹H NMR (400 MHz, METHANOL-d4) δ ppm 7.99 (d, J = 1.7 Hz, 1 H), 7.83 (dd, J = 7.4, 1.6 Hz, 1 H), 7.27-7.36 (m, 2 H), 4.43 (d, J = 5.9 Hz, 2 H), 4.04-4.15 (m, 4 H), 3.62- 3.71 (m, 2 H), 3.46-3.56 (m, 4 H), 3.15-3.31 (m, 2 H), 2.97-3.09 (m, 2 H), 2.14-2.28 (m, 3 H), 1.72 (br d, J = 14.0 Hz, 2 H), 1.32-1.41 (m, 3 H). |
| 64 | 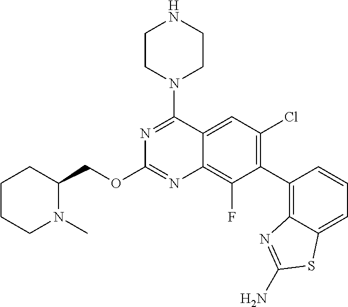 [SMILES: CN1CCCC[C@H]1COc1nc(N2CCNCC2)c2cc(Cl)c(-c3cccc4sc(N)nc34)c(F)c2n1] | 4-(6-chloro-8- fluoro-2-(((S)- 1- methyl- piperidin-2- yl)methoxy)-4- (piperazin-1- yl)quinazolin- 7- yl)benzo[d] thiazol-2-amine | 542.2 (M + H)⁺ | ¹H NMR (400 MHz, METHANOL-d4) δ ppm 8.03 (d, J = 1.5 Hz, 1 H), 7.85 (dt, J = 7.5, 1.3 Hz, 1 H), 7.31-7.39 (m, 2 H), 4.88-4.99 (m, 1 H), 4.64- 4.76 (m, 1 H), 4.08-4.16 (m, 4 H), 3.48-3.60 (m, 6 H), 3.13-3.24 (m, 1 H), 3.04 (d, J = 1.5 Hz, 3 H), 2.13 (br d, J = 13.6 Hz, 1 H), 1.79-2.05 (m, 4 H), 1.63-1.75 (m, 1 H). |
| 65 | 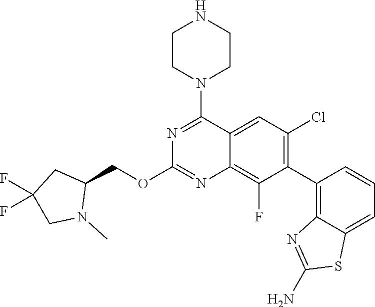 [SMILES: CN1CC(F)(F)C[C@H]1COc1nc(N2CCNCC2)c2cc(Cl)c(-c3cccc4sc(N)nc34)c(F)c2n1] | 4-(6-chloro-2- (((S)-4,4- difluoro-1- methyl- pyrrolidin-2- yl)methoxy)-8- fluoro-4- (piperazin-1- yl)quinazolin- 7- yl)benzo[d] thiazol-2-amine | 564.0 (M + H)⁺ | ¹H NMR (400 MHz, METHANOL-d4) δ ppm 8.03 (t, J = 1.8 Hz, 1 H), 7.83 (dt, J = 7.4, 1.9 Hz, 1 H), 7.28-7.36 (m, 2 H), 4.88-5.02 (m, 1 H), 4.73- 4.83 (m, 1 H), 4.06-4.21 (m, 6 H), 3.73 (q, J = 13.4 Hz, 1 H), 3.46-3.57 (m, 4 H), 3.09-3.31 (m, 3 H), 2.88-3.01 (m, 1 H), 2.71 (qd, J = 15.1, 10.0 Hz, 1 H). |
| 66 | 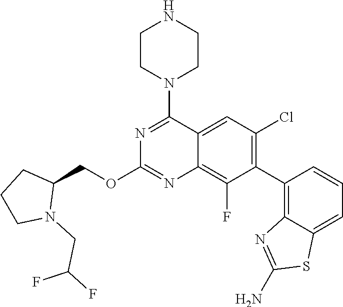 [SMILES: Nc1nc2c(-c3c(Cl)cc4c(N5CCNCC5)nc(OC[C@@H]5CCCN5CC(F)F)nc4c3F)cccc2s1] | 4-(6-chloro-2- (((S)-1-(2,2- difluoroethyl) pyrrolidin-2- yl)methoxy)-8- fluoro-4- (piperazin-1- yl)quinazolin- 7- yl)benzo[d] thiazol-2-amine | 578.2 (M + H)⁺ | ¹H NMR (400 MHz, METHANOL-d4) δ ppm 8.03 (s, 1 H), 7.84 (d, J = 7.5 Hz, 1 H), 7.29-7.37 (m, 2 H), 6.43 (br s, 1 H), 6.43 (br t, J = 53.5 Hz, 1 H), 4.91 (dt, J = 13.0, 3.5 Hz, 1 H), 4.74 (ddd, J = 12.9, 7.0, 2.1 Hz, 1 H), 4.09-4.24 (m, 6 H), 3.68- 3.91 (m, 2 H), 3.36-3.57 (m, 5 H), 2.37-2.48 (m, 1 H), 2.04-2.29 (m, 3 H). |
| 67 | 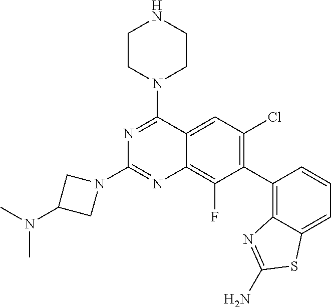 [SMILES: CN(C)C1CN(c2nc(N3CCNCC3)c3cc(Cl)c(-c4cccc5sc(N)nc45)c(F)c3n2)C1] | 4-(6-chloro-2- (3- (dimethylamino) azetidin-1- yl)-8-fluoro-4- (piperazin-1- yl)quinazolin- 7- yl)benzo[d] thiazol-2-amine | 513.2 (M + H)⁺ | ¹H NMR (400 MHz, METHANOL-d4) δ ppm 7.93 (d, J = 1.3 Hz, 1 H), 7.87 (dd, J = 7.7, 1.5 Hz, 1 H), 7.33-7.42 (m, 2 H), 4.57-4.82 (m, 2 H), 4.48 (br dd, J = 10.7, 4.8 Hz, 2 H), 4.23-4.33 (m, 1 H), 4.11 (br dd, J = 5.4, 3.8 Hz, 4 H), 3.35-3.55 (m, 4 H), 2.98 (s, 6 H). |
| 68 | 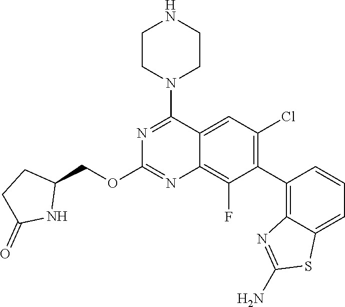 [SMILES: Nc1nc2c(-c3c(Cl)cc4c(N5CCNCC5)nc(OC[C@@H]5CCC(=O)N5)nc4c3F)cccc2s1] | (5S)-5-(((7-(2- aminobenzo[d] thiazol-4-yl)-6- chloro-8- fluoro-4- (piperazin-1- yl)quinazolin- 2- yl)oxy)methyl) pyrrolidin-2- one | 527.6 (M + H)⁺ | ¹H NMR (400 MHz, METHANOL-d4) δ ppm 8.03 (d, J = 1.7 Hz, 1 H), 7.90 (dt, J = 7.7, 1.3 Hz, 1 H), 7.37-7.46 (m, 2 H), 4.76-4.85 (m, 1 H), 4.55- 4.65 (m, 1 H), 4.46 (ddd, J = 11.1, 6.2, 4.7 Hz, 1 H), 4.08-4.20 (m, 5 H), 3.46- 3.57 (m, 4 H), 2.32-2.54 (m, 3 H), 2.01-2.11 (m, 1 H). |
| 69 | 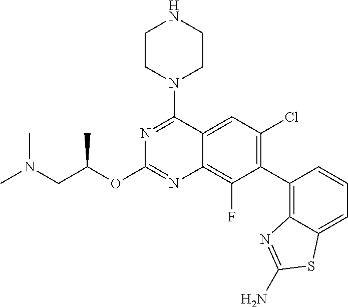 [SMILES: C[C@H](CN(C)C)Oc1nc(N2CCNCC2)c2cc(Cl)c(-c3cccc4sc(N)nc34)c(F)c2n1] | 4-(6-chloro-2- (((R)-1- (dimethylamino) propan-2- yl)oxy)-8- fluoro-4- (piperazin-1- yl)quinazolin- 7- yl)benzo[d] thiazol-2-amine | 516.2 (M + H)⁺ | ¹H NMR (400 MHz, METHANOL-d4) δ ppm 8.03 (s, 1 H), 7.85-7.90 (m, 1 H), 7.34-7.43 (m, 2 H), 5.67-5.75 (m, 1 H), 4.08-4.18 (m, 4 H), 3.63 (dd, J = 13.6, 9.8 Hz, 1 H), 3.42-3.56 (m, 5 H), 3.01 (s, 6 H), 1.49-1.55 (m, 3 H). |
| 70 | 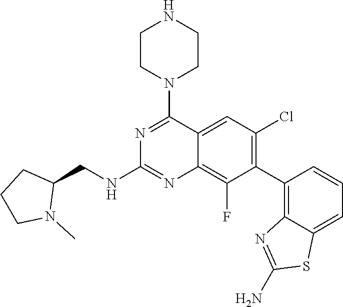 [SMILES: CN1CCC[C@H]1CNc1nc(N2CCNCC2)c2cc(Cl)c(-c3cccc4sc(N)nc34)c(F)c2n1] | 4-(6-chloro-8- fluoro-2-((((S)- 1- methyl- pyrrolidin-2- yl)methyl) amino)-4- (piperazin-1- yl)quinazolin- 7- yl)benzo[d] thiazol-2-amine | 527.2 (M + H)⁺ | ¹H NMR (400 MHz, METHANOL-d4) δ ppm 7.91 (s, 1 H), 7.84 (ddd, J = 7.0, 4.8, 2.2 Hz, 1 H), 7.30-7.38 (m, 2 H), 3.93- 4.15 (m, 5 H), 3.45-3.70 (m, 7 H), 3.06-3.21 (m, 1 H), 3.02 (d, J = 6.1 Hz, 3 H), 2.26 (br dd, J = 12.0, 6.8 Hz, 1 H), 2.04-2.19 (m, 2 H), 1.88-2.01 (m, 1 H). |
| 71 | 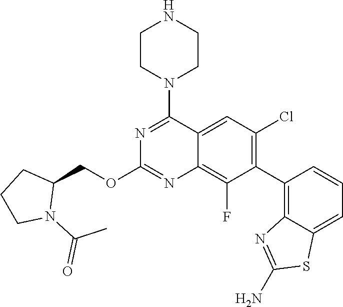 [SMILES: CC(=O)N1CCC[C@H]1COc1nc(N2CCNCC2)c2cc(Cl)c(-c3cccc4sc(N)nc34)c(F)c2n1] | 1-((2S)-2-(((7- (2- aminobenzo[d] thiazol-4-yl)-6- chloro-8- fluoro-4- (piperazin-1- yl)quinazolin- 2- yl)oxy)methyl) pyrrolidin-1- yl)ethan-1-one | 556.0 (M + H)⁺ | ¹H NMR (400 MHz, METHANOL-d4) δ ppm 8.00-8.04 (m, 1 H), 7.84- 7.89 (m, 1 H), 7.33-7.42 (m, 2 H), 4.89-4.97 (m, 1 H), 4.63-4.84 (m, 1 H), 4.44-4.56 (m, 2 H), 4.17- 4.25 (m, 3 H), 4.03-4.16 (m, 1 H), 3.45-3.65 (m, 6 H), 2.28 (d, J = 4.2 Hz, 1 H), 1.95-2.23 (m, 5 H). |
| 72 | 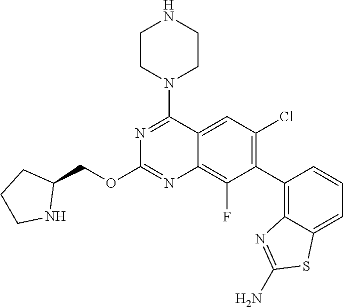 [SMILES: Nc1nc2c(-c3c(Cl)cc4c(N5CCNCC5)nc(OC[C@@H]5CCCN5)nc4c3F)cccc2s1] | 4-(6-chloro-8- fluoro-4- (piperazin-1- yl)-2-(((S)- pyrrolidin-2- yl)methoxy) quinazolin-7- yl)benzo[d] thiazol-2-amine | 514.2 (M + H)⁺ | ¹H NMR (400 MHz, METHANOL-d4) δ ppm 8.03 (d, J = 1.3 Hz, 1 H), 7.87 (dd, J = 7.6, 1.6 Hz, 1 H), 7.33-7.44 (m, 2 H), 4.77-4.84 (m, 1 H), 4.58- 4.68 (m, 1 H), 4.05-4.18 (m, 5 H), 3.46-3.55 (m, 4 H), 3.36-3.45 (m, 2 H), 2.25-2.39 (m, 1 H), 2.02- 2.23 (m, 2 H), 1.90-2.02 (m, 1 H). |
| 73 | 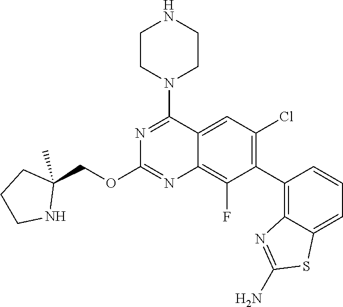 [SMILES: C[C@@]1(COc2nc(N3CCNCC3)c3cc(Cl)c(-c4cccc5sc(N)nc45)c(F)c3n2)CCCN1] | 4-(6-chloro-8- fluoro-2-(((S)- 2- methyl- pyrrolidin-2- yl)methoxy)-4- (piperazin-1- yl)quinazolin- 7- yl)benzo[d] thiazol-2-amine | 528.3 (M + H)⁺ | ¹H NMR (400 MHz, METHANOL-d4) δ ppm 8.01-8.06 (m, 1 H), 7.86- 7.91 (m, 1 H), 7.35-7.46 (m, 2 H), 4.57-4.69 (m, 2 H), 4.06-4.20 (m, 4 H), 3.40-3.57 (m, 6 H), 2.13- 2.27 (m, 3 H), 1.96-2.11 (m, 1 H), 1.54-1.64 (m, 3 H). |
| 74 | 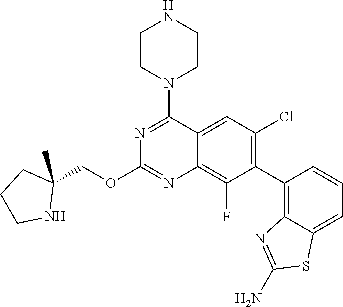 [SMILES: C[C@]1(COc2nc(N3CCNCC3)c3cc(Cl)c(-c4cccc5sc(N)nc45)c(F)c3n2)CCCN1] | 4-(6-chloro-8- fluoro-2-(((R)- 2- methyl- pyrrolidin-2- yl)methoxy)-4- (piperazin-1- yl)quinazolin- 7- yl)benzo[d] thiazol-2-amine | 528.3 (M + H)⁺ | ¹H NMR (400 MHz, METHANOL-d4) δ ppm 8.04 (d, J = 1.5 Hz, 1 H), 7.89-7.95 (m, 1 H), 7.40- 7.51 (m, 2 H), 4.60-4.71 (m, 2 H), 4.05-4.20 (m, 4 H), 3.41-3.58 (m, 6 H), 2.12-2.25 (m, 3 H), 1.98- 2.08 (m, 1 H), 1.55-1.63 (m, 3 H). |
| 75 | 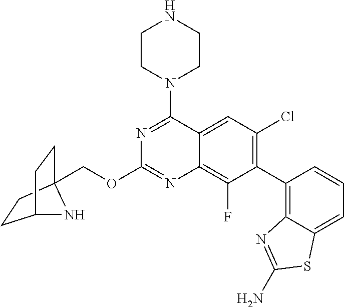 [SMILES: Nc1nc2c(-c3c(Cl)cc4c(N5CCNCC5)nc(OCC56CCC(CC5)N6)nc4c3F)cccc2s1] | 4-(2-(((1s,4s)- 7- azabicyclo[2.2.1] heptan-1- yl)methoxy)-6- chloro-8- fluoro-4- (piperazin-1- yl)quinazolin- 7- yl)benzo[d] thiazol-2-amine | 540.0 (M + H)⁺ | ¹H NMR (400 MHz, METHANOL-d4) δ ppm 8.03 (d, J = 1.7 Hz, 1 H), 7.84-7.89 (m, 1 H), 7.33- 7.42 (m, 2 H), 4.21-4.29 (m, 1 H), 4.07-4.18 (m, 4 H), 3.46-3.57 (m, 4 H), 2.11-2.22 (m, 4 H), 1.90- 2.00 (m, 4 H). |

### 4-(6-Chloro-2-(3-(dimethylamino)-3-methyl)azetidin-1-y)-8-fluoro-4(piperazin-1-yl)quinazolin-7-yl)benzo[d]thiazol-2-amine (Example 76

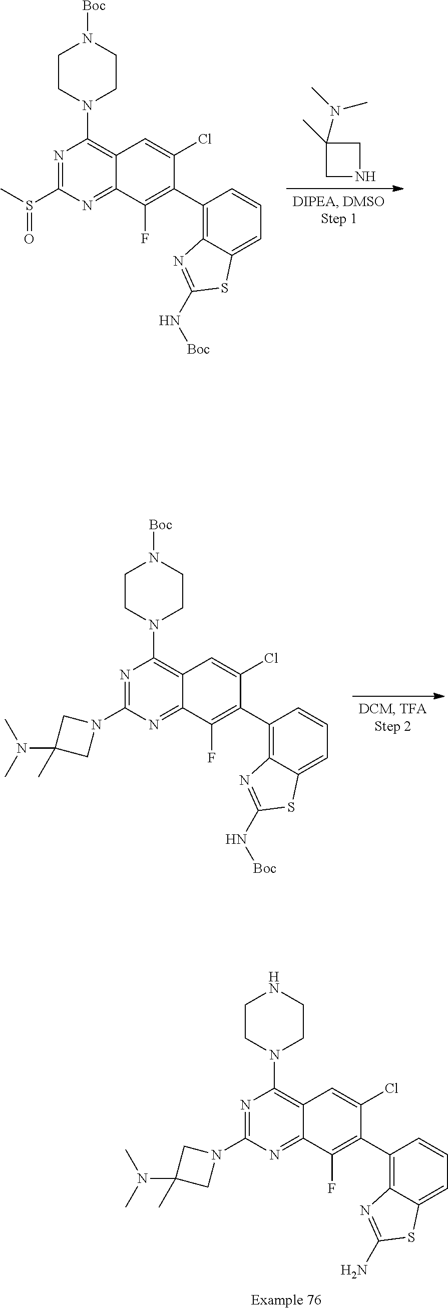

Step 1: tert-Butyl 4-(7-(2-((tert-butoxycarbonyl)amino)benzo[d]thiazol-4-yl)-6-chloro-2-(3-(dimethylamino)-3-methyl)azetidin-1-yl)-8-fluoroquinazolin-4-yl)piperazine-1-carboxylate. To a solution of tert-butyl 4-(7-(2-((tert-butoxycarbonyl)amino)benzo[d]thiazol-4-yl)-6-chloro-8-fluoro-2-(methylsulfinyl)quinazolin-4-yl)piperazine-1-carboxylate (1 equiv., 100 mM solution in DMSO) was added a solution of N,N,3-trimethyl)azetidin-3-amine (1 equiv, 100 mM solution in DMSO), followed by DIPEA (4.75 equiv). The mixture was shaken at 80° C. overnight, then 100° C. for 4 h. Thereafter, volatiles were removed under reduced pressure, affording tert-butyl 4-(7-(2-((tert-butoxycarbonyl)amino)benzo[d]thiazol-4-yl)-6-chloro-2-(3-(dimethylamino)-3-methyl)azetidin-1-yl)-8-fluoroquinazolin-4-yl)piperazine-1-carboxylate, which was used directly in the following step.

Step 2: 4-(6-Chloro-2-(3-(dimethylamino)-3-methyl)azetidin-1-yl)-8-fluoro-4-(piperazin-1-yl)quinazolin-7-yl)benzo[d]thiazol-2-amine. tert-Butyl 4-(7-(2-((tert-butoxycarbonyl)amino)benzo[d]thiazol-4-yl)-6-chloro-2-(3-(dimethylamino)-3-methyl)azetidin-1-yl)-8-fluoroquinazolin-4-yl)piperazine-1-carboxylate was dissolved in 30% TFA in DCM (24 mM). The reaction was shaken for 2 h at room temperature. Volatiles were removed under air flow to give crude product that was thereafter purified by HPLC to yield the final product, 4-(6-chloro-2-(3-(dimethylamino)-3-methyl)azetidin-1-yl)-8-fluoro-4-(piperazin-1-yl)quinazolin-7-yl)benzo[d]thiazol-2-amine, with 95% purity by UV. m/z (ESI): 527.2 (M+H)⁺. R.T.: 1.64 min.

TABLE 4
Examples prepared in a manner similar to that described above for Example 76.

| | | | MS | |
|---|---|---|---|---|
| Ex. # | Structure | Name | m/z (ESI, +ve ion) | R.T. |
| 77 | 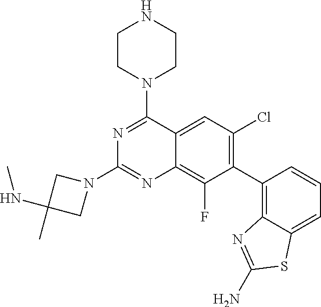 [SMILES: CNC1(C)CN(c2nc(N3CCNCC3)c3cc(Cl)c(-c4cccc5sc(N)nc45)c(F)c3n2)C1] | 4-[6-chloro-8-fluoro-2-[3-methyl-3- (methylamino)azetidin-1-yl]-4-piperazin- 1-yl-quinazolin-7-yl]-1,3-benzothiazol-2- amine | 513.2 | 1.63 |
| 78 | 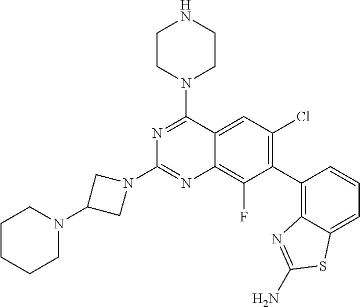 [SMILES: Nc1nc2c(-c3c(Cl)cc4c(N5CCNCC5)nc(N5CC(N6CCCCC6)C5)nc4c3F)cccc2s1] | 4-[6-chloro-8-fluoro-4-piperazin-1-yl-2- [3-(1-piperidyl)azetidin-1-yl]quinazolin- 7-yl]-1,3-benzothiazol-2-amine | 553.2 | 1.53 |
| 79 | 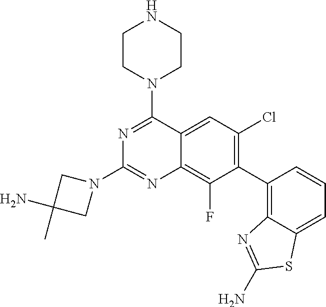 [SMILES: CC1(N)CN(c2nc(N3CCNCC3)c3cc(Cl)c(-c4cccc5sc(N)nc45)c(F)c3n2)C1] | 4-[2-(3-amino-3-methyl-azetidin-1-yl)-6- chloro-8-fluoro-4-piperazin-1-yl- quinazolin-7-yl]-1,3-benzothiazol-2- amine | 499.2 | 1.62 |
| 80 | 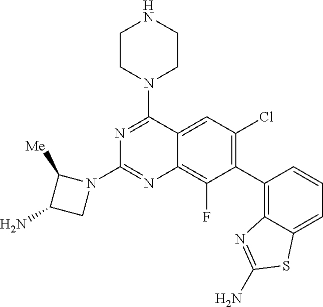 [SMILES: C[C@@H]1[C@@H](N)CN1c1nc(N2CCNCC2)c2cc(Cl)c(-c3cccc4sc(N)nc34)c(F)c2n1] | 4-[2-[(2R,3S)-3-amino-2-methyl- azetidin-1-yl]-6-chloro-8-fluoro-4- piperazin-1-yl-quinazolin-7-yl]-1,3- benzothiazol-2-amine | 499.2 | 1.73 |
| 81 | 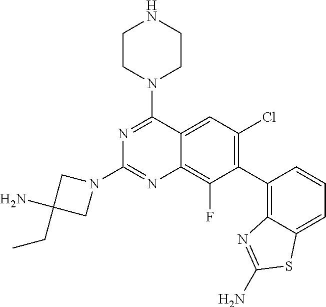 [SMILES: CCC1(N)CN(c2nc(N3CCNCC3)c3cc(Cl)c(-c4cccc5sc(N)nc45)c(F)c3n2)C1] | 4-[2-(3-amino-3-ethyl-azetidin-1-yl)-6- chloro-8-fluoro-4-piperazin-1-yl- quinazolin-7-yl]-1,3-benzothiazol-2- amine | 513.2 | 1.79 |
| 82 | 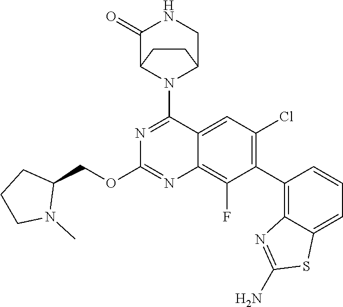 [SMILES: CN1CCC[C@H]1COc1nc(N2C3CCC2C(=O)NC3)c2cc(Cl)c(-c3cccc4sc(N)nc34)c(F)c2n1] | 8-(7-(2-aminobenzo[d]thiazol-4-yl)-6- chloro-8-fluoro-2-(((S)-1- methylpyrrolidin-2- yl)methoxy)quinazolin-4-yl)-3,8- diazabicyclo[3.2.1]octan-2-one | 568.2 | 2.31 and 2.61 |
| 83 | 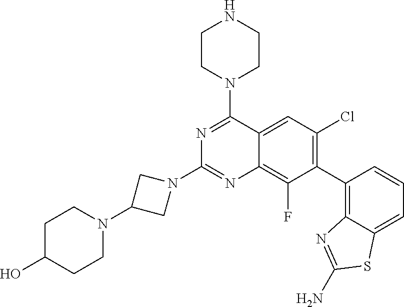 [SMILES: Nc1nc2c(-c3c(Cl)cc4c(N5CCNCC5)nc(N5CC(N6CCC(O)CC6)C5)nc4c3F)cccc2s1] | 1-[1-[7-(2-amino-1,3-benzothiazol-4-yl)- 6-chloro-8-fluoro-4-piperazin-1-yl- quinazolin-2-yl]azetidin-3-yl]piperidin-4- ol | 569.2 | 1.54 |
| 84 | 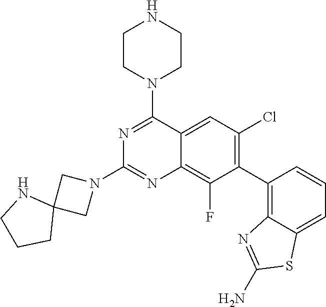 [SMILES: Nc1nc2c(-c3c(Cl)cc4c(N5CCNCC5)nc(N5CC6(CCCN6)C5)nc4c3F)cccc2s1] | 4-[6-chloro-2-(2,5-diazaspiro[3.4]octan- 2-yl)-8-fluoro-4-piperazin-1-yl- quinazolin-7-yl]-1,3-benzothiazol-2- amine | 525.2 | 1.68 |
| 85 | 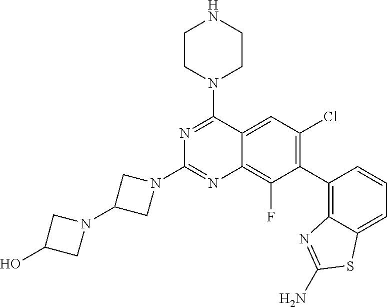 [SMILES: Nc1nc2c(-c3c(Cl)cc4c(N5CCNCC5)nc(N5CC(N6CC(O)C6)C5)nc4c3F)cccc2s1] | 1-[1-[7-(2-amino-1,3-benzothiazol-4-yl)- 6-chloro-8-fluoro-4-piperazin-1-yl- quinazolin-2-yl]azetidin-3-yl]azetidin-3- ol | 541.2 | 1.57 |
| 86 | 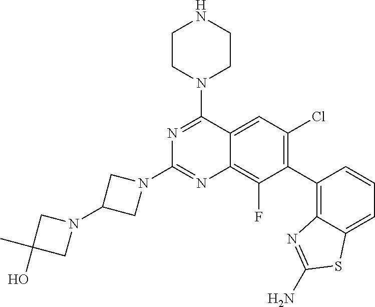 [SMILES: CC1(O)CN(C2CN(c3nc(N4CCNCC4)c4cc(Cl)c(-c5cccc6sc(N)nc56)c(F)c4n3)C2)C1] | 1-[1-[7-(2-amino-1,3-benzothiazol-4-yl)- 6-chloro-8-fluoro-4-piperazin-1-yl- quinazolin-2-yl]azetidin-3-yl]-3-methyl- azetidin-3-ol | 555.2 | 1.56 |
| 87 | 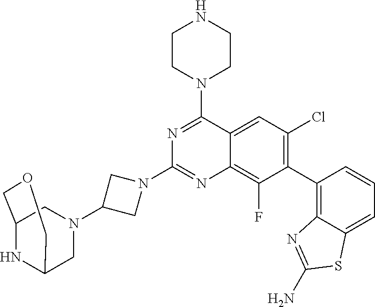 [SMILES: Nc1nc2c(-c3c(Cl)cc4c(N5CCNCC5)nc(N5CC(N6CC7COCC(C6)N7)C5)nc4c3F)cccc2s1] | 4-(2-(3-((1R,5S)-3-oxa-7,9- diazabicyclo[3.3.1]nonan-7-yl)azetidin-1- yl)-6-chloro-8-fluoro-4-(piperazin-1- yl)quinazolin-7-yl)benzo[d]thiazol-2- amine | 596.2 | 1.47 |
| 88 | 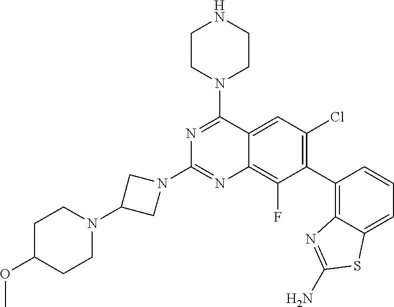 [SMILES: COC1CCN(C2CN(c3nc(N4CCNCC4)c4cc(Cl)c(-c5cccc6sc(N)nc56)c(F)c4n3)C2)CC1] | 4-[6-chloro-8-fluoro-2-[3-(4-methoxy-1- piperidyl)azetidin-1-yl]-4-piperazin-1-yl- quinazolin-7-yl]-1,3-benzothiazol-2- amine | 583.2 | 1.67 |
| 89 | 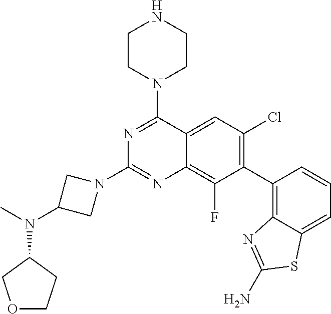 [SMILES: CN(C1CN(c2nc(N3CCNCC3)c3cc(Cl)c(-c4cccc5sc(N)nc45)c(F)c3n2)C1)[C@@H]1CCOC1] | 4-[6-chloro-8-fluoro-2-[3-[methyl-[(3R)- tetrahydrofuran-3-yl]aminoJazetidin-1- yl]-4-piperazin-1-yl-quinazolin-7-yl]-1,3- benzothiazol-2-amine | 569.2 | 1.68 |
| 90 | 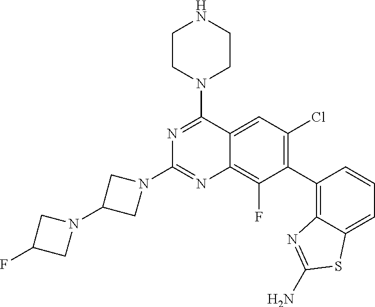 [SMILES: Nc1nc2c(-c3c(Cl)cc4c(N5CCNCC5)nc(N5CC(N6CC(F)C6)C5)nc4c3F)cccc2s1] | 4-[6-chloro-8-fluoro-2-[3-(3- fluoroazetidin-1-yl)azetidin-1-yl]-4- piperazin-1-yl-quinazolin-7-yl]-1,3- benzothiazol-2-amine | 543.2 | 1.66 |
| 91 | 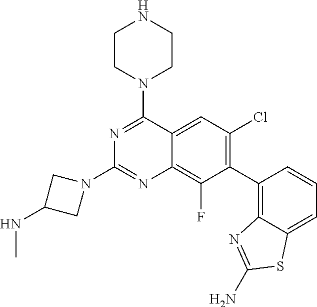 [SMILES: CNC1CN(c2nc(N3CCNCC3)c3cc(Cl)c(-c4cccc5sc(N)nc45)c(F)c3n2)C1] | 4-[6-chloro-8-fluoro-2-[3- (methylamino)azetidin-1-yl]-4-piperazin- 1-yl-quinazolin-7-yl]-1,3-benzothiazol-2- amine | 499.2 | 1.53 |
| 92 | 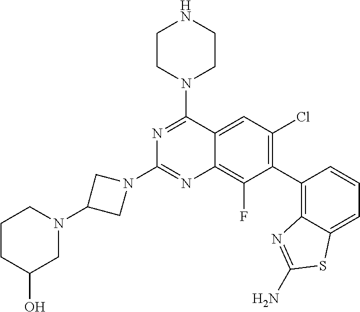 [SMILES: Nc1nc2c(-c3c(Cl)cc4c(N5CCNCC5)nc(N5CC(N6CCCC(O)C6)C5)nc4c3F)cccc2s1] | 1-[1-[7-(2-amino-1,3-benzothiazol-4-yl)- 6-chloro-8-fluoro-4-piperazin-1-yl- quinazolin-2-yl]azetidin-3-yl]piperidin-3- ol | 569.2 | 1.54 |
| 93 | 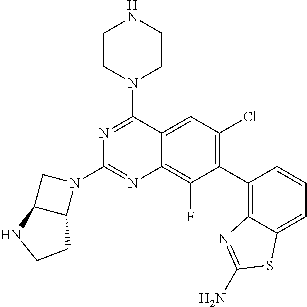 [SMILES: Nc1nc2c(-c3c(Cl)cc4c(N5CCNCC5)nc(N5C[C@@H]6NCC[C@H]65)nc4c3F)cccc2s1] | 4-[6-chloro-2-[(1S,5R)-2,6- diazabicyclo[3.2.0]heptan-6-yl]-8-fluoro- 4-piperazin-1-yl-quinazolin-7-yl]-1,3- benzothiazol-2-amine | 511.2 | 1.56 and 1.63 |
| 94 | 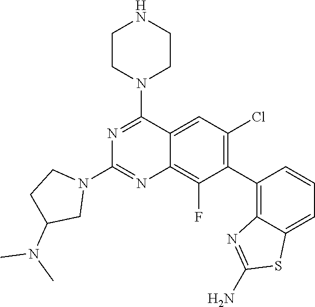 [SMILES: CN(C)C1CCN(c2nc(N3CCNCC3)c3cc(Cl)c(-c4cccc5sc(N)nc45)c(F)c3n2)C1] | 4-[6-chloro-8-fluoro-4-piperazin-1-yl-2- [3-(dimethylamino)pyrrolidin-1- yl]quinazolin-7-yl]-1,3-benzothiazol-2- amine | 527.2 | 1.63 |
| 95 | 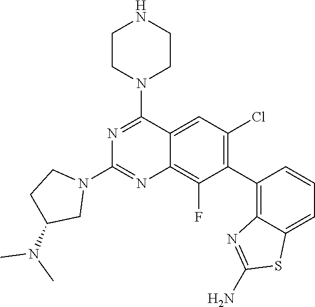 [SMILES: CN(C)[C@@H]1CCN(c2nc(N3CCNCC3)c3cc(Cl)c(-c4cccc5sc(N)nc45)c(F)c3n2)C1] | 4-[6-chloro-2-[(3R)-3- (dimethylamino)pyrrolidin-1-yl]-8- fluoro-4-piperazin-1-yl-quinazolin-7-yl]- 1,3-benzothiazol-2-amine | 527.2 | 1.62 |
| 96 | 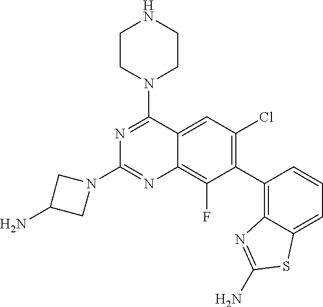 [SMILES: Nc1nc2c(-c3c(Cl)cc4c(N5CCNCC5)nc(N5CC(N)C5)nc4c3F)cccc2s1] | 4-[2-(3-aminoazetidin-1-yl)-6-chloro-8- fluoro-4-piperazin-1-yl-quinazolin-7-yl]- 1,3-benzothiazol-2-amine | 485.1 | 1.46 |
| 97 | 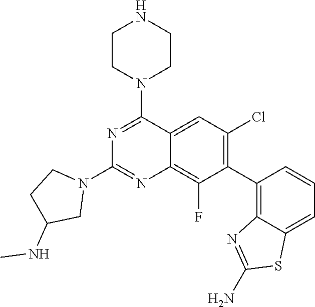 [SMILES: CNC1CCN(c2nc(N3CCNCC3)c3cc(Cl)c(-c4cccc5sc(N)nc45)c(F)c3n2)C1] | 4-[6-chloro-8-fluoro-4-piperazin-1-yl-2- [3-(methylamino)pyrrolidin-1- yl]quinazolin-7-yl]-1,3-benzothiazol-2- amine | 513.2 | 1.63 |
| 98 | 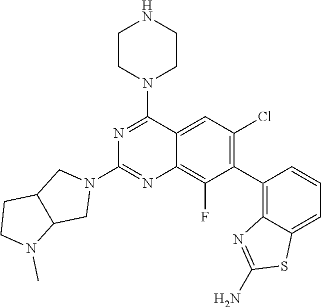 [SMILES: CN1CCC2CN(c3nc(N4CCNCC4)c4cc(Cl)c(-c5cccc6sc(N)nc56)c(F)c4n3)CC21] | 4-(6-chloro-8-fluoro-2-(1- methylhexahydropyrrolo[3,4-b]pyrrol- 5(1H)-yl)-4-(piperazin-1-yl)quinazolin-7- yl)benzo[d]thiazol-2-amine | 539.2 | 1.69 |
| 99 | 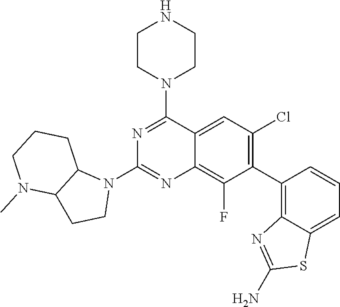 [SMILES: CN1CCCC2C1CCN2c1nc(N2CCNCC2)c2cc(Cl)c(-c3cccc4sc(N)nc34)c(F)c2n1] | 4-(6-chloro-8-fluoro-2-(4- methyloctahydro-1H-pyrrolo[3,2- b]pyridin-1-yl)-4-(piperazin-1- yl)quinazolin-7-yl)benzo[d]thiazol-2- amine | 553.2 | 1.81 |
| 100 | 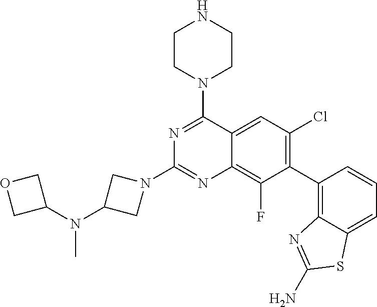 [SMILES: CN(C1COC1)C1CN(c2nc(N3CCNCC3)c3cc(Cl)c(-c4cccc5sc(N)nc45)c(F)c3n2)C1] | 4-[6-chloro-8-fluoro-2-[3- [methyl(oxetan-3-yl)amino]azetidin-1- yl]-4-piperazin-1-yl-quinazolin-7-yl]-1,3- benzothiazol-2-amine | 555.2 | 1.88 |
| 101 | 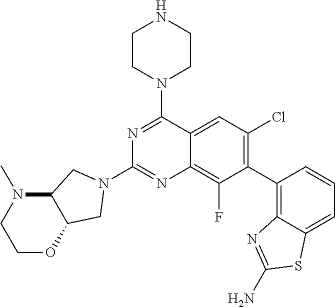 [SMILES: CN1CCO[C@H]2CN(c3nc(N4CCNCC4)c4cc(Cl)c(-c5cccc6sc(N)nc56)c(F)c4n3)C[C@@H]21] | 4-(6-chloro-8-fluoro-2-(trans-4- methylhexahydropyrrolo[3,4- b][1,4]oxazin-6(2H)-yl)-4-(piperazin-1- yl)quinazolin-7-yl)benzo[d]thiazol-2- amine | 555.2 | 1.82 |
| 102 | 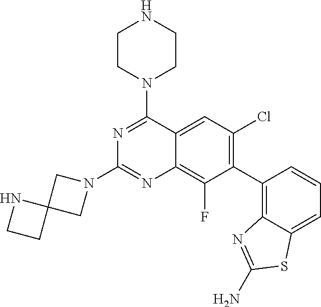 [SMILES: Nc1nc2c(-c3c(Cl)cc4c(N5CCNCC5)nc(N5CC6(CCN6)C5)nc4c3F)cccc2s1] | 4-[6-chloro-2-(1,6-diazaspiro[3.3]heptan- 6-yl)-8-fluoro-4-piperazin-1-yl- quinazolin-7-yl]-1,3-benzothiazol-2- amine | 511.2 | 1.57 |
| 103 | 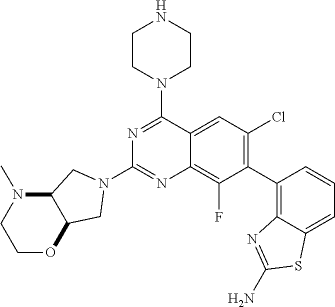 | 4-(6-chloro-8-fluoro-2-(cis-4- methylhexahydropyrrolo[3,4- b][1,4]oxazin-6(2H)-yl)-4-(piperazin-1- yl)quinazolin-7-yl)benzo[d]thiazol-2- amine | 555.2 | 1.72 and 1.80 |
| 104 | 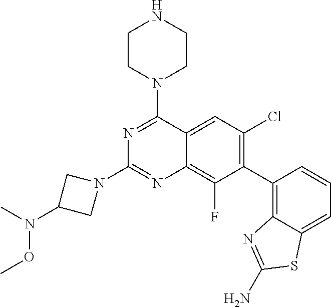 [SMILES: CON(C)C1CN(c2nc(N3CCNCC3)c3cc(Cl)c(-c4cccc5sc(N)nc45)c(F)c3n2)C1] | 4-[6-chloro-8-fluoro-2-[3- [methoxy(methyl)amino]azetidin-1-yl]-4- piperazin-1-yl-quinazolin-7-yl]-1,3- benzothiazol-2-amine | 529.2 | 2.12 |
| 105 | 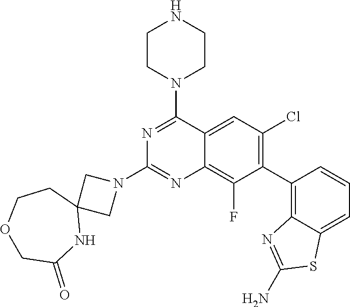 [SMILES: Nc1nc2c(-c3c(Cl)cc4c(N5CCNCC5)nc(N5CC6(CCOCC(=O)N6)C5)nc4c3F)cccc2s1] | 2-[7-(2-amino-1,3-benzothiazol-4-yl)-6- chloro-8-fluoro-4-piperazin-1-yl- quinazolin-2-yl]-8-oxa-2,5- diazaspiro[3.6]decan-6-one | 569.2 | 1.99 |
| 106 | 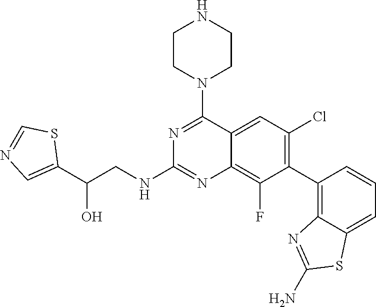 [SMILES: Nc1nc2c(-c3c(Cl)cc4c(N5CCNCC5)nc(NCC(O)c5cncs5)nc4c3F)cccc2s1] | 2-[[7-(2-amino-1,3-benzothiazol-4-yl)-6- chloro-8-fluoro-4-piperazin-1-yl- quinazolin-2-yl]amino]-1-thiazol-5-yl- ethanol | 557.1 | 1.81 |
| 107 | 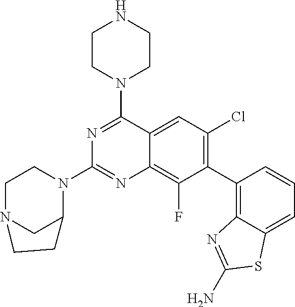 [SMILES: Nc1nc2c(-c3c(Cl)cc4c(N5CCNCC5)nc(N5CCN6CCC5C6)nc4c3F)cccc2s1] | 4-[6-chloro-8-fluoro-4-piperazin-1-yl-2- [1,4-diazabicyclo[3.2.1]octan-4- yl]quinazolin-7-yl]-1,3-benzothiazol-2- amine | 525.2 | 1.76 and 1.79 |
| 108 | 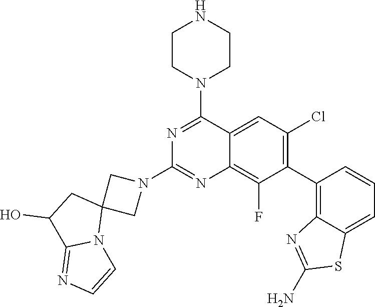 [SMILES: Nc1nc2c(-c3c(Cl)cc4c(N5CCNCC5)nc(N5CC6(CC(O)c7nccn76)C5)nc4c3F)cccc2s1] | 1′-[7-(2-amino-1,3-benzothiazol-4-yl)-6- chloro-8-fluoro-4-piperazin-1-yl- quinazolin-2-yl]spiro[6,7- dihydropyrrolo[1,2-a]imidazole-5,3′- azetidine]-7-ol | 578.2 | 1.71 |
| 109 | 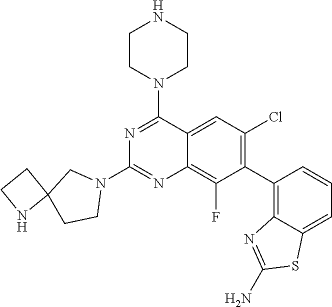 [SMILES: Nc1nc2c(-c3c(Cl)cc4c(N5CCNCC5)nc(N5CCC6(CCN6)C5)nc4c3F)cccc2s1] | 4-[6-chloro-8-fluoro-4-piperazin-1-yl-2- [1,7-diazaspiro[3.4]octan-7- yl]quinazolin-7-yl]-1,3-benzothiazol-2- amine | 525.2 | 1.72 |
| 110 | 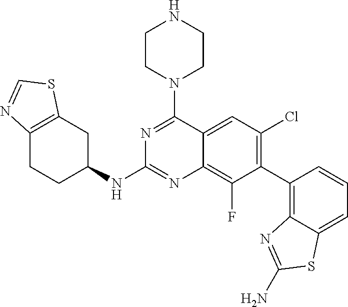 [SMILES: Nc1nc2c(-c3c(Cl)cc4c(N5CCNCC5)nc(N[C@H]5CCc6ncsc6C5)nc4c3F)cccc2s1] | 4-[6-chloro-8-fluoro-4-piperazin-1-yl-2- [[(6S)-4,5,6,7-tetrahydro-1,3- benzothiazol-6-yl]amino]quinazolin-7- yl]-1,3-benzothiazol-2-amine | 567.1 | 2.18 |
| 111 | 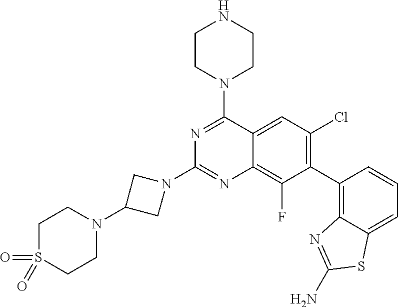 [SMILES: Nc1nc2c(-c3c(Cl)cc4c(N5CCNCC5)nc(N5CC(N6CCS(=O)(=O)CC6)C5)nc4c3F)cccc2s1] | 4-[6-chloro-2-[3-(1,1-dioxo-1,4- thiazinan-4-yl)azetidin-1-yl]-8-fluoro-4- piperazin-1-yl-quinazolin-7-yl]-1,3- benzothiazol-2-amine | 603.2 | 1.90 |
| 112 | 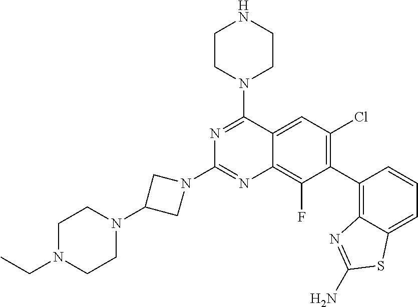 [SMILES: CCN1CCN(C2CN(c3nc(N4CCNCC4)c4cc(Cl)c(-c5cccc6sc(N)nc56)c(F)c4n3)C2)CC1] | 4-[6-chloro-2-[3-(4-ethylpiperazin-1- yl)azetidin-1-yl]-8-fluoro-4-piperazin-1- yl-quinazolin-7-yl]-1,3-benzothiazol-2- amine | 582.2 | 1.51 |
| 113 | 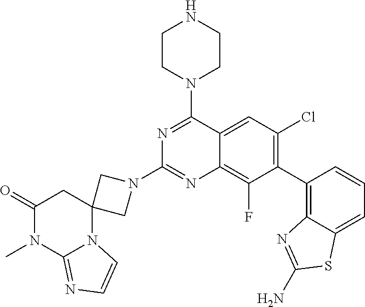 [SMILES: CN1C(=O)CC2(CN(c3nc(N4CCNCC4)c4cc(Cl)c(-c5cccc6sc(N)nc56)c(F)c4n3)C2)n2ccnc21] | 1′-[7-(2-amino-1,3-benzothiazol-4-yl)-6- chloro-8-fluoro-4-piperazin-1-yl- quinazolin-2-yl]-8-methyl-spiro[6H- imidazo[1,2-a]pyrimidine-5,3′-azetidine]- 7-one | 605.2 | 2.16 |
| 114 | 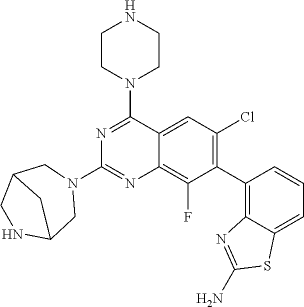 [SMILES: Nc1nc2c(-c3c(Cl)cc4c(N5CCNCC5)nc(N5CC6CNC(C6)C5)nc4c3F)cccc2s1] | 4-[6-chloro-8-fluoro-4-piperazin-1-yl-2- [3,6-diazabicyclo[3.2.1]octan-3- yl]quinazolin-7-yl]-1,3-benzothiazol-2- amine | 525.2 | 1.82 |
| 115 | 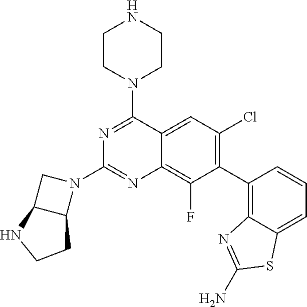 [SMILES: Nc1nc2c(-c3c(Cl)cc4c(N5CCNCC5)nc(N5C[C@@H]6NCC[C@@H]65)nc4c3F)cccc2s1] | 4-[6-chloro-2-[(1S,5S)-2,6- diazabicyclo[3.2.0]heptan-6-yl]-8-fluoro- 4-piperazin-1-yl-quinazolin-7-yl]-1,3- benzothiazol-2-amine | 511.2 | 1.67 |
| 116 | 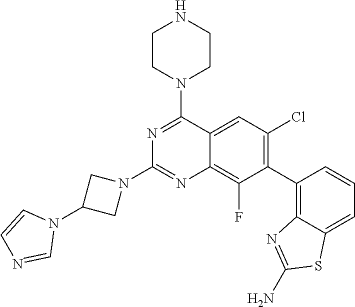 [SMILES: Nc1nc2c(-c3c(Cl)cc4c(N5CCNCC5)nc(N5CC(n6ccnc6)C5)nc4c3F)cccc2s1] | 4-[6-chloro-8-fluoro-2-(3-imidazol-1- ylazetidin-1-yl)-4-piperazin-1-yl- quinazolin-7-yl]-1,3-benzothiazol-2- amine | 536.2 | 1.57 |
| 117 | 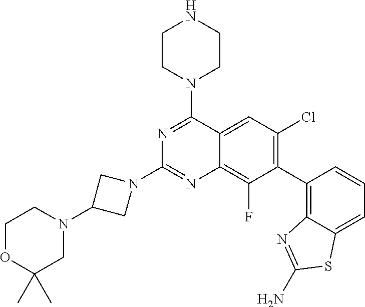 [SMILES: CC1(C)CN(C2CN(c3nc(N4CCNCC4)c4cc(Cl)c(-c5cccc6sc(N)nc56)c(F)c4n3)C2)CCO1] | 4-[6-chloro-2-[3-(2,2-dimethylmorpholin- 4-yl)azetidin-1-yl]-8-fluoro-4-piperazin- 1-yl-quinazolin-7-yl]-1,3-benzothiazol-2- amine | 583.23 | 2.11 |
| 118 | 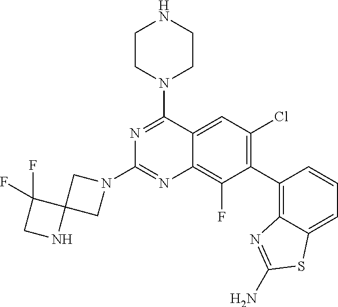 [SMILES: Nc1nc2c(-c3c(Cl)cc4c(N5CCNCC5)nc(N5CC6(C5)NCC6(F)F)nc4c3F)cccc2s1] | 4-[6-chloro-2-(3,3-difluoro-1,6- diazaspiro[3.3]heptan-6-yl)-8-fluoro-4- piperazin-1-yl-quinazolin-7-yl]-1,3- benzothiazol-2-amine | 547.1 | 2.18 |
| 119 | 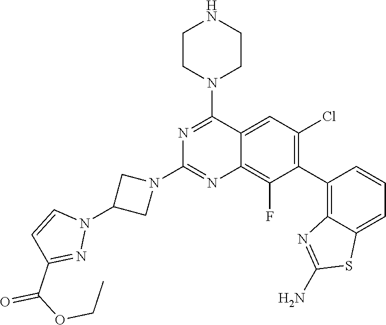 [SMILES: CCOC(=O)c1ccn(C2CN(c3nc(N4CCNCC4)c4cc(Cl)c(-c5cccc6sc(N)nc56)c(F)c4n3)C2)n1] | ethyl 1-[1-[7-(2-amino-1,3-benzothiazol- 4-yl)-6-chloro-8-fluoro-4-piperazin-1-yl- quinazolin-2-yl]azetidin-3-yl]pyrazole-3- carboxylate | 608.2 | 2.36 |
| 120 | 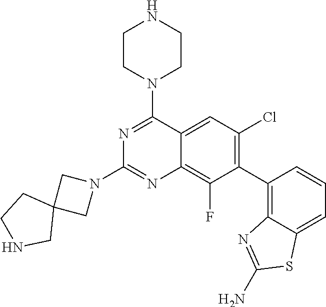 [SMILES: Nc1nc2c(-c3c(Cl)cc4c(N5CCNCC5)nc(N5CC6(CCNC6)C5)nc4c3F)cccc2s1] | 4-[6-chloro-2-(2,7-diazaspiro[3.4]octan- 2-yl)-8-fluoro-4-piperazin-1-yl- quinazolin-7-yl]-1,3-benzothiazol-2- amine | 525.2 | 1.55 |
| 121 | 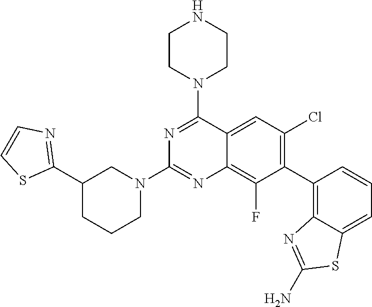 [SMILES: Nc1nc2c(-c3c(Cl)cc4c(N5CCNCC5)nc(N5CCCC(c6nccs6)C5)nc4c3F)cccc2s1] | 4-[6-chloro-8-fluoro-4-piperazin-1-yl-2- [3-thiazol-2-yl-1-piperidyl]quinazolin-7- yl]-1,3-benzothiazol-2-amine | 581.1 | 2.66 |

### 4-(4-((1R,5S)-3-Oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (Example 122

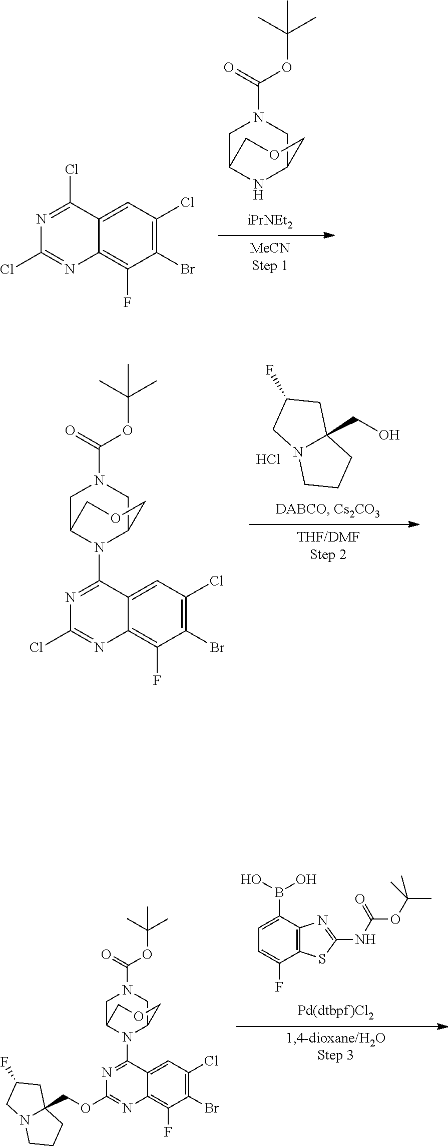

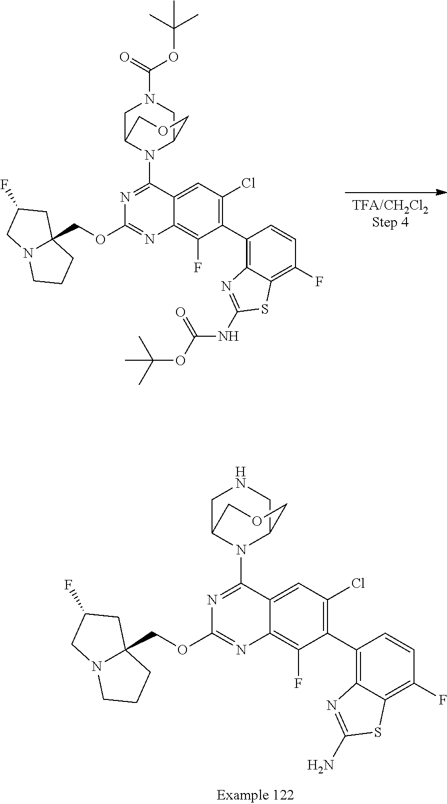 [SMILES: CC(C)(C)OC(=O)Nc1nc2c(-c3c(Cl)cc4c(N5C6COCC5CN(C(=O)OC(C)(C)C)C6)nc(OC[C@@]56CCCN5C[C@H](F)C6)nc4c3F)ccc(F)c2s1 ; "TFA/CH2Cl2Step 4" ; Nc1nc2c(-c3c(Cl)cc4c(N5C6CNCC5COC6)nc(OC[C@@]56CCCN5C[C@H](F)C6)nc4c3F)ccc(F)c2s1]

Step 1: tert-Butyl (1R,5S)-9-(7-bromo-2,6-dichloro-8-fluoroquinazolin-4-yl)-3-oxa-7,9-diazabicyclo[3.3.1]nonane-7-carboxylate. To a stirred solution of tert-butyl 3-oxa-7,9-diazabicyclo[3.3.1]nonane-7-carboxylate (0.28 g, 1.21 mmol, Aurum Pharmatech LLC) in acetonitrile (6 mL) was added 1,1′-dimethyltriethylamine (0.47 g, 0.63 mL, 3.63 mmol, Sigma-Aldrich Corporation) and 7-bromo-2,4,6-trichloro-8-fluoroquinazoline (0.40 g, 1.21 mmol, Advanced ChemBlocks Inc.). The reaction was stirred at room temperature for 30 min. The reaction mixture was diluted with water and extracted with DCM. The organic layer was dried with sodium sulfate and evaporated in vacuo to afford tert-butyl (1R,5S)-9-(7-bromo-2,6-dichloro-8-fluoroquinazolin-4-yl)-3-oxa-7,9-diazabicyclo[3.3.1]nonane-7-carboxylate (0.64 g, 1.23 mmol, 100% yield) as yellow solid, which was used directly for next step without further purification. m/z (ESI, +ve ion): 522.4 (M+H)⁺.

Step 2: tert-Butyl (1R,5S)-9-(7-bromo-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate. A mixture of tert-butyl (1R,5S)-9-(7-bromo-2,6-dichloro-8-fluoroquinazolin-4-yl)-3-oxa-7,9-diazabicyclo[3.3.1]nonane-7-carboxylate (71 mg, 0.14 mmol), ((2R,7aS)-2-fluorotetrahydro-1H-pyrrolizin-7a(5H)-yl)methanol hydrochloride (53 mg, 0.27 mmol, PharmaBlock), 1,4-diazabicyclo[2.2.2]octane (3.1 mg, 0.027 mmol, Sigma-Aldrich Corporation), and caesium carbonate (0.13 g, 0.41 mmol, Sigma-Aldrich Corporation) was stirred in N,N-dimethylformamide (0.4 mL) and tetrahydrofuran (2 mL) at room temperature for 3 h. The reaction mixture was diluted with water and extracted with DCM. The organic layer was concentrated under reduced pressure, and purified by column chromatography on silica gel eluting with a gradient of 0-50% (20% MeOH in DCM)/DCM, followed by reverse phase HPLC to afford tert-butyl (1R,5S)-9-(7-bromo-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate (38 mg, 0.059 mmol, 43% yield) as white solid. m/z (ESI, +ve ion): 644.0 (M+H)⁺.

Step 3: tert-Butyl (1R,5S)-9-(7-(2-((tert-butoxycarbonyl)amino)-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate. In an 8-mL vial, the mixture of tert-butyl (1R,5S)-9-(7-bromo-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate (38 mg, 0.059 mmol), [2-(tert-butoxycarbonylamino)-7-fluoro-1,3-benzothiazol-4-yl]boronic acid (37 mg, 0.12 mmol, Synnovator, Inc.), [1,1′-bis(di-tert-butylphosphino)ferrocene]dichloropalladium(II) (19 mg, 0.029 mmol, Sigma-Aldrich Corporation), and potassium phosphate tribasic (38 mg, 0.18 mmol, Acros Organics) in 1,4-dioxane (1 mL) and water (0.1 mL) was stirred at 90° C. for 2 h. The reaction mixture was purified by reverse phase HPLC to afford tert-butyl (1R,5S)-9-(7-(2-((tert-butoxycarbonyl)amino)-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate as white solid. m/z (ESI, +ve ion): 832.2 (M+H)⁺.

Step 4: 4-(4-((1R,5S)-3-Oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine. tert-Butyl (1R,5S)-9-(7-(2-((tert-butoxycarbonyl)amino)-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate was dissolved in 0.5 mL DCM. Trifluoroacetic acid (0.77 g, 0.5 mL, 6.71 mmol, Sigma-Aldrich Corporation) was added and the reaction mixture was stirred at room temperature for 1 h. The reaction mixture was concentrated under reduced pressure and purified by reverse phase HPLC to afford 4-(4-((1R,5S)-3-oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine (28 mg, 0.044 mmol, 75% yield) as white solid. m/z (ESI, +ve ion): 632.2 (M+H)⁺. ¹H NMR (400 MHz, METHANOL-d4) δ ppm 7.87 (d, J=1.5 Hz, 1H), 7.24 (ddd, J=8.4, 5.4, 1.5 Hz, 1H), 7.05 (dd, J=9.1, 8.5 Hz, 1H), 5.45-5.73 (m, 1H), 4.79-4.83 (m, 2H), 4.67-4.76 (m, 2H), 4.22-4.33 (m, 4H), 3.85-4.07 (m, 3H), 3.72-3.82 (m, 4H), 3.46-3.54 (m, 1H), 2.57-2.81 (m, 2H), 2.31-2.49 (m, 3H), 2.13-2.26 (m, 1H).

TABLE 5
Additional Examples. Prepared in an Analogous Manner to Example 122.

| | | | MS | |
|---|---|---|---|---|
| | | | m/z | |
| | | | (ESI, | |
| Ex. # | Structure | Name | +ve ion) | ¹H NMR |
| 123 | 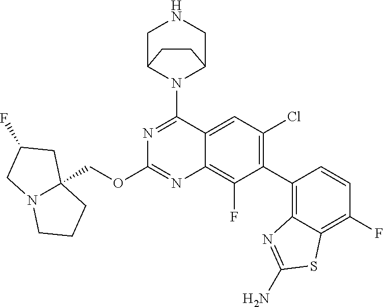 [SMILES: Nc1nc2c(-c3c(Cl)cc4c(N5C6CCC5CNC6)nc(OC[C@]56CCCN5C[C@H](F)C6)nc4c3F)ccc(F)c2s1] | 4-(4-((1R,5S)-3,8- diazabicyclo[3.2.1]octan- 8-yl)-6-chloro-8- fluoro-2-(((2R,7aR)-2- fluorotetrahydro-1H- pyrrolizin-7a(5H)- yl)methoxy)quinazolin- 7-yl)-7- fluorobenzo[d]thiazol- 2-amine | 616.2 (M + H)⁺ | ¹H NMR (400 MHz, METHANOL-d4) δ ppm 8.04 (d, J = 1.5 Hz, 1 H), 7.24-7.29 (m, 1 H), 7.05 (t, J = 8.8 Hz, 1 H), 5.42-5.64 (m, 1 H), 5.19 (br s, 2 H), 4.72-4.84 (m, 2 H), 4.01- 4.11 (m, 1 H), 3.54-3.75 (m, 4 H), 3.48 (br d, J = 13.0 Hz, 4 H), 2.67-2.79 (m, 1 H), 2.29-2.54 (m, 5 H), 2.12-2.22 (m, 3 H). |
| 124 | 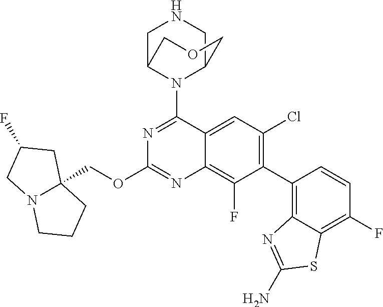 [SMILES: Nc1nc2c(-c3c(Cl)cc4c(N5C6CNCC5COC6)nc(OC[C@]56CCCN5C[C@H](F)C6)nc4c3F)ccc(F)c2s1] | 4-(4-((1R,5S)-3-oxa- 7,9- diazabicyclo[3.3.1]nonan- 9-yl)-6-chloro-8- fluoro-2-(((2R,7aR)-2- fluorotetrahydro-1H- pyrrolizin-7a(5H)- yl)methoxy)quinazolin- 7-yl)-7- fluorobenzo[d]thiazol- 2-amine | 632.0 (M + H)⁺ | ¹H NMR (400 MHz, METHANOL-d4) δ ppm 8.01 (t, J = 1.7 Hz, 1 H), 7.84 (dt, J = 7.7, 1.4 Hz, 1 H), 7.30-7.38 (m, 2 H), 5.39 (dd, J = 6.8, 3.9 Hz, 1 H), 4.13 (br t, J = 4.5 Hz, 4 H), 3.43-3.53 (m, 4 H), 2.77 (d, J = 2.3 Hz, 3 H), 1.63 (dd, J = 6.9, 0.8 Hz, 3 H). |
| 125 | 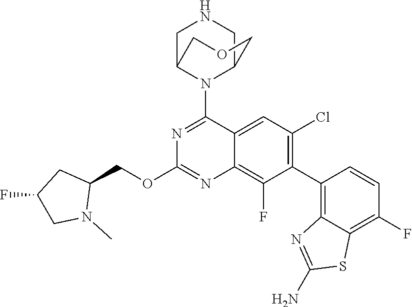 [SMILES: CN1C[C@H](F)C[C@H]1COc1nc(N2C3CNCC2COC3)c2cc(Cl)c(-c3ccc(F)c4sc(N)nc34)c(F)c2n1] | 4-(4-((1R,5S)-3-oxa- 7,9- diazabicyclo[3.3.1]nonan- 9-yl)-6-chloro-8- fluoro-2-(((2S,4R)-4- fluoro-1- methylpyrrolidin-2- yl)methoxy)quinazolin- 7-yl)-7- fluorobenzo[d]thiazol- 2-amine | 606.2 (M + H)⁺ | ¹H NMR (400 MHz, METHANOL-d4) δ ppm 7.87 (t, J = 1.6 Hz, 1 H), 7.25 (ddd, J = 8.5, 5.4, 3.0 Hz, 1 H), 7.05 (ddd, J = 9.1, 8.5, 1.7 Hz, 1 H), 5.36-5.61 (m, 1 H), 4.96-5.06 (m, 1 H), 4.73-4.81 (m, 3 H), 4.21- 4.33 (m, 5 H), 4.02-4.17 (m, 1 H), 3.64-3.84 (m, 5 H), 3.14-3.31 (m, 3 H), 2.63-2.75 (m, 1 H), 2.31- 2.53 (m, 1 H). |
| 126 | 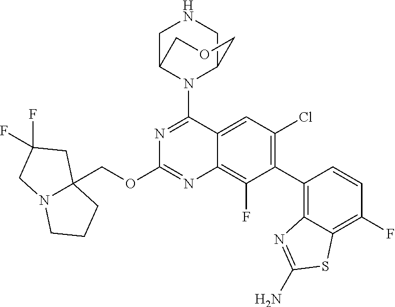 [SMILES: Nc1nc2c(-c3c(Cl)cc4c(N5C6CNCC5COC6)nc(OCC56CCCN5CC(F)(F)C6)nc4c3F)ccc(F)c2s1] | 4-(4-((1R,5S)-3-oxa- 7,9- diazabicyclo[3.3.1]nonan- 9-yl)-6-chloro-2- ((2,2- difluorotetrahydro-1H- pyrrolizin-7a(5H)- yl)methoxy)-8- fluoroquinazolin-7-yl)- 7- fluorobenzo[d]thiazol- 2-amine | 650.0 (M + H)⁺ | ¹H NMR (400 MHz, METHANOL-d4) δ ppm 7.87 (s, 1 H), 7.26 (ddd, J = 8.5, 5.4, 1.1 Hz, 1 H), 7.06 (t, J = 8.8 Hz, 1 H), 4.74- 4.84 (m, 4 H), 4.21-4.35 (m, 5 H), 3.75-4.01 (m, 6 H), 3.45-3.55 (m, 1 H), 2.80-3.06 (m, 2 H), 2.23- 2.51 (m, 4 H). |
| 127 | 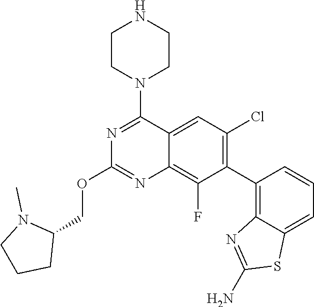 [SMILES: CN1CCC[C@H]1COc1nc(N2CCNCC2)c2cc(Cl)c(-c3cccc4sc(N)nc34)c(F)c2n1] | 4-(6-chloro-8-fluoro-2- (((S)-1- methylpyrrolidin-2- yl)methoxy)-4- (piperazin-1- yl)quinazolin-7- yl)benzo[d]thiazol-2- amine | 527.8 (M + H)⁺ | ¹H NMR (400 MHz, METHANOL-d4) δ ppm 8.05 (s, 1 H), 7.91 (dt, J = 7.7, 1.8 Hz, 1 H), 7.38- 7.48 (m, 2 H), 4.93 (td, J = 13.5, 2.7 Hz, 1 H), 4.71 (dt, J = 12.9, 6.4 Hz, 1 H), 4.15 (br dd, J = 6.3, 3.3 Hz, 4 H), 3.85-3.98 (m, 1 H), 3.76 (br dd, J = 10.9, 5.4 Hz, 1 H), 3.35-3.56 (m, 4 H), 3.20-3.31 (m, 1 H), 3.06- 3.16 (m, 3 H), 2.35-2.48 (m, 1 H), 2.04-2.28 (m, 3 H). |
| 128 | 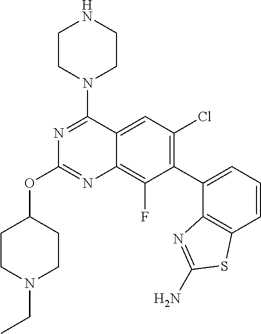 [SMILES: CCN1CCC(Oc2nc(N3CCNCC3)c3cc(Cl)c(-c4cccc5sc(N)nc45)c(F)c3n2)CC1] | 4-(6-chloro-2-((1- ethylpiperidin-4- yl)oxy)-8-fluoro-4- (piperazin-1- yl)quinazolin-7- yl)benzo[d]thiazol-2- amine | 542.0 (M + H)⁺ | ¹H NMR (400 MHz, METHANOL-d4) δ ppm 7.99-8.03 (m, 1 H), 7.88 (dd, J = 7.6, 1.4 Hz, 1 H), 7.35-7.44 (m, 2 H), 5.59 (br s, 1 H), 4.05-4.17 (m, 4 H), 3.72 (br d, J = 12.8 Hz, 1 H), 3.49-3.59 (m, 5 H), 3.15-3.31 (m, 4 H), 2.51- 2.61 (m, 1 H), 2.35-2.47 (m, 1 H), 2.14-2.27 (m, 1 H), 1.98-2.08 (m, 1 H), 1.36-1.44 (m, 3 H). |
| 129 | 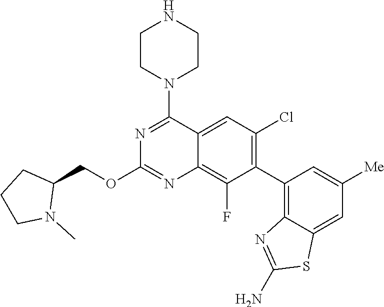 [SMILES: Cc1cc(-c2c(Cl)cc3c(N4CCNCC4)nc(OC[C@@H]4CCCN4C)nc3c2F)c2nc(N)sc2c1] | 4-(6-chloro-8-fluoro-2- (((S)-1- methylpyrrolidin-2- yl)methoxy)-4- (piperazin-1- yl)quinazolin-7-yl)-6- methylbenzo[d]thiazol- 2-amine | 542.2 (M + H)⁺ | ¹H NMR (400 MHz, METHANOL-d4) δ ppm 7.99-8.20 (m, 1 H), 7.54- 7.78 (m, 1 H), 7.25-7.37 (m, 1 H), 4.91-5.02 (m, 1 H), 4.63-4.78 (m, 1 H), 4.07-4.25 (m, 4 H), 3.86- 4.02 (m, 1 H), 3.68-3.83 (m, 1 H), 3.51 (br s, 4 H), 3.20-3.30 (m, 1 H), 3.04- 3.18 (m, 3 H), 2.52 (s, 4 H), 2.02-2.32 (m, 3 H). |
| 130 | 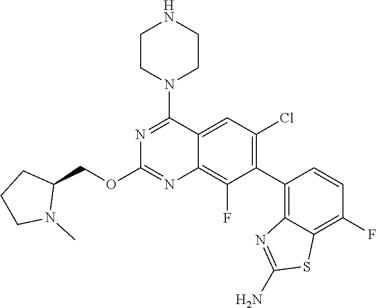 [SMILES: CN1CCC[C@H]1COc1nc(N2CCNCC2)c2cc(Cl)c(-c3ccc(F)c4sc(N)nc34)c(F)c2n1] | 4-(6-chloro-8-fluoro-2- (((S)-1- methylpyrrolidin-2- yl)methoxy)-4- (piperazin-1- yl)quinazolin-7-yl)-7- fluorobenzo[d]thiazol- 2-amine | 546.2 (M + H)⁺ | ¹H NMR (400 MHz, DMSO-d6) δ ppm 9.10 (s, 1 H), 7.98-8.08 (m, 1 H), 7.81-7.95 (m, 2 H), 7.18- 7.28 (m, 1 H), 7.02-7.14 (m, 1 H), 4.56-4.82 (m, 2 H), 3.92-4.06 (m, 4 H), 3.79-3.90 (m, 2 H), 3.30- 3.42 (m, 4 H), 3.09-3.22 (m, 1 H), 2.93-3.01 (m, 3 H), 2.21-2.32 (m, 1 H), 2.02-2.15 (m, 1 H), 1.81- 2.01 (m, 2 H). |
| 131 | 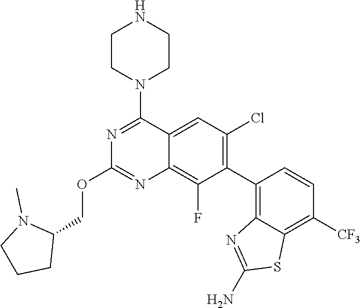 [SMILES: CN1CCC[C@H]1COc1nc(N2CCNCC2)c2cc(Cl)c(-c3ccc(C(F)(F)F)c4sc(N)nc34)c(F)c2n1] | 4-(6-chloro-8-fluoro-2- (((S)-1- methylpyrrolidin-2- yl)methoxy)-4- (piperazin-1- yl)quinazolin-7-yl)-7- (trifluoromethyl)benzo [d]thiazol-2-amine | 596.150 (M + H)⁺ | ¹H NMR (400 MHz, METHANOL-d4) δ ppm 8.00-8.08 (m, 1 H), 7.52- 7.59 (m, 1 H), 7.34- 7.46 (m, 1 H), 4.95 (br d, J = 3.3 Hz, 1 H), 4.63-4.75 (m, 1 H), 4.14 (br s, 4 H), 3.89-3.97 (m, 1 H), 3.71-3.80 (m, 1 H), 3.52 (br s, 4 H), 3.24-3.31 (m, 1 H), 3.12 (s, 3 H), 2.36- 2.51 (m, 1 H), 2.19-2.31 (m, 1 H), 2.05- 2.17 (m, 2 H) |
| 132 | 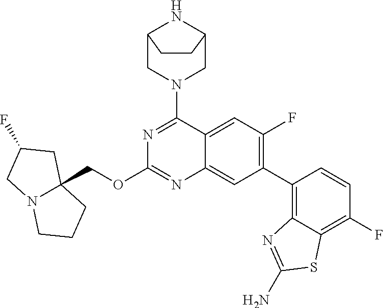 [SMILES: Nc1nc2c(-c3cc4nc(OC[C@@]56CCCN5C[C@H](F)C6)nc(N5CC6CCC(C5)N6)c4cc3F)ccc(F)c2s1] | 4-(4-((1R,5S)-3,8- diazabicyclo[3.2.1]octan- 3-yl)-6-fluoro-2- (((2R,7aS)-2- fluorotetrahydro-1H- pyrrolizin-7a(5H)- yl)methoxy)quinazolin- 7-yl)-7- fluorobenzo[d]thiazol- 2-amine | 582.20 (M + H)⁺ | ¹H NMR (400 MHz, METHANOL-d4) δ ppm 7.92 (d, J = 6.9 Hz, 1 H), 7.84 (d, J = 10.7 Hz, 1 H), 7.41 (ddd, J = 8.5, 5.5, 1.3 Hz, 1 H), 7.03 (t, J = 8.9 Hz, 1 H), 5.47-5.74 (m, 1 H), 4.69-4.80 (m, 4 H), 4.28 (br s, 2 H), 3.87-4.05 (m, 5 H), 3.47-3.55 (m, 1 H), 2.54-2.85 (m, 2 H), 2.33- 2.51 (m, 3 H), 2.10-2.27 (m, 5 H). |
| 133 | 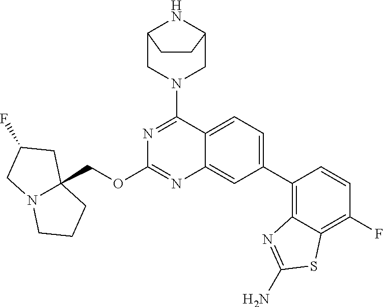 [SMILES: Nc1nc2c(-c3ccc4c(N5CC6CCC(C5)N6)nc(OC[C@@]56CCCN5C[C@H](F)C6)nc4c3)ccc(F)c2s1] | 4-(4-((1R,5S)-3,8- diazabicyclo[3.2.1]octan- 3-yl)-2-(((2R,7aS)-2- fluorotetrahydro-1H- pyrrolizin-7a(5H)- yl)methoxy)quinazolin- 7-yl)-7- fluorobenzo[d]thiazol- 2-amine | 564.40 (M + H)⁺ | ¹H NMR (400 MHz, METHANOL-d4) δ ppm 8.20 (d, J = 1.7 Hz, 1 H), 8.16 (d, J = 8.8 Hz, 1 H), 7.99 (dd, J = 8.9, 1.8 Hz, 1 H), 7.55 (dd, J = 8.6, 5.6 Hz, 1 H), 7.03 (t, J = 8.8 Hz, 1 H), 5.46-5.80 (m, 1 H), 4.87-4.95 (m, 2 H), 4.79- 4.82 (m, 2 H), 4.30 (br s, 2 H), 3.91-4.06 (m, 5 H), 3.48-3.56 (m, 1 H), 2.60- 2.84 (m, 2 H), 2.35-2.53 (m, 3 H), 2.22-2.33 (m, 1 H), 2.09-2.22 (m, 4 H). |
| 134 | 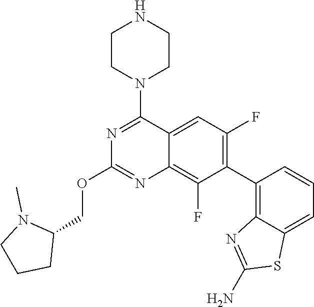 [SMILES: CN1CCC[C@H]1COc1nc(N2CCNCC2)c2cc(F)c(-c3cccc4sc(N)nc34)c(F)c2n1] | 4-(6,8-difluoro-2-(((S)- 1-methylpyrrolidin-2- yl)methoxy)-4- (piperazin-1- yl)quinazolin-7- yl)benzo[d]thiazol-2- amine | 512.2 (M + H)⁺ | ¹H NMR (400 MHz, METHANOL-d4) δ ppm 7.77-7.94 (m, 1 H), 7.69- 7.77 (m, 1 H), 7.38-7.43 (m, 1 H), 7.31-7.37 (m, 1 H), 4.89-5.02 (m, 1 H), 4.64-4.77 (m, 1 H), 4.10 (br dd, J = 6.5, 3.6 Hz, 4 H), 3.86-3.98 (m, 1 H), 3.71- 3.83 (m, 1 H), 3.45-3.59 (m, 4 H), 3.20-3.31 (m, 1 H), 3.13 (d, J = 1.5 Hz, 3 H), 2.38-2.51 (m, 1 H), 2.18- 2.31 (m, 1 H), 2.11 (br dd, J = 7.5, 5.2 Hz, 2 H). |
| 135 | 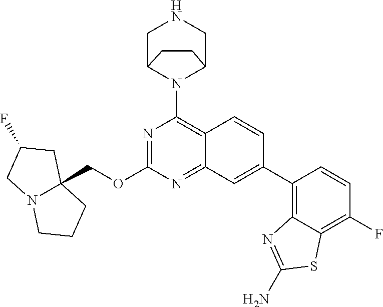 [SMILES: Nc1nc2c(-c3ccc4c(N5C6CCC5CNC6)nc(OC[C@@]56CCCN5C[C@H](F)C6)nc4c3)ccc(F)c2s1] | 4-(4-((1R,5S)-3,8- diazabicyclo[3.2.1]octan- 8-yl)-2-(((2R,7aS)-2- fluorotetrahydro-1H- pyrrolizin-7a(5H)- yl)methoxy)quinazolin- 7-yl)-7- fluorobenzo[d]thiazol- 2-amine | 564.25 (M + H)⁺ | ¹H NMR (400 MHz, METHANOL-d4) δ ppm 8.23 (d, J = 8.8 Hz, 1 H), 8.18 (d, J = 1.7 Hz, 1 H), 8.00 (dd, J = 8.9, 1.8 Hz, 1 H), 7.54 (dd, J = 8.6, 5.4 Hz, 1 H), 7.04 (t, J = 8.8 Hz, 1 H), 5.50-5.76 (m, 1 H), 5.43 (br s, 2 H), 4.77-4.84 (m, 2 H), 3.91-4.05 (m, 3 H), 3.73 (br d, J = 11.7 Hz, 2 H), 3.48-3.56 (m, 3 H), 2.61-2.80 (m, 2 H), 2.32- 2.52 (m, 5 H), 2.15-2.32 (m, 3 H). |
| 136 | 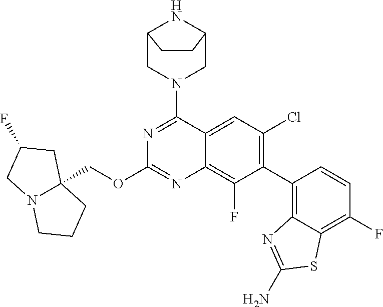 [SMILES: Nc1nc2c(-c3c(Cl)cc4c(N5CC6CCC(C5)N6)nc(OC[C@]56CCCN5C[C@H](F)C6)nc4c3F)ccc(F)c2s1] | 4-(4-((1R,5S)-3,8- diazabicyclo[3.2.1]octan- 3-yl)-6-chloro-8- fluoro-2-(((2R,7aR)-2- fluorotetrahydro-1H- pyrrolizin-7a(5H)- yl)methoxy)quinazolin- 7-yl)-7- fluorobenzo[d]thiazol- 2-amine | 616.2 (M + H)⁺ | ¹H NMR (400 MHz, METHANOL-d4) δ ppm 7.97 (d, J = 1.7 Hz, 1 H), 7.26 (ddd, J = 8.5, 5.4, 2.2 Hz, 1 H), 7.05 (t, J = 8.9 Hz, 1 H), 5.39-5.69 (m, 1 H), 4.69-4.82 (m, 4 H), 4.27 (br s, 2 H), 4.01-4.11 (m, 1 H), 3.91 (dt, J = 14.4, 7.2 Hz, 2 H), 3.63-3.73 (m, 1 H), 3.44-3.60 (m, 2 H), 2.67- 2.79 (m, 1 H), 2.39-2.56 (m, 2 H), 2.25-2.37 (m, 2 H), 2.05-2.21 (m, 5 H). |
| 137 | 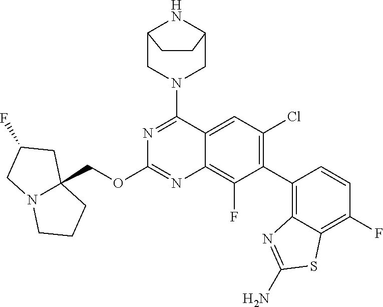 [SMILES: Nc1nc2c(-c3c(Cl)cc4c(N5CC6CCC(C5)N6)nc(OC[C@@]56CCCN5C[C@H](F)C6)nc4c3F)ccc(F)c2s1] | 4-(4-((1R,5S)-3,8- diazabicyclo[3.2.1]octan- 3-yl)-6-chloro-8- fluoro-2-(((2R,7aS)-2- fluorotetrahydro-1H- pyrrolizin-7a(5H)- yl)methoxy)quinazolin- 7-yl)-7- fluorobenzo[d]thiazol- 2-amine | 616.2 (M + H)⁺ | ¹H NMR (400 MHz, METHANOL-d4) δ ppm 7.98 (d, J = 1.7 Hz, 1 H), 7.25 (ddd, J = 8.4, 5.4, 1.3 Hz, 1 H), 7.05 (t, J = 8.8 Hz, 1 H), 5.41-5.78 (m, 1 H), 4.66-4.81 (m, 4 H), 4.27 (br s, 2 H), 3.85-4.07 (m, 5 H), 3.45-3.54 (m, 1 H), 2.56-2.77 (m, 2 H), 2.32- 2.49 (m, 3 H), 2.09-2.23 (m, 5 H). |
| 138 | 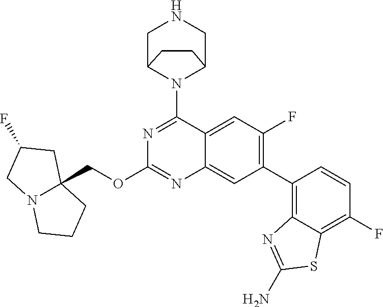 [SMILES: Nc1nc2c(-c3cc4nc(OC[C@@]56CCCN5C[C@H](F)C6)nc(N5C6CCC5CNC6)c4cc3F)ccc(F)c2s1] | 4-(4-((1R,5S)-3,8- diazabicyclo[3.2.1]octan- 8-yl)-6-fluoro-2- (((2R,7aS)-2- fluorotetrahydro-1H- pyrrolizin-7a(5H)- yl)methoxy)quinazolin- 7-yl)-7- fluorobenzo[d]thiazol- 2-amine | 582.2 (M + H)⁺ | ¹H NMR (400 MHz, METHANOL-d4) δ ppm 7.90-7.98 (m, 2 H), 7.43 (ddd, J = 8.5, 5.5, 1.3 Hz, 1 H), 7.04 (t, J = 8.8 Hz, 1 H), 5.44-5.78 (m, 1 H), 5.34 (br s, 2 H), 4.74-4.80 (m, 2 H), 3.87-4.04 (m, 3 H), 3.72 (br d, J = 11.5 Hz, 2 H), 3.35-3.56 (m, 3 H), 2.57- 2.82 (m, 2 H), 2.33-2.51 (m, 5 H), 2.14-2.32 (m, 3 H). |
| 139 | 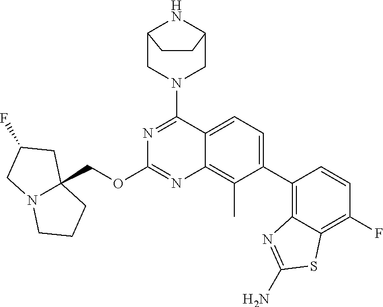 [SMILES: Cc1c(-c2ccc(F)c3sc(N)nc23)ccc2c(N3CC4CCC(C3)N4)nc(OC[C@@]34CCCN3C[C@H](F)C4)nc12] | 4-(4-((1R,5S)-3,8- diazabicyclo[3.2.1]octan- 3-yl)-2-(((2R,7aS)-2- fluorotetrahydro-1H- pyrrolizin-7a(5H)- yl)methoxy)-8- methylquinazolin-7-yl)- 7- fluorobenzo[d]thiazol- 2-amine | 578.2 (M + H)⁺ | ¹H NMR (400 MHz, METHANOL-d4) δ ppm 7.90 (d, J = 8.8 Hz, 1 H), 7.37 (d, J = 8.6 Hz, 1 H), 7.20 (dd, J = 8.4, 5.4 Hz, 1 H), 7.03 (dd, J = 9.1, 8.5 Hz, 1 H), 0.00 (ddt, J = 52.7, 51.8, 4.0, 4.0 Hz, 1 H), 4.70- 4.76 (m, 2 H), 4.60-4.67 (m, 2 H), 4.25 (br s, 2 H), 3.91-4.09 (m, 3 H), 3.81 (br d, J = 14.6 Hz, 2 H), 3.47- 3.56 (m, 1 H), 2.59-2.85 (m, 2 H), 2.42 (s, 12 H). |
| 140 | 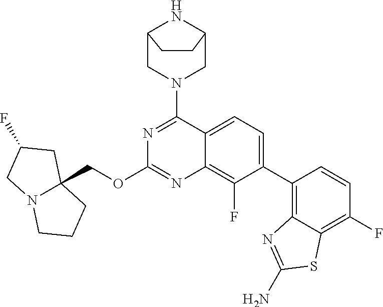 [SMILES: Nc1nc2c(-c3ccc4c(N5CC6CCC(C5)N6)nc(OC[C@@]56CCCN5C[C@H](F)C6)nc4c3F)ccc(F)c2s1] | 4-(4-((1R,5S)-3,8- diazabicyclo[3.2.1]octan- 3-yl)-8-fluoro-2- (((2R,7aS)-2- fluorotetrahydro-1H- pyrrolizin-7a(5H)- yl)methoxy)quinazolin- 7-yl)-7- fluorobenzo[d]thiazol- 2-amine | 582.2 (M + H)⁺ | ¹H NMR (400 MHz, METHANOL-d4) δ ppm 7.95 (d, J = 8.8 Hz, 1 H), 7.61-7.68 (m, 1 H), 7.43- 7.48 (m, 1 H), 7.09 (s, 1 H), 5.55-5.73 (m, 1 H), 4.78 (d, J = 2.9 Hz, 4 H), 4.32 (br s, 2 H), 3.88-4.18 (m, 5 H), 3.50-3.63 (m, 1 H), 2.64- 2.86 (m, 2 H), 2.34-2.58 (m, 3 H), 2.15-2.30 (m, 5 H). |
| 141 | 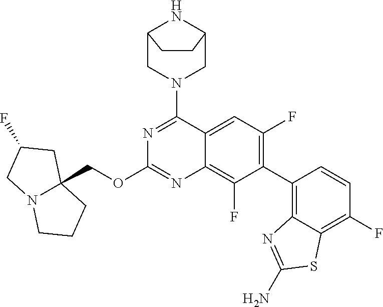 [SMILES: Nc1nc2c(-c3c(F)cc4c(N5CC6CCC(C5)N6)nc(OC[C@@]56CCCN5C[C@H](F)C6)nc4c3F)ccc(F)c2s1] | 4-(4-((1R,5S)-3,8- diazabicyclo [3.2.1]octan- 3-yl)-6,8-difluoro-2- (((2R,7aS)-2- fluorotetrahydro-1H- pyrrolizin-7a(5H)- yl)methoxy)quinazolin- 7-yl)-7- fluorobenzo[d]thiazol- 2-amine | 600.1 (M + H)⁺ | ¹H NMR (400 MHz, METHANOL-d4) δ ppm 7.67 (d, J = 9.95 Hz, 1 H) 7.30-7.42 (m, 1 H) 7.06 (t, J = 8.81 Hz, 1 H) 5.45-5.73 (m, 1 H) 4.63-4.80 (m, 4 H) 4.27 (br s, 2 H) 3.82- 4.12 (m, 5 H) 3.45-3.60 (m, 1 H) 2.55-2.79 (m, 2 H) 2.31-2.53 (m, 3 H) 2.17 (br s, 5 H). |
| 142 | 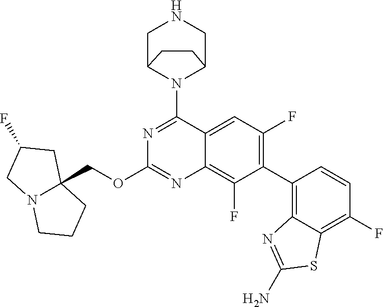 [SMILES: Nc1nc2c(-c3c(F)cc4c(N5C6CCC5CNC6)nc(OC[C@@]56CCCN5C[C@H](F)C6)nc4c3F)ccc(F)c2s1] | 4-(4-((1R,5S)-3,8- diazabicyclo[3.2.1]octan- 8-yl)-6,8-difluoro-2- (((2R,7aS)-2- fluorotetrahydro-1H- pyrrolizin-7a(5H)- yl)methoxy)quinazolin- 7-yl)-7- fluorobenzo[d]thiazol- 2-amine | 600.1 (M + H)⁺ | ¹H NMR (400 MHz, METHANOL-d4) ä ppm 7.70-7.76 (m, 1 H), 7.30- 7.37 (m, 1 H), 7.04 (s, 1 H), 5.44-5.70 (m, 1 H), 5.12-5.24 (m, 2 H), 4.69 (s, 2 H), 3.80-4.09 (m, 3 H), 3.61-3.76 (m, 2 H), 3.46 (br d, J = 1.0 Hz, 3 H), 2.53- 2.80 (m, 2 H), 2.27-2.49 (m, 5 H), 2.16 (s, 3 H). |
| 143 | 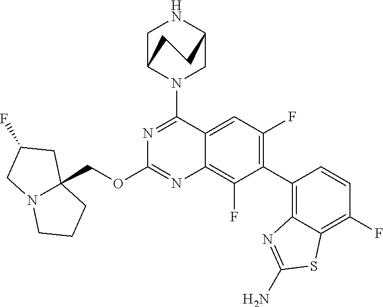 [SMILES: Nc1nc2c(-c3c(F)cc4c(N5C[C@@H]6CC[C@H]5CN6)nc(OC[C@@]56CCCN5C[C@H](F)C6)nc4c3F)ccc(F)c2s1] | 4-(4-((1S,4S)-2,5- diazabicyclo[2.2.2]octan- 2-yl)-6,8-difluoro-2- (((2R,7aS)-2- fluorotetrahydro-1H- pyrrolizin-7a(5H)- yl)methoxy)quinazolin- 7-yl)-7- fluorobenzo[d]thiazol- 2-amine | 600.1 (M + H)⁺ | ¹H NMR (400 MHz, METHANOL-d4) ä ppm 7.75-7.83 (m, 1 H), 7.28- 7.36 (m, 1 H), 6.98-7.08 (m, 1 H), 5.40-5.67 (m, 1 H), 5.07-5.19 (m, 1 H), 4.68 (s, 2 H), 4.46-4.57 (m, 1 H), 4.31-4.44 (m, 1 H), 3.76-4.10 (m, 5 H), 3.54- 3.62 (m, 1 H), 3.40-3.52 (m, 1 H), 2.04-2.77 (m, 10 H). |

### 4-(6-Chloro-8-fluoro-2-(((S)-1-methylpyrrolidin-2-yl)methoxy)-4-(1,2,3,6-tetrahydropyridin-4-yl)quinazolin-7-yl)benzo[d]thiazol-2-amine (Example 144)

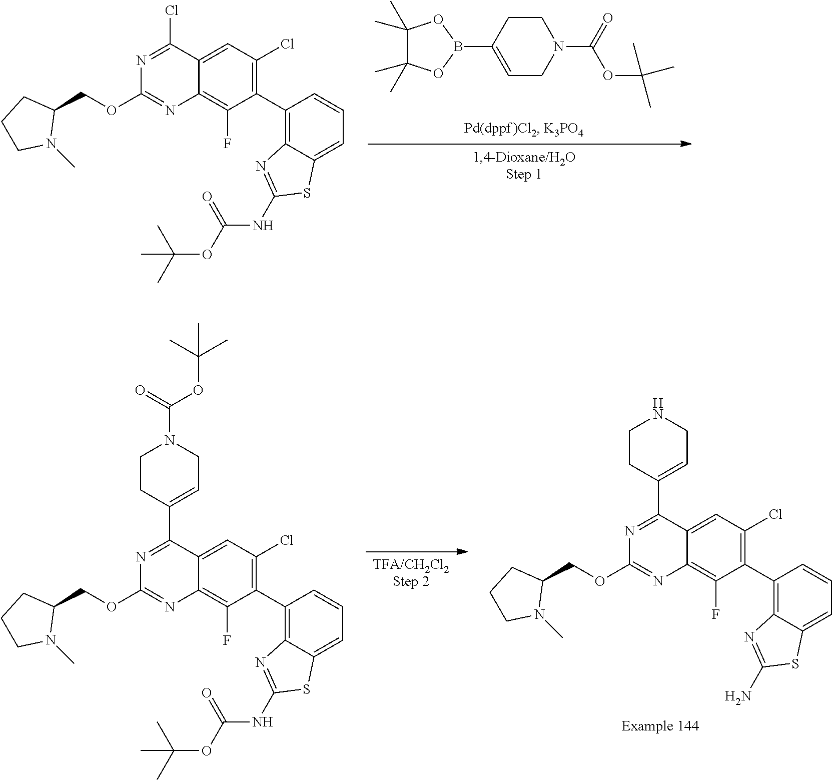

Step 1: tert-Butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)-3,6-dihydropyridine-1(2H)-carboxylate. An 8-mL vial was charged with [1,1′-bis(diphenylphosphino)ferrocene]dichloropalladium(II) (25 mg, 0.035 mmol, Sigma-Aldrich Corporation), (1-tert-butoxycarbonyl-1,2,3,6-tetrahydropyridin-4-yl)boronic acid pinacol ester (80 mg, 0.26 mmol, Combi-Blocks Inc.), potassium phosphate tribasic (0.11 g, 0.52 mmol, Acros Organics), tert-butyl (4-(4,6-dichloro-8-fluoro-2-(((S)-1-methylpyrrolidin-2-yl)methoxy)quinazolin-7-yl)benzo[d]thiazol-2-yl)carbamate (0.10 g, 0.17 mmol), 1,4-dioxane (1.4 mL) and water (0.35 mL). The reaction was stirred at 90° C. for 1 h. The crude mixture was purified by column chromatography on silica gel, eluting with 0-50% EtOAc/ethanol in heptane with 2% triethylamine additive to yield tert-butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)-3,6-dihydropyridine-1(2H)-carboxylate (0.10 g, 0.14 mmol, 80% yield). m/z (ESI, +ve ion): 725.3 (M+H)⁺.

Step 2: 4-(6-Chloro-8-fluoro-2-(((S)-1-methylpyrrolidin-2-yl)methoxy)-4-(1,2,3,6-tetrahydropyridin-4-yl)quinazolin-7-yl)benzo[d]thiazol-2-amine. tert-Butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)-3,6-dihydropyridine-1(2H)-carboxylate (27 mg, 0.037 mmol) was stirred in trifluoroacetic acid (93 μL) and DCM (93 μL) at room temperature for 1 h. Solvents were removed under reduced pressure. The crude product was purified by reverse phase HPLC to yield 4-(6-chloro-8-fluoro-2-(((S)-1-methylpyrrolidin-2-yl)methoxy)-4-(1,2,3,6-tetrahydropyridin-4-yl)quinazolin-7-yl)benzo[d]thiazol-2-amine (14 mg, 0.026 mmol, 70% yield). m/z (ESI, +ve ion): 525.2 (M+H)⁺. ¹H NMR (400 MHz, METHANOL-d4) δ ppm 8.25-8.31 (m, 1H), 7.83-7.90 (m, 1H), 7.32-7.43 (m, 2H), 6.40-6.49 (m, 1H), 4.96-5.05 (m, 1H), 4.74-4.82 (m, 1H), 4.05-4.14 (m, 2H), 3.91-4.03 (m, 1H), 3.71-3.84 (m, 1H), 3.57-3.68 (m, 2H), 3.24-3.30 (m, 1H), 3.11-3.18 (m, 3H), 2.97-3.08 (m, 2H), 2.41-2.53 (m, 1H), 2.06-2.32 (m, 3H).

TABLE 6
Additional Examples. Prepared in an Analogous Manner to Example 144.

| | | | MS | |
|---|---|---|---|---|
| | | | m/z (ESI, | |
| Ex. # | Structure | Name | +ve ion) | ¹H NMR |
| 145 | 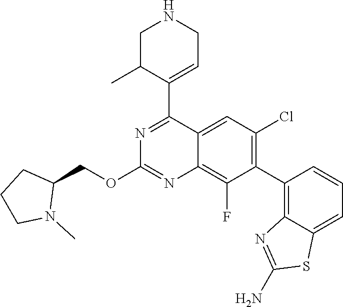 [SMILES: CC1CNCC=C1c1nc(OC[C@@H]2CCCN2C)nc2c(F)c(-c3cccc4sc(N)nc34)c(Cl)cc12] | 4-(6-chloro-8-fluoro- 4-(3-methyl-1,2,3,6- tetrahydropyridin-4- yl)-2-(((S)-1- methylpyrrolidin-2- yl)methoxy)quinazolin- 7-yl)benzo[d]thiazol- 2-amine Diastereoisomer A | 539.2 (M + H)⁺ | ¹H NMR (400 MHz, METHANOL-d4) δ ppm 8.21- 8.28 (m, 1 H), 7.81-7.87 (m, 1 H), 7.28-7.38 (m, 2 H), 6.26- 6.35 (m, 1 H), 4.95-5.07 (m, 1 H), 4.70-4.82 (m, 1 H), 4.08- 4.18 (m, 1 H), 3.92-4.04 (m, 2 H), 3.74-3.85 (m, 1 H), 3.66- 3.73 (m, 1 H), 3.47-3.62 (m, 1 H), 3.35-3.41 (m, 1 H), 3.24- 3.30 (m, 1 H), 3.11-3.19 (m, 3 H), 2.40-2.58 (m, 1 H), 2.05- 2.33 (m, 3 H), 1.06-1.17 (m, 3 H) |
| 146 | 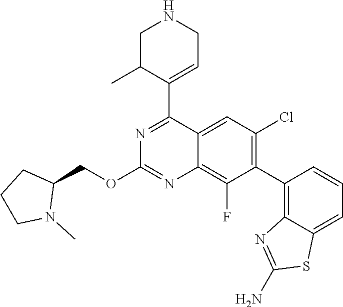 [SMILES: CC1CNCC=C1c1nc(OC[C@@H]2CCCN2C)nc2c(F)c(-c3cccc4sc(N)nc34)c(Cl)cc12] | 4-(6-chloro-8-fluoro- 4-(3-methyl-1,2,3,6- tetrahydropyridin-4- yl)-2-(((S)-1- methylpyrrolidin-2- yl)methoxy)quinazolin- 7-yl)benzo[d]thiazol- 2-amine Diastereoisomer B | 539.2 (M + H)⁺ | ¹H NMR (400 MHz, METHANOL-d4) δ ppm 8.23- 8.27 (m, 1 H), 7.81-7.87 (m, 1 H), 7.33 (s, 2 H), 6.27-6.36 (m, 1 H), 4.96-5.07 (m, 1 H), 4.72- 4.82 (m, 1 H), 4.08-4.18 (m, 1 H), 3.91-4.05 (m, 2 H), 3.65- 3.83 (m, 2 H), 3.49-3.62 (m, 1 H), 3.36-3.40 (m, 1 H), 3.25- 3.30 (m, 1 H), 3.12-3.20 (m, 3 H), 2.41-2.53 (m, 1 H), 2.05- 2.33 (m, 3 H), 1.05-1.14 (m, 3 H). |
| 147 | 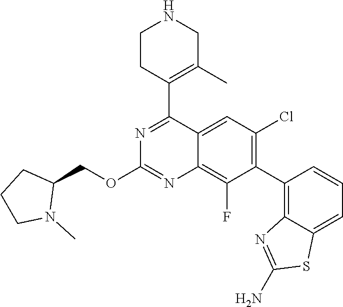 [SMILES: CC1=C(c2nc(OC[C@@H]3CCCN3C)nc3c(F)c(-c4cccc5sc(N)nc45)c(Cl)cc23)CCNC1] | 4-(6-chloro-8-fluoro- 4-(5-methyl-1,2,3,6- tetrahydropyridin-4- yl)-2-(((S)-1- methylpyrrolidin-2- yl)methoxy)quinazolin- 7-yl)benzo[d]thiazol- 2-amine | 539.0 (M + H)⁺ | ¹H NMR (400 MHz, METHANOL-d4) δ ppm 8.05 (d, J = 1.3 Hz, 1 H), 7.81-7.86 (m, 1 H), 7.28-7.37 (m, 2 H), 4.97-5.07 (m, 1 H), 4.72-4.82 (m, 1 H), 3.96-4.05 (m, 1 H), 3.93 (br s, 2 H), 3.71-3.85 (m, 1 H), 3.54-3.66 (m, 2 H), 3.24- 3.30 (m, 1 H), 3.12-3.20 (m, 3 H), 2.73-2.85 (m, 2 H), 2.42- 2.55 (m, 1 H), 2.06-2.32 (m, 3 H), 1.60-1.67 (m, 3 H). |
| 148 | 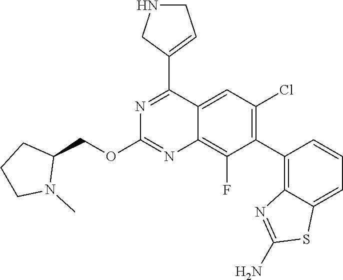 [SMILES: CN1CCC[C@H]1COc1nc(C2=CCNC2)c2cc(Cl)c(-c3cccc4sc(N)nc34)c(F)c2n1] | 4-(6-chloro-4-(2,5- dihydro-1H-pyrrol-3- yl)-8-fluoro-2-(((S)-1- methylpyrrolidin-2- yl)methoxy)quinazolin- 7-yl)benzo[d]thiazol- 2-amine | 512.0 (M + H)⁺ | ¹H NMR (400 MHz, METHANOL-d4) δ ppm 8.34- 8.43 (m, 1 H), 7.80-7.90 (m, 1 H), 7.29-7.40 (m, 2 H), 7.01- 7.10 (m, 1 H), 4.94-5.06 (m, 1 H), 4.72-4.82 (m, 3 H), 4.50- 4.62 (m, 2 H), 3.91-4.04 (m, 1 H), 3.73-3.85 (m, 1 H), 3.23- 3.30 (m, 1 H), 3.09-3.17 (m, 3 H), 2.40-2.53 (m, 1 H), 2.05- 2.32 (m, 3 H). |

### 4-(8-Fluoro-2-(((S)-1-methylpyrrolidin-2-yl)methoxy) 4-(piperazin-1-yl)-&-vinylquinazolin-7-yl)benzo[d]thiazol-2-amine (Example 149

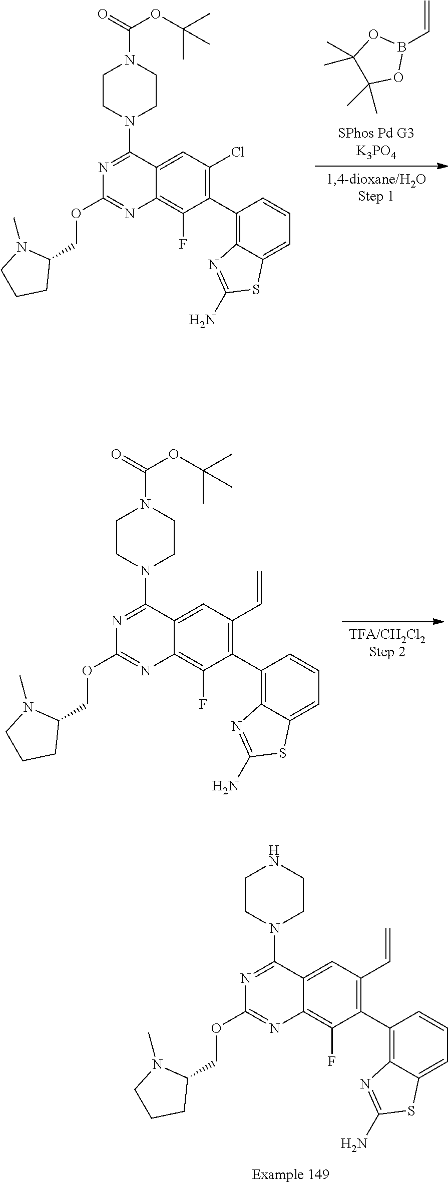

Step 1: tert-Butyl 4-(7-(2-aminobenzo[d]thiazol-4-yl)-8-fluoro-2-(((S)-1-methylpyrrolidin-2-yl)methoxy)-6-vinylquinazolin-4-yl)piperazine-1-carboxylate. A microwave vial was charged with tert-butyl 4-(7-(2-aminobenzo[d]thiazol-4-yl)-6-chloro-8-fluoro-2-(((S)-1-methylpyrrolidin-2-yl)methoxy)quinazolin-4-yl)piperazine-1-carboxylate (35 mg, 0.056 mmol), potassium phosphate (62 mg, 0.29 mmol), and (2-dicyclohexylphosphino-2′,6′-dimethoxybiphenyl) [2-(2′-amino-1,1′-biphenyl)]palladium(II) methanesulfonate (17 mg, 0.022 mmol). The vial was then purged with nitrogen gas for 5 min, and vinylboronic acid pinacol ester (44 mg, 48 μL, 0.28 mmol) was added. The resulting mixture was suspended in 1,4-dioxane (0.45 mL) and water (0.11 mL). The reaction was then heated to 150° C. via microwave irradiation. After 1.5 h, the reaction was cooled to room temperature and diluted with water (3 mL). The aqueous layer was extracted with EtOAc (3×3 mL). The organic layers were combined, dried over anhydrous sodium sulfate, filtered, and concentrated under reduced pressure. The crude oil was then purified by column chromatography on silica gel eluting with a gradient of 0-50% of a 3:1 EtOAc:EtOH mixture in heptane with 2% triethylamine, to provide tert-butyl 4-(7-(2-aminobenzo[d]thiazol-4-yl)-8-fluoro-2-(((S)-1-methylpyrrolidin-2-yl)methoxy)-6-vinylquinazolin-4-yl)piperazine-1-carboxylate. m/z (ESI, +ve ion): 620.2 (M+H)⁺.

Step 2: 4-(S-Fluoro-2-(((S)-1-methylpyrrolidin-2-yl)methoxy)-4-(piperazin-1-yl)-6-vinylquinazolin-7-yl)benzo[d]thiazol-2-amine. tert-Butyl 4-(7-(2-aminobenzo[d]thiazol-4-yl)-8-fluoro-2-(((S)-1-methylpyrrolidin-2-yl)methoxy)-6-vinylquinazolin-4-yl)piperazine-1-carboxylate was dissolved in DCM (2.4 mL). Trifluoroacetic acid (0.47 g, 0.32 mL, 4.2 mmol) was added and the reaction mixture was stirred at room temperature for 3 h. The reaction mixture was concentrated under reduced pressure and purified by reverse phase HPLC to provide 4-(8-fluoro-2-(((S)-1-methylpyrrolidin-2-yl)methoxy)-4-(piperazin-1-yl)-6-vinylquinazolin-7-yl)benzo[d]thiazol-2-amine (5.3 mg, 10.2 μmol, 18% yield over two steps) as white solid. m/z (ESI, +ve ion): 520.2 (M+H)⁺. ¹H NMR (400 MHz, METHANOL-d4) δ ppm 7.92-8.01 (m, 1H), 7.66-7.75 (m, 1H), 7.15-7.23 (m, 1H), 7.08-7.14 (m, 1H), 6.23-6.52 (m, 1H), 5.59-5.74 (m, 1H), 5.00-5.19 (m, 1H), 4.45-4.56 (m, 1H), 4.32-4.43 (m, 1H), 3.83-4.00 (m, 4H), 3.01-3.12 (m, 5H), 2.75-2.83 (m, 1H), 2.52 (s, 3H), 2.30-2.40 (m, 1H), 2.06-2.17 (m, 1H), 1.70-1.89 (m, 3H).

TABLE 7
Additional Examples. Prepared in an Analogous Manner to Example 149.

| | | | MS | |
|---|---|---|---|---|
| | | | m/z (ESI, | |
| Ex. # | Structure | Name | +ve ion) | ¹H NMR |
| 150 | 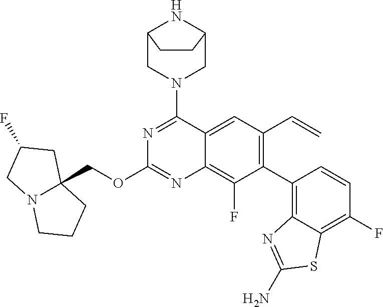 [SMILES: C=Cc1cc2c(N3CC4CCC(C3)N4)nc(OC[C@@]34CCCN3C[C@H](F)C4)nc2c(F)c1-c1ccc(F)c2sc(N)nc12] | 4-(4-((1R,5S)-3,8- diazabicyclo[3.2.1] octan-3-yl)-8-fluoro-2- (((2R,7aS)-2- fluorotetrahydro-1H- pyrrolizin-7a(5H)- yl)methoxy)-6- vinylquinazolin-7-yl)- 7- fluorobenzo[d]thiazol- 2-amine | 608.2 (M + H)⁺ | ¹H NMR (400 MHz, METHANOL-d4) δ ppm 8.03 (s, 1 H), 7.18 (ddd, J = 8.4, 5.5, 1.6 Hz, 1 H), 7.05 (t, J = 8.8 Hz, 1 H), 6.47 (dd, J = 17.6, 11.1 Hz, 1 H), 5.78 (d, J = 17.3 Hz, 1 H), 5.43- 5.69 (m, 1 H), 5.26 (d, J = 11.1 Hz, 1 H), 4.84-4.92 (m, 1 H), 4.67-4.80 (m, 3 H), 4.28 (br s, 2 H), 3.83-4.07 (m, 5 H), 3.44- 3.53 (m, 1 H), 2.56-2.77 (m, 2 H), 2.31-2.50 (m, 3 H), 2.09- 2.23 (m, 5 H). |
| 151 | 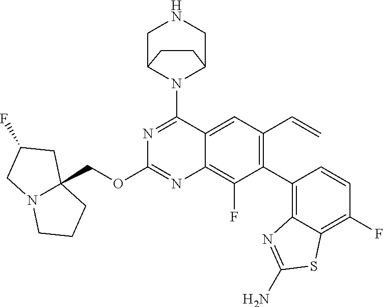 [SMILES: C=Cc1cc2c(N3C4CCC3CNC4)nc(OC[C@@]34CCCN3C[C@H](F)C4)nc2c(F)c1-c1ccc(F)c2sc(N)nc12] | 4-(4-((1R,5S)-3,8- diazabicyclo[3.2.1] octan-8-yl)-8-fluoro-2- (((2R,7aS)-2- fluorotetrahydro-1H- pyrrolizin-7a(5H)- yl)methoxy)-6- vinylquinazolin-7-yl)- 7- fluorobenzo[d]thiazol- 2-amine | 608.4 (M + H)⁺ | ¹H NMR (400 MHz, METHANOL-d4) δ ppm 8.06 (s, 1 H), 7.18 (ddd, J = 8.4, 5.5, 1.8 Hz, 1 H), 7.04 (t, J = 8.8 Hz, 1 H), 6.48 (dd, J = 17.3, 11.1 Hz, 1 H), 5.76-5.83 (m, 1 H), 5.46-5.67 (m, 1 H), 5.21-5.30 (m, 3 H), 4.66-4.77 (m, 2 H), 3.83-4.07 (m, 3 H), 3.73 (br d, J = 12.8 Hz, 2 H), 3.45-3.54 (m, 3 H), 2.56- 2.77 (m, 2 H), 2.29-2.49 (m, 5 H), 2.12-2.25 (m, 3 H). |

### 4-(6-Ethyl-8-fluoro-2-(((S)-1-methylpyrrolidin-2-yl)methoxy)-4-(piperazin-1-yl)quinazolin-7-yl)benzo[d]thiazol-2-amine (Example 152

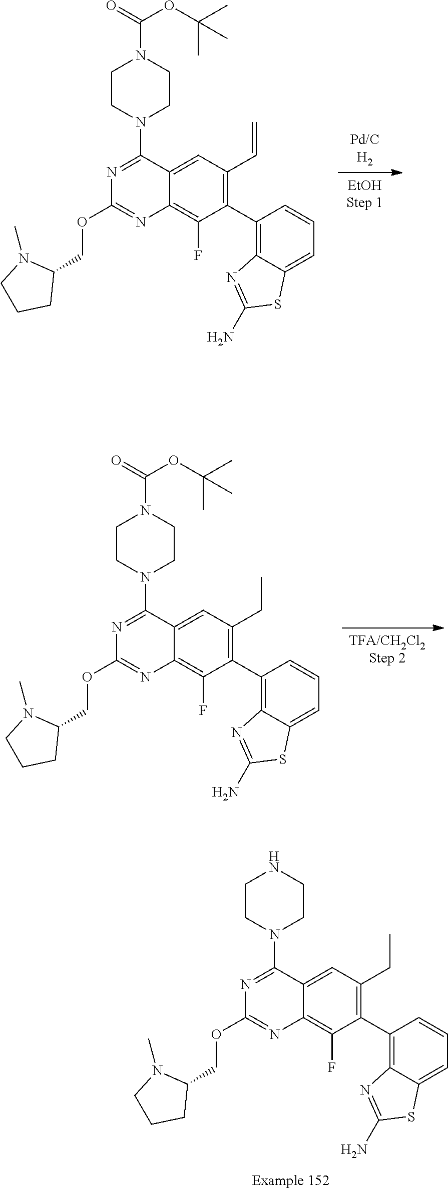 [SMILES: C=Cc1cc2c(N3CCN(C(=O)OC(C)(C)C)CC3)nc(OC[C@@H]3CCCN3C)nc2c(F)c1-c1cccc2sc(N)nc12 ; "EtOHStep 1" ; "Pd/CH2" ; CCc1cc2c(N3CCN(C(=O)OC(C)(C)C)CC3)nc(OC[C@@H]3CCCN3C)nc2c(F)c1-c1cccc2sc(N)nc12 ; "TFA/CH2Cl2Step 2" ; CCc1cc2c(N3CCNCC3)nc(OC[C@@H]3CCCN3C)nc2c(F)c1-c1cccc2sc(N)nc12]

Step 1: tert-Butyl 4-(7-(2-aminobenzo[d]thiazol-4-yl)-6-ethyl-8-fluoro-2-(((S)-1-methylpyrrolidin-2-yl)methoxy)quinazolin-4-yl)piperazine-1-carboxylate. A reaction tube was charged with tert-butyl 4-(7-(2-aminobenzo[d]thiazol-4-yl)-8-fluoro-2-(((S)-1-methylpyrrolidin-2-yl)methoxy)-6-vinylquinazolin-4-yl)piperazine-1-carboxylate (45 mg, 0.072 mmol, 60% purity). The solid was dissolved in ethanol (2.5 mL). To this solution 5% Pd/carbon (15 mg, 7.18 μmol) was added and the reaction was placed under an atmosphere of hydrogen gas (1 atm) at room temperature. The reaction was vigorously stirred for 1.5 h, and then the pressure was increased to 3 atm. After 2 days, the reaction was filtered through a plug of celite, and the plug was washed with MeOH. The resultant solution was then concentrated under reduced pressure and purified via reverse phase HPLC to provide tert-butyl 4-(7-(2-aminobenzo[d]thiazol-4-yl)-6-ethyl-8-fluoro-2-(((S)-1-methylpyrrolidin-2-yl)methoxy)quinazolin-4-yl)piperazine-1-carboxylate as white solid. m/z (ESI, +ve ion): 622.2 (M+H)⁺.

Step 2: 4-(6-Ethyl-8-fluoro-2-(((S)-1-methylpyrrolidin-2-yl)methoxy)-4-(piperazin-1-yl)quinazolin-7-yl)benzo[d]thiazol-2-amine. tert-Butyl 4-(7-(2-aminobenzo[d]thiazol-4-yl)-6-ethyl-8-fluoro-2-(((S)-1-methylpyrrolidin-2-yl)methoxy)quinazolin-4-yl)piperazine-1-carboxylate (13 mg, 0.021 mmol) was dissolved in DCM (1.8 mL). Trifluoroacetic acid (0.35 g, 0.24 mL, 3.10 mmol) was added and the reaction mixture was stirred at room temperature for 1 h. The reaction mixture was concentrated under reduced pressure and purified by reverse phase HPLC to provide 4-(6-ethyl-8-fluoro-2-(((S)-1-methylpyrrolidin-2-yl)methoxy)-4-(piperazin-1-yl)quinazolin-7-yl)benzo[d]thiazol-2-amine (12 mg, 0.024 mmol, 56% yield over two steps) as white solid. m/z (ESI, +ve ion): 522.2 (M+H)⁺. ¹H NMR (400 MHz, METHANOL-d4) δ ppm 7.83-7.92 (m, 1H), 7.74-7.79 (m, 1H), 7.39-7.47 (m, 1H), 7.33-7.38 (m, 1H), 4.90-5.01 (m, 1H), 4.66-4.76 (m, 1H), 4.18 (br s, 4H), 3.84-3.98 (m, 1H), 3.69-3.83 (m, 1H), 3.53 (br s, 4H), 3.19-3.30 (m, 1H), 3.12 (br s, 3H), 2.49-2.73 (m, 2H), 2.36-2.49 (m, 1H), 2.18-2.28 (m, 1H), 2.05-2.18 (m, 2H), 1.09 (d, J=1.3 Hz, 3H).

TABLE 8
Additional Examples. Prepared in an Analogous Manner to Example 152.

| | | | MS | |
|---|---|---|---|---|
| | | | m/z (ESI, | |
| Ex. # | Structure | Name | +ve ion) | ¹H NMR |
| 153 | 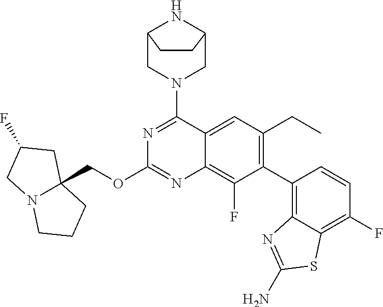 [SMILES: CCc1cc2c(N3CC4CCC(C3)N4)nc(OC[C@@]34CCCN3C[C@H](F)C4)nc2c(F)c1-c1ccc(F)c2sc(N)nc12] | 4-(4-((1R,5S)-3,8- diazabicyclo[3.2.1] octan-3-yl)-6-ethyl-8- fluoro-2-(((2R,7aS)-2- fluorotetrahydro-1H- pyrrolizin-7a(5H)- yl)methoxy)quinazolin- 7-yl)-7- fluorobenzo[d]thiazol- 2-amine | 610.2 (M + H)⁺ | ¹H NMR (400 MHz, METHANOL-d4) δ ppm 7.72 (s, 1 H), 7.21 (ddd, J = 8.3, 5.5, 1.9 Hz, 1 H), 7.05 (t, J = 8.8 Hz, 1 H), 5.46-5.68 (m, 1 H), 4.66-4.80 (m, 5 H), 4.28 (br s, 2 H), 3.83- 4.06 (m, 5 H), 3.44-3.52 (m, 1 H), 2.52-2.72 (m, 4 H), 2.32- 2.50 (m, 3 H), 2.11-2.22 (m, 5 H), 1.09 (t, J = 7.5 Hz, 3 H). |
| 154 | 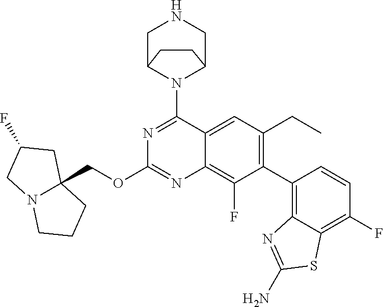 [SMILES: CCc1cc2c(N3C4CCC3CNC4)nc(OC[C@@]34CCCN3C[C@H](F)C4)nc2c(F)c1-c1ccc(F)c2sc(N)nc12] | 4-(4-((1R,5S)-3,8- diazabicyclo[3.2.1] octan-8-yl)-6-ethyl-8- fluoro-2-(((2R,7aS)-2- fluorotetrahydro-1H- pyrrolizin-7a(5H)- yl)methoxy)quinazolin- 7-yl)-7- fluorobenzo[d]thiazol- 2-amine | 610.2 (M + H)⁺ | ¹H NMR (400 MHz, METHANOL-d4) δ ppm 7.76 (s, 1 H), 7.21 (ddd, J = 8.2, 5.7, 2.2 Hz, 1 H), 7.05 (t, J = 8.8 Hz, 1 H), 5.39-5.74 (m, 1 H), 5.24 (br s, 2 H), 4.66-4.80 (m, 3 H), 3.80- 4.11 (m, 3 H), 3.71 (br dd, J = 12.9, 2.2 Hz, 2 H), 3.44-3.53 (m, 3 H), 2.52-2.74 (m, 4 H), 2.28-2.48 (m, 5 H), 2.10-2.24 (m, 3 H), 1.09 (t, J = 7.5 Hz, 3 H). |

### 4-(8-Fluoro-6-methyl-2-(((S)-1-methylpyrrolidin-2-yl)methoxy)-4-(piperazin-1-yl)quinazolin-7-yl)benzo[d]thiazol-2-amine (Example 155

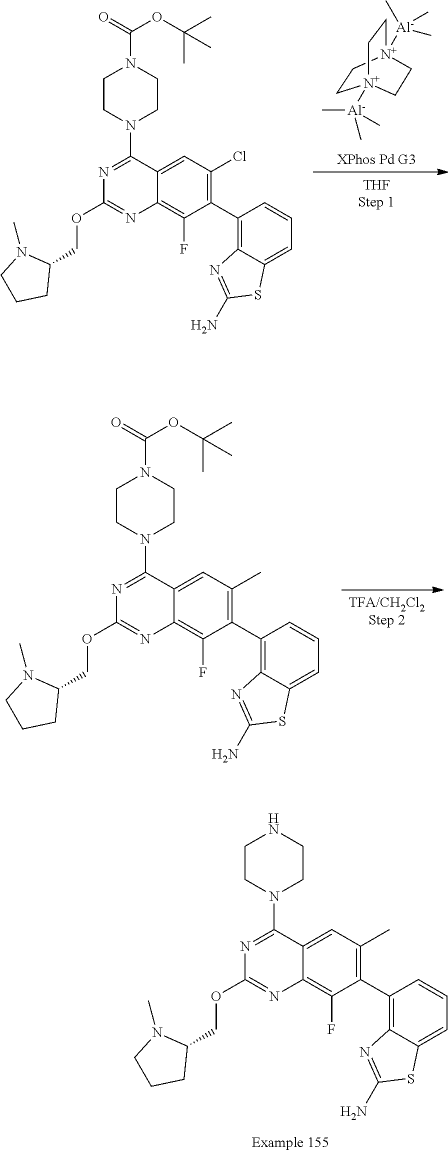

Step 1: tert-Butyl 4-(7-(2-aminobenzo[d]thiazol-4-yl)-8-fluoro-6-methyl-2-(((S)-1-methylpyrrolidin-2-yl)methoxy)quinazolin-4-yl)piperazine-1-carboxylate. An 8-mL vial was charged with tert-butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)piperazine-1-carboxylate (40 mg, 0.055 mmol), 1,4-diazabicyclo[2.2.2]octane bis(trimethylalumane) (14 mg, 0.055 mmol), and (2-dicyclohexylphosphino-2′,4′,6′-triisopropyl-1,1′-biphenyl)[2-(2′-amino-1,1′-biphenyl)]palladium(II) methanesulfonate (7.0 mg, 8.24 μmol). The vial was purged with nitrogen gas. The solids were suspended in tetrahydrofuran (1.1 mL), and the reaction mixture was then heated to 70° C. After 1 h, the reaction was cooled to room temperature and diluted with water (1.5 mL), saturated aqueous ammonium chloride (1.5 mL), and EtOAc (3 mL). The layers were separated and aqueous layer was then extracted with EtOAc (3×3 mL). The combined organic layers were then dried over anhydrous sodium sulfate, filtered, and concentrated under reduced pressure. The crude residue was purified sequentially using column chromatography on silica gel eluting with a gradient of 0-50% of a 3:1 EtOAc:EtOH mixture in heptane with 2% triethylamine, followed by reverse phase HPLC to provide tert-butyl 4-(7-(2-aminobenzo[d]thiazol-4-yl)-8-fluoro-6-methyl-2-(((S)-1-methylpyrrolidin-2-yl)methoxy)quinazolin-4-yl)piperazine-1-carboxylate. m/z (ESI, +ve ion): 607.9 (M+H)⁺.

Step 2: 4-(8-Fluoro-6-methyl-2-(((S)-1-methylpyrrolidin-2-yl)methoxy)-4-(piperazin-1-yl)quinazolin-7-yl)benzo[d]thiazol-2-amine. tert-Butyl 4-(7-(2-aminobenzo[d]thiazol-4-yl)-8-fluoro-6-methyl-2-(((S)-1-methylpyrrolidin-2-yl)methoxy)quinazolin-4-yl)piperazine-1-carboxylate was dissolved in DCM (2.4 mL). Trifluoroacetic acid (0.47 g, 0.32 mL, 4.16 mmol) was added and the reaction mixture was stirred at room temperature for 5 h. The reaction mixture was concentrated under reduced pressure and purified by reverse phase HPLC to provide 4-(8-fluoro-6-methyl-2-(((S)-1-methylpyrrolidin-2-yl)methoxy)-4-(piperazin-1-yl)quinazolin-7-yl)benzo[d]thiazol-2-amine (2.54 mg, 5.00 μmol, 9% yield over two steps) as yellow solid. m/z (ESI, +ve ion): 507.850 (M+H)⁺. ¹H NMR (400 MHz, CHLOROFORM-d) δ ppm 7.62-7.69 (m, 1H), 7.48-7.53 (m, 1H), 7.18-7.25 (m, 2H), 5.43-5.66 (m, 1H), 4.52-4.68 (m, 1H), 4.25-4.40 (m, 1H), 3.72-3.91 (m, 4H), 3.03-3.16 (m, 5H), 2.68-2.83 (m, 1H), 2.52 (d, J=2.1 Hz, 3H), 2.19 (s, 4H), 1.99-2.13 (m, 1H), 1.79-1.91 (m, 2H), 0.84-1.18 (m, 3H).

### 7-(2-Aminobenzo[d]thiazol-4-yl)-8-fluoro-2-(((S)-1-methylpyrrolidin-2-yl)methoxy)-4-(piperazin-1-yl)quinazoline-6-carbonitrile (Example 156

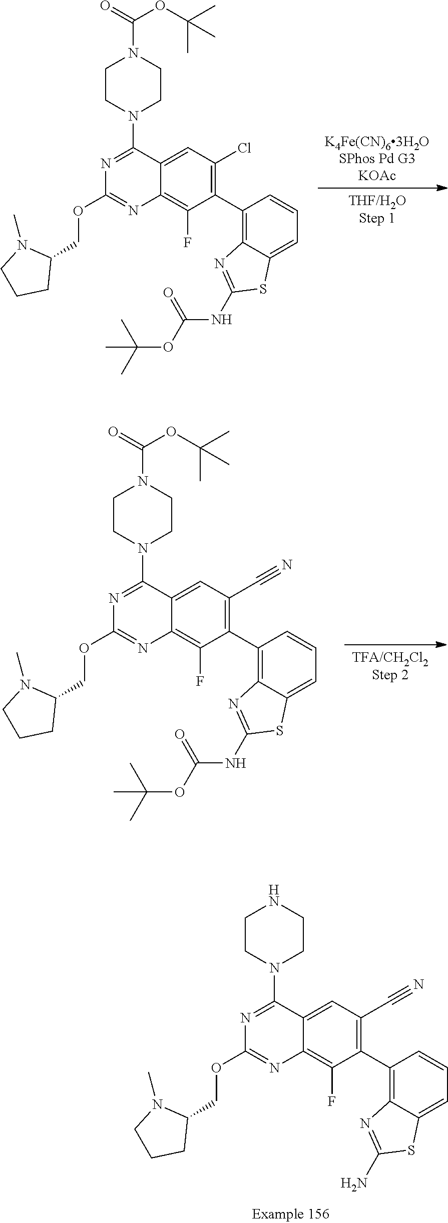 [SMILES: CN1CCC[C@H]1COc1nc(N2CCN(C(=O)OC(C)(C)C)CC2)c2cc(Cl)c(-c3cccc4sc(NC(=O)OC(C)(C)C)nc34)c(F)c2n1 ; "K4Fe(CN)6·3H2OSPhos Pd G3KOAc" ; "THF/H2OStep 1" ; CN1CCC[C@H]1COc1nc(N2CCN(C(=O)OC(C)(C)C)CC2)c2cc(C#N)c(-c3cccc4sc(NC(=O)OC(C)(C)C)nc34)c(F)c2n1 ; "TFA/CH2Cl2Step 2" ; CN1CCC[C@H]1COc1nc(N2CCNCC2)c2cc(C#N)c(-c3cccc4sc(N)nc34)c(F)c2n1]

Step 1: tert-Butyl 4-(7-(2-((tert-butoxycarbonyl)amino)benzo[d]thiazol-4-yl)-6-cyano-8-fluoro-2-(((S)-1-methylpyrrolidin-2-yl)methoxy)quinazolin-4-yl)piperazine-1-carboxylate. A 4-mL vial was charged with tert-butyl 4-(7-(2-((tert-butoxycarbonyl)amino)benzo[d]thiazol-4-yl)-6-chloro-8-fluoro-2-(((S)-1-methylpyrrolidin-2-yl)methoxy)quinazolin-4-yl)piperazine-1-carboxylate (48 mg, 0.066 mmol), potassium acetate (19 mg, 0.20 mmol), potassium ferrocyanide trihydrate (0.11 g, 0.26 mmol), and (2-dicyclohexylphosphino-2′,6′-dimethoxybiphenyl) [2-(2′-amino-1,1′-biphenyl)]palladium(II) methanesulfonate (21 mg, 0.026 mmol). The vial was purged with nitrogen gas and then the solids were suspended in tetrahydrofuran (0.33 mL) and water (0.33 mL) was added. The reaction was then sealed and stirred at 100° C. After 2.5 h, the reaction was cooled to room temperature, and diluted with water (3 mL) and EtOAc (3 mL). The solution was filtered through celite. The layers were separated, and then the aqueous layer was extracted with EtOAc (3×3 mL). The combined organic layers were then dried over anhydrous sodium sulfate, filtered, and concentrated under reduced pressure. The resultant crude oil was then purified via column chromatography on silica gel eluting with a gradient of 0-50% of a 3:1 EtOAc:EtOH mixture in heptane with 2% triethylamine to provide tert-butyl 4-(7-(2-((tert-butoxycarbonyl)amino)benzo[d]thiazol-4-yl)-6-cyano-8-fluoro-2-(((S)-1-methylpyrrolidin-2-yl)methoxy)quinazolin-4-yl)piperazine-1-carboxylate as yellow solid. m/z (ESI, +ve ion): 719.2 (M+H)⁺.

Step 2: 7-(2-Aminobenzo[d]thiazol-4-yl)-8-fluoro-2-(((S)-1-methylpyrrolidin-2-yl)methoxy)-4-(piperazin-1-yl)quinazoline-6-carbonitrile. tert-Butyl 4-(7-(2-((tert-butoxycarbonyl)amino)benzo[d]thiazol-4-yl)-6-cyano-8-fluoro-2-(((S)-1-methylpyrrolidin-2-yl)methoxy)quinazolin-4-yl)piperazine-1-carboxylate was dissolved in DCM (3.0 mL). Trifluoroacetic acid (0.56 g, 0.38 mL, 4.90 mmol) was added and the reaction mixture was stirred at room temperature for 1.5 h. The reaction mixture was concentrated under reduced pressure and purified by reverse phase HPLC to 7-(2-aminobenzo[d]thiazol-4-yl)-8-fluoro-2-(((S)-1-methylpyrrolidin-2-yl)methoxy)-4-(piperazin-1-yl)quinazoline-6-carbonitrile (2.7 mg, 5.11 μmol, 8% yield over two steps) as white solid. m/z (ESI, +ve ion): 518.850 (M+H)⁺. ¹H NMR (400 MHz, METHANOL-d4) δ ppm 8.39-8.43 (m, 1H), 7.81-7.86 (m, 1H), 7.34-7.40 (m, 1H), 7.27-7.33 (m, 1H), 4.93-5.00 (m, 1H), 4.68-4.77 (m, 1H), 4.17-4.22 (m, 4H), 3.89-4.01 (m, 1H), 3.74-3.85 (m, 1H), 3.51-3.56 (m, 4H), 3.21-3.29 (m, 1H), 3.10-3.16 (m, 3H), 2.32-2.51 (m, 1H), 2.21-2.32 (m, 1H), 2.08-2.18 (m, 2H).

### 7-(2-Amino-7-fluorobenzo[d]thiazol-4-yl)-6-chloro-8-fluoro-2-(((S)-1-methylpyrrolidin-2-yl)methoxy)-4-(piperazin-1-yl)quinoline-3-carbonitrile (Example 157

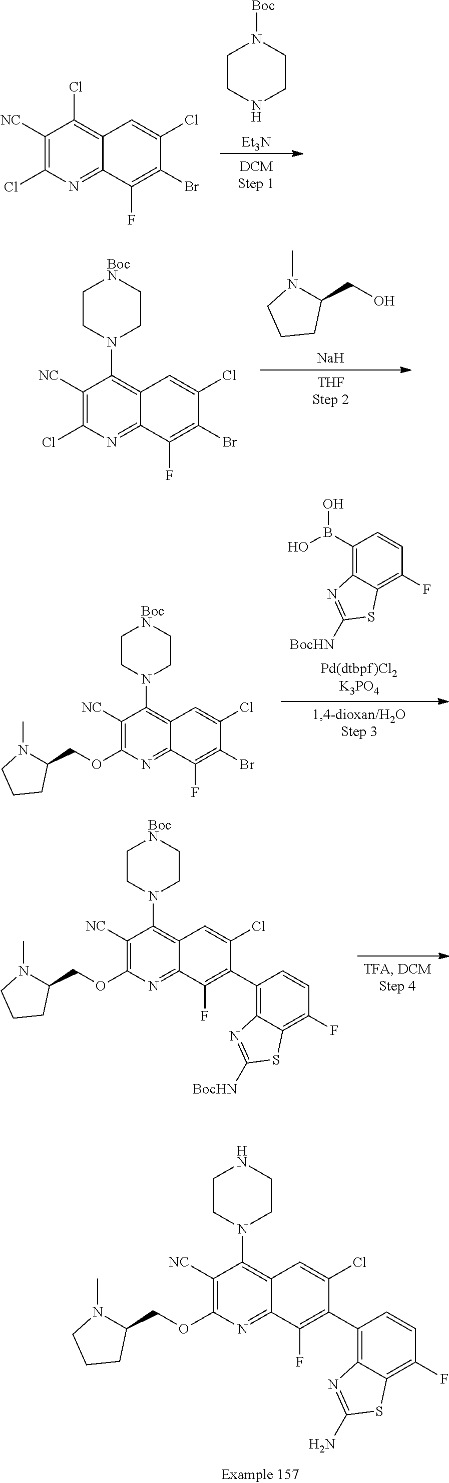

Step 1: tert-Butyl 4-(7-bromo-2,6-dichloro-3-cyano-8-fluoroquinolin-4-yl)piperazine-1-carboxylate. To the suspension of 7-bromo-2,4,6-trichloro-8-fluoroquinoline-3-carbonitrile (0.35 g, 1.0 mmol, Enamine) in DCM (6.7 mL) was added triethylamine (0.56 mL, 4.0 mmol, Sigma-Aldrich Corporation), followed by tert-butyl piperazine-1-carboxylate (0.37 g, 2 mmol, Combi-Blocks Inc.). The reaction mixture was stirred at room temperature for 1 h. The reaction mixture was quenched with saturated NaHCO₃ solution and partitioned between EtOAc and water. The precipitate formed was collected by filtration, dried in a vacuum oven at 60° C. to give tert-butyl 4-(7-bromo-2,6-dichloro-3-cyano-8-fluoroquinolin-4-yl)piperazine-1-carboxylate (0.31 g, 0.61 mmol, 61% yield) as off-white solid, which was used directly in the following step. m/z (ESI, +ve ion): 505.0 (M+H⁺).

Step 2: tert-Butyl (S)-4-(7-bromo-6-chloro-3-cyano-8-fluoro-2-((1-methylpyrrolidin-2-yl)methoxy)quinolin-4-yl)piperazine-1-carboxylate. To the mixture of tert-butyl 4-(7-bromo-2,6-dichloro-3-cyano-8-fluoroquinolin-4-yl)piperazine-1-carboxylate (81 mg, 0.16 mmol) and (2S)-1-methyl-2-pyrrolidinemethanol (23 μL, 0.19 mmol, Sigma-Aldrich Corporation) in tetrahydrofuran (0.8 mL) was added sodium hydride (9.6 mg, 0.24 mmol, Sigma-Aldrich Corporation). The reaction mixture was stirred at room temperature for 1 h. The reaction mixture was partitioned between EtOAc and water. The aqueous layer was back-extracted with EtOAc (3×) and the combined organics was dried over anhydrous Na₂SO₄ and concentrated. The crude material was purified by column chromatography on silica gel eluting with a gradient of 10% to 40% 3:1 EtOAc/EtOH in heptane, to provide tert-butyl (S)-4-(7-bromo-6-chloro-3-cyano-8-fluoro-2-((1-methylpyrrolidin-2-yl)methoxy)quinolin-4-yl)piperazine-1-carboxylate (55 mg, 0.09 mmol, 58% yield) as white solid. m/z (ESI, +ve ion): 582.1 (M+H⁺).

Step 3: tert-Butyl 4-(7-(2-((tert-butoxycarbonyl)amino)-7-fluorobenzo[d]thiazol-4-yl)-6-chloro-3-cyano-8-fluoro-2-(((S)-1-methylpyrrolidin-2-yl)methoxy)quinolin-4-yl)piperazine-1-carboxylate. A mixture of tert-butyl (S)-4-(7-bromo-6-chloro-3-cyano-8-fluoro-2-((1-methylpyrrolidin-2-yl)methoxy)quinolin-4-yl)piperazine-1-carboxylate (35 mg, 0.06 mmol), (2-((tert-butoxycarbonyl)amino)-7-fluorobenzo[d]thiazol-4-yl)boronic acid (37 mg, 0.12 mmol, e-Novation), [1,1′-bis(di-tert-butylphosphino)ferrocene]dichloropalladium(II) (20 mg, 0.03 mmol, Strem Chemicals, Inc.), and potassium phosphate tribasic (38 mg, 0.18 mmol, Acros Organics) in a vial was deoxygenated under vacuum, and then flushed with nitrogen. 1,4-Dioxane (0.4 mL) and water (57 μL) were added and the reaction mixture was stirred at 90° C. for 1.5 h. Sodium sulfate was added to the reaction mixture. The crude material was purified by column chromatography on silica gel, eluting with a gradient of 0% to 40% of 3:1 EtOAc/EtOH in heptane, to provide tert-butyl 4-(7-(2-((tert-butoxycarbonyl)amino)-7-fluorobenzo[d]thiazol-4-yl)-6-chloro-3-cyano-8-fluoro-2-(((S)-1-methylpyrrolidin-2-yl)methoxy)quinolin-4-yl)piperazine-1-carboxylate (10 mg, 13 μmol, 21% yield) as off-white solid. m/z (ESI, +ve ion): 770.5 (M+H⁺).

Step 4: 7-(2-Amino-7-fluorobenzo[d]thiazol-4-yl)-6-chloro-8-fluoro-2-(((S)-1-methylpyrrolidin-2-yl)methoxy)-4-(piperazin-1-yl)quinoline-3-carbonitrile. To a solution of tert-butyl 4-(7-(2-((tert-butoxycarbonyl)amino)-7-fluorobenzo[d]thiazol-4-yl)-6-chloro-3-cyano-8-fluoro-2-(((S)-1-methylpyrrolidin-2-yl)methoxy)quinolin-4-yl)piperazine-1-carboxylate (10 mg, 13 μmol) in DCM (50 uL) was added trifluoroacetic acid (20 μL, 0.2 mmol, Sigma-Aldrich Corporation). The reaction mixture was stirred at room temperature for 2 h, and the solvent was removed under vacuum. The crude material was dissolved in DMSO, and purified by reverse-phase preparative HPLC using a Phenomenex Gemini column, 10 micron, C18, 110 Å, 150×30 mm, 0.1% TFA in CH₃CN/H₂O, gradient 0% to 70% over 15 min to provide 7-(2-amino-7-fluorobenzo[d]thiazol-4-yl)-6-chloro-8-fluoro-2-(((S)-1-methylpyrrolidin-2-yl)methoxy)-4-(piperazin-1-yl)quinoline-3-carbonitrile (9 mg, 0.011 mmol, 86% yield) as white solid. m/z (ESI, +ve ion): 570.1 (M+H). ¹H NMR (400 MHz, METHANOL-d₄) δ8.00 (d, J=1.25 Hz, 1H), 7.24 (dd, J=5.64, 8.36 Hz, 1H), 7.04 (t, J=8.88 Hz, 1H), 5.05 (td, J=2.90, 13.01 Hz, 1H), 4.68 (ddd, J=5.12, 7.52, 12.85 Hz, 1H), 3.95-4.10 (m, 5H), 3.73-3.84 (S, 1H), 3.61 (br t, J=5.02 Hz, 5H), 3.20 (s, 3H), 2.40-2.56 (m, 1H), 2.06-2.32 (m, 3H).

TABLE 9
Additional Examples. Prepared in an Analogous Manner to Example 157.

| | | | MS | |
|---|---|---|---|---|
| | | | m/z | |
| | | | (ESI, | |
| Ex. # | Structure | Name | +ve ion) | ¹H NMR |
| 158 | 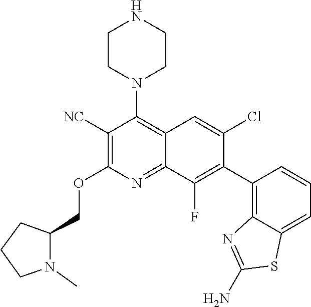 [SMILES: CN1CCC[C@H]1COc1nc2c(F)c(-c3cccc4sc(N)nc34)c(Cl)cc2c(N2CCNCC2)c1C#N] | 7-(2- aminobenzo[d]thiazol- 4-yl)-6-chloro-8-fluoro- 2-(((S)-1- methylpyrrolidin-2- yl)methoxy)-4- (piperazin-1- yl)quinoline-3- carbonitrile | 552.1 (M + H)⁺ | ¹H NMR (400 MHz, METHANOL-d4) δ 8.01 (s, 1H), 7.78 (dd, J = 1.67, 7.32 Hz, 1H), 7.19-7.29 (m, 2H), 5.05 (br dd, J = 2.82, 12.85 Hz, 1H), 4.69 (tdd, J = 4.02, 8.26, 12.54 Hz, 1H), 3.96-4.09 (m, 5H), 3.72- 3.86 (m, 1H), 3.62 (br t, J = 5.02 Hz, 5H), 3.20 (s, 3H), 2.39-2.54 (m, 1H), 2.03-2.34 (m, 3H). |
| 159 | 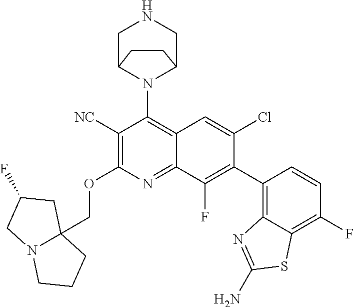 [SMILES: N#Cc1c(OCC23CCCN2C[C@H](F)C3)nc2c(F)c(-c3ccc(F)c4sc(N)nc34)c(Cl)cc2c1N1C2CCC1CNC2] | 7-(2-amino-7- fluorobenzo[d]thiazol- 4-yl)-4-((1R,5S)-3,8- diazabicyclo[3.2.1]octan- 8-yl)-6-chloro-8- fluoro-2-(((2R)-2- fluorotetrahydro-1H- pyrrolizin-7a(5H)- yl)methoxy)quinoline- 3-carbonitrile | 640.1 (M + H)⁺ | ¹H NMR (400 MHz, METHANOL-d4) δ 7.92 (s, 1H), 7.24 (dd, J = 5.43, 8.36 Hz, 1H), 7.04 (t, J = 8.78 Hz, 1H), 5.53- 5.78 (m, 1H), 4.99-5.10 (m, 2H), 4.66-4.80 (m, 3H), 3.93-4.14 (m, 3H), 3.81 (br t, J = 11.50 Hz, 2H), 3.46-3.58 (m, 3H), 2.75-2.87 (m, 1H), 2.61-2.74 (m, 1H), 2.32- 2.58 (m, 4H), 2.26 (br d, J = 8.78 Hz, 3H). |
| 160 | 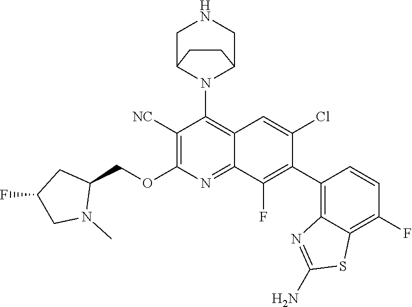 [SMILES: CN1C[C@H](F)C[C@H]1COc1nc2c(F)c(-c3ccc(F)c4sc(N)nc34)c(Cl)cc2c(N2C3CCC2CNC3)c1C#N] | 7-(2-amino-7- fluorobenzo[d]thiazol- 4-yl)-4-((1R,5S)-3,8- diazabicyclo[3.2.1]octan- 8-yl)-6-chloro-8- fluoro-2-(((2S,4R)-4- fluoro-1- methylpyrrolidin-2- yl)methoxy)quinoline- 3-carbonitrile | 614.1 (M + H)⁺ | ¹H NMR (400 MHz, METHANOL-d4) δ 7.91 (br s, 1H), 7.24 (br dd, J = 5.54, 7.84 Hz, 1H), 7.03 (br t, J = 8.78 Hz, 1H), 5.44-5.65 (m, 1H), 5.06 (br d, J = 11.08 Hz, 4H), 4.37 (br s, 1H), 3.66-3.88 (m, 4H), 3.53 (br d, J = 12.33 Hz, 2H), 3.27 (br s, 3H), 2.64-2.83 (m, 1H), 2.43- 2.61 (m, 3H), 2.28 (br d, J = 8.36 Hz, 2H). |

### 4-(4-((1R,5S)-3,8-Diazabicyclo[3.2.1]octan-3-y)-6-chloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-amine (Example 161

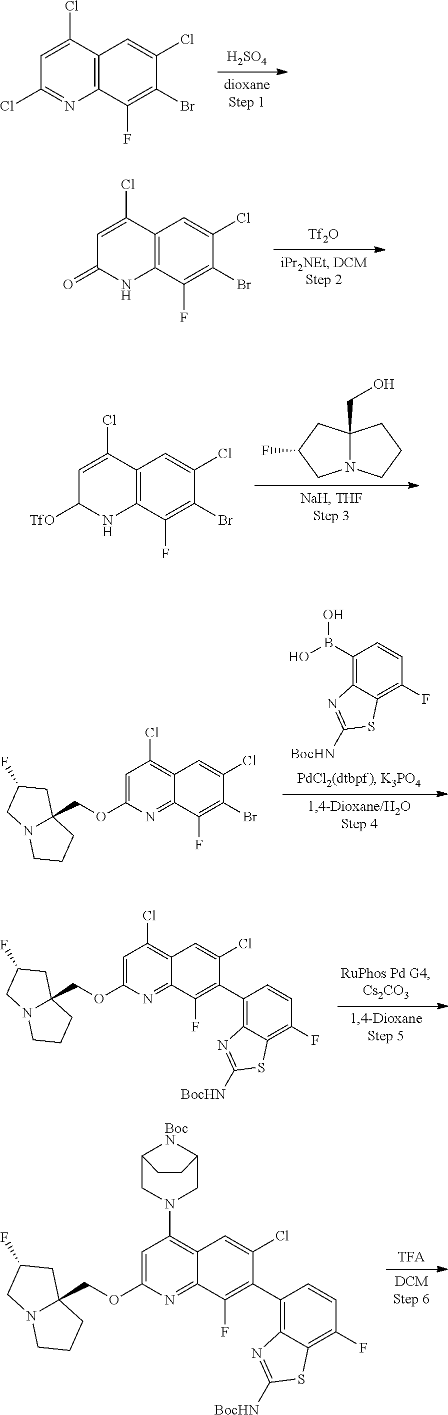

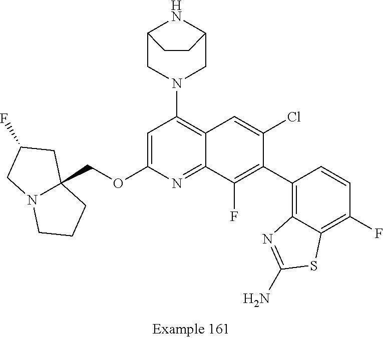 [SMILES: Nc1nc2c(-c3c(Cl)cc4c(N5CC6CCC(C5)N6)cc(OC[C@@]56CCCN5C[C@H](F)C6)nc4c3F)ccc(F)c2s1]

Step 1: 7-Bromo-4,6-dichloro-8-fluoroquinolin-2(1H)-one. A mixture of 7-bromo-2,4,6-trichloro-8-fluoroquinoline (1.00 g, 3.04 mmol) in sulfuric acid (4.94 g, 4.99 mL, 50.4 mmol) diluted to 20% with water, and 1,4-dioxane (25.3 mL) was stirred at 100° C. for 30 h. After cooling to room temperature, the reaction mixture was diluted with water. The solid was collected by filtration, washed with water and dried in a vacuum oven at 50° C. over 2-d to give 7-bromo-4,6-dichloro-8-fluoroquinolin-2(1H)-one (0.855 g, 2.75 mmol, 91% yield) as off-white solid, which was used directly in the subsequent step. m/z (ESI, +ve ion): 310.0 (M+H⁺).

Step 2: 7-Bromo-4,6-dichloro-8-fluoroquinolin-2-yl trifluoromethanesulfonate. To a suspension of 7-bromo-4,6-dichloro-8-fluoroquinolin-2(1H)-one (0.84 g, 2.71 mmol) in dichloromethane (9 mL) was added 1,1′-dimethyltriethylamine (0.53 g, 0.71 mL, 4.07 mmol, Sigma-Aldrich Corporation), followed by dropwise addition of trifluoromethanesulfonic anhydride solution, 1M in methylene chloride (3 mL, 2.99 mmol, Sigma-Aldrich Corporation). The reaction mixture was stirred at room temperature for 1 h. The solvent was evaporated and the residue solid was used directly in the subsequent step. m/z (ESI, +ve ion): 441.8 (M+H⁺).

Step 3: 7-Bromo-4,6-dichloro-8-fluoro-2-(((2R,7aS)-2-fluorotetrahydro-1H-pyrrolizin-7a(5H)-yl)methoxy)quinoline. To a solution of ((2R,7aS)-2-fluorotetrahydro-1H-pyrrolizin-7a(5H)-yl)methanol (0.86 g, 5.42 mmol, PharmaBlock) in THF (6.5 mL) was added sodium hydride (0.23 g, 5.69 mmol, TCI America). The reaction mixture was stirred at rt for 25 min and was added dropwise to the solution of 7-bromo-4,6-dichloro-8-fluoroquinolin-2-yl trifluoromethanesulfonate (1.20 g, 2.71 mmol) in THF (6 mL). The alkoxide vial was rinsed with THF (1 mL) and added to the reaction at once. The reaction mixture was stirred at rt for 10 min. The reaction mixture was heated at 60° C. for 40 min and at 55° C. overnight. After cooling to rt, water was added to quench the reaction and the aqueous layer was back extracted with EtOAc (2×) and the combined organics was dried (Na₂SO₄) and concentrated. The crude material was purified by chromatography through a Redi-Sep pre-packed silica gel column (80 g), eluting with a gradient of 0% to 30% 3:1 EtOAc/EtOH in heptane, to provide 7-bromo-4,6-dichloro-8-fluoro-2-(((2R,7aS)-2-fluorotetrahydro-1H-pyrrolizin-7a(5H)-yl)methoxy)quinoline (0.37 g, 0.83 mmol, 31% yield) as tan solid, which was used directly in the subsequent step. m/z (ESI, +ve ion): 450.7 (M+H⁺).

Step 4: tert-Butyl (4-(4,6-dichloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-yl)carbamate. A mixture of 7-bromo-4,6-dichloro-8-fluoro-2-(((2R,7aS)-2-fluorotetrahydro-1H-pyrrolizin-7a(5H)-yl)methoxy)quinoline (0.18 g, 0.40 mmol), (2-((tert-butoxycarbonyl)amino)-7-fluorobenzo[d]thiazol-4-yl)boronic acid (0.13 g, 0.40 mmol, Synnovator), [1,1′-bis(di-tert-butylphosphino)ferrocene]dichloropalladium(II) (0.10 g, 0.16 mmol, Strem Chemicals, Inc.), and potassium phosphate tribasic (0.17 g, 0.80 mmol, Acros Organics) in a round-bottomed flask was deoxygenated under vacuum, and then flushed with nitrogen. 1,4-dioxane (2.3 mL) and water (0.38 mL) were added and the reaction mixture was stirred at 80° C. for 1 h. Na₂SO₄ was added to the reaction. The crude material was purified by chromatography through a silica gel column (40 g), eluting with a gradient of 0% to 20% of 3:1 EtOAc/EtOH in heptane, to provide tert-butyl (4-(4,6-dichloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-yl)carbamate (44 mg, 0.069 mmol, 17% yield) as tan solid. m/z (ESI, +ve ion): 640.7 (M+H⁺).

Step 5: tert-Butyl (1R,5S)-3-(7-(2-((tert-butoxycarbonyl)amino)-7-fluorobenzo[d]thiazol-4-yl)-6-chloro-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate. A mixture of tert-butyl (4-(4,6-dichloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-yl)carbamate (44 mg, 0.069 mmol), 8-boc-3,8-diaza-bicyclo[3.2.1]octane (29 mg, 0.14 mmol, Chem-Impex International, Inc.), RuPhos Pd G4 (12 mg, 0.014 mmol, Sigma-Aldrich Corporation), and cesium carbonate (67 mg, 0.21 mmol, Strem Chemicals, Inc.) in a round-bottomed flask was deoxygenated, then flushed with nitrogen. 1,4-Dioxane (0.34 mL) was added and the reaction mixture was stirred at 85° C. for 1 h. More amine (15 mg) and RuPhos G4 (6 mg) was added and the reaction mixture was stirred at 85° C. for an additional 1 h. After cooling to rt, the crude material was purified by chromatography through a silica gel column (12 g), eluting with a gradient of 0% to 40% 3:1 EtOAc/EtOH in heptane, to provide tert-butyl (1R,5S)-3-(7-(2-((tert-butoxycarbonyl)amino)-7-fluorobenzo[d]thiazol-4-yl)-6-chloro-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (4 mg, 4.91 μmol, 7.1% yield) as white solid. m/z (ESI, +ve ion): 815.5 (M+H⁺).

Step 6: 4-(4-((1R,5S)-3,8-Diazabicyclo[3.2.1]octan-3-yl)-6-chloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-amine bis(2,2,2-trifluoroacetate). To a solution of tert-butyl (1R,5S)-3-(7-(2-((tert-butoxycarbonyl)amino)-7-fluorobenzo[d]thiazol-4-yl)-6-chloro-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (4 mg, 4.91 μmol) in dichloromethane (16 μL) was added 1,1,1-trifluoroacetic acid (15 mg, 10 μL, 0.13 mmol, Sigma-Aldrich Corporation) dropwise. The reaction mixture was stirred at rt for 1 h. The solvent was evaporated in vacuo and the residue was dissolved in DMSO and purified by reverse-phase preparative HPLC using a Phenomenex Gemini column, 10 micron, C18, 110 Å, 150×30 mm, 0.1% TFA in CH₃CN/H₂O, gradient 5% to 80% over 15 min to provide 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-amine bis(2,2,2-trifluoroacetate) (2.5 mg, 2.97 μmol, 60% yield) as white solid. ¹H NMR (400 MHz, METHANOL-d4) δ 7.99 (s, 1H), 7.21-7.30 (m, 1H), 6.99-7.08 (m, 1H), 6.87 (s, 1H), 4.26-4.33 (m, 2H), 3.91-3.94 (m, 1H), 3.82-3.91 (m, 3H), 3.65-3.74 (m, 4H), 3.49-3.52 (m, 4H), 2.61-2.65 (m, 1H), 2.49 (d, J=7.52 Hz, 2H), 2.28-2.38 (m, 4H), 1.29-1.42 (m, 8H). m/z (ESI, +ve ion): 614.8 (M+H⁺).

### Biological Evaluation

Provided in this section is the biological evaluation of the specific examples provided herein.

### KRAS G12D TR-FRET Assay

Compounds of interest were prepared in a dose-response titration in DMSO, and 80 nL were added via Labcyte Echo to each well of a 384-well plate (Perkin Elmer 6008280). The His-tagged KRAS G12D protein (Amgen) was diluted to 20 nM in Assay Buffer (20 mM HEPES, pH 7.4, 10 mM MgCl₂, 50 mM NaCl, 0.1% BSA, 0.01% Tween-20, 10 μM GDP) and 2 uL was added to the appropriate wells of the 384-well plate. The plate was incubated for 30 minutes at rt. Biotinylated KRPep-2d substrate (Amgen) was diluted to 20 nM in Assay Buffer and 2 μL was added to all wells and incubated for 1 hour at room temperature. Detection Reagent (0.4 nM LANCE Eu-W1024 Anti-6xHis (Perkin Elmer AD0401), 5 nM streptavidin-d2 (Cisbio 610SADLA)) was prepared in Assay Buffer, then 4 μL was added to the plate and incubated for 1 h at rt. Plates were read using PerkinElmer EnVision (ex: 320 nm, em1: 665 nm, em2: 615 nm) and em1/em2 data was used to generate curve fits using a 4-parameter logistic model to calculate IC₅₀ values.

### KRAS G12D Coupled Nucleotide Exchange Assay

Purified GDP-bound KRAS protein (aa 1-169), containing both G12D and C118A amino acid substitutions and an N-terminal His-tag, was pre-incubated in assay buffer (25 mM HEPES pH 7.4, 10 mM MgCl₂, and 0.01% Triton X-100) with a compound dose-response titration for 2 h. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 10 min. The assay plates were then read on a PerkinElmer EnVision Multilabel Reader, using AlphaScreen® technology, and data were analyzed using a 4-parameter logistic model to calculate IC₅₀ values.

### Phospho-ERK1/2 MSD Assay

A-427 (ATCC® HTB-53™) cells were cultured in RPMI 1640 Medium (ThermoFisher Scientific 11875093) containing 10% fetal bovine serum (ThermoFisher Scientific 16000044) and 1× penicillin-streptomycin-glutamine (ThermoFisher Scientific 10378016). Sixteen hours prior to compound treatment, A-427 cells were seeded in 96-well cell culture plates at a density of 25,000 cells/well and incubated at 37° C., 5% CO₂. A compound dose-response titration was diluted in growth media, added to appropriate wells of a cell culture plate, and then incubated at 37° C., 5% CO₂ for 2 h. Following compound treatment, cells were washed with ice-cold Dulbecco's phosphate-buffered saline, no Ca²⁺ or Mg²⁺ (ThermoFisher Scientific 14190144), and then lysed in RIPA buffer (50 mM Tris-HCl pH 7.5, 1% Igepal, 0.5% sodium deoxycholate, 150 mM NaCl, and 0.5% sodium dodecyl sulfate) containing protease inhibitors (Roche 4693132001) and phosphatase inhibitors (Roche 4906837001). Phosphorylation of ERK1/2 in compound-treated lysates was assayed using Phospho-ERK1/2 Whole Cell Lysate kits (Meso Scale Discovery K151DWD) according to the manufacturer's protocol. Assay plates were read on a Meso Scale Discovery Sector Imager 6000, and data were analyzed using a 4-parameter logistic model to calculate IC₅₀ values.

TABLE 10
Biochemical and cellular activity of examples

| | TR-FRET | Avg 2 h Coupled | pERK (A427 |
|---|---|---|---|
| | KRAS G12D IC₅₀ | Exchange IC₅₀ | cells) IC₅₀ |
| Ex. # | (μM) | (μM) | (μM) |
| 1 | 11.3 | 3.655 | NT |
| 2 | 0.005 | 0.004 | 0.023 |
| 3 | 0.006 | 0.005 | 0.194 |
| 4 | 0.01 | 0.005 | 4.9 |
| 5 | 0.01 | 0.005 | 2.63 |
| 6 | 0.005 | 0.005 | 0.364 |
| 7 | 0.012 | 0.006 | 0.531 |
| 8 | 0.009 | 0.007 | NT |
| 9 | 0.007 | 0.009 | 0.403 |
| 10 | 0.014 | 0.01 | NT |
| 11 | 0.029 | 0.011 | 8.25 |
| 12 | 0.018 | 0.014 | NT |
| 13 | 0.022 | 0.014 | NT |
| 14 | 0.012 | 0.014 | 1.5 |
| 15 | 0.021 | 0.014 | 2.3 |
| 16 | 0.1 | 0.023 | 4.22 |
| 17 | 0.135 | 0.03 | NT |
| 18 | 0.158 | 0.049 | >25 |
| 19 | 0.085 | 0.062 | NT |
| 20 | 0.168 | 0.063 | 2.51 |
| 21 | 0.097 | 0.067 | NT |
| 22 | 0.168 | 0.114 | NT |
| 23 | 0.678 | 0.166 | NT |
| 24 | 0.158 | 0.172 | NT |
| 25 | 0.239 | 0.181 | NT |
| 26 | 1.21 | 0.409 | NT |
| 27 | 1.215 | 0.429 | NT |
| 28 | 0.008 | 0.003 | 2.3 |
| 29 | 0.107 | 0.013 | 4.73 |
| 30 | 0.096 | 0.015 | 7.91 |
| 31 | 0.006 | 0.005 | 1.02 |
| 32 | 0.054 | 0.016 | 0.164 |
| 33 | 0.063 | 0.01 | 0.879 |
| 34 | 0.011 | 0.004 | 3.1 |
| 35 | 0.336 | 0.065 | >10 |
| 36 | 5.85 | 2.25 | NT |
| 37 | 1.97 | 0.388 | NT |
| 38 | 0.006 | 0.013 | 1.09 |
| 39 | 0.011 | 0.014 | 0.977 |
| 40 | 0.051 | 0.048 | 5.61 |
| 41 | 0.079 | 0.065 | NT |
| 42 | 0.012 | 0.012 | NT |
| 43 | 3.19 | 3.29 | NT |
| 44 | 0.03 | 0.014 | NT |
| 45 | 0.02 | 0.018 | NT |
| 46 | 0.041 | 0.023 | 3.95 |
| 47 | 0.081 | 0.026 | >25 |
| 48 | 0.159 | 0.026 | NT |
| 49 | 0.141 | 0.038 | NT |
| 50 | 0.055 | 0.042 | 20.2 |
| 51 | 0.053 | 0.042 | 16.7 |
| 52 | 0.134 | 0.059 | 16 |
| 53 | 0.414 | 0.075 | >25 |
| 54 | 0.087 | 0.079 | NT |
| 55 | 0.285 | 0.106 | NT |
| 56 | 0.266 | 0.118 | 5.67 |
| 57 | 0.265 | 0.141 | >25 |
| 58 | 0.444 | 0.176 | 18.2 |
| 59 | 0.687 | 0.321 | NT |
| 60 | 0.48 | 0.343 | >25 |
| 61 | 0.476 | 0.361 | >25 |
| 62 | 0.673 | 0.394 | NT |
| 63 | 0.473 | 0.429 | NT |
| 64 | 1.19 | 0.446 | NT |
| 65 | 1.84 | 0.504 | NT |
| 66 | 1.62 | 0.549 | NT |
| 67 | 1.62 | 0.67 | NT |
| 68 | 0.829 | 0.718 | NT |
| 69 | 7.72 | 1.89 | NT |
| 70 | 10.7 | 2.14 | NT |
| 71 | 3.98 | 2.53 | NT |
| 72 | 0.112 | 0.063 | >10 |
| 73 | 0.06 | 0.036 | >10 |
| 74 | 0.228 | 0.122 | >10 |
| 75 | 1.85 | 0.797 | NT |
| 76 | NT | 0.172 | NT |
| 77 | NT | 0.201 | NT |
| 78 | NT | 0.28 | NT |
| 79 | NT | 0.359 | NT |
| 80 | NT | 0.377 | NT |
| 81 | NT | 0.437 | NT |
| 82 | NT | 0.471 | NT |
| 83 | NT | 0.496 | NT |
| 84 | NT | 0.567 | NT |
| 85 | NT | 0.633 | NT |
| 86 | NT | 0.648 | NT |
| 87 | NT | 0.701 | NT |
| 88 | NT | 0.706 | NT |
| 89 | NT | 0.895 | NT |
| 90 | NT | 1.01 | NT |
| 91 | NT | 1.013 | NT |
| 92 | NT | 1.02 | NT |
| 93 | NT | 1.14 | NT |
| 94 | NT | 1.14 | NT |
| 95 | NT | 1.18 | NT |
| 96 | NT | 1.251 | NT |
| 97 | NT | 1.27 | NT |
| 98 | NT | 1.31 | NT |
| 99 | NT | 1.4 | NT |
| 100 | NT | 1.5 | NT |
| 101 | NT | 1.53 | NT |
| 102 | NT | 1.55 | NT |
| 103 | NT | 1.71 | NT |
| 104 | NT | 1.73 | NT |
| 105 | NT | 1.85 | NT |
| 106 | NT | 1.9 | NT |
| 107 | NT | 1.9 | NT |
| 108 | NT | 1.97 | NT |
| 109 | NT | 2.17 | NT |
| 110 | NT | 2.18 | NT |
| 111 | NT | 2.3 | NT |
| 112 | NT | 2.46 | NT |
| 113 | NT | 2.48 | NT |
| 114 | NT | 2.77 | NT |
| 115 | NT | 2.86 | NT |
| 116 | NT | 3.11 | NT |
| 117 | NT | 3.32 | NT |
| 118 | NT | 3.37 | NT |
| 119 | NT | 3.67 | NT |
| 120 | NT | 3.9 | NT |
| 121 | NT | 3.95 | NT |
| 122 | 0.004 | 0.003 | 0.167 |
| 123 | 0.005 | 0.006 | 0.069 |
| 124 | 0.007 | 0.006 | 0.287 |
| 125 | 0.006 | 0.009 | 0.369 |
| 126 | 0.012 | 0.01 | 0.896 |
| 127 | 0.125 | 0.046 | NT |
| 128 | 1.483 | 0.411 | NT |
| 129 | 5.3 | 1.09 | NT |
| 130 | 0.076 | 0.017 | NT |
| 131 | 1.4 | 0.735 | NT |
| 132 | 0.003 | 0.004 | 0.44 |
| 133 | 0.074 | 0.006 | 4.44 |
| 134 | 2.39 | 0.391 | NT |
| 135 | 0.207 | 0.064 | >10 |
| 136 | 0.001 | 0.001 | 0.033 |
| 137 | 0.003 | 0.002 | 0.008 |
| 138 | 0.03 | 0.011 | 1.7 |
| 139 | 0.011 | 0.003 | NT |
| 140 | 0.012 | 0.003 | NT |
| 141 | 0.002 | 0.002 | NT |
| 142 | 0.004 | 0.007 | NT |
| 143 | 0.010 | 0.008 | NT |
| 144 | 0.079 | 0.037 | 2.3 |
| 145 | 2.97 | 0.803 | NT |
| 146 | 4.09 | 1.56 | NT |
| 147 | 11.2 | 3.55 | NT |
| 148 | 0.056 | 0.074 | 3.41 |
| 149 | 0.121 | 0.025 | 6.93 |
| 150 | 0.003 | 0.006 | 0.011 |
| 151 | 0.005 | 0.005 | 0.033 |
| 152 | 0.217 | 0.095 | >25 |
| 153 | 0.004 | 0.006 | 0.011 |
| 154 | 0.004 | 0.004 | 0.055 |
| 155 | 0.187 | 0.04 | NT |
| 156 | 0.365 | 0.189 | NT |
| 157 | 0.073 | 0.023 | NT |
| 158 | 0.414 | 0.087 | 3.29 |
| 159 | 0.174 | 0.088 | >10 |
| 160 | 0.882 | 0.578 | NT |
| 161 | 0.057 | 0.024 | 5.79 |

NT: not tested.

### REFERENCES

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of formula (I): 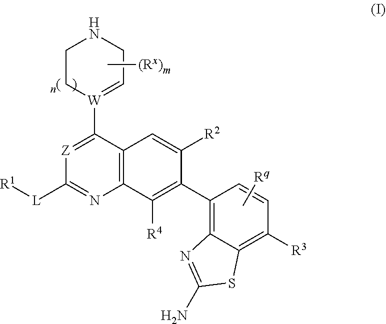
or tautomer thereof, or a pharmaceutically acceptable salt of said compound or said tautomer,
wherein; [symbol]
is a single bond or a double bond;
W is C, CH or N, wherein when W is N, [symbol] is a single bond;
n is 0, 1, 2, or 3;
m is 0, 1, 2, 3 or 4;
each R^{x} is hydroxyl, oxo, cyano, C₁₋₄ alkyl, C₁₋₄ haloalkyl, C₁₋₄ alkoxy, —T—R^{y} or two R^{x} taken together with the carbon atoms to which they are attached can form a C₃₋₈ cycloalkyl or a bridged ring, wherein the bridge atoms are selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—, wherein each bridge or C₃₋₈ cycloalkyl is substituted with 0-3 occurrences of R^{v};
Z is CH, CR′ or N;
R′ is halogen, cyano or C₁₋₄ alkyl;
L is a bond, —C₁₋₄ alkylene, —O—C₁₋₄ alkylene, —S—C₁₋₄ alkylene, —NR^{z}—, —N^{z}—C₁₋₄ alkylene, —O— or —S—, wherein each —C₁₋₄ alkylene, —O—C₁₋₄ alkylene or —S—C₁₋₄ alkylene could be substituted by 0-2 occurrences of R^{b};
R¹ is —N(R^{a})₂, aryl, heteroaryl, C₃₋₈ cycloalkyl or heterocycloalkyl wherein each aryl, heteroaryl, cycloalkyl or heterocycloalkyl is further substituted with 0-3 occurrences of R⁵;
R² is hydrogen, halogen, C₁₋₄ alkyl, C₂₋₄ alkenyl or cyano;
R³ hydrogen, halogen, cyano, C₁₋₄ alkyl, C₁₋₄ haloalkyl or C₂₋₄ alkynyl;
R⁴ is hydrogen or halogen;
each R⁵ is halogen, C₁₋₄ alkyl, C₁₋₄ alkoxy, C₁₋₄ haloalkyl, —N(R^{w})₂, —(CH₂)ₚ—OH, —C(O)—R^{z}, heteroaryl or heterocycloalkyl or two R⁵ taken together with the same carbon atom can form a spirocyclic heteroaryl or heterocycloalkyl wherein each heteroaryl or heterocycloalkyl is further substituted with 0-3 occurrences of R⁷;
p is 1, 2 or 3;
each R⁷ is hydroxyl, oxo, halogen, C₁₋₄ alkyl, C₁₋₄ alkoxy, —C(O)R^{z} or —C(O)OR^{z};
T is C₁₋₄ alkylene, —O— or —S—;
each R^{a} is hydrogen, C₁₋₄ alkyl or C₁₋₄ alkoxy;
each R^{b} is hydroxyl or C₁₋₄ alkyl;
each R^{v} is halogen or C₁₋₄ alkyl;
each R^{w} is hydrogen, C₁₋₄ alkyl, C₁₋₄ alkoxy or heterocycloalkyl;
R^{q} is hydrogen, halogen or C₁₋₄ alkyl;
R^{y} is halogen, hydroxyl, cyano or amino; and
R^{z} is hydrogen or C₁₋₄ alkyl.

2. The compound of claim 1, wherein Z is CH or N

3. The compound of claim 1, wherein W is N or C.

4-5. (canceled)

6. The compound of claim 1, wherein n is 1 and m is 1; n is 1 and m is 2; n is 1 and m is 3; n is 1 and m is 4; n is 2 and m is 1; n is 1 and m is 0 or n is 2 and m is 0.

7-12. (canceled)

13. The compound of claim 1, wherein R^{x} is C₁₋₄ alkyl, oxo, haloalkyl, or —T—R^{y}.

14. (canceled)

15. The compound of claim 1, wherein two R^{x} taken together with the carbon atoms to which they are attached form a C₃₋₈ cycloalkyl ring or form a bridged ring.

16-18. (canceled)

19. The compound of claim 1, wherein one R^{x} is C₁₋₄ alkyl, cyano, oxo or —T—R^{y} and the other two R^{x} are taken together to form a bridged ring, wherein the bridge is —C₁₋₄ alkylene or —C₁₋₄ alkylene-O—C₁₋₄ alkylene-.

20. The compound of claim 19, wherein one R^{x} is methyl, ethyl, cyano, oxo, —CH₂OH or —CH₂CN and the other two R^{x} are taken together to form a bridged ring, wherein the bridge is methylene, ethylene or methylene-O-methylene.

21. The compound of claim 1, wherein two R^{x} are each independently C₁₋₄ alkyl and the other two R^{x} are taken together to form a bridged ring, wherein the bridge is C₁₋₄ alkylene.

22. The compound of claim 21, wherein two R^{x} are both methyl and the other two R^{x} are taken together to form a bridged ring, wherein the bridge is methylene or ethylene.

23. The compound of claim 1, wherein 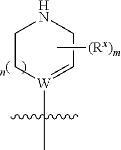
is

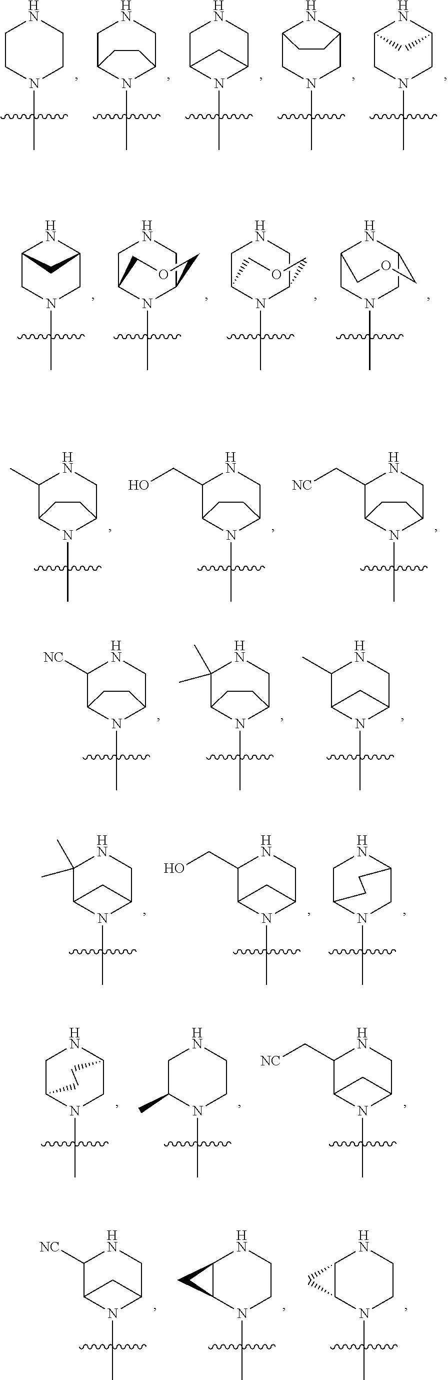

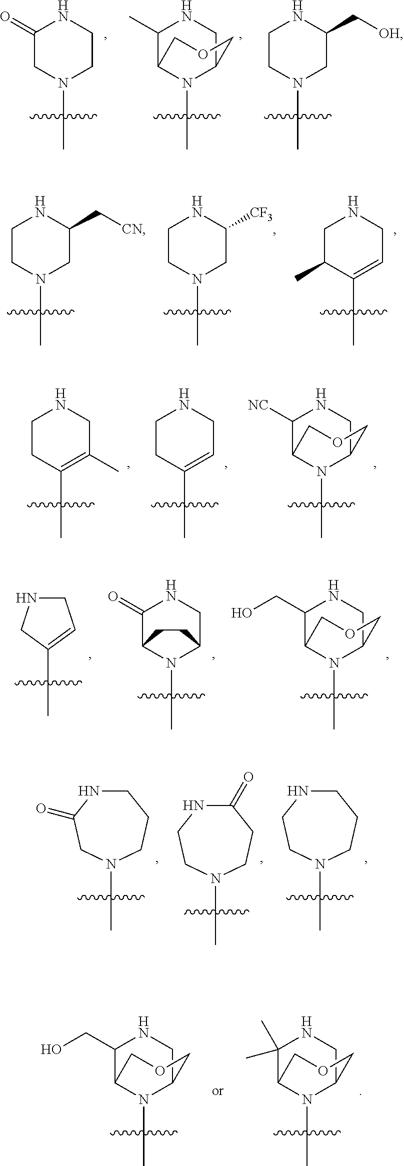

24. The compound of claim 1, wherein L is a bond, —C₁₋₄ alkylene, —NR^{z}—C₁₋₄ alkylene, —NR^{z}—, —O— or —O—C₁₋₄ alkylene substituted with 0-2 occurrences of R^{b}.

25. (canceled)

26. The compound of claim 24, wherein R^{b} is methyl or hydroxyl.

27. The compound of claim 24, wherein R^{z} is hydrogen.

28. The compound of claim 1, wherein R¹ is heterocycloalkyl, heteroaryl or —N(R^{a})₂, wherein each heterocycloalkyl or heteroaryl is substituted with 0-3 occurrences of R⁵.

29-32. (canceled)

33. The compound of claim 28, wherein R⁵ is oxo, halogen, —(CH₂)ₚ—OH, C₁₋₄ alkyl, —C(O)—R^{z}, C₁₋₄ haloalkyl, —N(R^{w})₂, heteroaryl or heterocycloalkyl, wherein each heteroaryl or heterocycloalkyl is substituted with 0-3 occurrences of R⁷.

34. The compound of claim 33, wherein each R^{w} is hydrogen, methyl, methoxy, 3-tetrahydrofuranyl or 2-oxetanyl.

35-51. (canceled)

52. The compound of claim 1, wherein —L—R¹ is

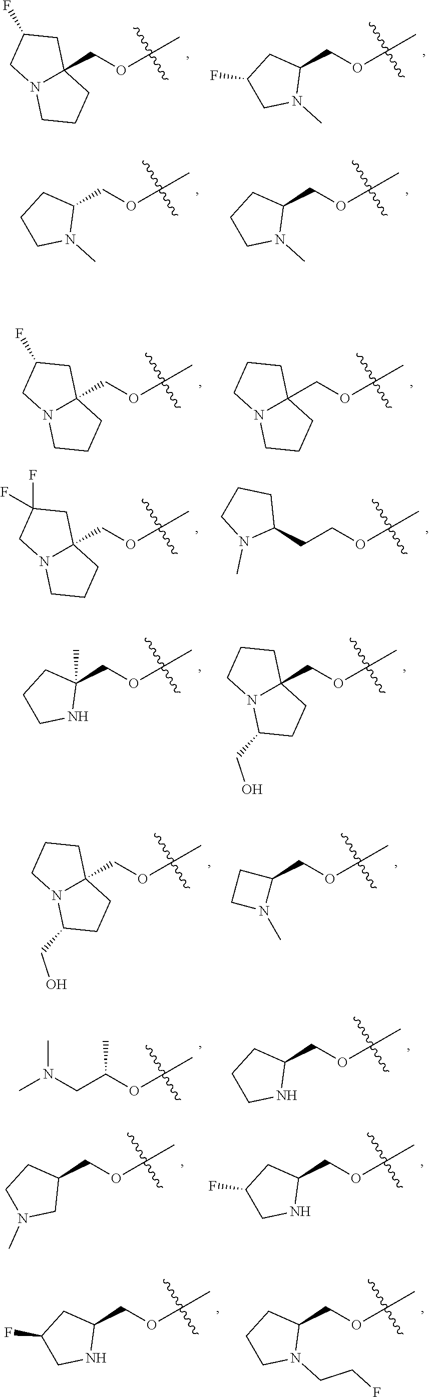

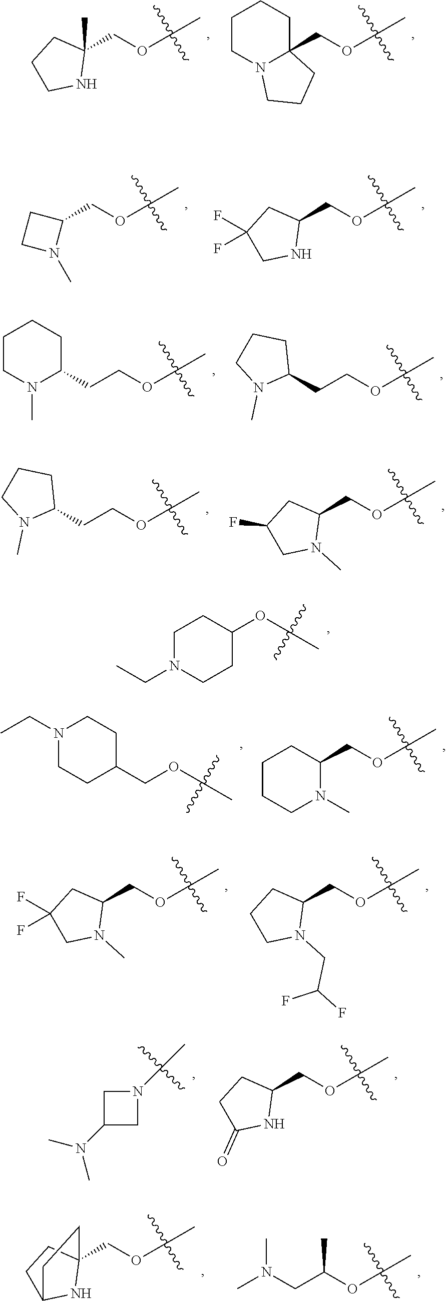

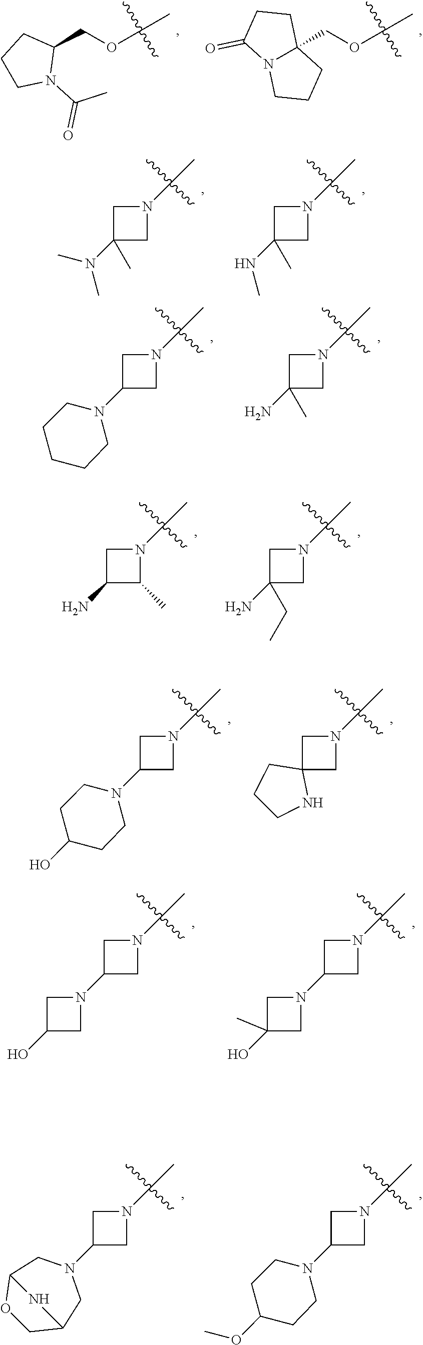

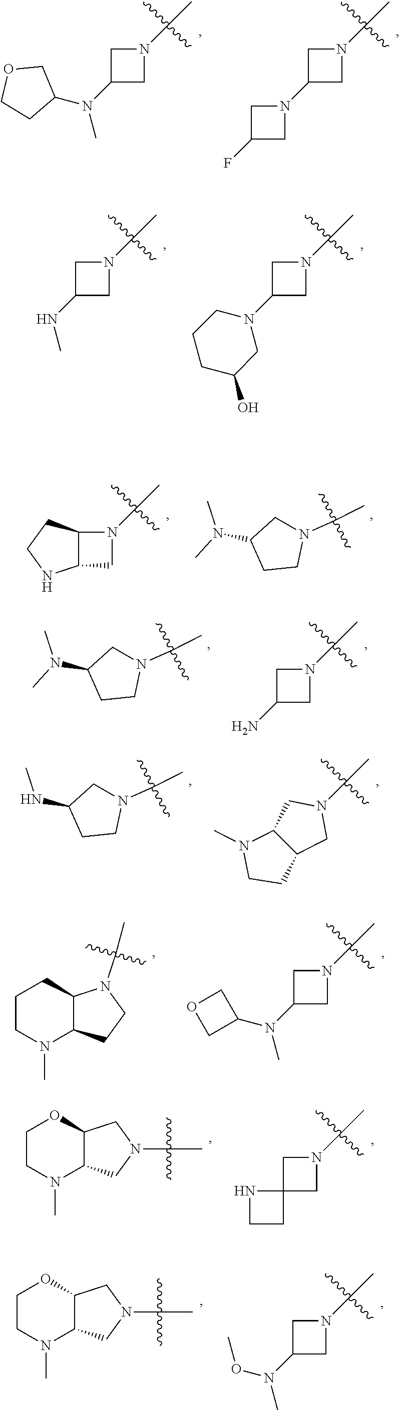

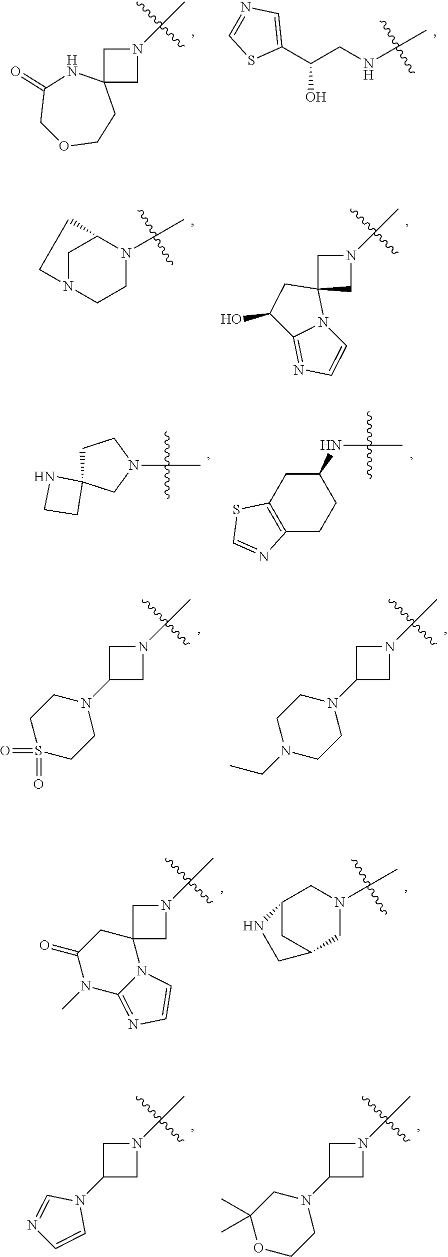

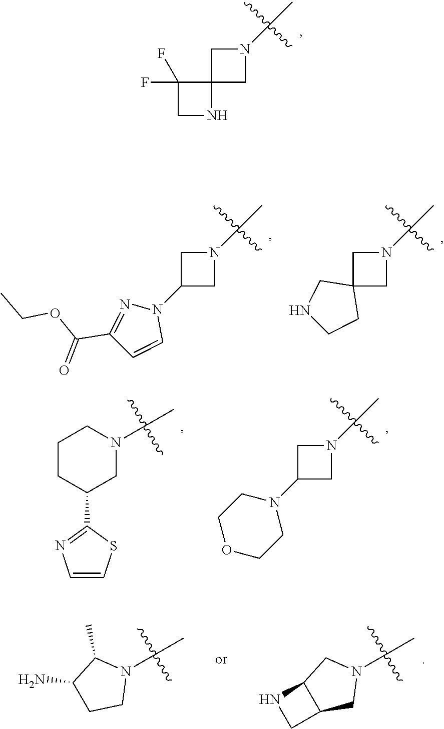

53. The compound of claim 1, wherein R² is halogen, C₁₋₄ alkyl, C₂₋₄ alkenyl or cyano.

54. The compound of claim 53, wherein R² is chlorine, methyl, ethyl, vinyl or cyano.

55. The compound of claim 1, wherein R⁴ is halogen (e.g., fluorine).

56. The compound of claim 1, wherein R³ is hydrogen or halogen.

57. The compound of claim 1, wherein R^{q} is hydrogen or halogen.

58. The compound of claim 1, wherein the compound is selected from one of the following compounds:
4-(6-Chloro-8-fluoro-2-(((S)-1-methylpyrrolidin-2-yl)methoxy)-4-(3-(trifluoromethyl)piperazin-1-yl)quinazolin-7-yl)benzo[d]thiazol-2-amine;
4-(4-((1R,5S)-3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(7-(2-amino-7-fluorobenzo[d]thiazol-4-yl)-6-chloro-8-fluoro-2-(((2R,7aS)-2-fluorotetrahydro-1H-pyrrolizin-7a(5H)-yl)methoxy)quinazolin-4-yl)-1,4-diazepan-2-one;
4-(4-(3,6-diazabicyclo[3.1.1]heptan-3-yl)-6-chloro-8-fluoro-2-(((S)-1-methylpyrrolidin-2-yl)methoxy)quinazolin-7-yl)benzo[d]thiazol-2-amine;
4-(4-((1R,5S)-3,6-diazabicyclo[3.1.1]heptan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,6-diazabicyclo[3.1.1]heptan-3-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(6-chloro-8-fluoro-4-(piperazin-1-yl)-2-((tetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-chloro-2-((2,2-difluorotetrahydro-1H-pyrrolizin-7a(5H)-yl)methoxy)-8-fluoroquinazolin-7-yl)-7-fluorobenzo[d]thiazol-2-amine;
4-(4-(3-oxa-7,9-diazabicyclo[3.3.1]nonan-9-yl)-6-chloro-8-fluoro-2-(((S)-1-methylpyrrolidin-2-yl)methoxy)quinazolin-7-yl)benzo[d]thiazol-2-amine;
4-(4-((1R,5S)-3,6-diazabicyclo[3.1.1]heptan-3-yl)-6-chloro-8-fluoro-2-(2-((S)-1-methylpyrrolidin-2-yl)ethoxy)quinazolin-7-yl)-7-fluorobenzo[d]thiazol-2-amine;
4-(7-(2-amino-7-fluorobenzo[d]thiazol-4-yl)-6-chloro-8-fluoro-2-((tetrahydro-1H-pyrrolizin-7a(5H)-yl)methoxy)quinazolin-4-yl)-1,4-diazepan-2-one;
1-(7-(2-amino-7-fluorobenzo[d]thiazol-4-yl)-6-chloro-8-fluoro-2-((tetrahydro-1H-pyrrolizin-7a(5H)-yl)methoxy)quinazolin-4-yl)-1,4-diazepan-5-one;
4-(4-((1R,5S)-3,6-diazabicyclo[3.1.1]heptan-3-yl)-6-chloro-2-((2,2-difluorotetrahydro-1H-pyrrolizin-7a(5H)-yl)methoxy)-8-fluoroquinazolin-7-yl)-7-fluorobenzo[d]thiazol-2-amine;
4-(4-((1S,4S)-2,5-diazabicyclo[2.2.2]octan-2-yl)-6-chloro-8-fluoro-2-(((S)-1-methylpyrrolidin-2-yl)methoxy)quinazolin-7-yl)benzo[d]thiazol-2-amine;
4-(6-chloro-8-fluoro-4-((S)-2-methylpiperazin-1-yl)-2-(((S)-1-methylpyrrolidin-2-yl)methoxy)quinazolin-7-yl)benzo[d]thiazol-2-amine;
4-(6-chloro-4-(1,4-diazepan-1-yl)-8-fluoro-2-(((S)-1-methylpyrrolidin-2-yl)methoxy)quinazolin-7-yl)benzo[d]thiazol-2-amine;
4-(4-(3,6-diazabicyclo[3.1.1]heptan-6-yl)-6-chloro-8-fluoro-2-(((S)-1-methylpyrrolidin-2-yl)methoxy)quinazolin-7-yl)benzo[d]thiazol-2-amine;
4-(7-(2-amino-7-fluorobenzo[d]thiazol-4-yl)-6-chloro-8-fluoro-2-(((2S,4R)-4-fluoro-1-methylpyrrolidin-2-yl)methoxy)quinazolin-4-yl)-1,4-diazepan-2-one;
4-(4-(2,5-diazabicyclo[4.1.0]heptan-2-yl)-6-chloro-8-fluoro-2-(((S)-1-methylpyrrolidin-2-yl)methoxy)quinazolin-7-yl)benzo[d]thiazol-2-amine;
1-(7-(2-amino-7-fluorobenzo[d]thiazol-4-yl)-6-chloro-8-fluoro-2-(((2S,4R)-4-fluoro-1-methylpyrrolidin-2-yl)methoxy)quinazolin-4-yl)-1,4-diazepan-5-one;
4-(7-(2-aminobenzo[d]thiazol-4-yl)-6-chloro-8-fluoro-2-(((S)-1-methylpyrrolidin-2-yl)methoxy)quinazolin-4-yl)-1,4-diazepan-2-one;
4-(7-(2-aminobenzo[d]thiazol-4-yl)-6-chloro-8-fluoro-2-(((S)-1-methylpyrrolidin-2-yl)methoxy)quinazolin-4-yl)piperazin-2-one;
1-(7-(2-aminobenzo[d]thiazol-4-yl)-6-chloro-8-fluoro-2-(((S)-1-methylpyrrolidin-2-yl)methoxy)quinazolin-4-yl)-1,4-diazepan-5-one;
((2R)-4-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)methanol;
2-((2S)-4-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)acetonitrile;
4-(4-((1R,4R)-2,5-diazabicyclo[2.2.2]octan-2-yl)-6-chloro-8-fluoro-2-(((S)-1-methylpyrrolidin-2-yl)methoxy)quinazolin-7-yl)benzo[d]thiazol-2-amine;
4-(6-chloro-4-(1,4-diazepan-1-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,6-diazabicyclo[3.2.1]octan-6-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,6-diazabicyclo[3.2.1]octan-6-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,6-diazabicyclo[3.2.2]nonan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,9-diazabicyclo[4.2.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(3,9-diazabicyclo[4.2.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(2,6-diazabicyclo[3.2.1]octan-6-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
2-((2S)-1-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)ethan-1-ol;
((2R)-1-(7-(2-aminobenzo[d]thiazol-4-yl)-6-chloro-8-fluoro-2-(((S)-1-methylpyrrolidin-2-yl)methoxy)quinazolin-4-yl)piperazin-2-yl)methanol;
4-(4-((1S,4S)-2,5-diazabicyclo[2.2.2]octan-2-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-(2,5-diazabicyclo[4.1.0]heptan-2-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(6-chloro-8-fluoro-2-(((2R,7aS)-2-fluorotetrahydro-1H-pyrrolizin-7a(5H)-yl)methoxy)-4-(octahydro-1H-cyclopenta[b]pyrazin-1-yl)quinazolin-7-yl)-7-fluorobenzo[d]thiazol-2-amine;
4-(4-((1R,5S)-3-oxa-7,9-diazabicyclo[3.3.1]nonan-7-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(4-((1R,5S)-3-oxa-7,9-diazabicyclo[3.3.1]nonan-7-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
7a-(((7-(2-aminobenzo[d]thiazol-4-yl)-6-chloro-8-fluoro-4-(piperazin-1-yl)quinazolin-2-yl)oxy)methyl)hexahydro-3H-pyrrolizin-3-one;
4-(6-chloro-8-fluoro-4-(piperazin-1-yl)-2-((tetrahydro-1H-pyrrolizin-7a(5H)-yl)methoxy)quinazolin-7-yl)benzo[d]thiazol-2-amine;
4-(6-chloro-8-fluoro-2-(((2R,7aS)-2-fluorotetrahydro-1H-pyrrolizin-7a(5H)-yl)methoxy)-4-(piperazin-1-yl)quinazolin-7-yl)benzo[d]thiazol-2-amine;
4-(6-chloro-8-fluoro-2-(((2R,7aR)-2-fluorotetrahydro-1H-pyrrolizin-7a(5H)-yl)methoxy)-4-(piperazin-1-yl)quinazolin-7-yl)benzo[d]thiazol-2-amine;
4-(6-chloro-8-fluoro-2-(((2S,4R)-4-fluoropyrrolidin-2-yl)methoxy)-4-(piperazin-1-yl)quinazolin-7-yl)benzo[d]thiazol-2-amine;
4-(6-chloro-2-(((S)-1-(dimethylamino)propan-2-yl)oxy)-8-fluoro-4-(piperazin-1-yl)quinazolin-7-yl)benzo[d]thiazol-2-amine;
4-(6-chloro-8-fluoro-2-(((2S,4R)-4-fluoro-1-methylpyrrolidin-2-yl)methoxy)-4-(piperazin-1-yl)quinazolin-7-yl)benzo[d]thiazol-2-amine;
((3R,7aR)-7a-(((7-(2-aminobenzo[d]thiazol-4-yl)-6-chloro-8-fluoro-4-(piperazin-1-yl)quinazolin-2-yl)oxy)methyl)hexahydro-1H-pyrrolizin-3-yl)methanol;
((3R,7aS)-7a-(((7-(2-aminobenzo[d]thiazol-4-yl)-6-chloro-8-fluoro-4-(piperazin-1-yl)quinazolin-2-yl)oxy)methyl)hexahydro-1H-pyrrolizin-3-yl)methanol;
4-(6-chloro-8-fluoro-2-(((S)-1-methyl)azetidin-2-yl)methoxy)-4-(piperazin-1-yl)quinazolin-7-yl)benzo[d]thiazol-2-amine;
4-(6-chloro-8-fluoro-2-(((R)-1-methylpyrrolidin-3-yl)methoxy)-4-(piperazin-1-yl)quinazolin-7-yl)benzo[d]thiazol-2-amine;
4-(6-chloro-2-((2,2-difluorotetrahydro-1H-pyrrolizin-7a(5H)-yl)methoxy)-8-fluoro-4-(piperazin-1-yl)quinazolin-7-yl)benzo[d]thiazol-2-amine;
4-(6-chloro-8-fluoro-2-(((2S,4S)-4-fluoropyrrolidin-2-yl)methoxy)-4-(piperazin-1-yl)quinazolin-7-yl)benzo[d]thiazol-2-amine;
4-(6-chloro-8-fluoro-2-(((S)-1-(2-fluoroethyl)pyrrolidin-2-yl)methoxy)-4-(piperazin-1-yl)quinazolin-7-yl)benzo[d]thiazol-2-amine;
4-(6-chloro-8-fluoro-2-((hexahydroindolizin-8a(1H)-yl)methoxy)-4-(piperazin-1-yl)quinazolin-7-yl)benzo[d]thiazol-2-amine;
4-(6-chloro-8-fluoro-2-(((R)-1-methyl)azetidin-2-yl)methoxy)-4-(piperazin-1-yl)quinazolin-7-yl)benzo[d]thiazol-2-amine;
4-(6-chloro-2-(((S)-4,4-difluoropyrrolidin-2-yl)methoxy)-8-fluoro-4-(piperazin-1-yl)quinazolin-7-yl)benzo[d]thiazol-2-amine;
4-(6-chloro-8-fluoro-2-(2-(1-methylpiperidin-2-yl)ethoxy)-4-(piperazin-1-yl)quinazolin-7-yl)benzo[d]thiazol-2-amine;
4-(6-chloro-8-fluoro-2-(2-((S)-1-methylpyrrolidin-2-yl)ethoxy)-4-(piperazin-1-yl)quinazolin-7-yl)benzo[d]thiazol-2-amine;
4-(6-chloro-8-fluoro-2-(((2S,4S)-4-fluoro-1-methylpyrrolidin-2-yl)methoxy)-4-(piperazin-1-yl)quinazolin-7-yl)benzo[d]thiazol-2-amine;
4-(6-chloro-2-((1-ethylpiperidin-4-yl)methoxy)-8-fluoro-4-(piperazin-1-yl)quinazolin-7-yl)benzo[d]thiazol-2-amine;
4-(6-chloro-8-fluoro-2-(((S)-1-methylpiperidin-2-yl)methoxy)-4-(piperazin-1-yl)quinazolin-7-yl)benzo[d]thiazol-2-amine;
4-(6-chloro-2-(((S)-4,4-difluoro-1-methylpyrrolidin-2-yl)methoxy)-8-fluoro-4-(piperazin-1-yl)quinazolin-7-yl)benzo[d]thiazol-2-amine;
4-(6-chloro-2-(((S)-1-(2,2-difluoroethyl)pyrrolidin-2-yl)methoxy)-8-fluoro-4-(piperazin-1-yl)quinazolin-7-yl)benzo[d]thiazol-2-amine;
4-(6-chloro-2-(3-(dimethylamino)azetidin-1-yl)-8-fluoro-4-(piperazin-1-yl)quinazolin-7-yl)benzo[d]thiazol-2-amine;
(5S)-5-(((7-(2-aminobenzo[d]thiazol-4-yl)-6-chloro-8-fluoro-4-(piperazin-1-yl)quinazolin-2-yl)oxy)methyl)pyrrolidin-2-one;
4-(6-chloro-2-(((R)-1-(dimethylamino)propan-2-yl)oxy)-8-fluoro-4-(piperazin-1-yl)quinazolin-7-yl)benzo[d]thiazol-2-amine;
4-(6-chloro-8-fluoro-2-((((S)-1-methylpyrrolidin-2-yl)methyl)amino)-4-(piperazin-1-yl)quinazolin-7-yl)benzo[d]thiazol-2-amine;
1-((2S)-2-(((7-(2-aminobenzo[d]thiazol-4-yl)-6-chloro-8-fluoro-4-(piperazin-1-yl)quinazolin-2-yl)oxy)methyl)pyrrolidin-1-yl)ethan-1-one;
4-(6-chloro-8-fluoro-4-(piperazin-1-yl)-2-(((S)-pyrrolidin-2-yl)methoxy)quinazolin-7-yl)benzo[d]thiazol-2-amine;
4-(6-chloro-8-fluoro-2-(((S)-2-methylpyrrolidin-2-yl)methoxy)-4-(piperazin-1-yl)quinazolin-7-yl)benzo[d]thiazol-2-amine;
4-(6-chloro-8-fluoro-2-(((R)-2-methylpyrrolidin-2-yl)methoxy)-4-(piperazin-1-yl)quinazolin-7-yl)benzo[d]thiazol-2-amine;
4-(2-(((1s,4s)-7-azabicyclo[2.2.1]heptan-1-yl)methoxy)-6-chloro-8-fluoro-4-(piperazin-1-yl)quinazolin-7-yl)benzo[d]thiazol-2-amine;
4-(6-Chloro-2-(3-(dimethylamino)-3-methyl)azetidin-1-yl)-8-fluoro-4-(piperazin-1-yl)quinazolin-7-yl)benzo[d]thiazol-2-amine;
4-[6-chloro-8-fluoro-2-[3-methyl-3-(methylamino)azetidin-1-yl]-4-piperazin-1-yl-quinazolin-7-yl]-1,3-benzothiazol-2-amine;
4-[6-chloro-8-fluoro-4-piperazin-1-yl-2-[3-(1-piperidyl)azetidin-1-yl]quinazolin-7-yl]-1,3-benzothiazol-2-amine;
4-[2-(3-amino-3-methyl-azetidin-1-yl)-6-chloro-8-fluoro-4-piperazin-1-yl-quinazolin-7-yl]-1,3-benzothiazol-2-amine;
4-[2-[(2R,3S)-3-amino-2-methyl-azetidin-1-yl]-6-chloro-8-fluoro-4-piperazin-1-yl-quinazolin-7-yl]-1,3-benzothiazol-2-amine;
4-[2-(3-amino-3-ethyl-azetidin-1-yl)-6-chloro-8-fluoro-4-piperazin-1-yl-quinazolin-7-yl]-1,3-benzothiazol-2-amine;
8-(7-(2-aminobenzo[d]thiazol-4-yl)-6-chloro-8-fluoro-2-(((S)-1-methylpyrrolidin-2-yl)methoxy)quinazolin-4-yl)-3,8-diazabicyclo[3.2.1]octan-2-one;
1-[1-[7-(2-amino-1,3-benzothiazol-4-yl)-6-chloro-8-fluoro-4-piperazin-1-yl-quinazolin-2-yl]azetidin-3-yl]piperidin-4-ol;
4-[6-chloro-2-(2,5-diazaspiro[3.4]octan-2-yl)-8-fluoro-4-piperazin-1-yl-quinazolin-7-yl]-1,3-benzothiazol-2-amine;
1-[1-[7-(2-amino-1,3-benzothiazol-4-yl)-6-chloro-8-fluoro-4-piperazin-1-yl-quinazolin-2-yl]azetidin-3-yl]azetidin-3-ol;
1-[1-[7-(2-amino-1,3-benzothiazol-4-yl)-6-chloro-8-fluoro-4-piperazin-1-yl-quinazolin-2-yl]azetidin-3-yl]-3-methyl-azetidin-3-ol;
4-(2-(3-((1R,5S)-3-oxa-7,9-diazabicyclo[3.3.1]nonan-7-yl)azetidin-1-yl)-6-chloro-8-fluoro-4-(piperazin-1-yl)quinazolin-7-yl)benzo[d]thiazol-2-amine;
4-[6-chloro-8-fluoro-2-[3-(4-methoxy-1-piperidyl)azetidin-1-yl]-4-piperazin-1-yl-quinazolin-7-yl]-1,3-benzothiazol-2-amine;
4-[6-chloro-8-fluoro-2-[3-[methyl-[(3R)-tetrahydrofuran-3-yl]amino]azetidin-1-yl]-4-piperazin-1-yl-quinazolin-7-yl]-1,3-benzothiazol-2-amine;
4-[6-chloro-8-fluoro-2-[3-(3-fluoroazetidin-1-yl)azetidin-1-yl]-4-piperazin-1-yl-quinazolin-7-yl]-1,3-benzothiazol-2-amine;
4-[6-chloro-8-fluoro-2-[3-(methylamino)azetidin-1-yl]-4-piperazin-1-yl-quinazolin-7-yl]-1,3-benzothiazol-2-amine;
1-[1-[7-(2-amino-1,3-benzothiazol-4-yl)-6-chloro-8-fluoro-4-piperazin-1-yl-quinazolin-2-yl]azetidin-3-yl]piperidin-3-ol;
4-[6-chloro-2-[(1S,5R)-2,6-diazabicyclo[3.2.0]heptan-6-yl]-8-fluoro-4-piperazin-1-yl-quinazolin-7-yl]-1,3-benzothiazol-2-amine;
4-[6-chloro-8-fluoro-4-piperazin-1-yl-2-[3-(dimethylamino)pyrrolidin-1-yl]quinazolin-7-yl]-1,3-benzothiazol-2-amine;
4-[6-chloro-2-[(3R)-3-(dimethylamino)pyrrolidin-1-yl]-8-fluoro-4-piperazin-1-yl-quinazolin-7-yl]-1,3-benzothiazol-2-amine;
4-[2-(3-aminoazetidin-1-yl)-6-chloro-8-fluoro-4-piperazin-1-yl-quinazolin-7-yl]-1,3-benzothiazol-2-amine;
4-[6-chloro-8-fluoro-4-piperazin-1-yl-2-[3-(methylamino)pyrrolidin-1-yl]quinazolin-7-yl]-1,3-benzothiazol-2-amine;
4-(6-chloro-8-fluoro-2-(1-methylhexahydropyrrolo[3,4-b]pyrrol-5(1H)-yl)-4-(piperazin-1-yl)quinazolin-7-yl)benzo[d]thiazol-2-amine;
4-(6-chloro-8-fluoro-2-(4-methyloctahydro-1H-pyrrolo[3,2-b]pyridin-1-yl)-4-(piperazin-1-yl)quinazolin-7-yl)benzo[d]thiazol-2-amine;
4-[6-chloro-8-fluoro-2-[3-[methyl(oxetan-3-yl)amino]azetidin-1-yl]-4-piperazin-1-yl-quinazolin-7-yl]-1,3-benzothiazol-2-amine;
4-(6-chloro-8-fluoro-2-(trans-4-methylhexahydropyrrolo[3,4-b][1,4]oxazin-6(2H)-yl)-4-(piperazin-1-yl)quinazolin-7-yl)benzo[d]thiazol-2-amine;
4-[6-chloro-2-(1,6-diazaspiro[3.3]heptan-6-yl)-8-fluoro-4-piperazin-1-yl-quinazolin-7-yl]-1,3-benzothiazol-2-amine;
4-(6-chloro-8-fluoro-2-(cis-4-methylhexahydropyrrolo[3,4-b][1,4]oxazin-6(2H)-yl)-4-(piperazin-1-yl)quinazolin-7-yl)benzo[d]thiazol-2-amine;
4-[6-chloro-8-fluoro-2-[3-[methoxy(methyl)amino]azetidin-1-yl]-4-piperazin-1-yl-quinazolin-7-yl]-1,3-benzothiazol-2-amine;
2-[7-(2-amino-1,3-benzothiazol-4-yl)-6-chloro-8-fluoro-4-piperazin-1-yl-quinazolin-2-yl]-8-oxa-2,5-diazaspiro[3.6]decan-6-one;
2-[[7-(2-amino-1,3-benzothiazol-4-yl)-6-chloro-8-fluoro-4-piperazin-1-yl-quinazolin-2-yl]amino]-1-thiazol-5-yl-ethanol;
4-[6-chloro-8-fluoro-4-piperazin-1-yl-2-[1,4-diazabicyclo[3.2.1]octan-4-yl]quinazolin-7-yl]-1,3-benzothiazol-2-amine;
1′-[7-(2-amino-1,3-benzothiazol-4-yl)-6-chloro-8-fluoro-4-piperazin-1-yl-quinazolin-2-yl]spiro[6,7-dihydropyrrolo[1,2-a]imidazole-5,3′-azetidine]-7-ol;
4-[6-chloro-8-fluoro-4-piperazin-1-yl-2-[1,7-diazaspiro[3.4]octan-7-yl]quinazolin-7-yl]-1,3-benzothiazol-2-amine;
4-[6-chloro-8-fluoro-4-piperazin-1-yl-2-[[(6S)-4,5,6,7-tetrahydro-1,3-benzothiazol-6-yl]amino]quinazolin-7-yl]-1,3-benzothiazol-2-amine;
4-[6-chloro-2-[3-(1,1-dioxo-1,4-thiazinan-4-yl)azetidin-1-yl]-8-fluoro-4-piperazin-1-yl-quinazolin-7-yl]-1,3-benzothiazol-2-amine;
4-[6-chloro-2-[3-(4-ethylpiperazin-1-yl)azetidin-1-yl]-8-fluoro-4-piperazin-1-yl-quinazolin-7-yl]-1,3-benzothiazol-2-amine;
1′-[7-(2-amino-1,3-benzothiazol-4-yl)-6-chloro-8-fluoro-4-piperazin-1-yl-quinazolin-2-yl]-8-methyl-spiro[6H-imidazo[1,2-a]pyrimidine-5,3′-azetidine]-7-one;
4-[6-chloro-8-fluoro-4-piperazin-1-yl-2-[3,6-diazabicyclo[3.2.1]octan-3-yl]quinazolin-7-yl]-1,3-benzothiazol-2-amine;
4-[6-chloro-2-[(1S,5S)-2,6-diazabicyclo[3.2.0]heptan-6-yl]-8-fluoro-4-piperazin-1-yl-quinazolin-7-yl]-1,3-benzothiazol-2-amine;
4-[6-chloro-8-fluoro-2-(3-imidazol-1-yl)azetidin-1-yl)-4-piperazin-1-yl-quinazolin-7-yl]-1,3-benzothiazol-2-amine;
4-[6-chloro-2-[3-(2,2-dimethylmorpholin-4-yl)azetidin-1-yl]-8-fluoro-4-piperazin-1-yl-quinazolin-7-yl]-1,3-benzothiazol-2-amine;
4-[6-chloro-2-(3,3-difluoro-1,6-diazaspiro[3.3]heptan-6-yl)-8-fluoro-4-piperazin-1-yl-quinazolin-7-yl]-1,3-benzothiazol-2-amine;
ethyl 1-[1-[7-(2-amino-1,3-benzothiazol-4-yl)-6-chloro-8-fluoro-4-piperazin-1-yl-quinazolin-2-yl]azetidin-3-yl]pyrazole-3-carboxylate;
4-[6-chloro-2-(2,7-diazaspiro[3.4]octan-2-yl)-8-fluoro-4-piperazin-1-yl-quinazolin-7-yl]-1,3-benzothiazol-2-amine;
4-[6-chloro-8-fluoro-4-piperazin-1-yl-2-[3-thiazol-2-yl-1-piperidyl]quinazolin-7-yl]-1,3-benzothiazol-2-amine;
4-(4-((1R,5S)-3-Oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-chloro-8-fluoro-2-(((2R,7aR)-2-fluorotetrahydro-1H-pyrrolizin-7a(5H)-yl)methoxy)quinazolin-7-yl)-7-fluorobenzo[d]thiazol-2-amine;
4-(4-((1R,5S)-3-oxa-7,9-diazabicyclo[3.3.1]nonan-9-yl)-6-chloro-8-fluoro-2-(((2R,7aR)-2-fluorotetrahydro-1H-pyrrolizin-7a(5H)-yl)methoxy)quinazolin-7-yl)-7-fluorobenzo[d]thiazol-2-amine;
4-(4-((1R,5S)-3-oxa-7,9-diazabicyclo[3.3.1]nonan-9-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine;
4-(4-((1R,5S)-3-oxa-7,9-diazabicyclo[3.3.1]nonan-9-yl)-6-chloro-2-((2,2-difluorotetrahydro-1H-pyrrolizin-7a(5H)-yl)methoxy)-8-fluoroquinazolin-7-yl)-7-fluorobenzo[d]thiazol-2-amine;
4-(6-chloro-8-fluoro-2-(((S)-1-methylpyrrolidin-2-yl)methoxy)-4-(piperazin-1-yl)quinazolin-7-yl)benzo[d]thiazol-2-amine;
4-(6-chloro-2-((1-ethylpiperidin-4-yl)oxy)-8-fluoro-4-(piperazin-1-yl)quinazolin-7-yl)benzo[d]thiazol-2-amine;
4-(6-chloro-8-fluoro-2-(((S)-1-methylpyrrolidin-2-yl)methoxy)-4-(piperazin-1-yl)quinazolin-7-yl)-6-methylbenzo[d]thiazol-2-amine;
4-(6-chloro-8-fluoro-2-(((S)-1-methylpyrrolidin-2-yl)methoxy)-4-(piperazin-1-yl)quinazolin-7-yl)-7-fluorobenzo[d]thiazol-2-amine;
4-(6-chloro-8-fluoro-2-(((S)-1-methylpyrrolidin-2-yl)methoxy)-4-(piperazin-1-yl)quinazolin-7-yl)-7-(trifluoromethyl)benzo[d]thiazol-2-amine;
4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)quinazolin-7-yl)-7-fluorobenzo[d]thiazol-2-amine;
4-(6,8-difluoro-2-(((S)-1-methylpyrrolidin-2-yl)methoxy)-4-(piperazin-1-yl)quinazolin-7-yl)benzo[d]thiazol-2-amine;
4-(4-((1R,5S)-3,8-diazabicyclo[3.2.1]octan-8-yl)-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-6-chloro-8-fluoro-2-(((2R,7aR)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-8-methylquinazolin-7-yl)-7-fluorobenzo[d]thiazol-2-amine;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6,8-difluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1S,4S)-2,5-diazabicyclo[2.2.2]octan-2-yl)-6,8-difluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(6-Chloro-8-fluoro-2-(((S)-1-methylpyrrolidin-2-yl)methoxy)-4-(1,2,3,6-tetrahydropyridin-4-yl)quinazolin-7-yl)benzo[d]thiazol-2-amine;
4-(6-chloro-8-fluoro-4-(3-methyl-1,2,3,6-tetrahydropyridin-4-yl)-2-(((S)-1-methylpyrrolidin-2-yl)methoxy)quinazolin-7-yl)benzo[d]thiazol-2-amine;
4-(6-chloro-8-fluoro-4-(3-methyl-1,2,3,6-tetrahydropyridin-4-yl)-2-(((S)-1-methylpyrrolidin-2-yl)methoxy)quinazolin-7-yl)benzo[d]thiazol-2-amine;
4-(6-chloro-8-fluoro-4-(5-methyl-1,2,3,6-tetrahydropyridin-4-yl)-2-(((S)-1-methylpyrrolidin-2-yl)methoxy)quinazolin-7-yl)benzo[d]thiazol-2-amine;
4-(6-chloro-4-(2,5-dihydro-1H-pyrrol-3-yl)-8-fluoro-2-(((S)-1-methylpyrrolidin-2-yl)methoxy)quinazolin-7-yl)benzo[d]thiazol-2-amine;
4-(8-Fluoro-2-(((S)-1-methylpyrrolidin-2-yl)methoxy)-4-(piperazin-1-yl)-6-vinylquinazolin-7-yl)benzo[d]thiazol-2-amine;
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-vinylquinazolin-7-yl)-7-fluorobenzo[d]thiazol-2-amine;
4-(4-((1R,5S)-3,8-diazabicyclo[3.2.1]octan-8-yl)-8-fluoro-2-(((2R,7aS)-2-fluorotetrahydro-1H-pyrrolizin-7a(5H)-yl)methoxy)-6-vinylquinazolin-7-yl)-7-fluorobenzo[d]thiazol-2-amine;
4-(6-Ethyl-8-fluoro-2-(((S)-1-methylpyrrolidin-2-yl)methoxy)-4-(piperazin-1-yl)quinazolin-7-yl)benzo[d]thiazol-2-amine;
4-(4-((1R,5S)-3,8-diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(4-((1R,5S)-3,8-diazabicyclo[3.2.1]octan-8-yl)-6-ethyl-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine;
4-(8-Fluoro-6-methyl-2-(((S)-1-methylpyrrolidin-2-yl)methoxy)-4-(piperazin-1-yl)quinazolin-7-yl)benzo[d]thiazol-2-amine;
7-(2-Aminobenzo[d]thiazol-4-yl)-8-fluoro-2-(((S)-1-methylpyrrolidin-2-yl)methoxy)-4-(piperazin-1-yl)quinazoline-6-carbonitrile;
7-(2-Amino-7-fluorobenzo[d]thiazol-4-yl)-6-chloro-8-fluoro-2-(((S)-1-methylpyrrolidin-2-yl)methoxy)-4-(piperazin-1-yl)quinoline-3-carbonitrile;
7-(2-aminobenzo[d]thiazol-4-yl)-6-chloro-8-fluoro-2-(((S)-1-methylpyrrolidin-2-yl)methoxy)-4-(piperazin-1-yl)quinoline-3-carbonitrile;
7-(2-amino-7-fluorobenzo[d]thiazol-4-yl)-4-((1R,5S)-3,8-diazabicyclo[3.2.1]octan-8-yl)-6-chloro-8-fluoro-2-(((2R)-2-fluorotetrahydro-1H-pyrrolizin-7a(5H)-yl)methoxy)quinoline-3-carbonitrile;
7-(2-amino-7-fluorobenzo[d]thiazol-4-yl)-4-((1R,5S)-3,8-diazabicyclo[3.2.1]octan-8-yl)-6-chloro-8-fluoro-2-(((2S,4R)-4-fluoro-1-methylpyrrolidin-2-yl)methoxy)quinoline-3-carbonitrile;
or
4-(4-((1R,5S)-3,8-Diazabicyclo[3.2.1]octan-3-yl)-6-chloro-8-fluoro-2-(((2R,7aS)-2-fluorotetrahydro-1H-pyrrolizin-7a(5H)-yl)methoxy)quinolin-7-yl)-7-fluorobenzo[d]thiazol-2-amine.

59. A pharmaceutical composition comprising the compound according to claim 1 or a pharmaceutically acceptable salt of said compound, and a pharmaceutically acceptable excipient.

60-66. (canceled)

67. A method of treating cancer in a subject in need thereof, the method comprising administering to the subject a therapeutically effective amount of the compound according to claim 1 or a pharmaceutically acceptable salt thereof or a pharmaceutical composition according to claim 59.

68-69. (canceled)

70. The method according to claim 67, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

71. The method according to claim 70, wherein the cancer is non-small cell lung cancer.

72. The method according to claim 70, wherein the cancer is colorectal cancer.

73. The method according to claim 70, wherein the cancer is pancreatic cancer.

74. (canceled)
